(19) United States
(12) Patent Application Publication
(10) Pub. No.: US 2024/0390383 A1
(43) Pub. Date: Nov. 28, 2024

(54) DIOXAZINES AND THEIR USE IN TREATMENT OF GBA-RELATED DISEASES

(71) Applicant: ZEVRA DENMARK A/S, Copenhagen N (DK)

(72) Inventors: Søren NEVE, Lyngby (DK); William Dalby BROWN, Søborg (DK); Kenneth THIRSTRUP, Søborg (DK)

(21) Appl. No.: 18/695,862

(22) PCT Filed: Sep. 27, 2022
(86) PCT No.: PCT/IB2022/059202

§ 371 Date: Mar. 27, 2024

(30) Foreign Application Priority Data
Sep. 28, 2021 (EP) 21199449.6

(51) Int. Cl. A61K 31/539; A61K 31/551; C07D 413/06; C07D 413/14; C07D 453/02; C07D 471/04; C07D 471/08; C07D 487/04; C07D 491/048; C07D 491/052; C07D 498/04; C07D 498/08

(52) U.S. Cl. CPC A61K 31/539; A61K 31/551; C07D 413/06; C07D 413/14; C07D 453/02; C07D 471/04; C07D 471/08; C07D 487/04; C07D 491/048; C07D 491/052; C07D 498/04; C07D 498/08

(57) ABSTRACT

The present invention relates to dioxazines, their synthesis, and their use for increasing GBA activity and/or levels as well as treatment of GBA-related diseases, such as Parkinson's disease.

### TECHNICAL FIELD

The present invention relates to dioxazines, their synthesis, and their use for increasing GBA activity and/or levels as well as treatment of GBA-related diseases, such as Parkinson's disease.

### BACKGROUND

The lysosome functions as a crucial re-processing center in human cells, breaking down proteins and fatty substances, such as glycosphingolipids, into their basic building blocks that are then recycled. A set of rare genetic diseases, called lysosomal storage diseases (LSD), are the result of carrying a distinct mutation in both copies of certain genes which encode various lysosomal enzymes. Gaucher disease, the most common lysosomal storage disease, is the result of a mutation in both copies of the GBA1 gene that codes for the Glucocerebrosidase (GCase) enzyme. Such homozygous mutations in both copies of the GBA1 gene cause a severe loss of up to 95% of GCase activity. As a result of this critical loss of enzyme activity, the metabolism of certain glycosphingolipids is significantly impaired in Gaucher disease patients, leading to accumulation of Glucosylceramide (GluCer), the GCase enzyme's substrate. This accumulation leads to serious health issues and organ pathology.

Many of these GBA mutations are also found in patients with Parkinson's disease (PD). Heterozygous mutations as found in GBA mutation carriers (having one mutated GBA gene) are found to predispose for development of Parkinson's disease (Gan-Or et al., Neurology, 2015). Mutations in GBA are now considered one of the main genetic risk factors for Parkinson's disease. It has been estimated that at least 8% of patients with Parkinson's disease have mutations in the GBA gene, both mild and severe GBA mutations, including L444P heterozygotes. Also secondary deficiencies of GBA activity may be linked to Parkinson's disease.

State of the art compounds, Ambroxol and LTI-291 have been shown to increase GBA activity, an important effect in treatment of GBA-mediated disorders. In order to meet the medical need of treating GBA-mediated disorders, more and better compounds are needed.

### SUMMARY

The present inventors have developed a series of compounds that effectively act as GBA inducers with completely different structural chemotype compared to state of the art compounds Ambroxol and LTI-291. This renders the compounds of the present disclosure promising candidates for treatment of GBA-mediated disorders

In a first aspect, a compound of formula (Ia) is provided,

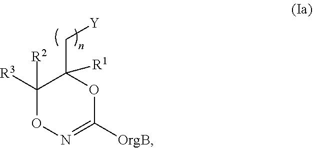

or a pharmaceutically acceptable salt thereof; wherein
n is 1 or 2;
R¹, R², and R³ are independently selected from the group consisting of: hydrogen, alkyl, and halogen;
Y is a nitrogen-containing ring or a nitrogen-containing chain;
OrgB is an organic basic moiety attached via a sp³ hybridised carbon to the rest of the compound; and
OrgB and Y are optionally substituted.

In a second aspect, a pharmaceutical composition is provided comprising a compound as defined herein, and one or more pharmaceutically acceptable adjuvants, excipients, carriers, buffers and/or diluents.

In a third aspect, a method for treating a disease in a subject is provided, comprising administering a compound as defined herein, wherein the disease is associated with reduced GBA levels and/or activity.

In a fourth aspect, a method of increasing the GBA activity and/or levels is provided comprising contacting GBA with a compound as defined herein.

In a fifth aspect, use of a compound as defined herein is provided for the manufacture of a medicament for the treatment of Parkinson's disease (PD).

### DETAILED DESCRIPTION

### Definitions

With reference to substituents, the term “independently” refers to the situation where when more than one substituent is possible, the substituents may be the same or different from each other.

As used herein, the term “pharmaceutically acceptable salt” refers to a salt used typically in the pharmaceutical field. Examples of

the pharmaceutically acceptable salt include sodium salts, hydrochloride salts, magnesium salts, calcium salts, trifluoroacetic acid salts and potassium salts, but are not limited thereto. Further exemplary salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, olcate, tannate, pantothenate, bitartrate, ascorbate, succinate, malcate, gentisinate, fumarate, gluconate, glucuronate, saccharate, formate, benzoate, glutamate, methanesulfonate, ethanesulfonate, benzenesulfonate, p-toluenesulfonate, and pamoate.

The potency, “EC_{1.5}” referred to herein is determined based on the dose response effects of the compounds as the concentration where “Percent GCase activity”=150% corresponding to at 1.5-fold induction of GCase activity.

The term “alkyl” refers to a straight or branched hydrocarbon chain radical consisting of carbon and hydrogen atoms, and may be straight or branched, substituted or unsubstituted. In some preferred embodiments, the alkyl group may consist of 1 to 12 carbon atoms, e.g. 1 carbon atom, 2 carbon atoms, 3 carbon atoms, 4 carbon atoms etc., up to and including 12 carbon atoms. Exemplary alkyl groups include, but are not limited to, methyl, ethyl, propyl, isopropyl, n-butyl, iso-butyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (iso-propyl), n-butyl, n-pentyl, 1,1-dimethylethyl (t-butyl) and 3-methylhexyl. Unless stated otherwise specifically in the specification, an alkyl group is optionally substituted by one or more of any suitable substituents. An alkyl group can be mono-, di-, tri- or tetra-valent, as appropriate to satisfy valence requirements.

The term “alkyl linker” as used herein refers to an alkyl, preferably a C1-C6 alkyl, capable of connecting one part of the molecule disclosed herein to another part of the molecule. An example of an alkyl linker is “methylene”. An alkyl linker may thus connect e.g. a monocyclic ring, a bicyclic ring, or a tricyclic ring to the cyclic oxime of formula (Ia) disclosed herein.

Generally, suitable substituents for substituted groups disclosed herein independently include, but are not limited to, alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a}, —N(R^{a})S(O)₂R^{a}, —S(O)OR^{a}, —S(O)₂OR^{a}, —S(O)N(R^{a})₂, —S(O)₂N(R^{a})₂, or PO₃(R^{a})₂ where each R^{a} is independently hydrogen, alkyl, haloalkyl, carbocyclyl, carbocyclylalkyl, aryl, aralkyl, heterocycloalkyl, heterocycloalkylalkyl, heteroaryl or heteroarylalkyl.

The term “cycloalkyl” refers to a monocyclic or polycyclic radical that contains carbon and hydrogen, and may be saturated, or partially unsaturated. In some preferred embodiments, cycloalkyl groups include groups having from 3 to 12 ring atoms (i.e. (C₃₋₁₂)cycloalkyl or C(₃₋₁₂)cycloalkyl). Whenever it appears herein, a numerical range such as “3 to 12” in (C₃₋₁₂)cycloalkyl or C(₃₋₁₂)cycloalkyl refers to each integer in the given range—e.g., “3 to 12 carbon atoms” means that the cycloalkyl group may consist of 3 carbon atoms, 4 carbon atoms, 5 carbon atoms, etc., up to and including 12 carbon atoms. Illustrative examples of cycloalkyl groups include, but are not limited to cyclopropyl, cyclobutyl, cyclopentyl, cyclopentenyl, cyclohexyl, cyclohexenyl, cycloseptyl, cyclooctyl, cyclononyl, cyclodecyl, norbornyl, and the like.

The term “alkoxy” refers to the group —O-alkyl. In some preferred embodiments, the alkoxy group contains from 1 to 12 carbon atoms of a straight, branched, cyclic configuration and combinations thereof attached to the parent structure through an oxygen. Examples of alkoxy include, but are not limited to, methoxy, ethoxy, propoxy, isopropoxy, cyclopropyloxy and cyclohexyloxy.

The term “acyl” refers to R^{c}—(C═O)— wherein R^{c} include, but is not limited to, alkyl, heteroalkyl, alkenyl, alkynyl, cycloalkyl, heterocycloalkyl, aryl, aralkyl, heteroaryl. The acyl is attached to the parent structure through the carbonyl functionality.

The term “amino” or “amine” refers to a —N(R^{a})₂ radical group, where each R^{a} is independently hydrogen, alkyl, (halo)alkyl, alkenyl, alkynyl, carbocyclyl, carbocyclylalkyl, aryl, aralkyl, heterocycloalkyl, heterocycloalkylalkyl, heteroaryl or heteroarylalkyl, unless stated otherwise. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl, 1-piperazinyl, and 4-morpholinyl.

The term “amide” or “amido” refers to a chemical moiety with formula —(C═O)N(R^{d})₂ or —NH(C═O)R^{d}, where R^{d} is selected from the group consisting of hydrogen, alkyl, alkenyl, alkynyl, carbocyclyl, carbocyclylalkyl, cycloalkyl, aryl, and heteroaryl. The R^{d} of —N(R^{d})₂ of the amide may optionally be taken together with the nitrogen to which it is attached to form a 4-, 5-, 6- or 7-membered ring. Unless stated otherwise specifically in the specification, an amide group is optionally substituted independently by one or more of the substituents as described herein as suitable substitution groups.

The term “haloalkyl” refers to an alkyl radical, as defined above, that is substituted by one or more halogen atoms. The term “alkyl” thus includes “haloalkyl”. Examples of haloalkyl include, but are not limited to, trifluoromethyl, difluoromethyl, 2,2,2-trifluoroethyl, 1-fluoromethyl-2-fluoroethyl, and the like.

The term “halo”, “halide”, or, alternatively, “halogen” is intended to mean fluoro, chloro, bromo or iodo.

The term “aromatic” means an unsaturated, cyclic and planar hydrocarbon group with a delocalized conjugated π system having 4n+2 π electrons, where n is an integer having a value of 0, 1, 2, 3, and so on. In some embodiments, the aromatic group is an “aryl” (abbreviated as Ar), which refers to an aromatic radical with six to ten ring atoms (e.g., (C₆₋₁₀)aromatic or (C₆₋₁₀)aryl) which has at least one ring having a conjugated pi electron system which is carbocyclic (e.g., phenyl, fluorenyl, and naphthyl).

The term “aralkyl” or “arylalkyl” refers to an (aryl)alkyl-radical where aryl and alkyl are as disclosed herein.

The term “heteroaryl” or “heteroaromatic refers to a 5- to 18-membered aromatic radical (e.g., (C₅₋₁₃)heteroaryl) that includes one or more ring heteroatoms selected from nitrogen, oxygen and sulfur, and which may be a monocyclic, bicyclic, tricyclic or tetracyclic ring system. Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl).

The term “tautomer” relate to structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond.

The symbol “ [symbol] ”, displayed perpendicular to a bond, indicates the point at which the displayed moiety is attached to the remainder of the molecule.

The term “organic basic moiety” refers to the combination of terms “organic base” and “moiety”. The term “moiety” refers to a part of a molecule, which is covalently connected to the rest of the molecule. An “organic base” is an organic compound which can act as a base. Organic bases usually contain nitrogen atoms, which can be protonated, for example amines have a lone pair of electrons on the nitrogen atom and can thus act as proton acceptors (bases). Amines and nitrogen-containing heterocyclic compounds are organic bases. An example of an organic base is piperidine. An “organic basic moiety” is thus an organic base, which is part of a molecule, wherein the basic function resides with the moiety. The organic basic moiety is referred to as “OrgB” herein.

### Compounds

In one embodiment, a compound of formula (Ia) is provided,

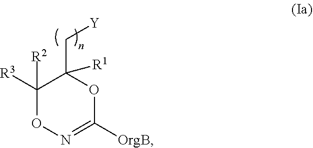

or a pharmaceutically acceptable salt thereof; wherein
n is 1 or 2;
R¹, R², and R³ are independently selected from the group consisting of: hydrogen, alkyl, and halogen;
Y is a nitrogen-containing ring or a nitrogen-containing chain;
OrgB is an organic basic moiety attached via a sp³ hybridised carbon to the rest of the compound; and
OrgB and Y are optionally substituted.

In one embodiment, the compound is of formula (Ib),

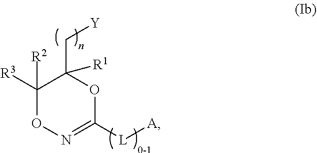

wherein
A is a monocyclic ring, a bicyclic ring, or a tricyclic ring, and A is attached via a sp³ hybridised carbon to the rest of the compound;
L is a C₁₋₆ alkyl linker or L is absent; if L is absent, A is directly attached to the cyclic oxime; and
A and Y are optionally substituted.

In one embodiment, the compound as defined herein is provided, wherein A is of formula (II):

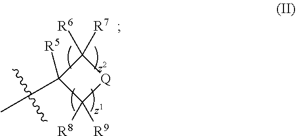

wherein
z¹ and z² are independently selected from the group consisting of: 0, 1, 2, and 3;
Q is selected from the group consisting of:

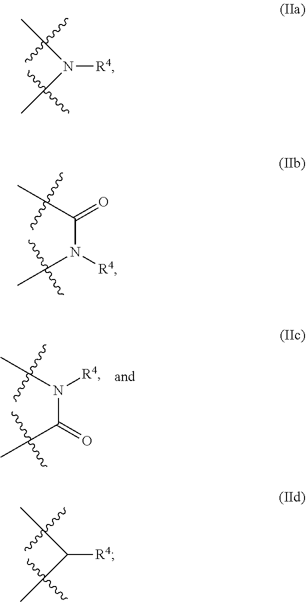

R⁴ is selected from the group consisting of: hydrogen, alkyl, amino, alkoxy, acyl, amido, aralkyl, aryl, heteroaryl, cycloalkyl, heterocycloalkyl;
R⁵ is selected from the group consisting of: hydrogen, alkyl, aralkyl, hydroxy, alkoxy, and amino;
each R⁶ is independently selected from the group consisting of: hydrogen, alkyl, alkoxy, hydroxy, amino, amido, and halogen;
each R⁷ is independently selected from the group consisting of: hydrogen, alkyl, alkoxy, hydroxy, amino, amido, and halogen;
each R⁸ is independently selected from the group consisting of: hydrogen, alkyl, alkoxy, hydroxy, amino, amido, and halogen; and
each R⁹ is independently selected from the group consisting of: hydrogen, alkyl, alkoxy, hydroxy, amino, amido, and halogen.

In one embodiment, the compound is provided, wherein A is of formula (II) and Q is of formula (IIa).

In one embodiment, the compound is provided, wherein L is of formula (III)

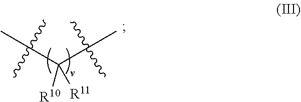

wherein
v is 0 or 1; if v is 0, L is absent;
each R¹⁰ is independently selected from the group consisting of: hydrogen and alkyl;
each R¹¹ is independently selected from the group consisting of: hydrogen and alkyl;
if both R¹⁰ and R¹¹ are alkyl, R¹⁰ and R¹¹ are optionally connected to form a C₃₋₆ ring.

In one embodiment, the compound is provided, wherein R⁵ is selected from the group consisting of:

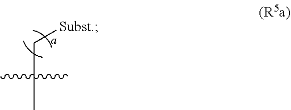

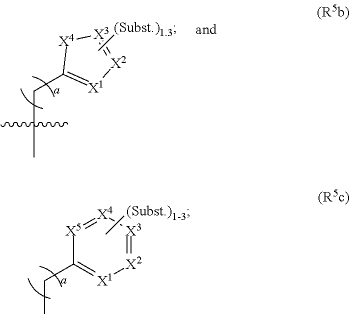

or any tautomer thereof,
   wherein a is 0, 1, 2, or 3;
   X¹, X², X³, X⁴, and X⁵ independently are selected from the group consisting of: C, CH, and N; and
   each one, two, or three Subst. is independently selected from the group consisting of: hydrogen, alkyl, halogen, hydroxy, alkoxy, amino, amido, acyl, cycloalkyl, and heterocycloalkyl.

In one embodiment, the compound is provided wherein A is selected from the group consisting of: a monocyclic ring and a bicyclic ring. In one embodiment, A is selected from the group consisting of: a monocyclic ring and a bicyclic ring; and
b) comprises 1, 2 or 3 nitrogen atoms; and/or
c) comprises 0, 1, 2 or 3 oxygen atoms.

In one embodiment, the compound is provided, wherein A comprises 1, 2 or 3 nitrogen atoms. In one embodiment, the compound is provided, wherein A comprises 0, 1, 2 or 3 oxygen atoms.

In one embodiment, the compound is provided wherein A is a cycle comprising 5-10 ring atoms. In one embodiment, A is a C₅₋₉ heterocycle. In one embodiment, A is a C₅-9 bicyclic heterocycle comprising pyrrolidine.

In one embodiment, the compound is provided wherein A is of formula (II) and L is of formula (III), wherein L-A is selected from the group consisting of:

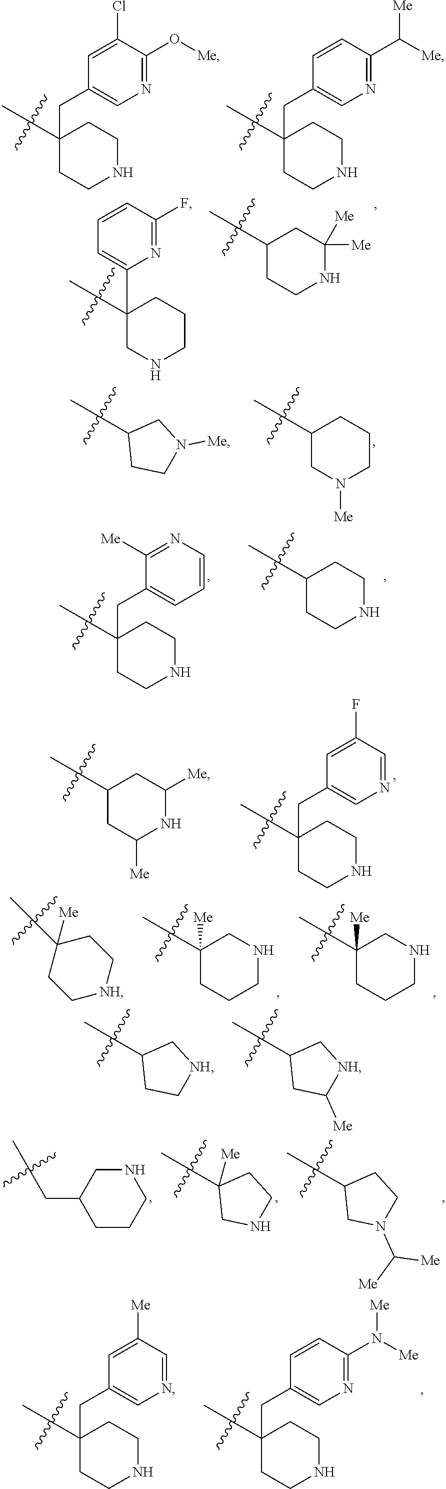

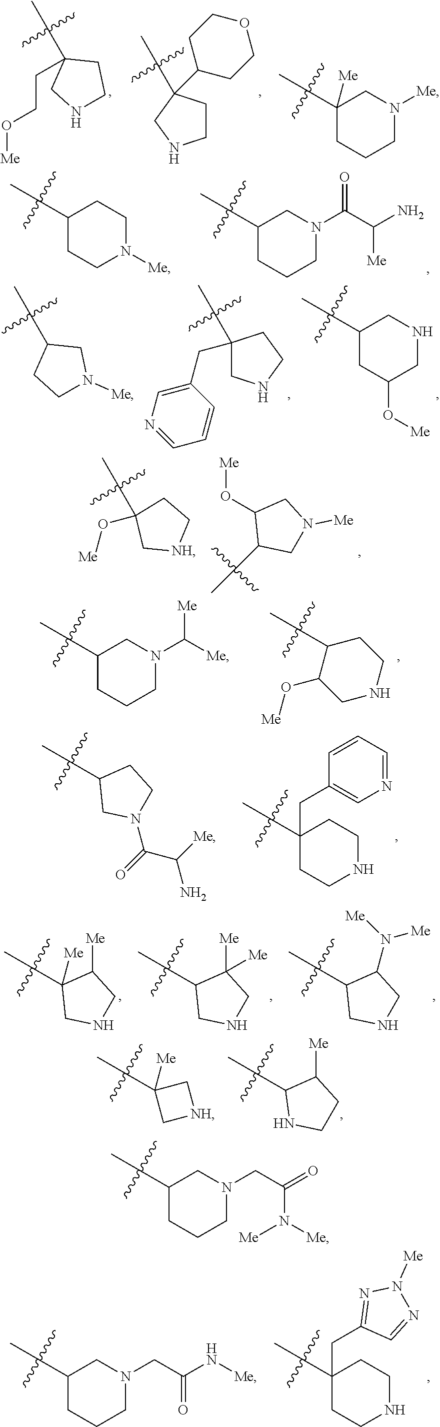

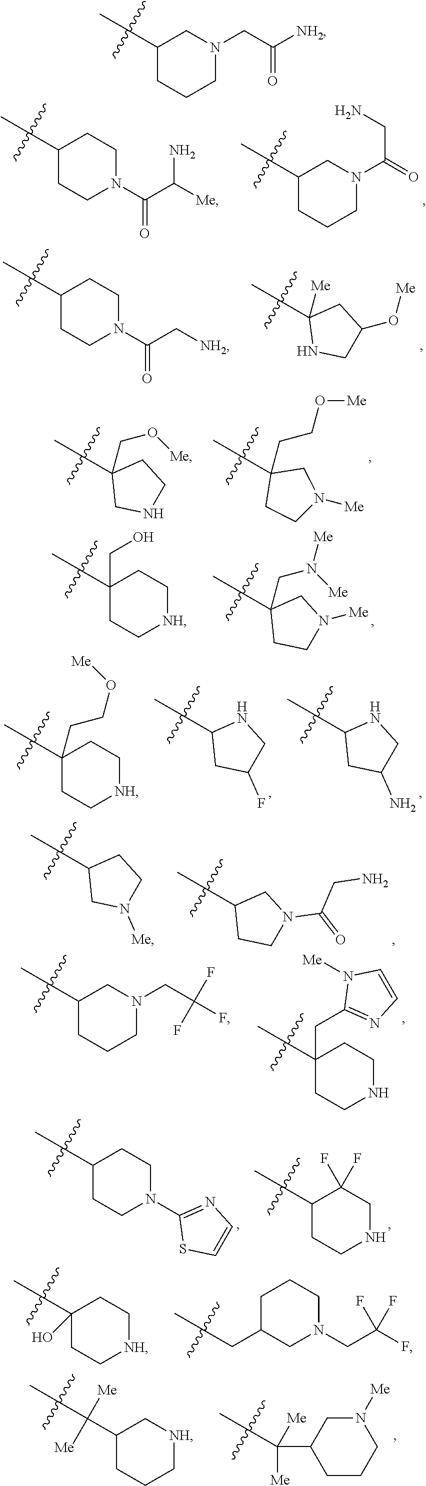

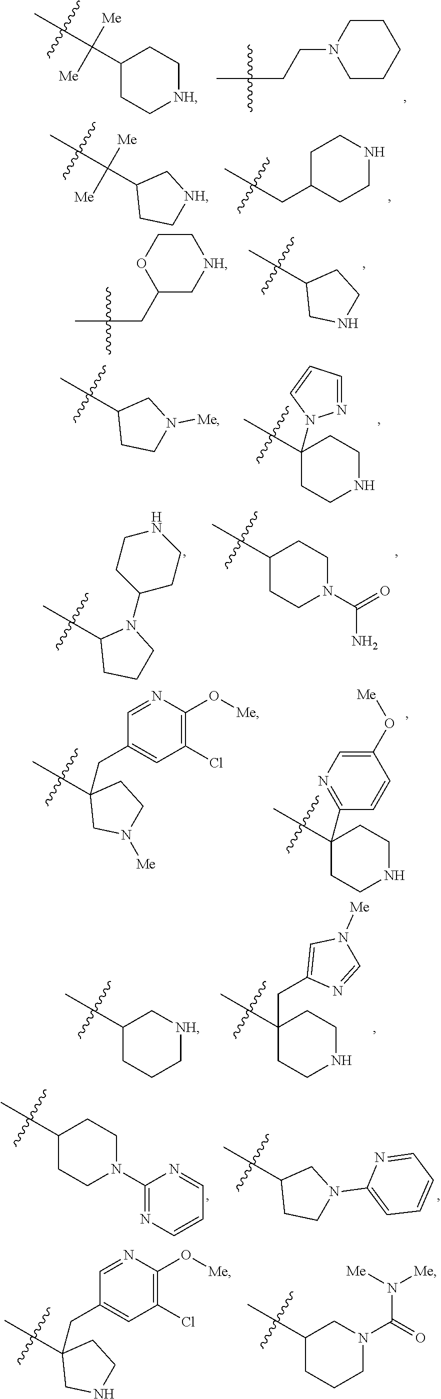

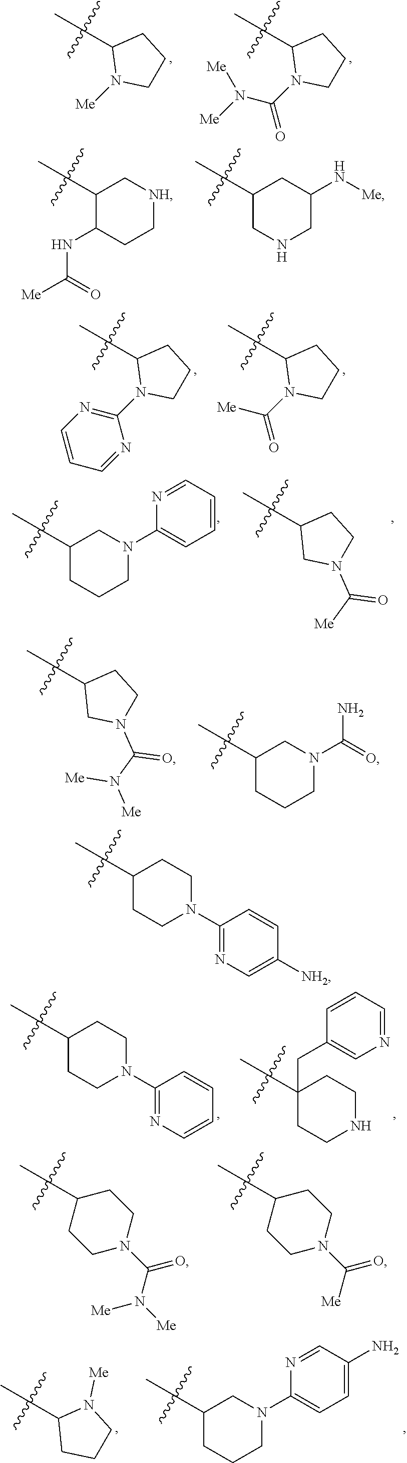

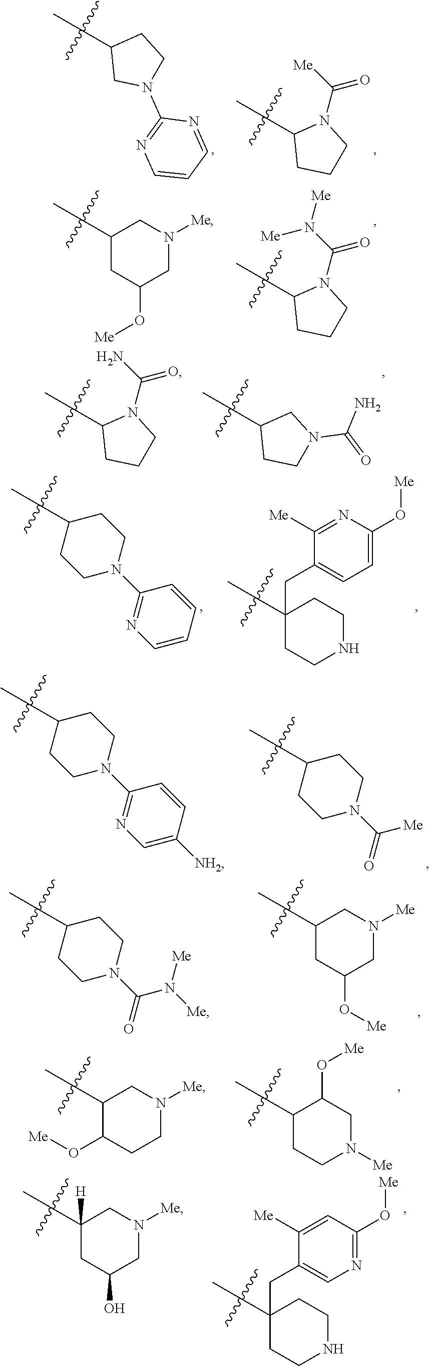

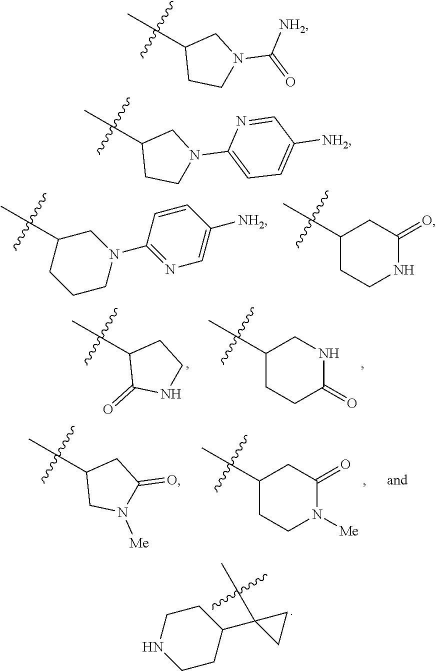

In one embodiment, the compound is provided, wherein A is selected from the group consisting of:

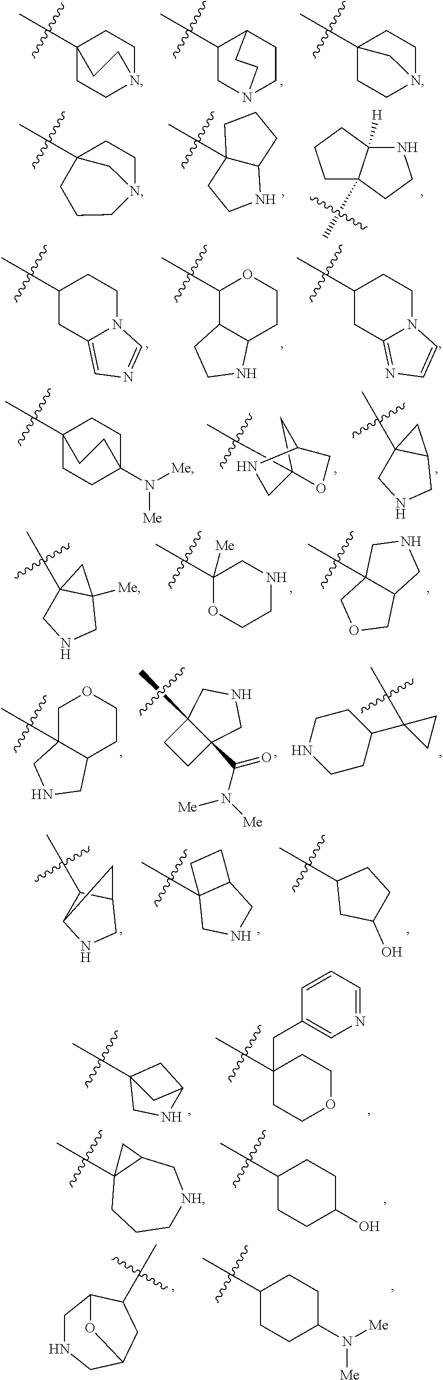

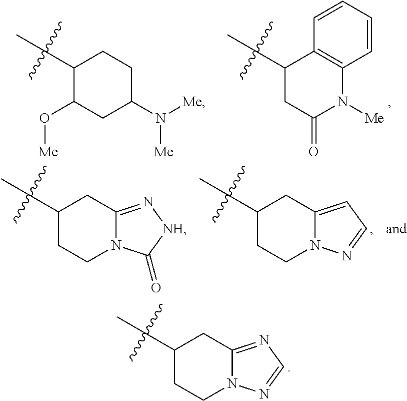

In one embodiment, the compound as defined herein is provided wherein OrgB is selected from the group consisting of:

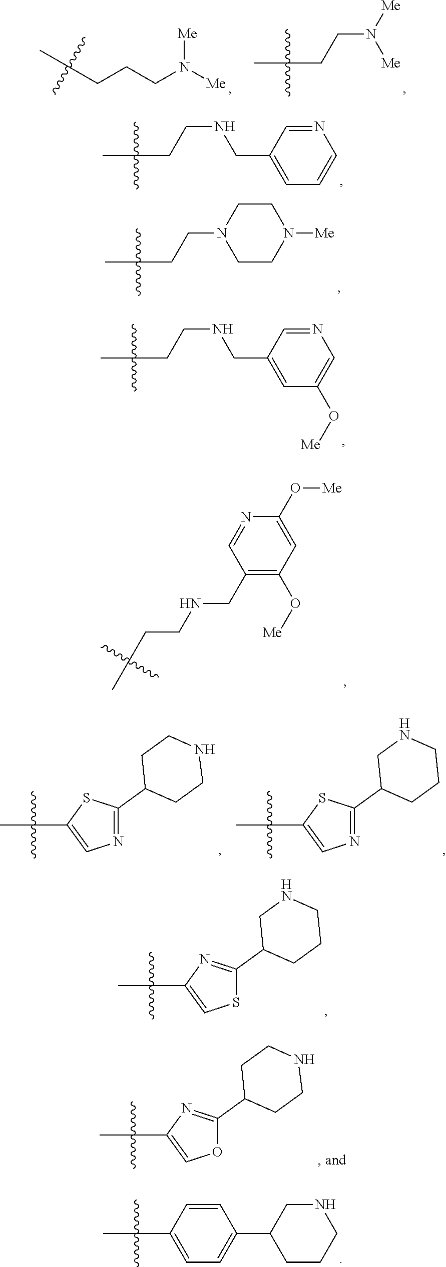

In one embodiment, the compound is provided wherein Y is a nitrogen-containing ring, wherein the nitrogen-containing ring is monocyclic or bicyclic.

In one embodiment, the compound is provided wherein Y is an optionally substituted piperidine, such a piperidine substituted by one, two, three or four methyl groups.

In one embodiment, the compound is provided wherein Y is an optionally substituted pyrrolidine. In one embodiment, Y is an optionally substituted piperazine.

In one embodiment, the compound is provided wherein Y is selected from the group consisting of:

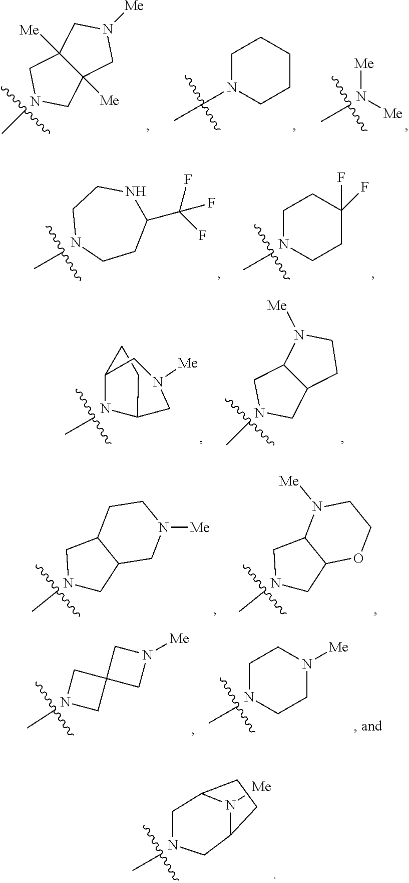

In one embodiment, the compound is provided wherein R¹, R², and R³ independently are selected from the group consisting of: hydrogen and alkyl. In one embodiment, the compound is provided wherein R¹ and R² are both hydrogen, and R³ is C₁₋₆ alkyl.

In one embodiment, the compound is provided wherein R³ is selected from the group consisting of: methyl, ethyl, propyl, isopropyl, cyclopropyl, cyclobutyl, cyclopentyl, and cyclohexyl. In one embodiment, R³ is selected from the group consisting of: methyl, isopropyl, and cyclohexyl.

In one embodiment, the compound is provided wherein R⁴ is selected from the group consisting of: hydrogen and alkyl. In one embodiment, R⁴ is alkyl, such as C₁₋₆ alkyl. In one embodiment, R⁴ is selected from the group consisting of methyl, ethyl, propyl, butyl, isopropyl, sec-butyl, cyclopropyl, cyclobutyl, and cyclopentyl. In one embodiment, R⁴ is methyl.

In one embodiment, the compound is provided wherein R⁵ is hydrogen or methyl.

In one embodiment, the compound is provided wherein R⁶ and R⁷ are both hydrogen.

In one embodiment, the compound is provided, wherein R⁸ and R⁹ are both hydrogen.

In one embodiment, the compound is provided wherein z¹ and z² are both 2. In one embodiment, z¹ and z² are both 2, and wherein R⁶, R⁷, R⁸, and R⁹ are hydrogen. In one embodiment, z¹ and z² are 2, and wherein R⁶, R⁷, R⁸, and R⁹ are hydrogen, and wherein R⁵ is methyl.

In one particular embodiment, the compound is selected from the group consisting of

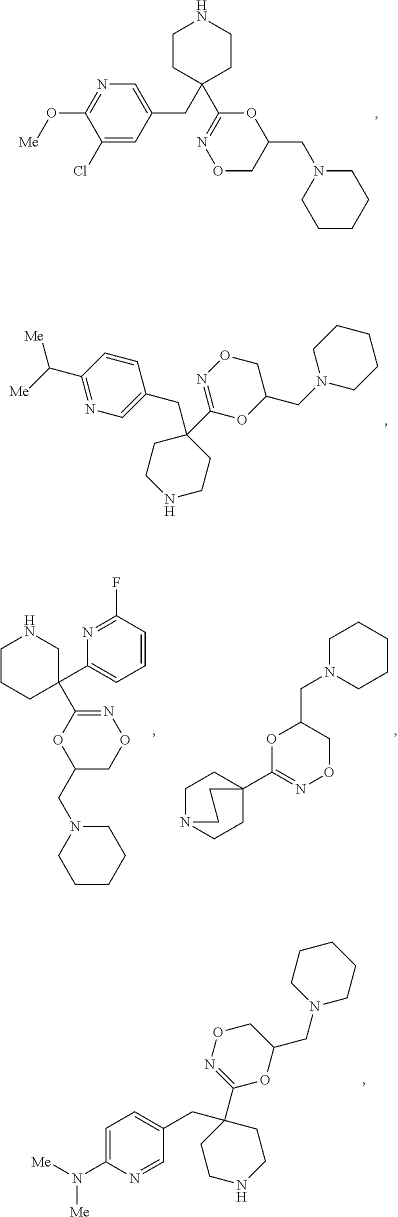 [SMILES: COc1ncc(CC2(C3=NOCC(CN4CCCCC4)O3)CCNCC2)cc1Cl ; CC(C)c1ccc(CC2(C3=NOCC(CN4CCCCC4)O3)CCNCC2)cn1 ; Fc1cccc(C2(C3=NOCC(CN4CCCCC4)O3)CCCNC2)n1 ; C1CCN(CC2CON=C(C34CCN(CC3)CC4)O2)CC1 ; CN(C)c1ccc(CC2(C3=NOCC(CN4CCCCC4)O3)CCNCC2)cn1]

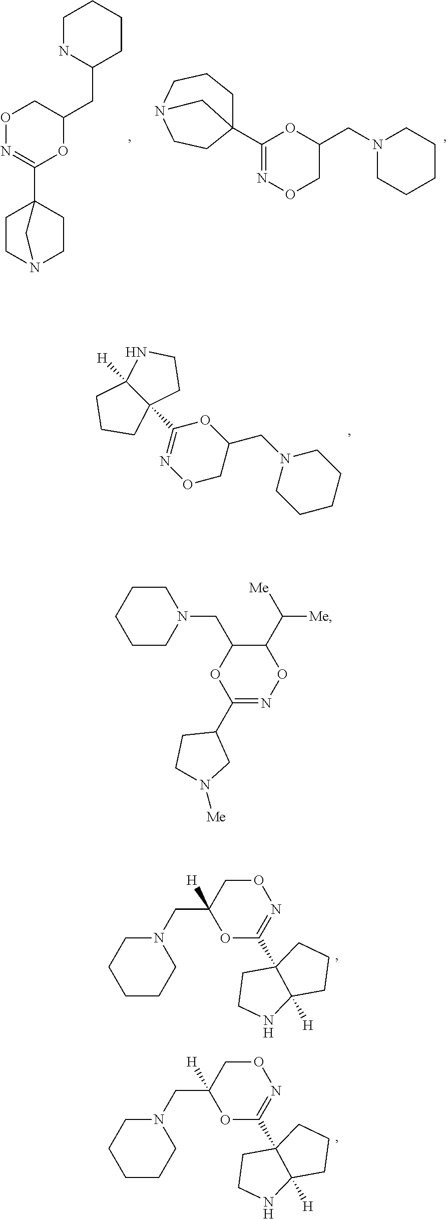

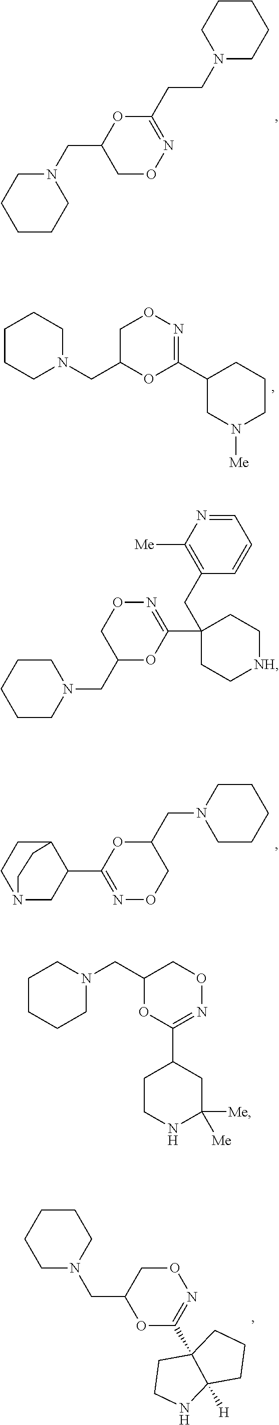 [SMILES: C1CCN(CCC2=NOCC(CN3CCCCC3)O2)CC1 ; CN1CCCC(C2=NOCC(CN3CCCCC3)O2)C1 ; Cc1ncccc1CC1(C2=NOCC(CN3CCCCC3)O2)CCNCC1 ; C1CCN(CC2CON=C(C3CN4CCC3CC4)O2)CC1 ; CC1(C)CC(C2=NOCC(CN3CCCCC3)O2)CCN1 ; C1CCN(CC2CON=C([C@]34CCC[C@H]3NCC4)O2)CC1]

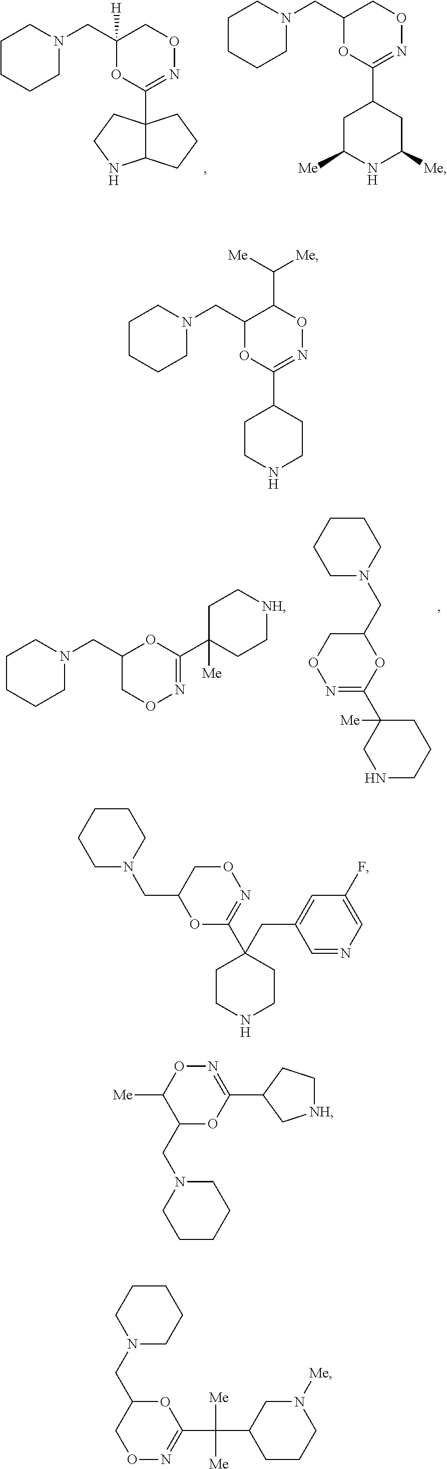

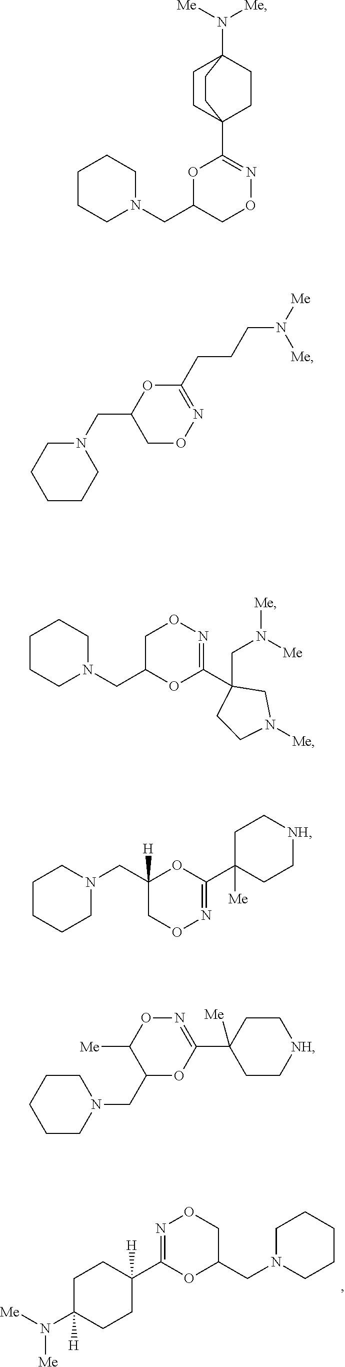 [SMILES: CN(C)C12CCC(C3=NOCC(CN4CCCCC4)O3)(CC1)CC2 ; CN(C)CCCC1=NOCC(CN2CCCCC2)O1 ; CN(C)CC1(C2=NOCC(CN3CCCCC3)O2)CCN(C)C1 ; CC1(C2=NOC[C@H](CN3CCCCC3)O2)CCNCC1 ; CC1ON=C(C2(C)CCNCC2)OC1CN1CCCCC1 ; CN(C)[C@H]1CC[C@@H](C2=NOCC(CN3CCCCC3)O2)CC1]

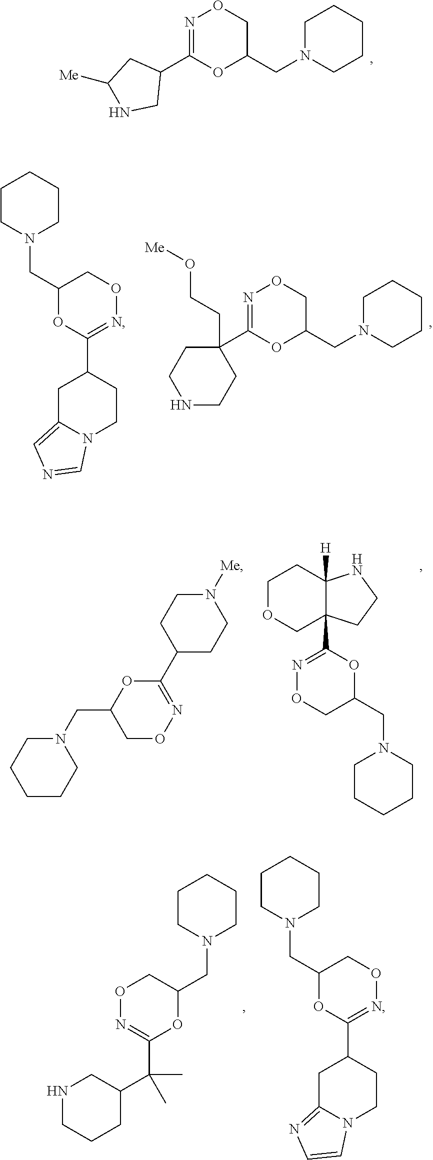

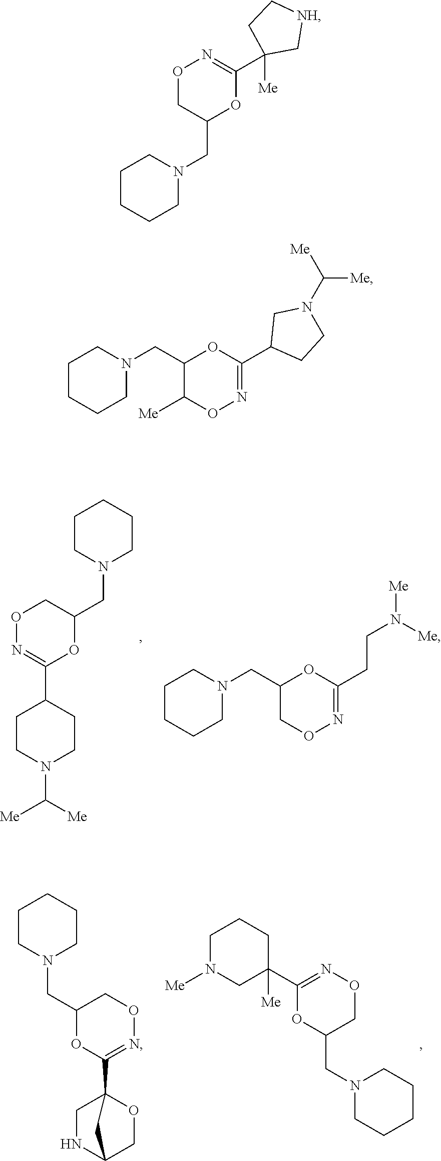 [SMILES: CC1(C2=NOCC(CN3CCCCC3)O2)CCNC1 ; CC1ON=C(C2CCN(C(C)C)C2)OC1CN1CCCCC1 ; CC(C)N1CCC(C2=NOCC(CN3CCCCC3)O2)CC1 ; CN(C)CCC1=NOCC(CN2CCCCC2)O1 ; C1CCN(CC2CON=C([C@@]34CN[C@@H](CO3)C4)O2)CC1 ; CN1CCCC(C)(C2=NOCC(CN3CCCCC3)O2)C1]

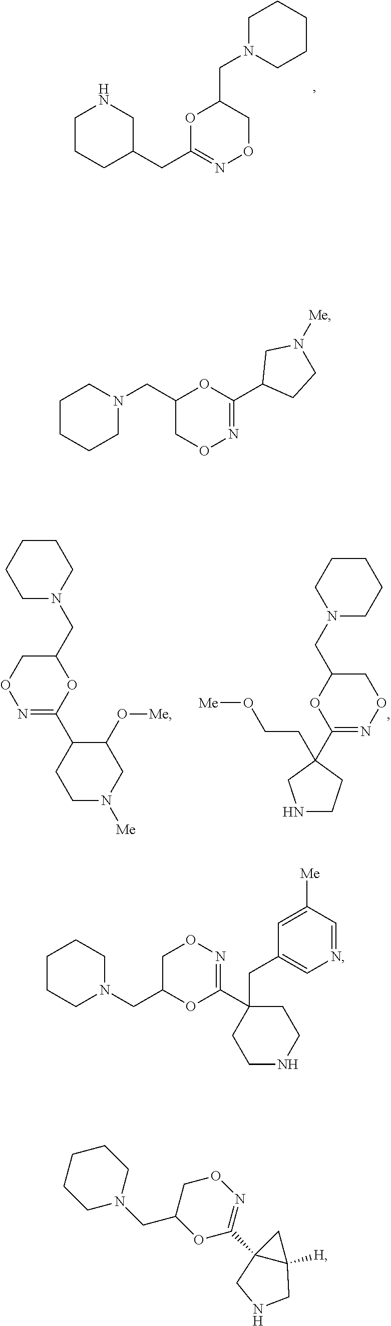

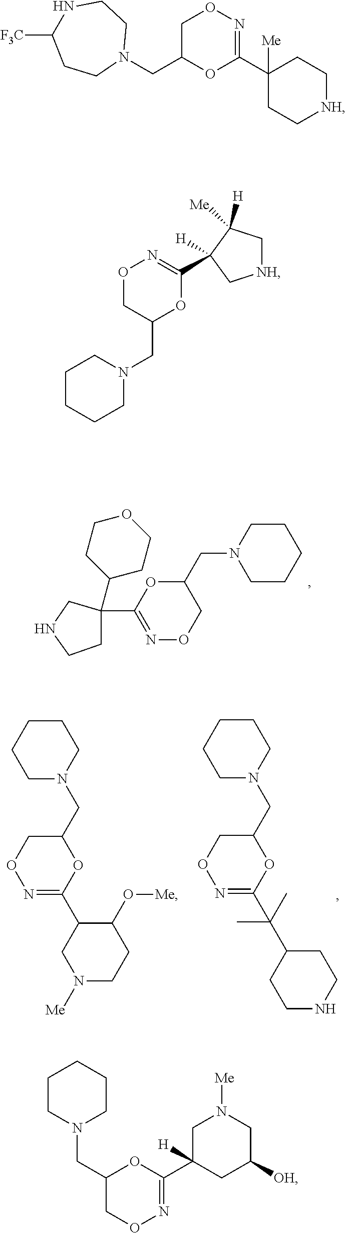 [SMILES: CC1(C2=NOCC(CN3CCNC(C(F)(F)F)CC3)O2)CCNCC1 ; C[C@@H]1CNC[C@H]1C1=NOCC(CN2CCCCC2)O1 ; C1CCN(CC2CON=C(C3(C4CCOCC4)CCNC3)O2)CC1 ; COC1CCN(C)CC1C1=NOCC(CN2CCCCC2)O1 ; CC(C)(C1=NOCC(CN2CCCCC2)O1)C1CCNCC1 ; CN1C[C@@H](O)C[C@H](C2=NOCC(CN3CCCCC3)O2)C1]

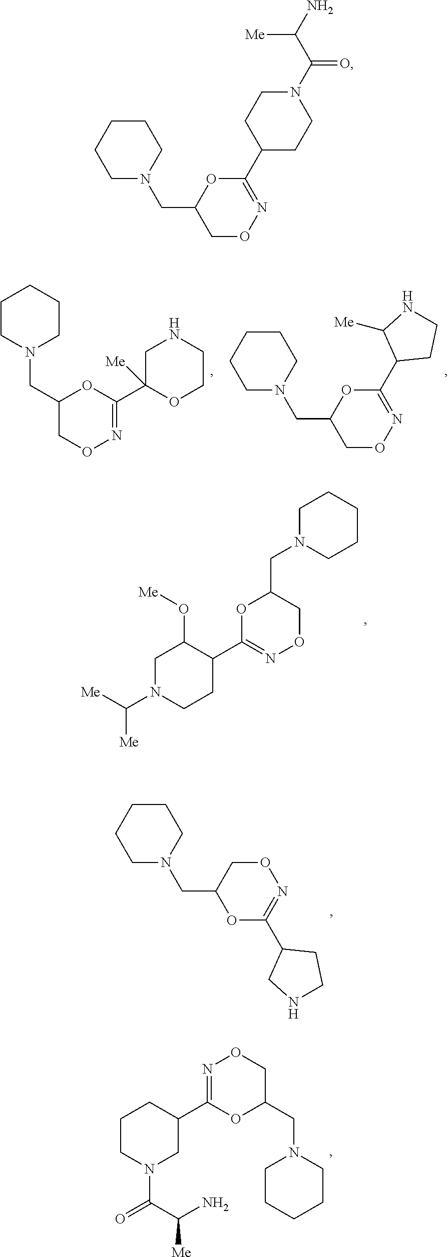

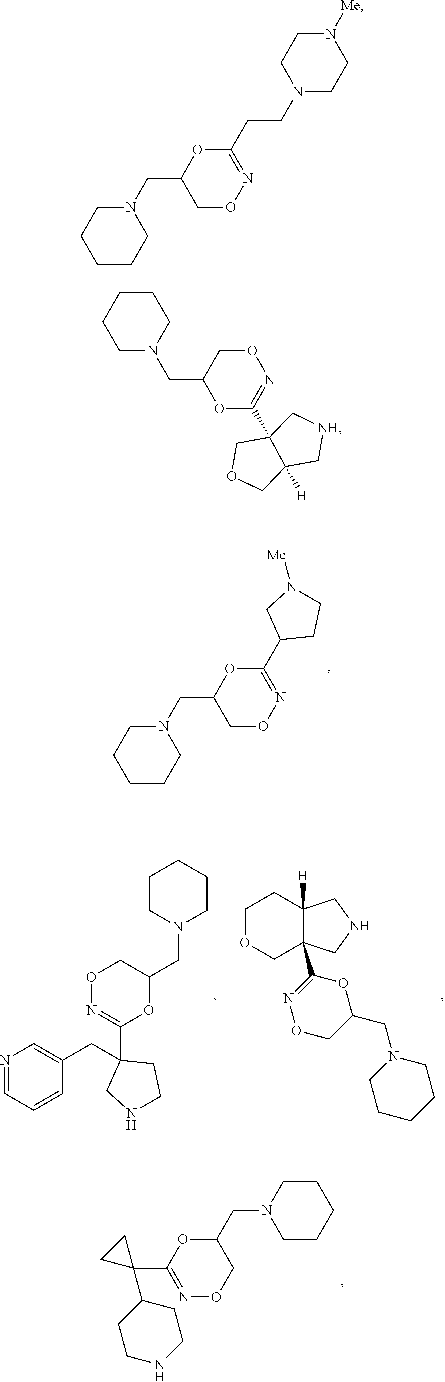

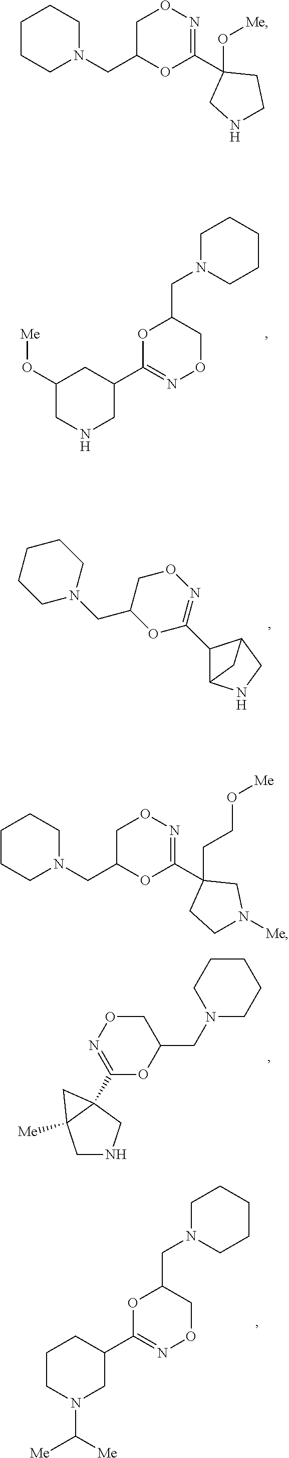

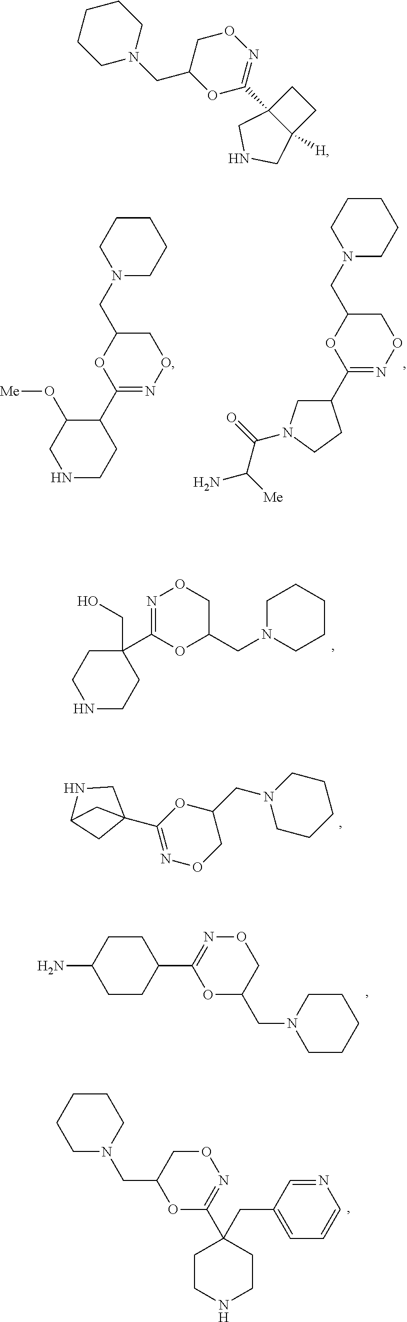

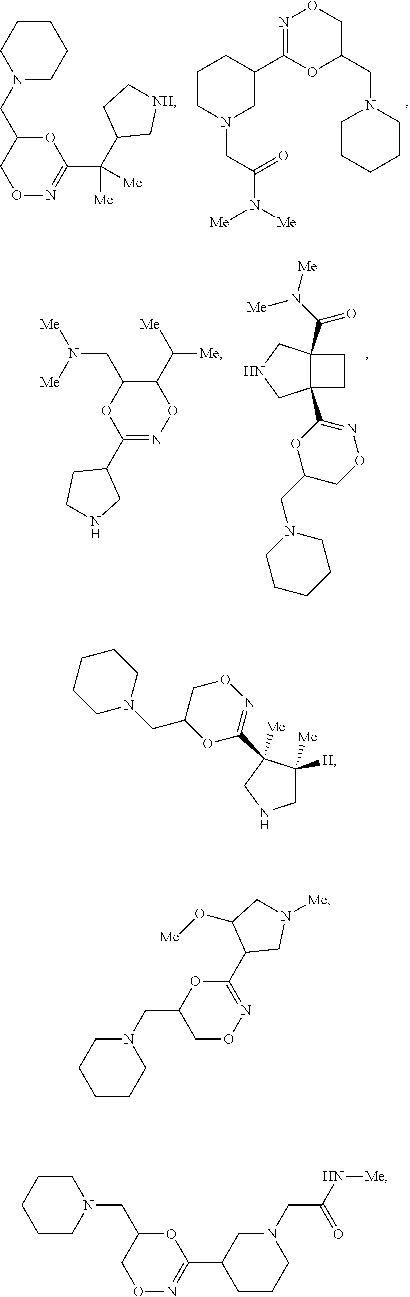

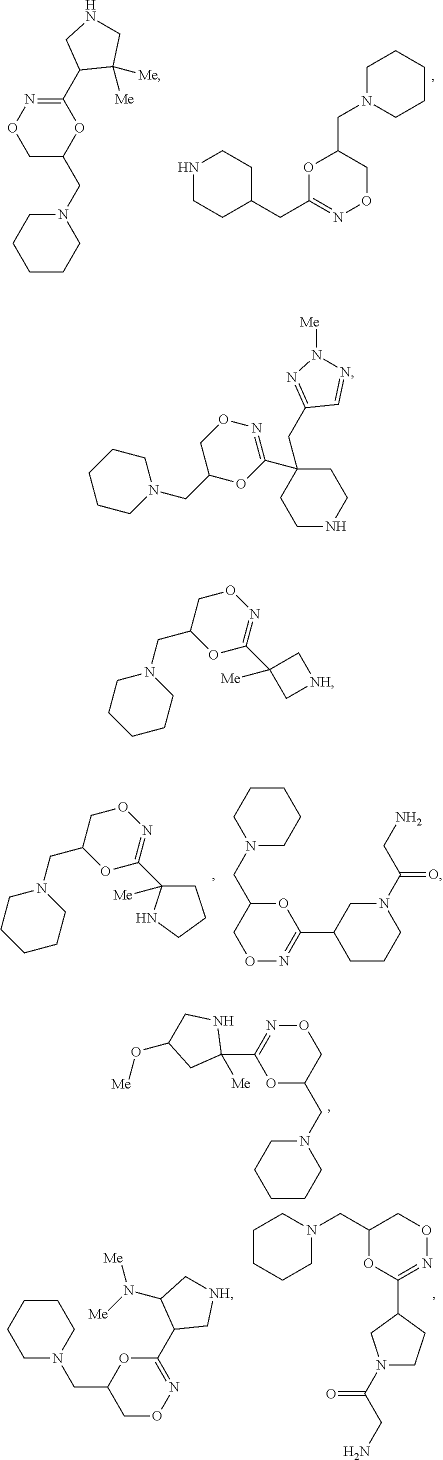

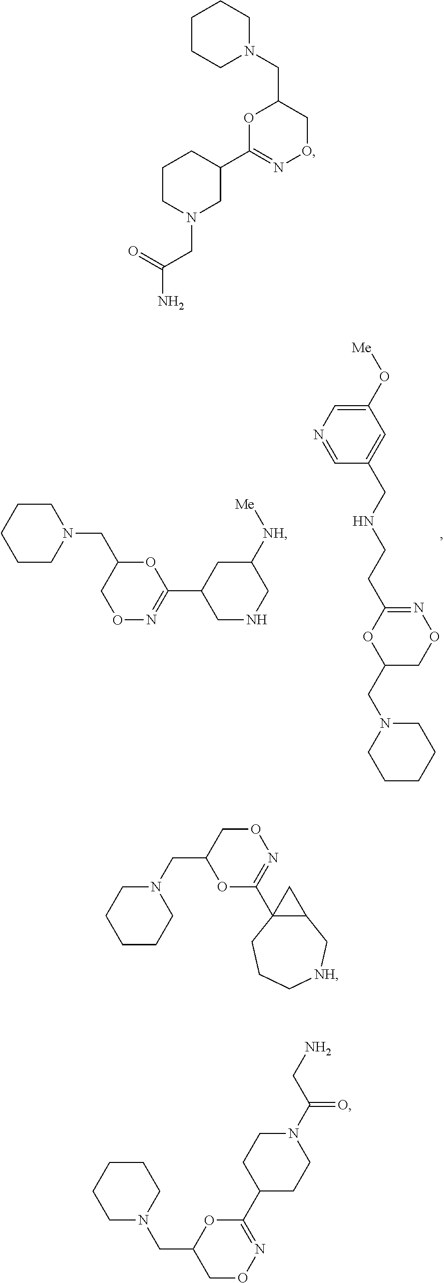 [SMILES: NC(=O)CN1CCCC(C2=NOCC(CN3CCCCC3)O2)C1 ; CNC1CNCC(C2=NOCC(CN3CCCCC3)O2)C1 ; COc1cncc(CNCCC2=NOCC(CN3CCCCC3)O2)c1 ; C1CCN(CC2CON=C(C34CCCNCC3C4)O2)CC1 ; NCC(=O)N1CCC(C2=NOCC(CN3CCCCC3)O2)CC1]

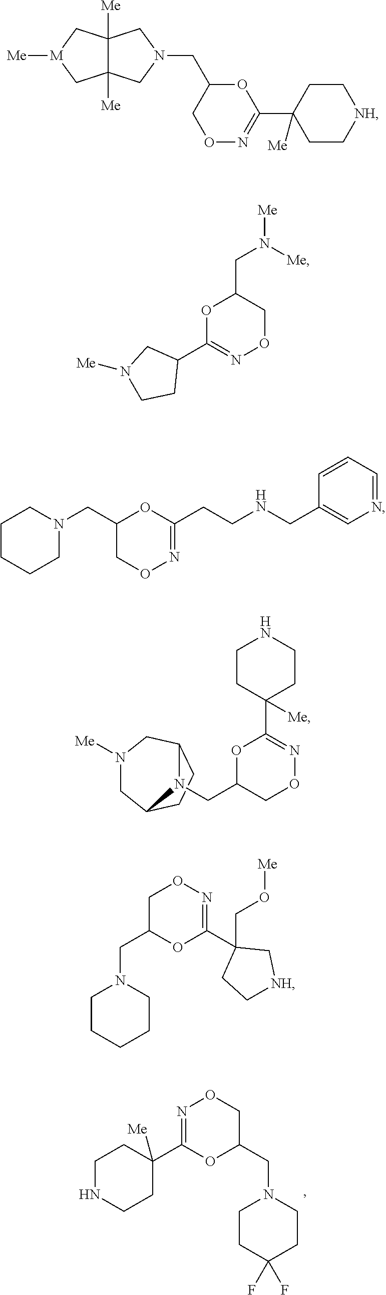

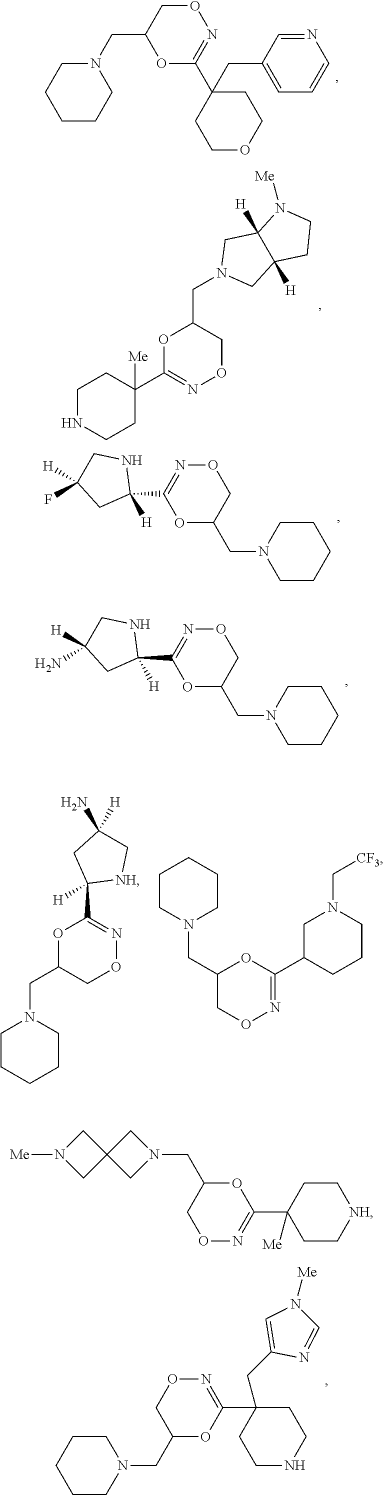

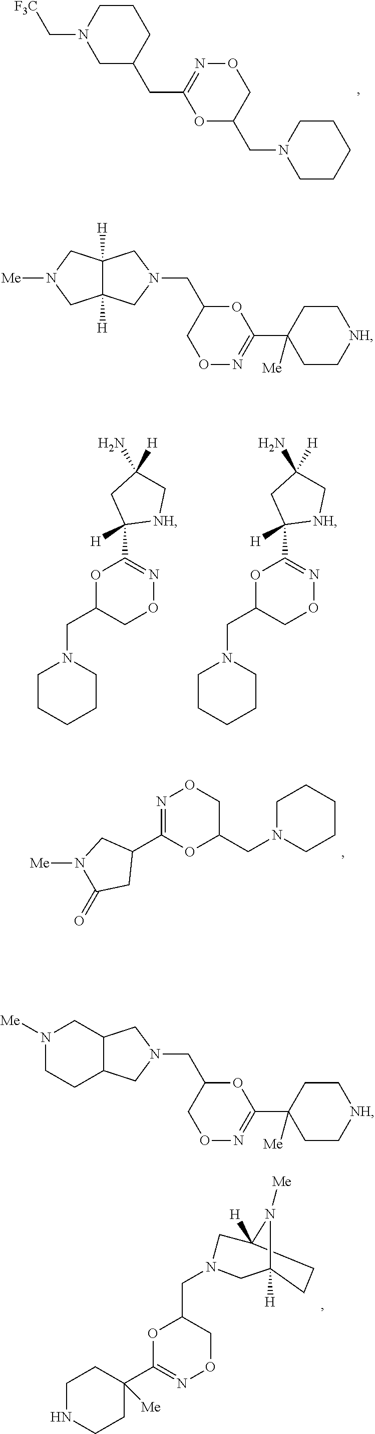

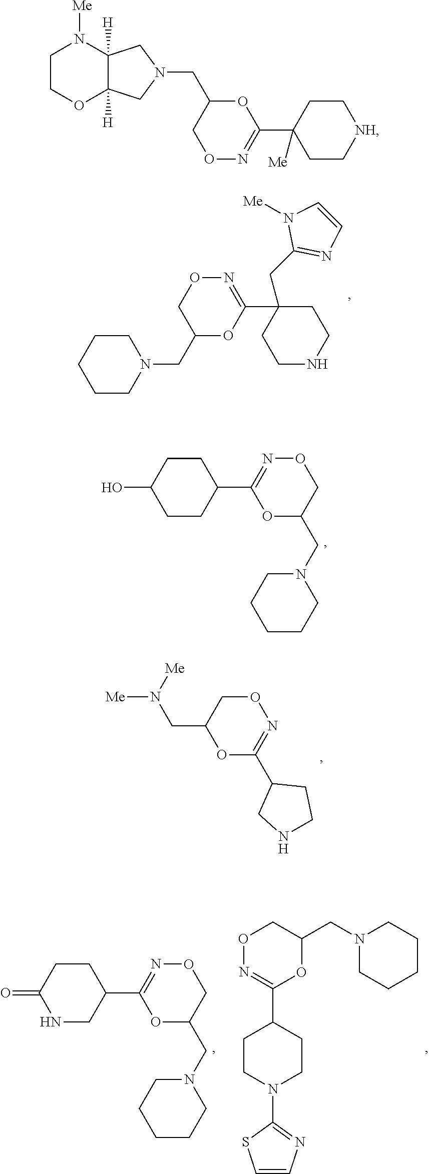

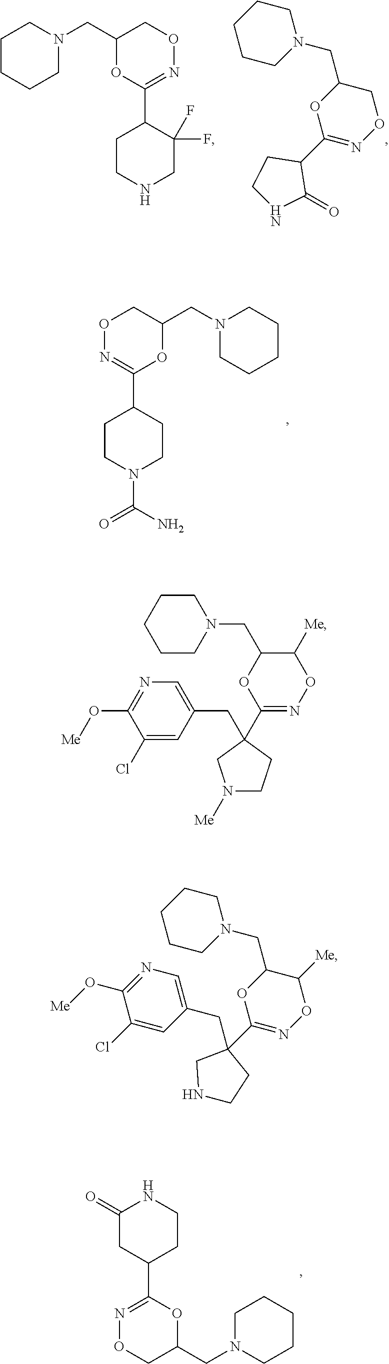 [SMILES: FC1(F)CNCCC1C1=NOCC(CN2CCCCC2)O1 ; O=C1NCCC1C1=NOCC(CN2CCCCC2)O1 ; NC(=O)N1CCC(C2=NOCC(CN3CCCCC3)O2)CC1 ; COc1ncc(CC2(C3=NOC(C)C(CN4CCCCC4)O3)CCN(C)C2)cc1Cl ; COc1ncc(CC2(C3=NOC(C)C(CN4CCCCC4)O3)CCNC2)cc1Cl ; O=C1CC(C2=NOCC(CN3CCCCC3)O2)CCN1]

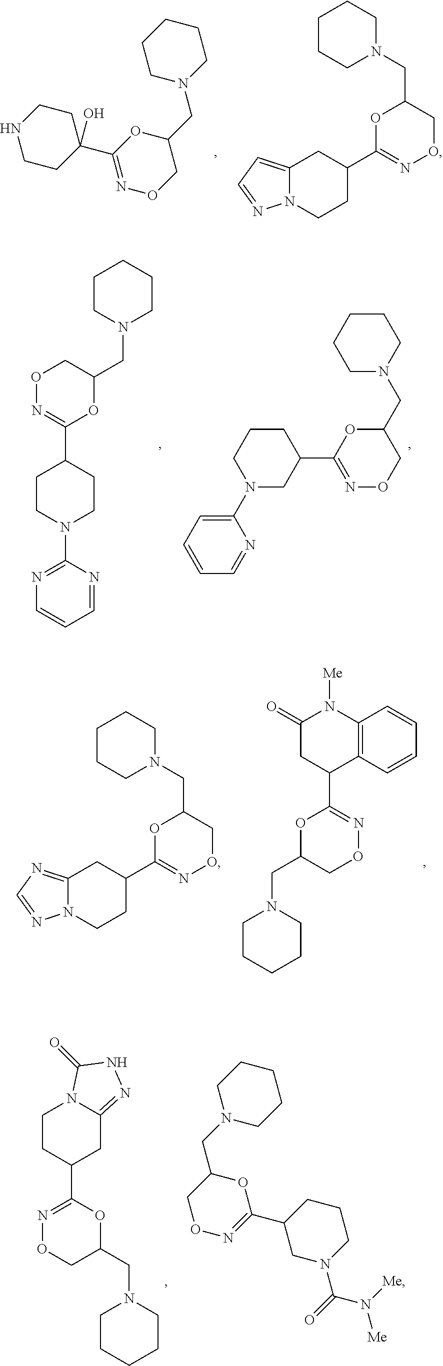

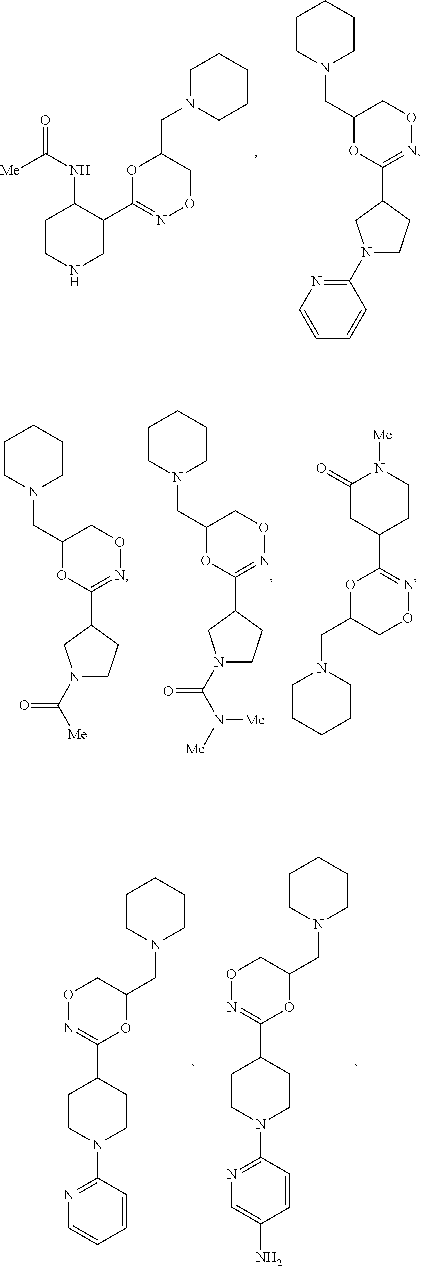

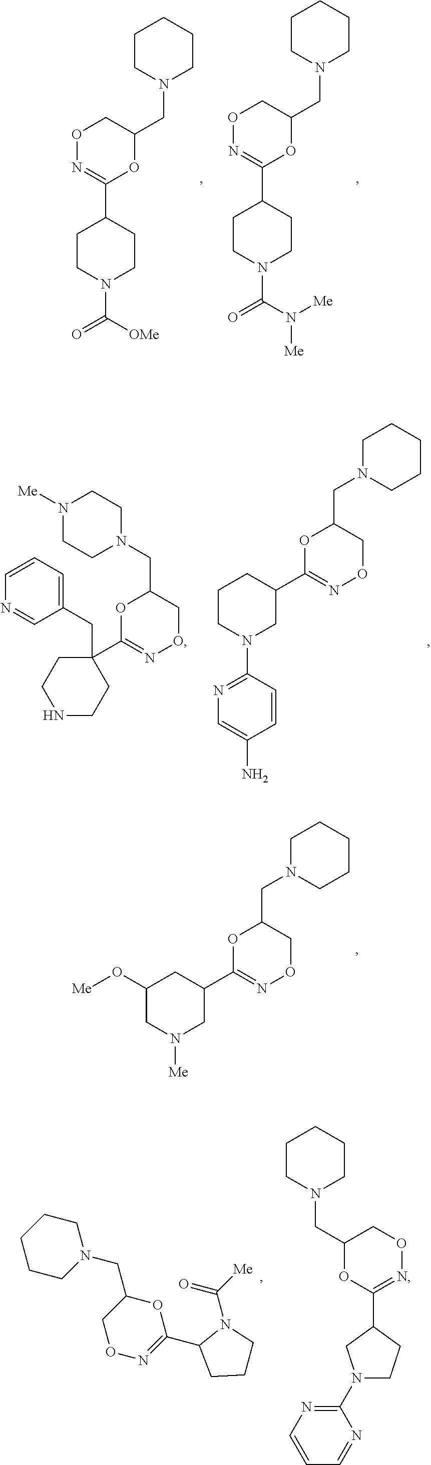

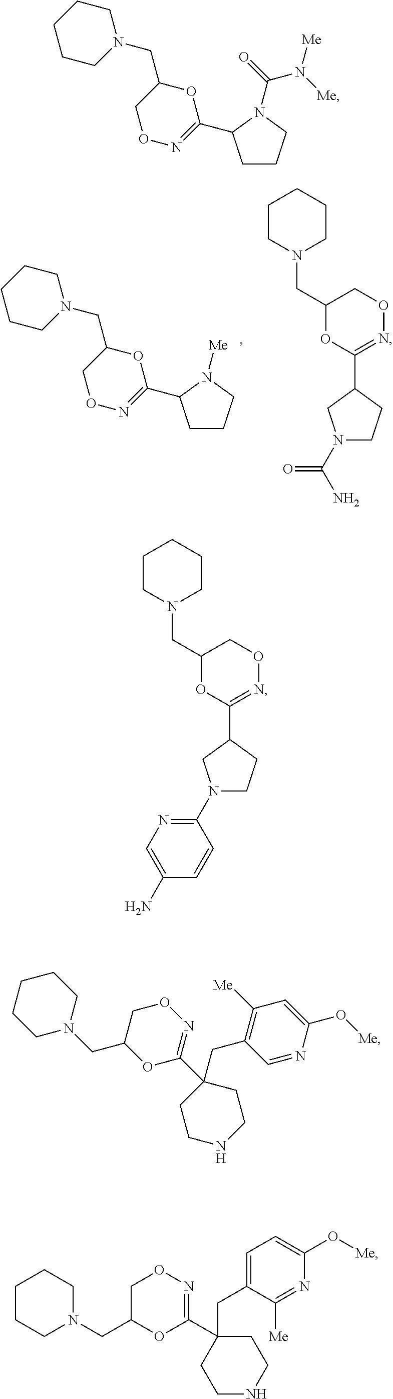

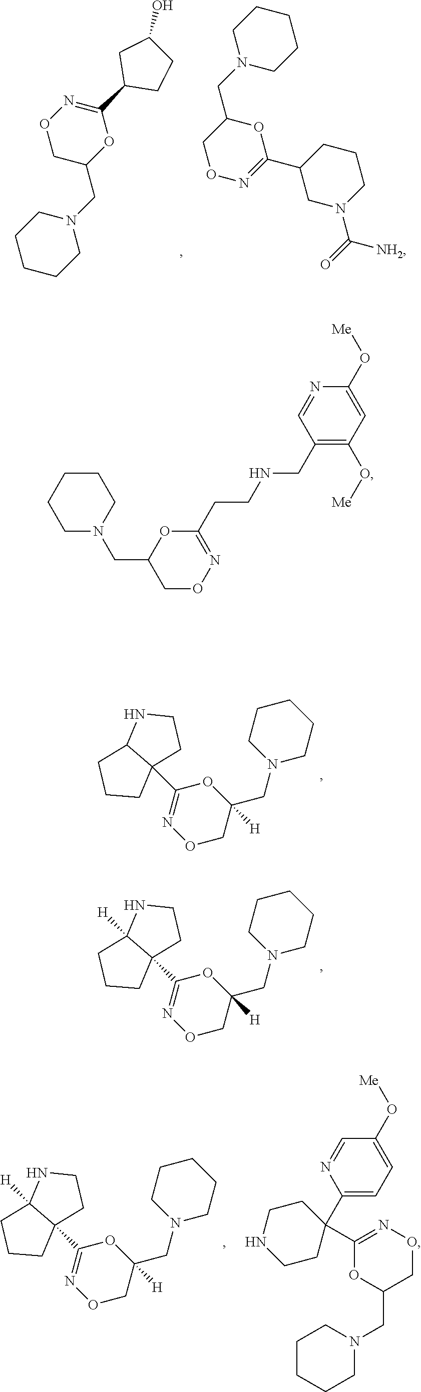

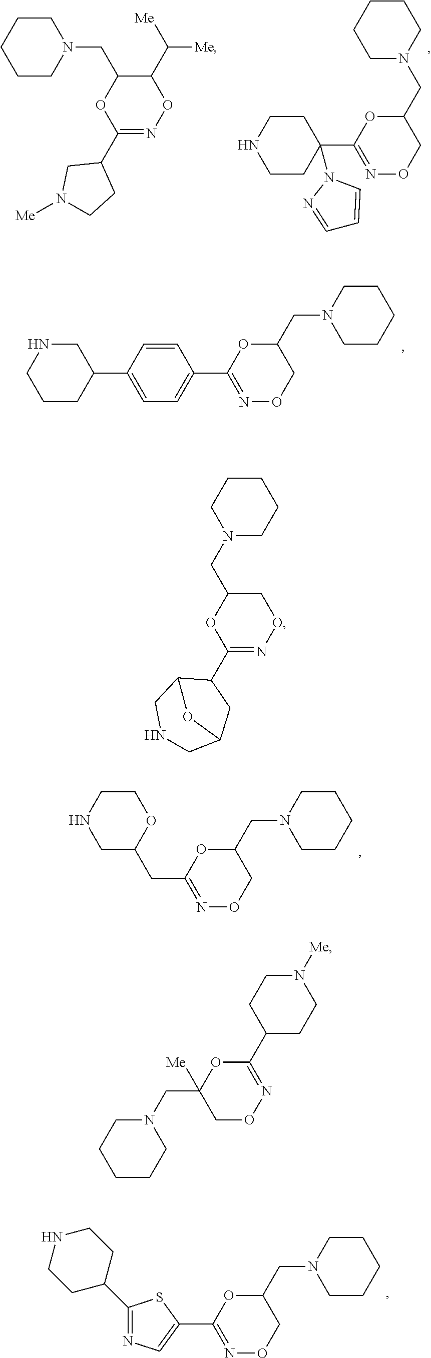

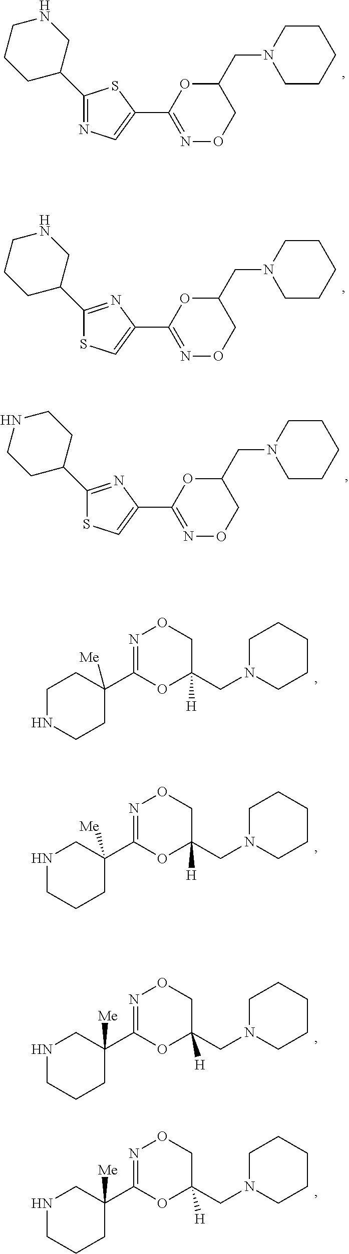

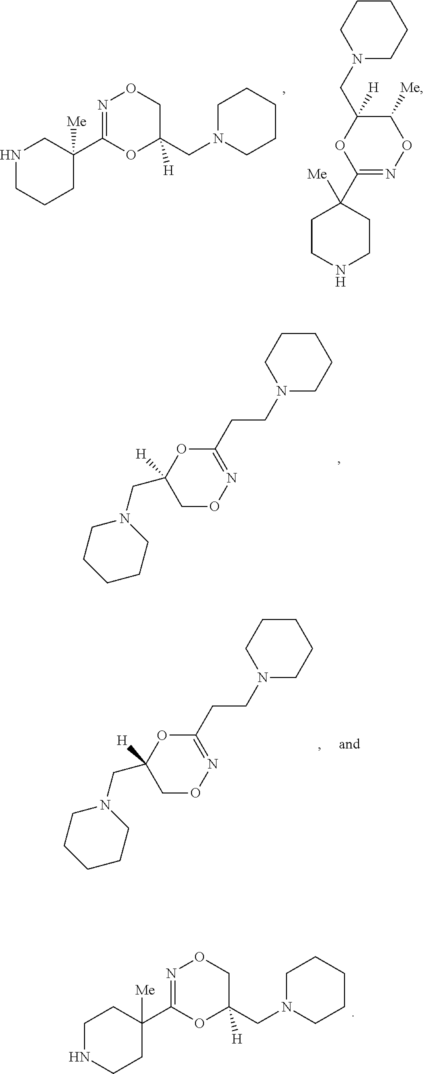 [SMILES: C[C@]1(C2=NOC[C@@H](CN3CCCCC3)O2)CCCNC1 ; C[C@@H]1ON=C(C2(C)CCNCC2)O[C@H]1CN1CCCCC1 ; C1CCN(CCC2=NOC[C@@H](CN3CCCCC3)O2)CC1 ; C1CCN(CCC2=NOC[C@H](CN3CCCCC3)O2)CC1 ; CC1(C2=NOC[C@@H](CN3CCCCC3)O2)CCNCC1]

### GBA Inducers

The compounds of the present disclosure are capable of inducing glucocerebrosidase (GBA) enzyme activity and/or GBA levels. Hence, the compounds of the present disclosure are GBA inducers, i.e. capable of inducing increased GBA enzyme levels and/or activity. In one embodiment, the compound provided is a GBA inducer.

In one embodiment, the compound is provided for use in a method of increasing GBA levels and/or activity. This effect can be readily determined using the assay provided in Example 2.

In one embodiment, the compound is provided which is capable of increasing said GBA activity at least 1.5-fold, such as at least 2-fold, for example at least 2.5-fold, such as at least 3-fold. In one embodiment, the method provides for increasing GBA activity at least 1.5-fold, such as at least 2-fold, for example at least 2.5-fold, such as at least 3-fold.

In one embodiment, the GBA activity is increased to 50% or more of hypothetical wild-type levels, such as 50-60%, such as 60-70%, such as 70-80%, such as 80-90%, such as 90-100%, such as 100-110%, such as 110-120%, such as 120-130%, such as 130-140%, such as 140-150% of hypothetical wild-type levels.

In one embodiment, the EC_{1.5} of the compound is 150 μM or less, such as 140 μM or less, such as 130 μM or less, such as 120 μM or less, such as 110 μM or less, such as 100 μM or less, such as 90 μM or less, such as 80 μM or less, such as 70 μM or less, such as 60 μM or less, preferably wherein the EC_{1.5} is 50 μM or less, such as 40 μM or less, such as 30 μM or less, such as 20 μM or less, such as 10 μM or less, such as 9 μM or less, such as 8 μM or less, such as 7 μM or less, such as 6 μM or less, such as 5 μM or less, such as 4 μM or less, such as 3 μM or less, such as 2 μM or less, such as 1 μM.

In one embodiment, the Emax % of the compound is 80% or more, such as 100% or more, such as 120% or more, such as 140% or more, such as 160% or more, such as 180% or more, such as 200% or more, such as 220% or more, such as 240% or more, such as 260% or more, such as 280% or more, such as 300% or more.

### Pharmaceutical Compositions

In one embodiment, a pharmaceutical composition is provided comprising a compound as defined herein, and one or more pharmaceutically acceptable adjuvants, excipients, carriers, buffers and/or diluents.

### Therapy

The compounds of the present disclosure are important for use in therapy. In one embodiment, a method for treating a disease in a subject comprising administering a compound as defined herein is provided, wherein the disease is associated with reduced GBA levels and/or activity.

In one embodiment, the method is provided wherein the disease treated is Parkinson's disease (PD). In one embodiment, a compound as defined herein is provided for use in the treatment of Parkinson's disease.

In one embodiment, use of a compound as defined herein is provided for the manufacture of a medicament for the treatment of Parkinson's disease (PD).

### Items

1. A compound of formula (Ia),

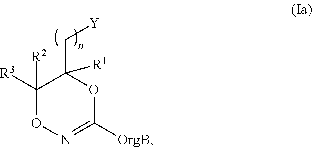

or a pharmaceutically acceptable salt thereof; wherein
n is 1 or 2;
R¹, R², and R³ are independently selected from the group consisting of: hydrogen, alkyl, and halogen;
Y is a nitrogen-containing ring or a nitrogen-containing chain;
OrgB is an organic basic moiety attached via a sp³ hybridised carbon to the rest of the compound; and
OrgB and Y are optionally substituted.

2. The compound according to any one of the preceding items, wherein the compound is of formula (Ib),

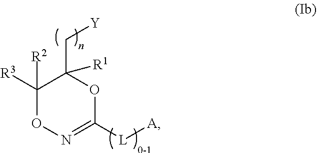

wherein
A is a monocyclic ring, a bicyclic ring, or a tricyclic ring, and A is attached via a sp³ hybridised carbon to the rest of the compound;
L is a C₁₋₆ alkyl linker or L is absent; if L is absent, A is directly attached to the cyclic oxime; and
A and Y are optionally substituted.

3. The compound according to any one of the preceding items, wherein A is of formula (II):

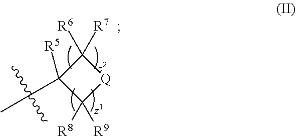

wherein
z¹ and z² are independently selected from the group consisting of: 0, 1, 2, and 3;
Q is selected from the group consisting of:

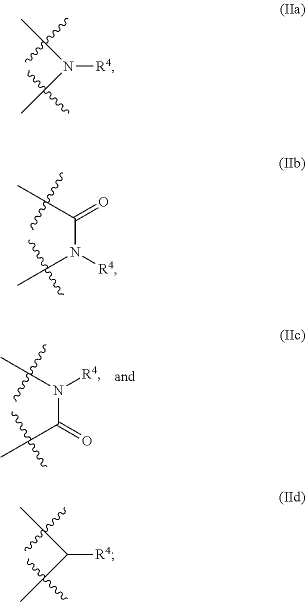

R⁴ is selected from the group consisting of: hydrogen, alkyl, amino, alkoxy, acyl, amido, aralkyl, aryl, heteroaryl, cycloalkyl, heterocycloalkyl;
R⁵ is selected from the group consisting of: hydrogen, alkyl, aralkyl, hydroxy, alkoxy, and amino;
each R⁶ is independently selected from the group consisting of: hydrogen, alkyl, alkoxy, hydroxy, amino, amido, and halogen;
each R⁷ is independently selected from the group consisting of: hydrogen, alkyl, alkoxy, hydroxy, amino, amido, and halogen;
each R⁸ is independently selected from the group consisting of: hydrogen, alkyl, alkoxy, hydroxy, amino, amido, and halogen; and
each R⁹ is independently selected from the group consisting of: hydrogen, alkyl, alkoxy, hydroxy, amino, amido, and halogen.

4. The compound according to any one of the preceding items, wherein A is of formula (II) and Q is of formula (IIa).

5. The compound according to any one of the preceding items, wherein L is of formula (III)

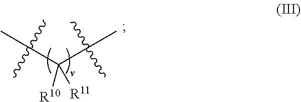

wherein
v is 0 or 1; if v is 0, L is absent;
each R¹⁰ is independently selected from the group consisting of: hydrogen and alkyl;
each R¹¹ is independently selected from the group consisting of: hydrogen and alkyl;
if both R¹⁰ and R¹¹ are alkyl, R¹⁰ and R¹¹ are optionally connected to form a C₃₋₆ ring.

6. The compound according to any one of the preceding items, wherein R⁵ is selected from the group consisting of:

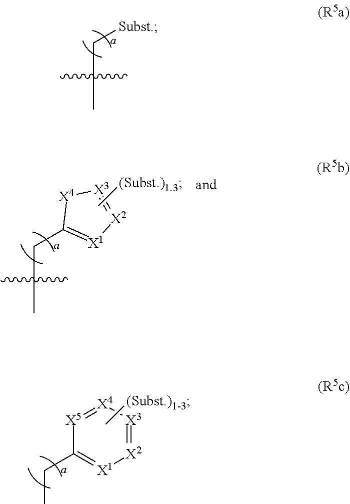

or any tautomer thereof,
wherein a is 0, 1, 2, or 3;
X¹, X², X³, X⁴, and X⁵ independently are selected from the group consisting of: C, CH, and N; and
each one, two, or three Subst. is independently selected from the group consisting of: hydrogen, alkyl, halogen, hydroxy, alkoxy, amino, amido, acyl, cycloalkyl, and heterocycloalkyl.

7. The compound according to any one of the preceding items, wherein A is selected from the group consisting of: a monocyclic ring and a bicyclic ring.

8. The compound according to any one of the preceding items, wherein A comprises 1, 2 or 3 nitrogen atoms.

9. The compound according to any one of the preceding items, wherein A comprises 0, 1, 2 or 3 oxygen atoms.

10. The compound according to any one of the preceding items, wherein A is a cycle comprising 5-10 ring atoms.

11. The compound according to any one of the preceding items, wherein A is a C₅₋₉ heterocycle.

12. The compound according to any one of the preceding items, wherein A is a C₅₋₉ bicyclic heterocycle comprising pyrrolidine.

13. The compound according to any one of the preceding items, wherein A is of formula (II) and L is of formula (III), wherein L-A is selected from the group consisting of:

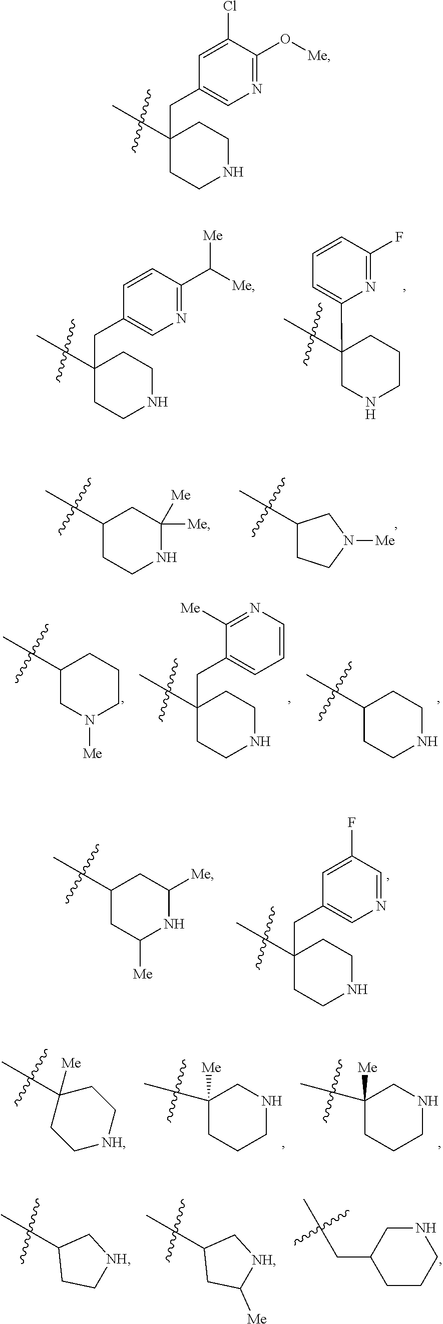

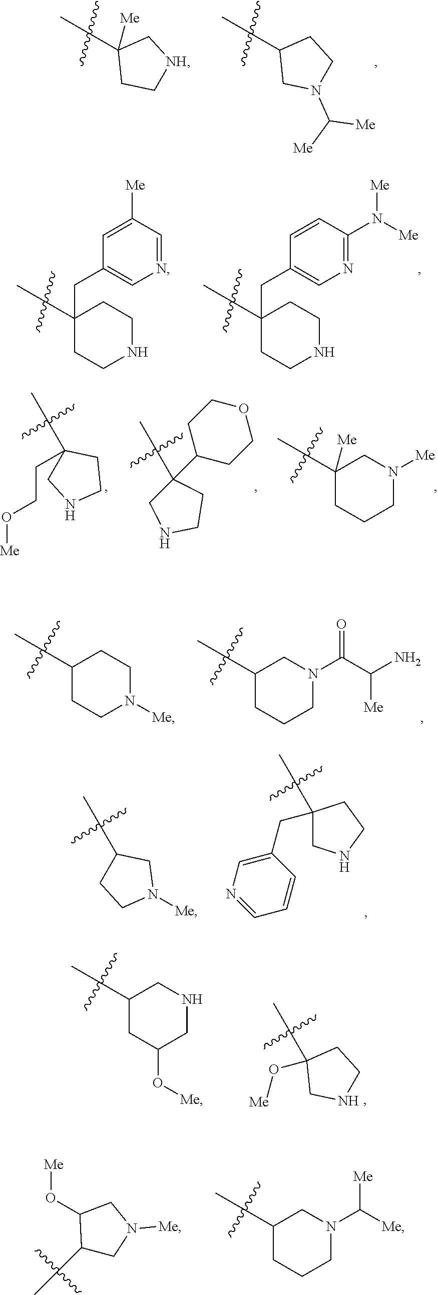

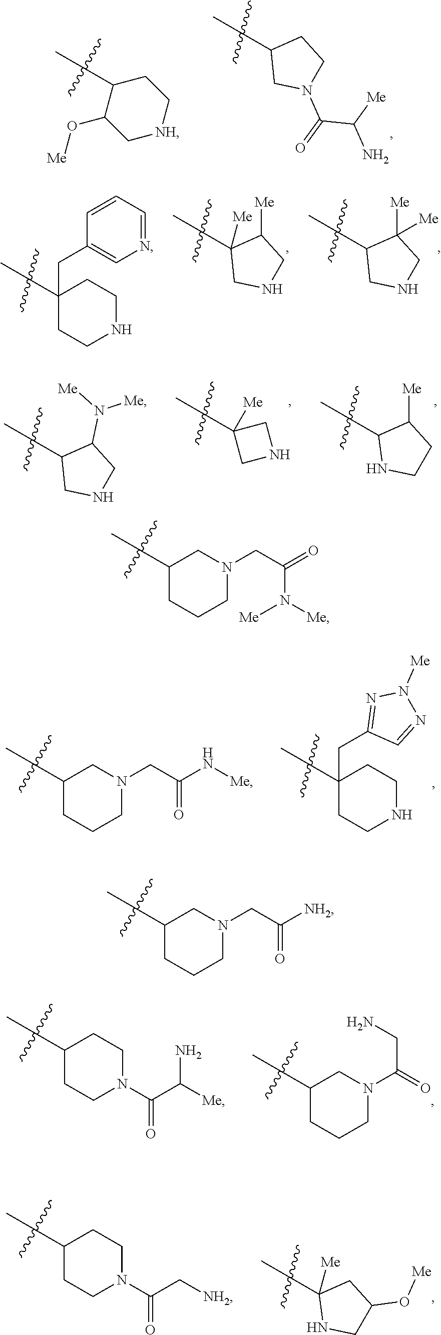

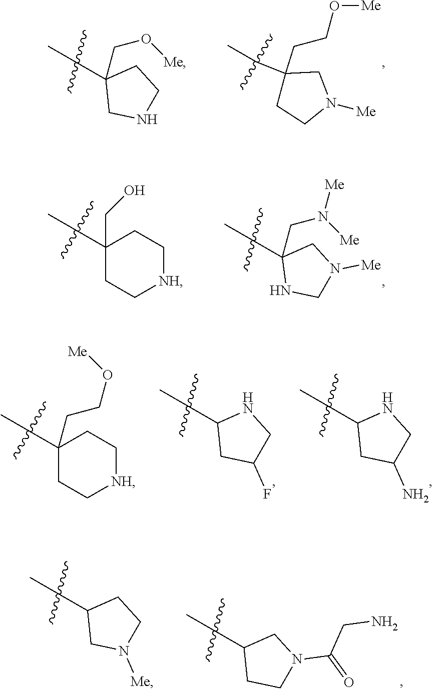

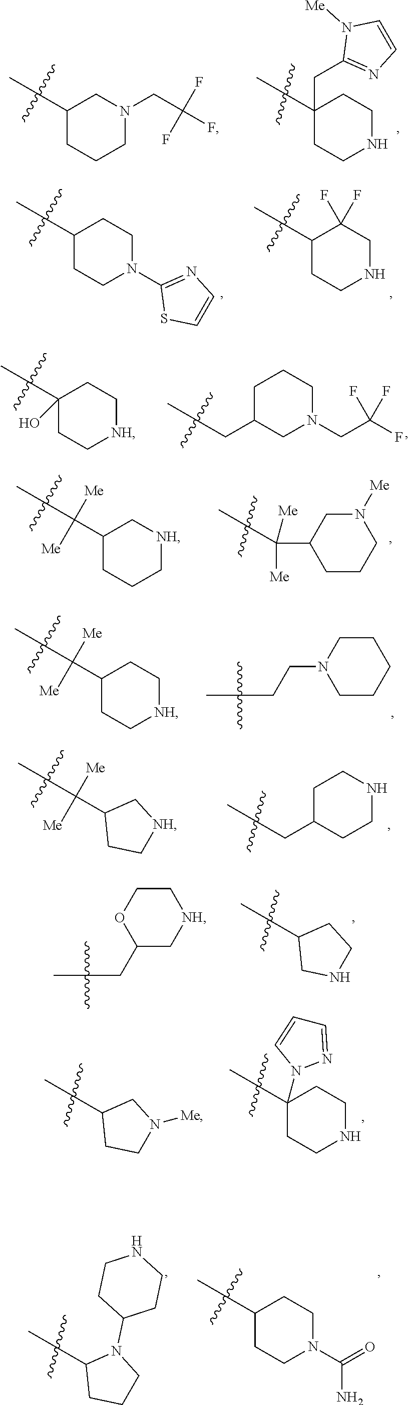

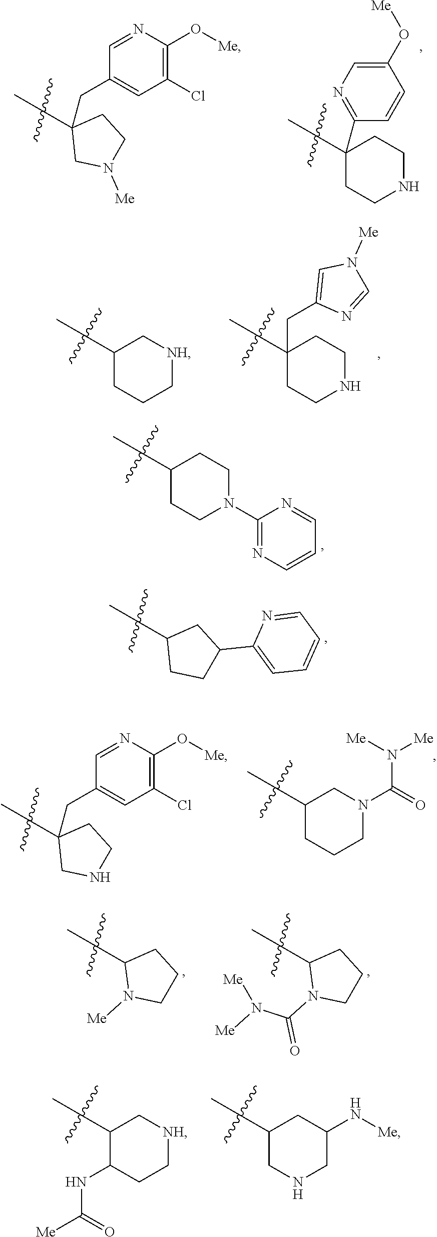

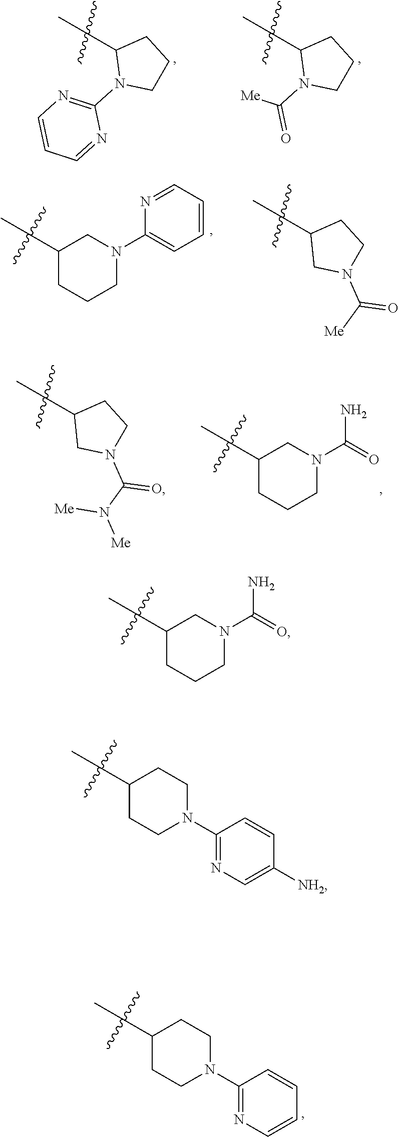

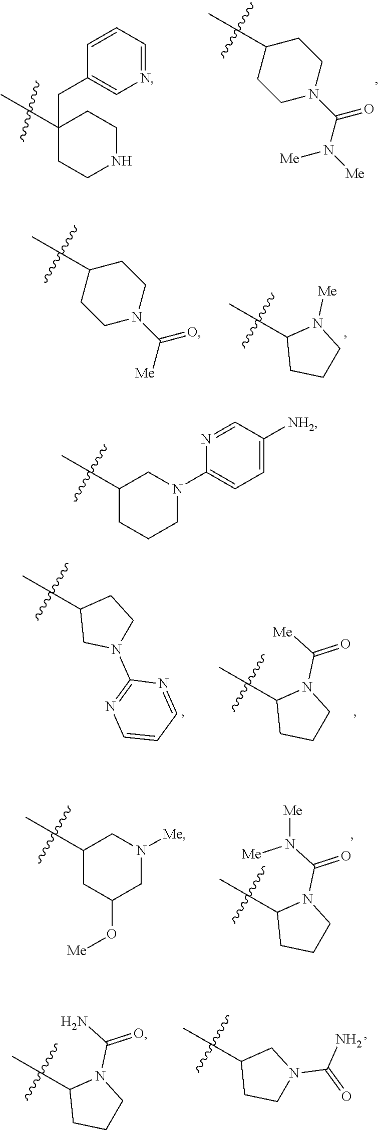

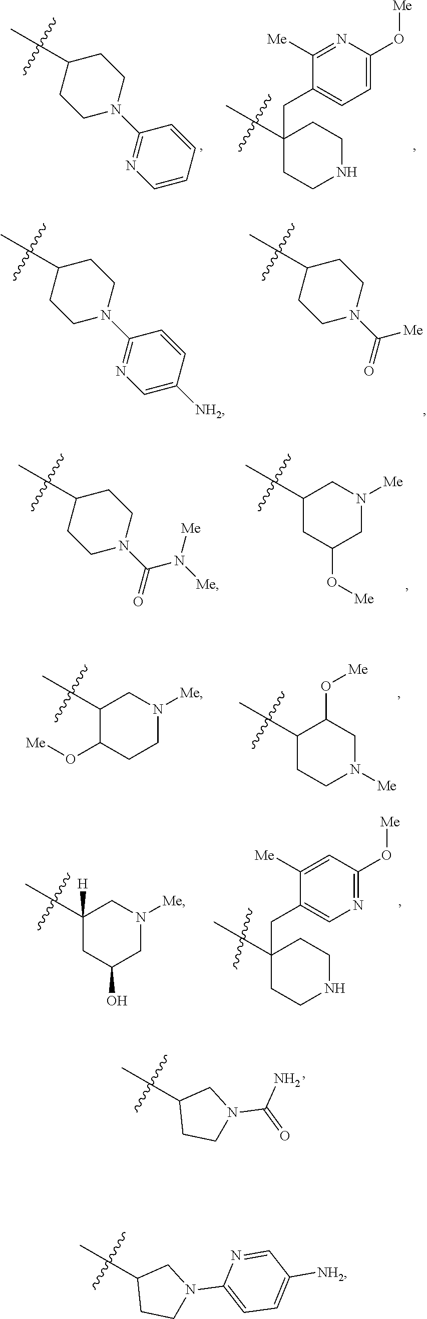

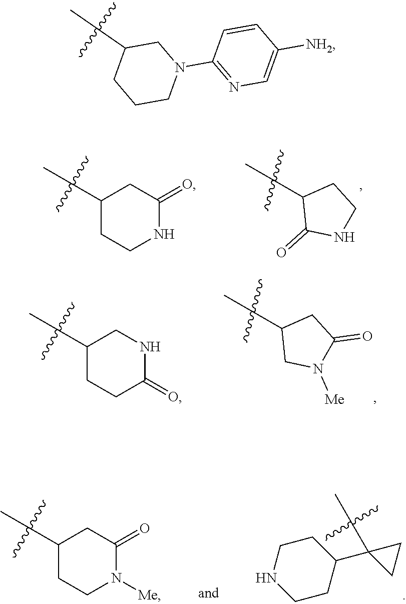

14. The compound according to any one of the preceding items, wherein A is selected from the group consisting of:

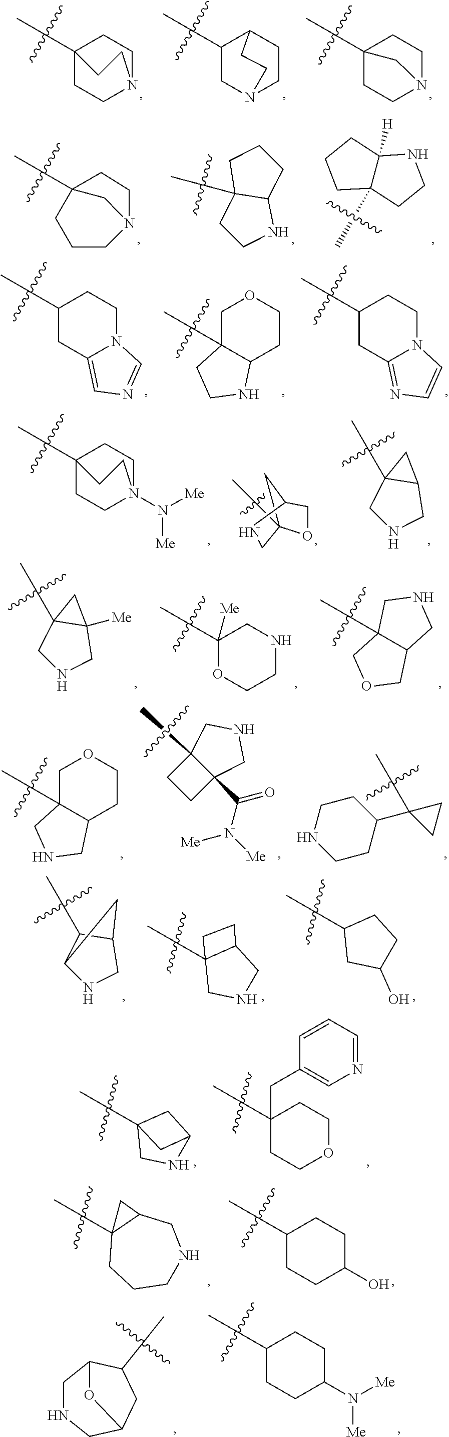

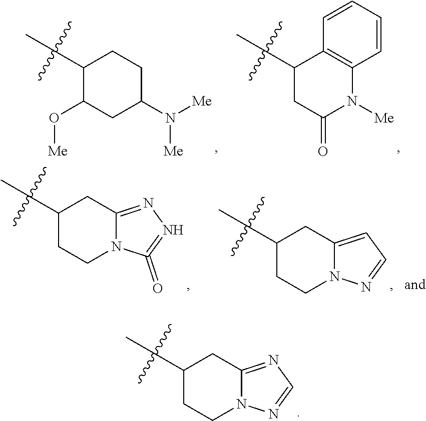

15. The compound according to any one of the preceding items, wherein OrgB is selected from the group consisting of:

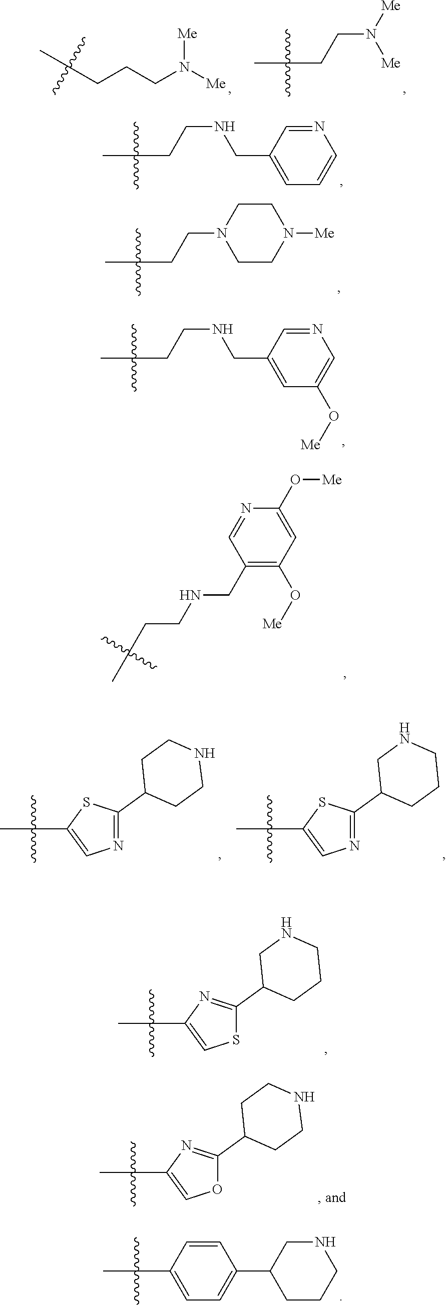

16. The compound according to any one of the preceding items, wherein Y is a nitrogen-containing ring, wherein the nitrogen-containing ring is monocyclic or bicyclic.

17. The compound according to any one of the preceding items, wherein Y is an optionally substituted piperidine, such a piperidine substituted by one, two, three or four methyl groups.

18. The compound according to any one of the preceding items, wherein Y is an optionally substituted pyrrolidine.

19. The compound according to any one of the preceding items, wherein Y is an optionally substituted piperazine.

20. The compound according to any one of the preceding items, wherein Y is selected from the group consisting of:

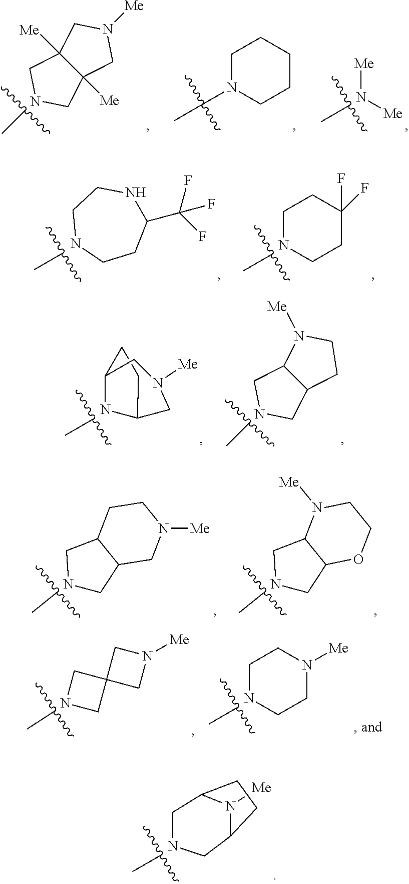

21. The compound according to any one of the preceding items, wherein R¹, R², and R³ independently are selected from the group consisting of: hydrogen and alkyl.

22. The compound according to any one of the preceding items, wherein R¹ and R² are both hydrogen, and R³ is C₁₋₆ alkyl.

23. The compound according to any one of the preceding items, wherein R³ is selected from the group consisting of: methyl, ethyl, propyl, isopropyl, cyclopropyl, cyclobutyl, cyclopentyl, and cyclohexyl.

24. The compound according to any one of the preceding items, wherein R³ is selected from the group consisting of: methyl, isopropyl, and cyclohexyl.

25. The compound according to any one of the preceding items, wherein R⁴ is selected from the group consisting of: hydrogen and alkyl.

26. The compound according to any one of the preceding items, wherein R⁴ is alkyl, such as C₁₋₆ alkyl.

27. The compound according to any one of the preceding items, wherein R⁴ is selected from the group consisting of methyl, ethyl, propyl, butyl, isopropyl, sec-butyl, cyclopropyl, cyclobutyl, and cyclopentyl.

28. The compound according to any one of the preceding items, wherein R⁴ is methyl.

29. The compound according to any one of the preceding items, wherein R⁵ is hydrogen or methyl.

30. The compound according to any one of the preceding items, wherein R⁶ and R⁷ are both hydrogen.

31. The compound according to any one of the preceding items, wherein R⁸ and R⁹ are both hydrogen.

32. The compound according to any one of the preceding items, wherein z¹ and z² are both 2.

33. The compound according to any one of the preceding items, wherein z¹ and z² are both 2, and wherein R⁶, R⁷, R⁸, and R⁹ are hydrogen.

34. The compound according to any one of the preceding items, wherein z¹ and z² are 2, and wherein R⁶, R⁷, R⁸, and R⁹ are hydrogen, and wherein R⁵ is methyl.

35. The compound according to any one of the preceding items, wherein the compound is selected from the group consisting of:

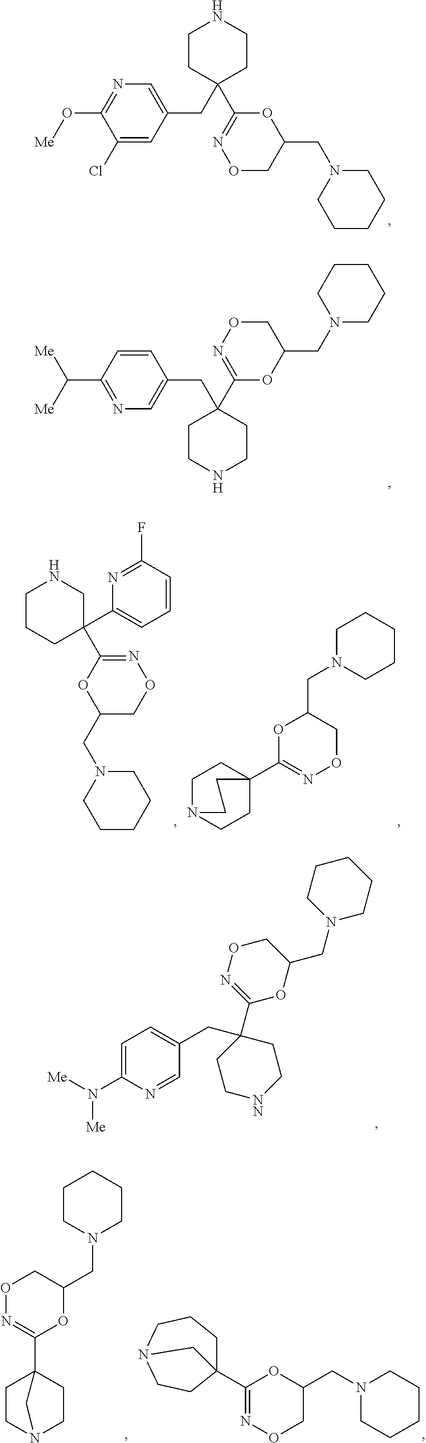

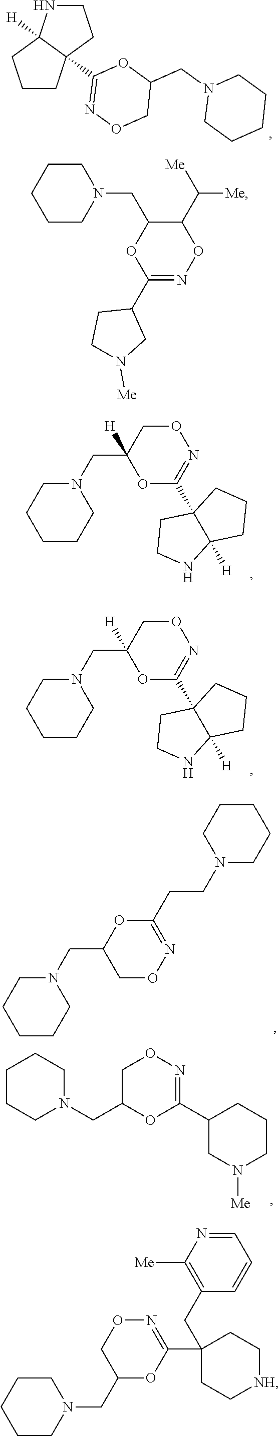

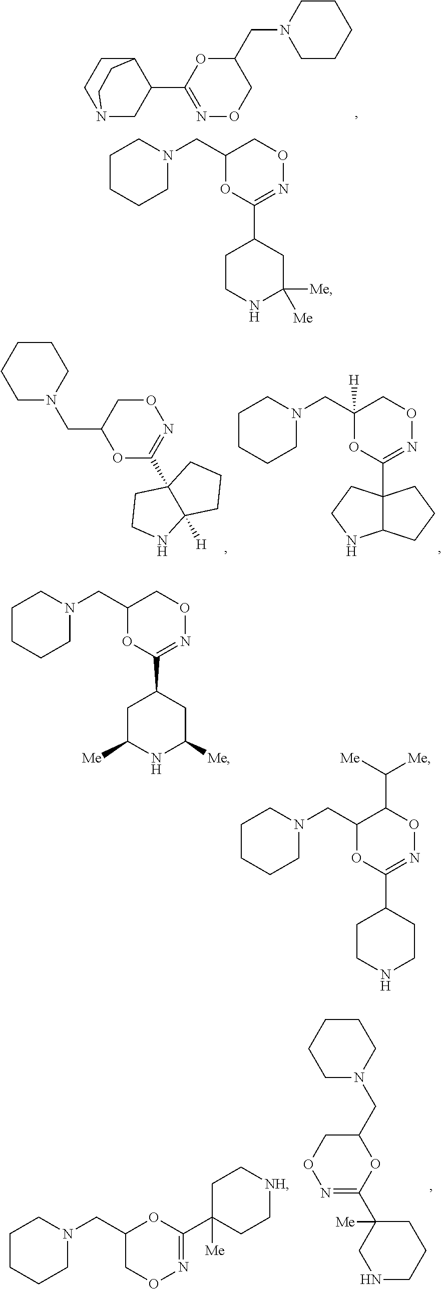

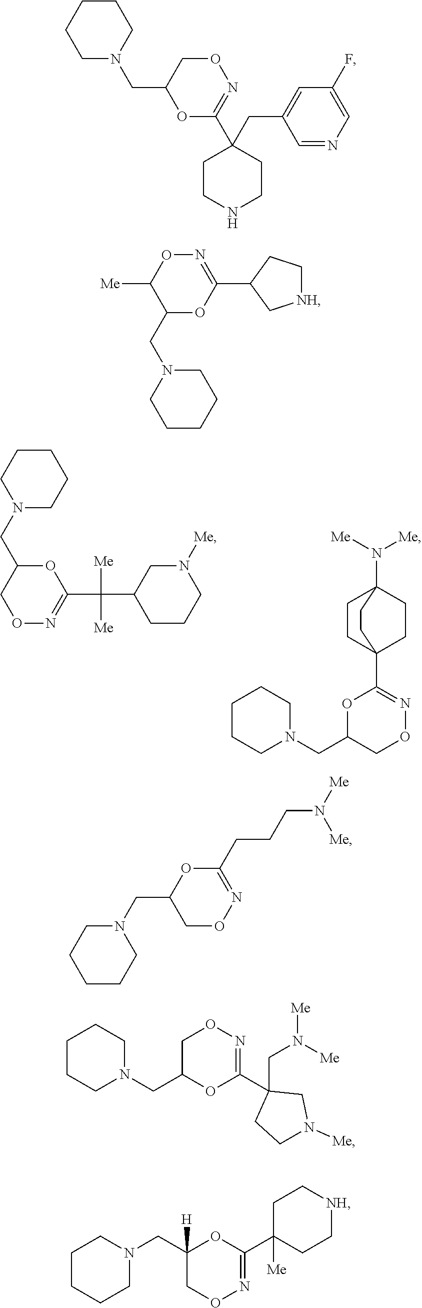

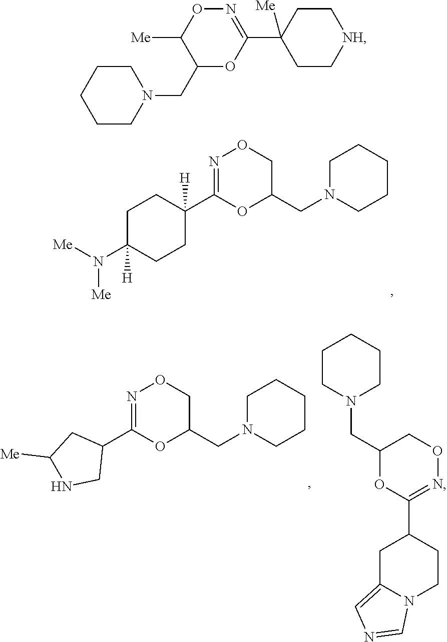

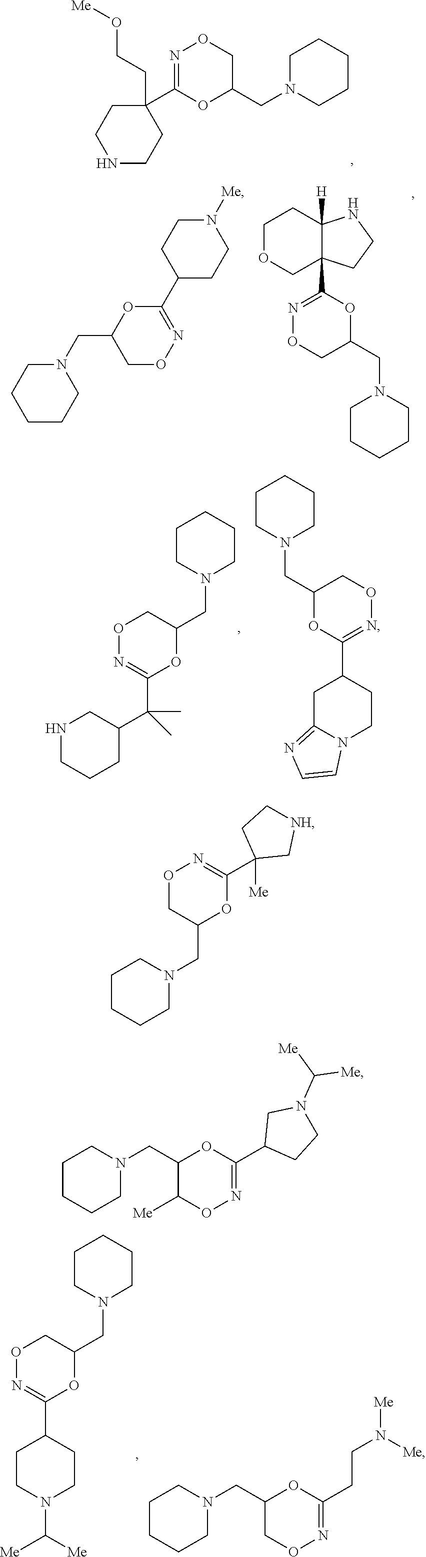

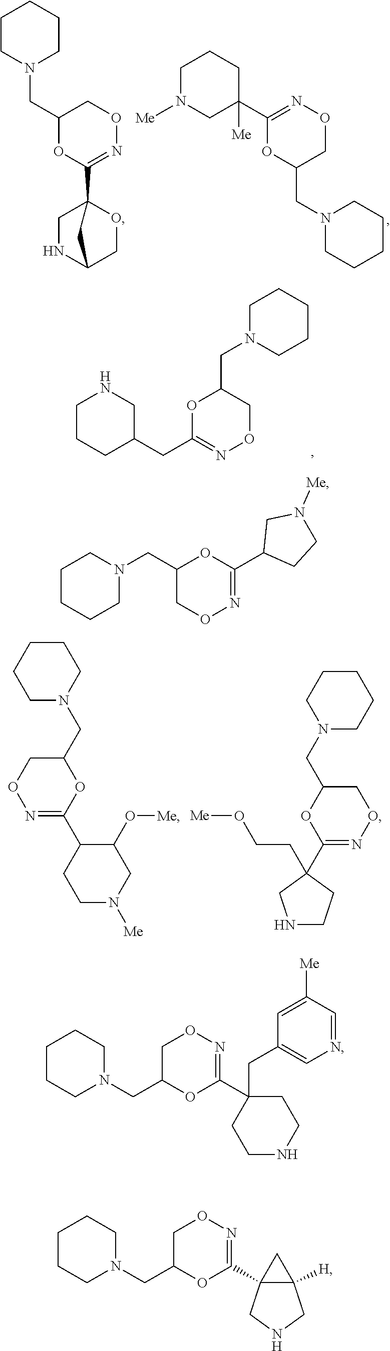

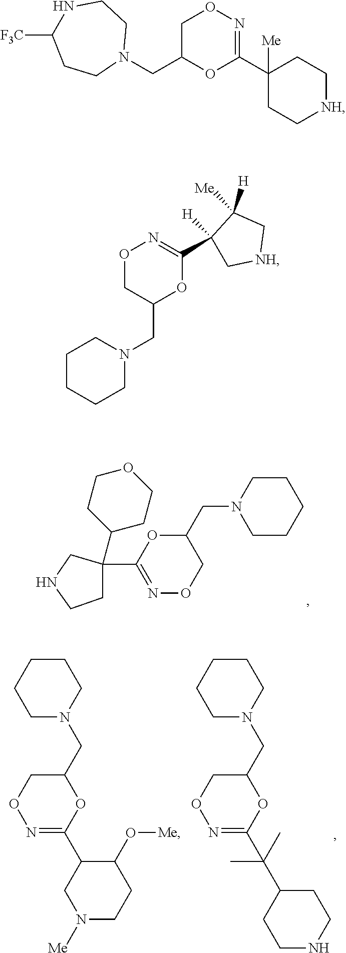

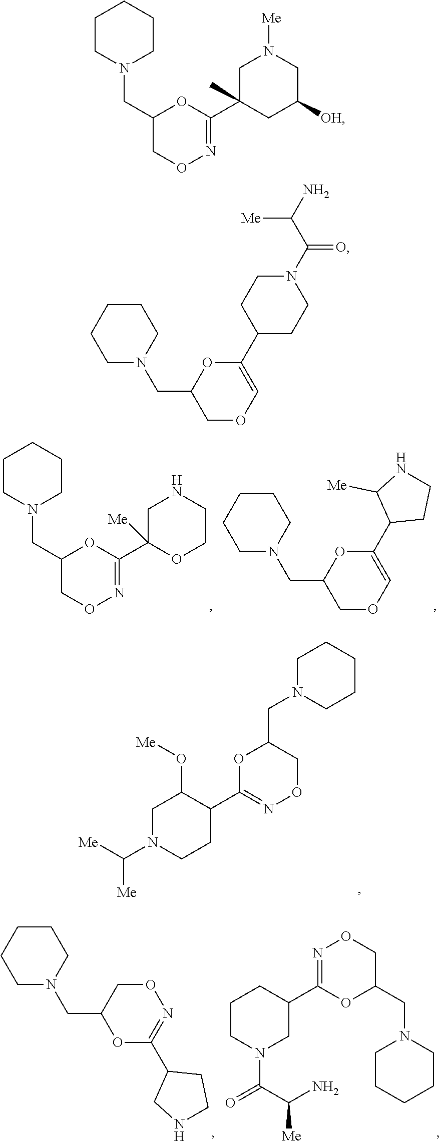

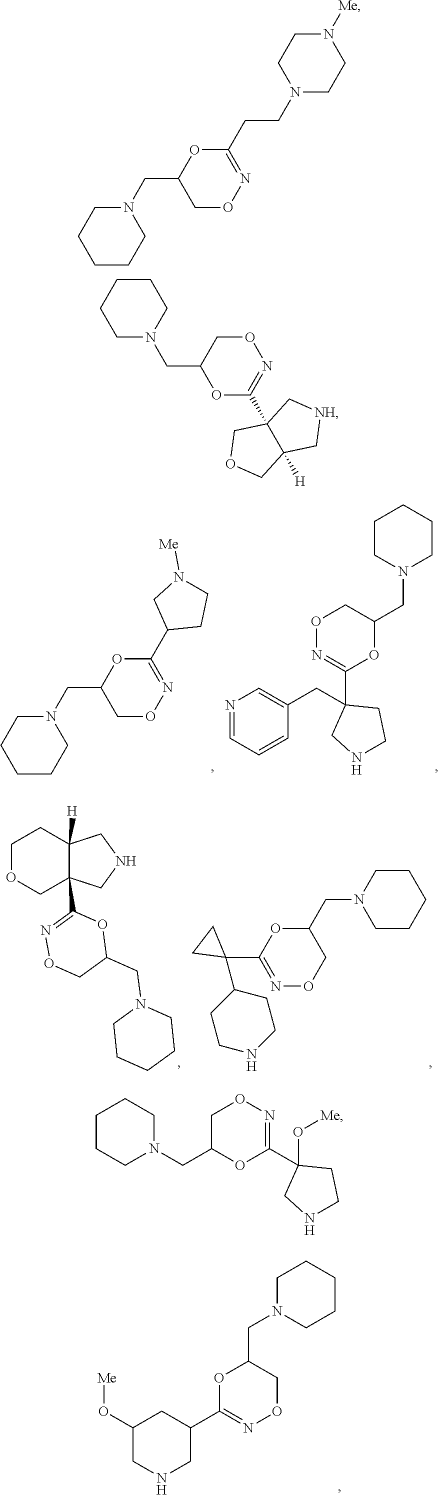

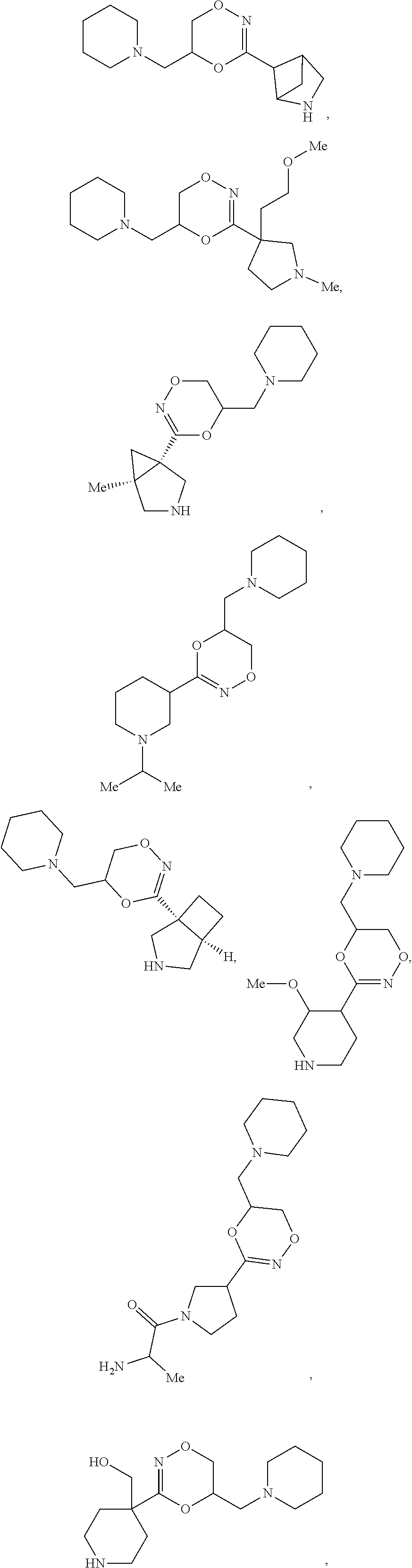

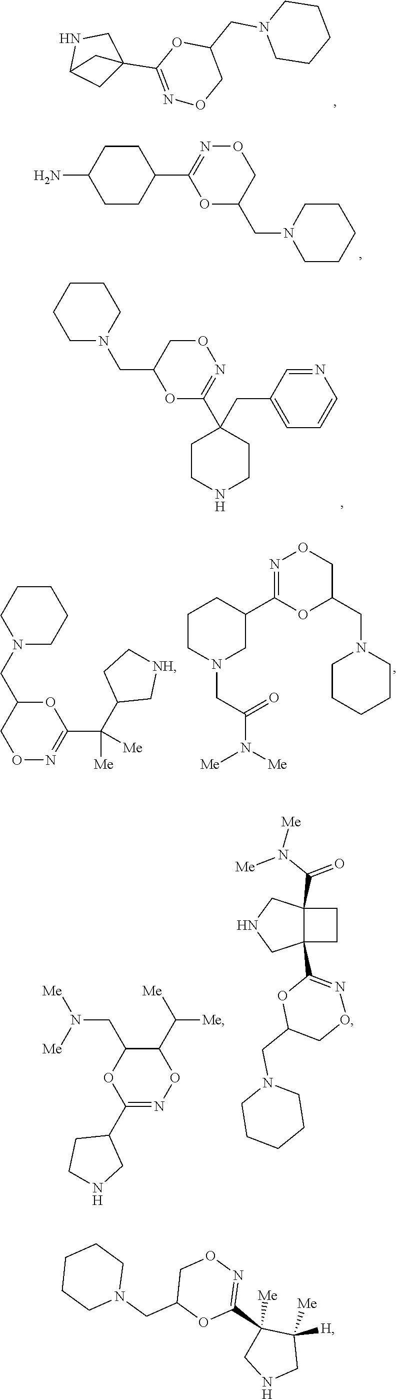

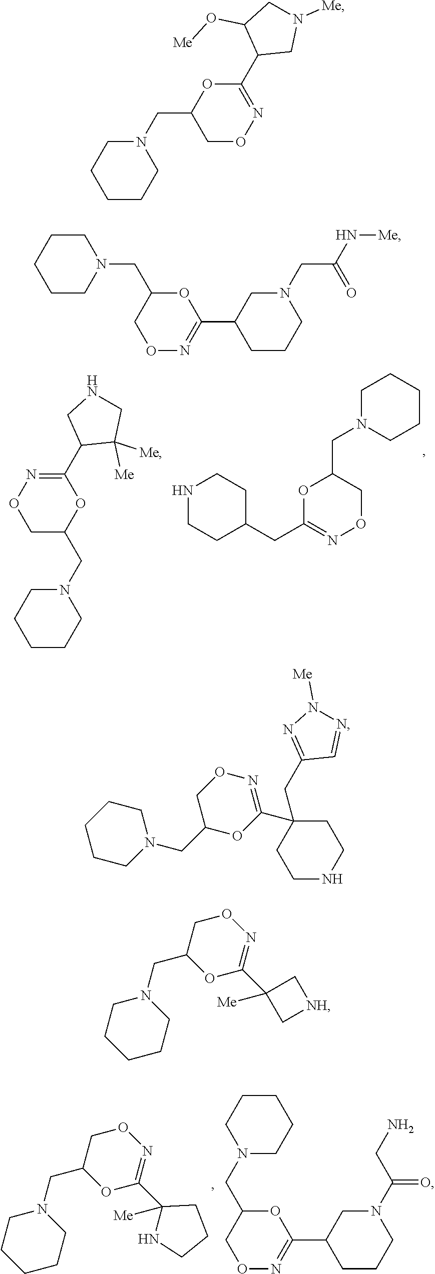

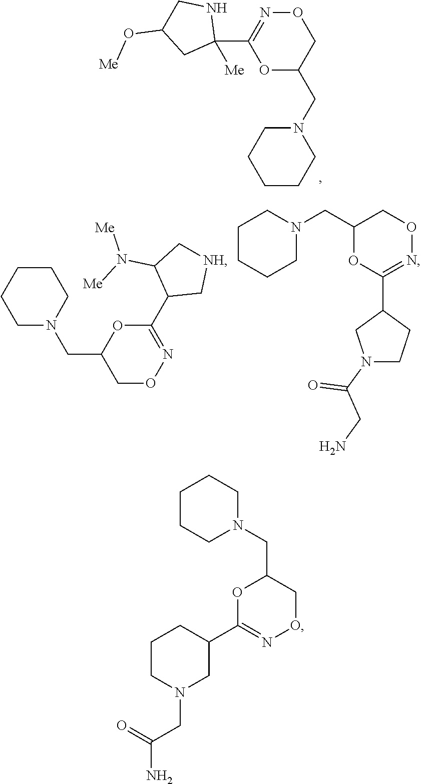

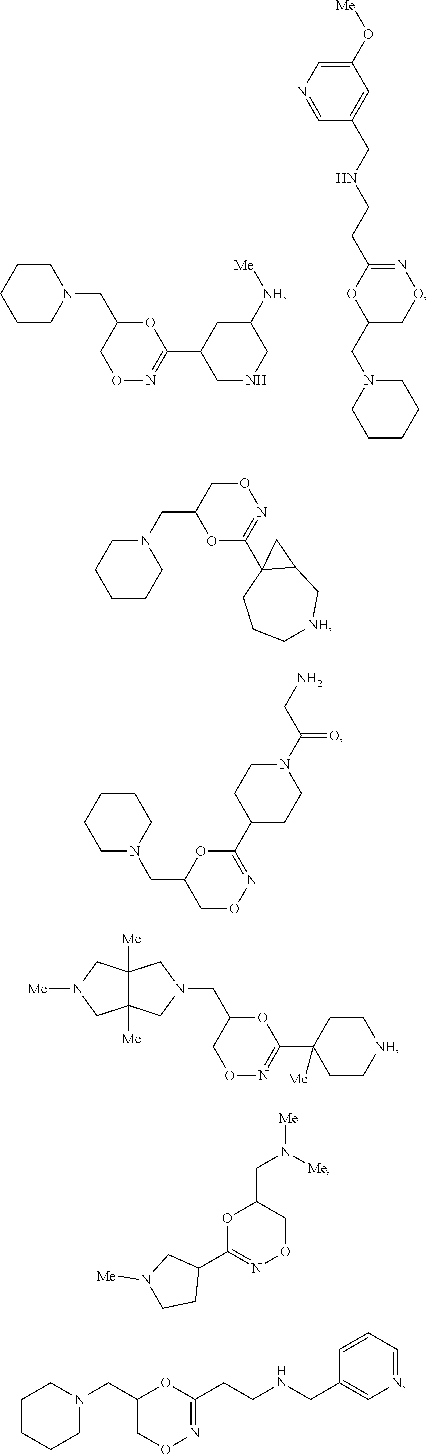

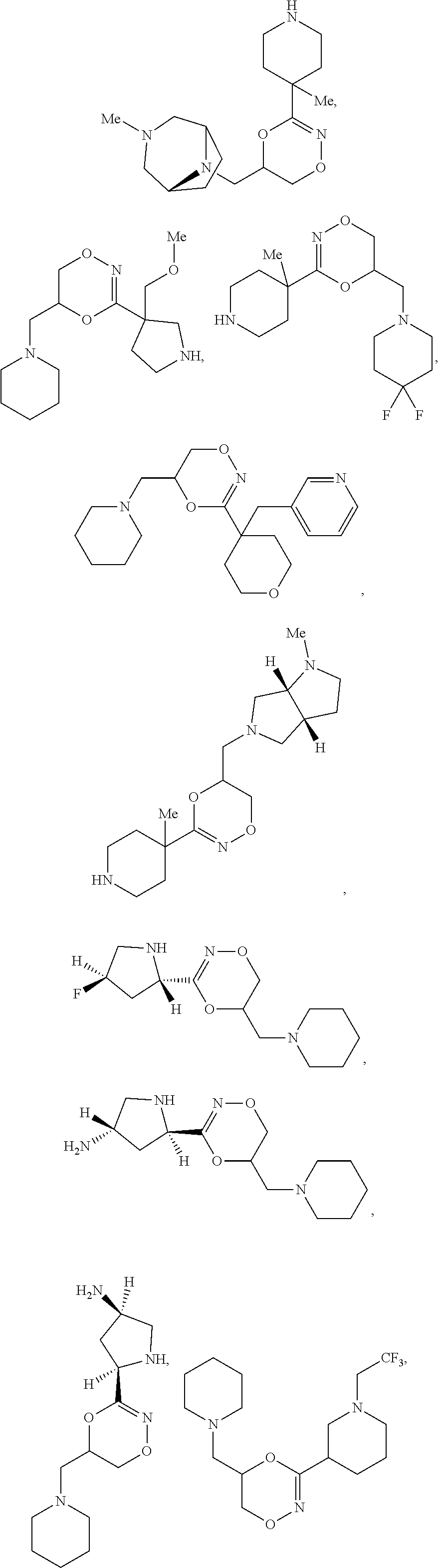

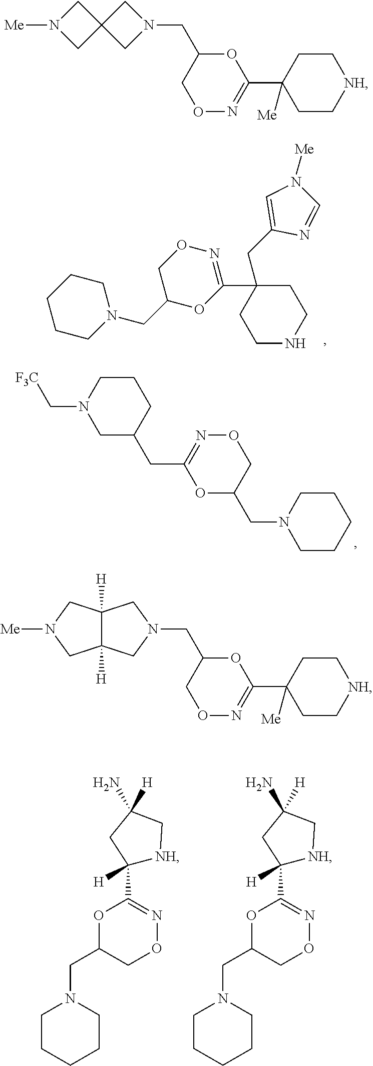

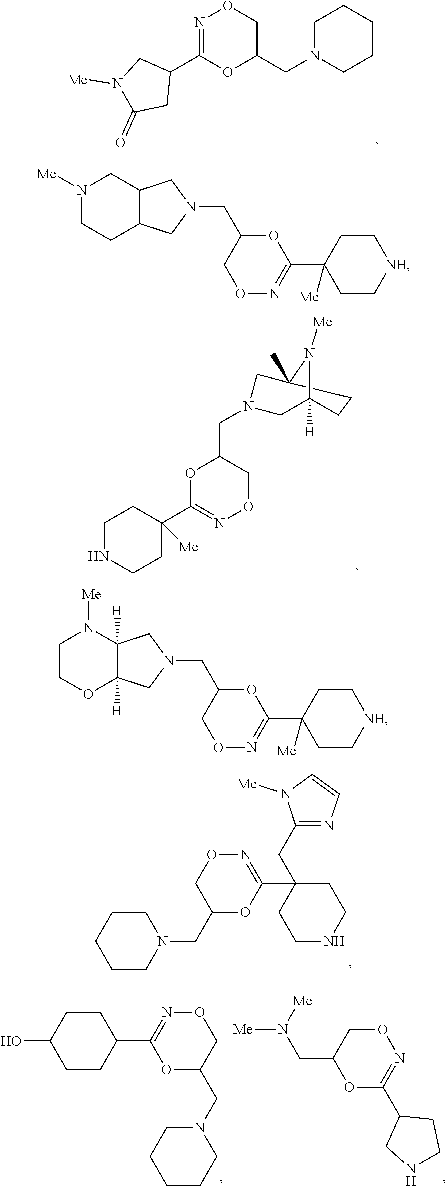

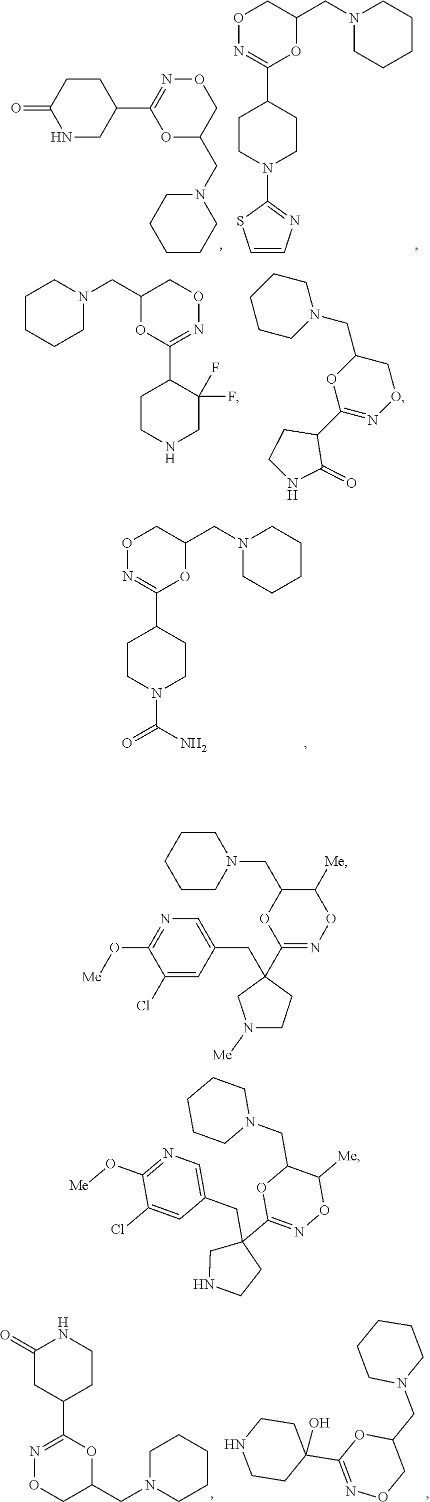

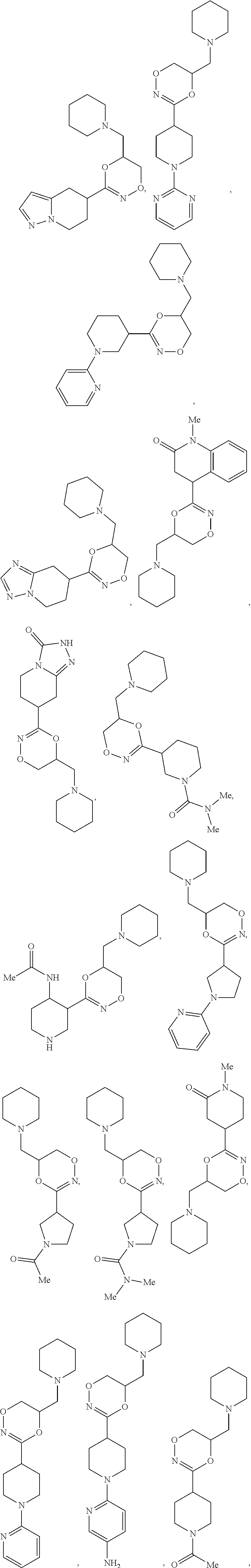

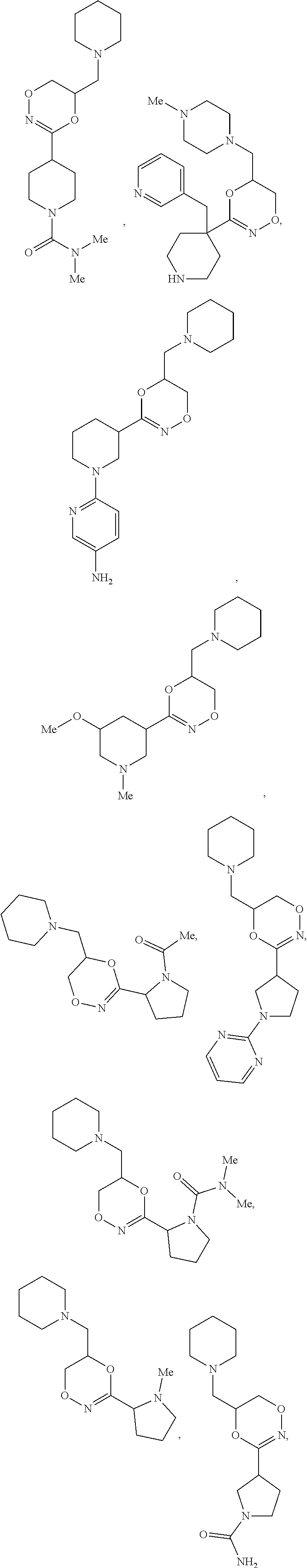

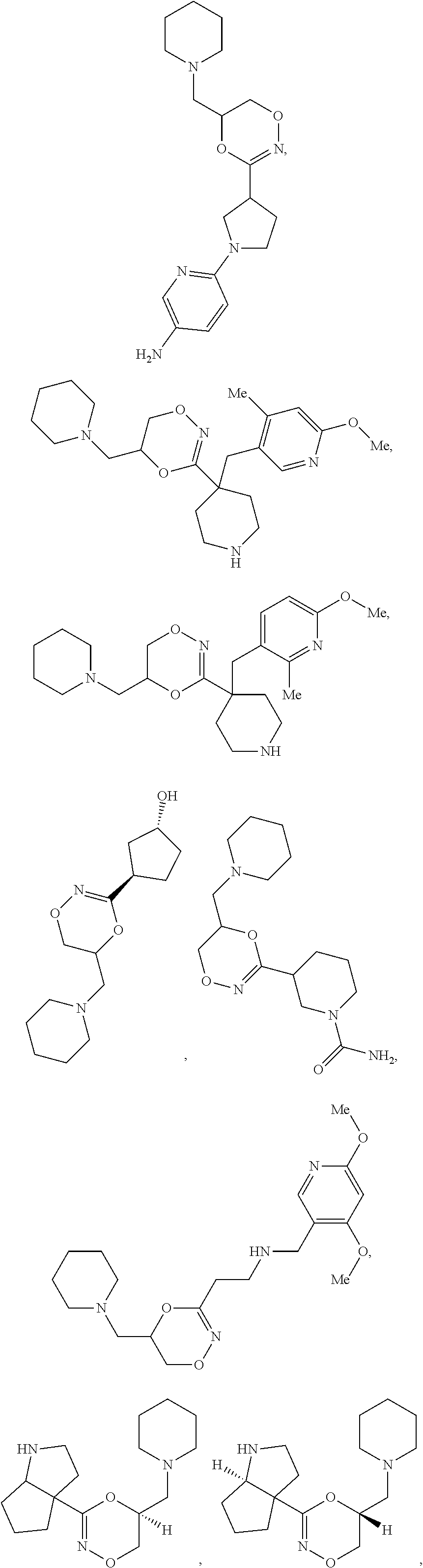

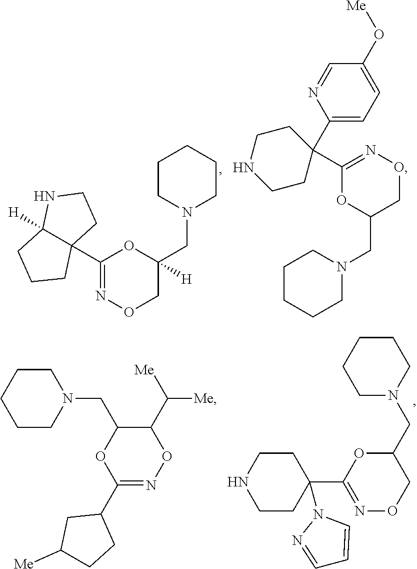

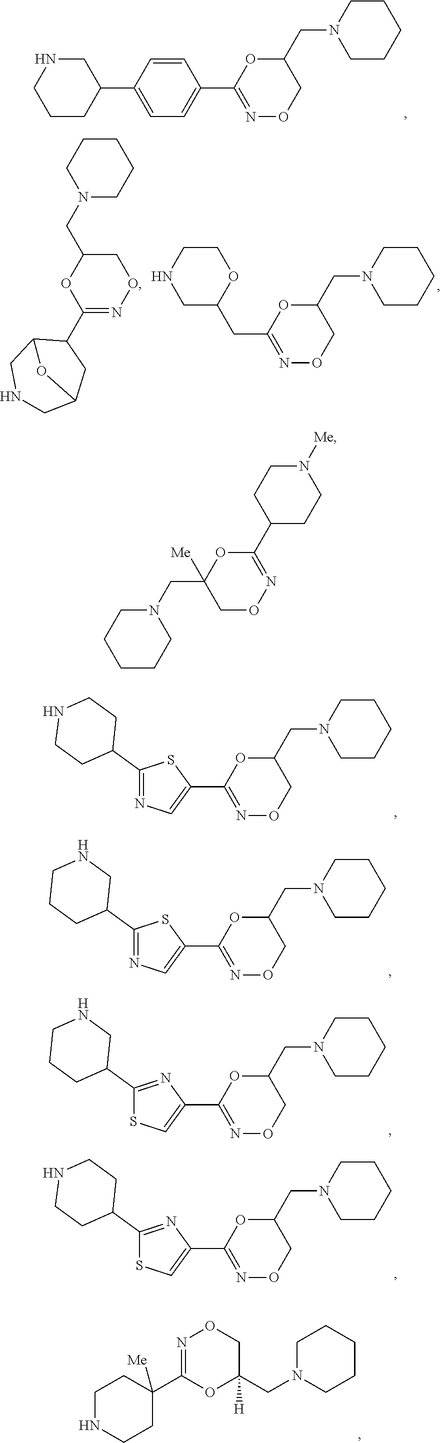

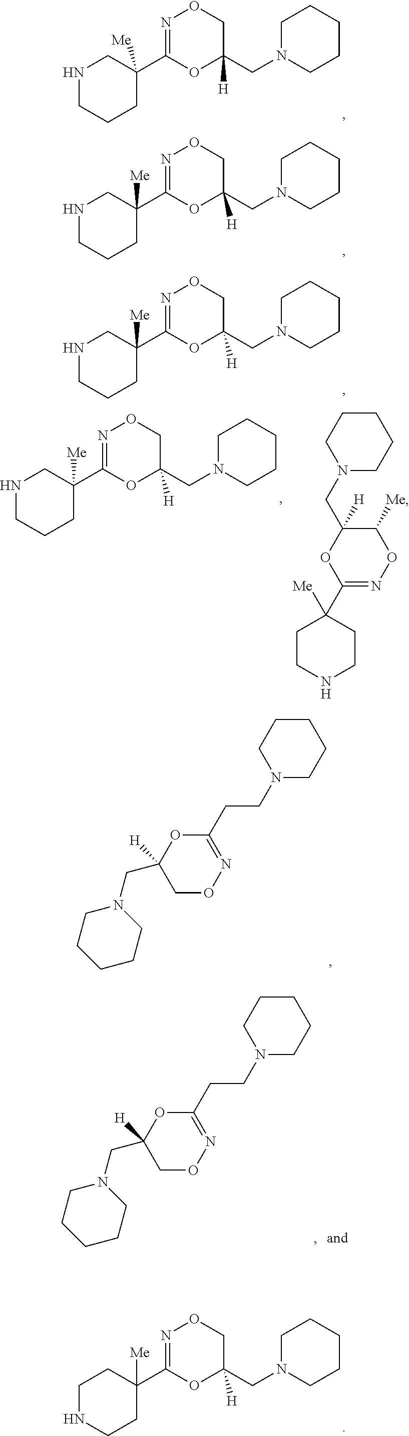

36. The compound according to any one of the preceding items, wherein the compound increases glucocerebrosidase (GBA) enzyme levels and/or GBA enzyme activity.

37. The compound according to any one of the preceding items, wherein the compound is a GBA inducer.

38. The compound according to any one of the preceding items, for use in a method of increasing GBA levels and/or activity.

39. The compound for use according to any one of the preceding items, wherein said GBA activity is increased at least 1.5-fold, such as at least 2-fold, for example at least 2.5-fold, such as at least 3-fold.

40. The compound for use according to any one of the preceding items, wherein said GBA activity is increased to 50% or more of hypothetical wild-type levels, such as 50-60%, such as 60-70%, such as 70-80%, such as 80-90%, such as 90-100%, such as 100-110%, such as 110-120%, such as 120-130%, such as 130-140%, such as 140-150% of hypothetical wild-type levels.

41. The compound for use according to any one of the preceding items, wherein the EC_{1.5} of the compound is 150 μM or less, such as 140 μM or less, such as 130 μM or less, such as 120 μM or less, such as 110 μM or less, such as 100 μM or less, such as 90 μM or less, such as 80 μM or less, such as 70 μM or less, such as 60 μM or less, preferably wherein the EC_{1.5} is 50 μM or less, such as 40 μM or less, such as 30 μM or less, such as 20 μM or less, such as 10 μM or less, such as 9 μM or less, such as 8 μM or less, such as 7 μM or less, such as 6 μM or less, such as 5 μM or less, such as 4 μM or less, such as 3 μM or less, such as 2 μM or less, such as 1 μM.

42. The compound for use according to any one of the preceding items, wherein the Emax % of the compound is 80% or more, such as 100% or more, such as 120% or more, such as 140% or more, such as 160% or more, such as 180% or more, such as 200% or more, such as 220% or more, such as 240% or more, such as 260% or more, such as 280% or more, such as 300% or more.

43. A pharmaceutical composition comprising a compound as defined in any one of the preceding items, and one or more pharmaceutically acceptable adjuvants, excipients, carriers, buffers and/or diluents.

44. A method for treating a disease in a subject comprising administering a compound as defined in any one of the preceding items, wherein the disease is associated with reduced GBA levels and/or activity.

45. The method according to any one of the preceding items, wherein the disease is Parkinson's disease (PD).

46. A method of increasing the GBA activity and/or levels comprising contacting GBA with a compound as defined in any one of the preceding items.

47. Use of a compound as defined in any one of the preceding items, for the manufacture of a medicament for the treatment of Parkinson's disease (PD).

### EXAMPLES

### Example 1: Synthesis of Dioxazines

### Materials and Abbreviations

| | |
|---|---|
| DCM | Dichloromethane |
| DCE | Dichloroethane |
| TFA | Trifluoroacetic acid |
| CDI | Carbonyldiimidazole |
| MeCN | Acetonitrile |
| HOAc | Acetic acid |
| MeOH | Methanol |
| H₂O | Water |
| NaBH₃CN | Sodium cyanoborohydride |
| NBS | 1-Bromo-2,5-pyrrolidinedione |
| K₂CO₃ | Potassium carbonate |
| DIPEA | N,N-Diisopropylethylamine |
| TEA | N,N-Diethylethanamine |
| HATU | 1-[Bis(dimethylamino)methylene]-1H- |
| | 1,2,3-triazolo[4,5-b]pyridinium 3-oxide |
| | hexafluorophosphate |
| DMF | Dimethylformamide |
| NaOH | Sodium hydroxide |
| THF | Tetrahydrofuran |
| LiHMDS | Lithium bis(trimethylsilyl)amide |
| DIAD | Diisopropyl azodicarboxylate |
| PPh₃ | Triphenylphosphine |
| TBAF | Tetra-n-butylammonium fluoride |
| NaBH(OAc)₃ | Sodium triacetoxyboranuide |
| MeNH₂ | Methanamine |
| Boc₂O | Di-tert-butyl dicarbonate |
| Paraform | Paraformaldehyde |
| t-BuONO | tert-Butyl nitrite |
| CuCl₂ | Copper (II) chloride |
| IPA | Propan-2-ol |
| NH₂OH*HCl | Hydroxylamine hydrochloride |
| Rac | Racemic (Some compounds may contain |
| | both known and unknown stereochemistry) |
| rel | “Relative”, in the meaning the exact enantio- |
| | isomeri or absolute configuration is unknown and |
| | the structures shown are proposals but may be |
| | enantiomers, diasteromers, or epimers of the |
| | proposed structure. For instance: rel-trans |
| | means that there's a known trans relationship, |
| | of (relative) unknown configuration. |
| r.t. | Room temperature |
| trans | trans-configuration |
| cis | cis-configuration |

A straight line towards a chiral center in the schemes and structures below indicate a material is racemic. If nothing else is noted, the structures are racemates.

Analytical and preparative instruments used. One or more of the following instruments were used in the process of analyzing composition of isolated material:

### LC/MS

### Instrument Specifications:

Agilent 1100 Series LC/MSD system with DAD\ELSD Alltech 2000ES and Agilent LC\MSD VL (G1956B), SL (G1956B) mass-spectrometer.
Agilent 1200 Series LC/MSD system with DAD\ELSD Alltech 3300 and Agilent LC\MSD G6130A, G6120B mass-spectrometer.
Agilent Technologies 1260 Infinity LC/MSD system with DAD\ELSD Alltech 3300 and Agilent
LC\MSD G6120B mass-spectrometer.
Agilent Technologies 1260 Infinity II LC/MSD system with DAD\ELSD G7102A 1290 Infinity II and Agilent LC\MSD G6120B mass-spectrometer.
Agilent 1260 Series LC/MSD system with DAD\ELSD and Agilent LC\MSD (G6120B) mass-spectrometer.
UHPLC Agilent 1290 Series LC/MSD system with DAD\ELSD and Agilent LC\MSD (G6125B) mass-spectrometer.

All the LC/MS data were obtained using positive/negative mode switching.

### H-NMR

Bruker AVANCE III 400
Varian UNITY INOVA 400

For chiral analysis or separation the following instruments were used:

### Analytical Separation:

Column: Chiralpak IA (250*4.6 mm, 5 mkm)

### HPLC Instrument:

Agilent Technologies HPLC Systems 1200 Series with DAD Detector (G1315B).

### Preparative Separation:

Column: Chiralpak IA (250*20 mm, 5 mkm)

### HPLC Instrument:

Agilent Technologies HPLC Preparative Systems 1260 Infinity II Series with DAD Detector (G7115B).

### Building Block Synthesis

### Preparation of 1-(tert-butoxycarbonyl)-5-((tert-butoxycarbonyl)(methyl)amino)piperidine-3-carboxylic acid

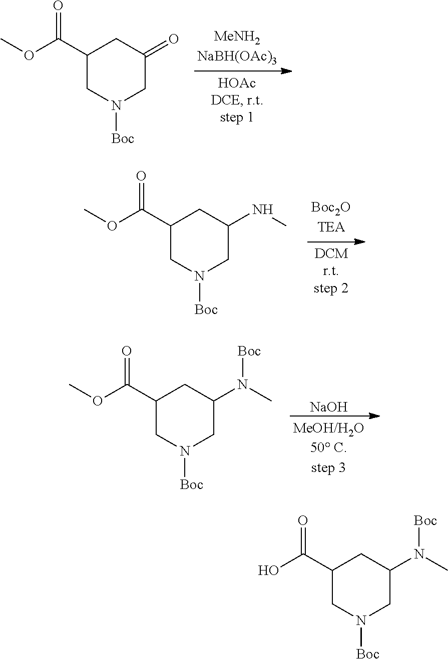

### Step 1. Synthesis of 1-(tert-butyl) 3-methyl 5-(methylamino)piperidine-1,3-dicarboxylate

1-(tert-butyl) 3-methyl 5-oxopiperidine-1,3-dicarboxylate (6 g, 23.32 mmol, 1 eq) was dissolved in dry DCE (100 ml), after that methanamine, 20% wt. solution in methanol (7.244 g, 46.64 mmol, 2 eq) was added to the resulting solution, followed by the addition of acetic acid (1 ml). The reaction mixture was stirred at room temperature for 15 minutes, after that sodium triacetoxyboranuide (14.828 g, 69.96 mmol, 3 eq) was added in portions while stirring. The reaction mixture was then left at room temperature for night. After 14 hours the reaction mixture was poured onto distilled water (150 ml) and sodium hydrogen carbonate (11.754 g, 6 eq) was added in portions while stirring. After the addition was completed the organic layer was separated, washed with brine (100 ml), dried over anhydrous sodium sulfate and filtered. The filtrate collected was concentrated under reduced pressure to afford the title product (5.25 g, 41.33%) as orange colored oil, which was used as such without any additional purification.

### Step 2. Synthesis of 1-(tert-butyl) 3-methyl 5-((tert-butoxycarbonyl)(methyl)amino)piperidine-1,3-dicarboxylate

The starting crude 1-(tert-butyl) 3-methyl 5-(methylamino)piperidine-1,3-dicarboxylate, obtained in the previous experiment (6.05 g, 11.107 mmol, 1 eq) was dissolved in dry DCM (75 ml) N,N-Diethylethanamine (3.096 ml, 2.248 g, 22.215 mmol, 2 eq) was added to the resulting solution, followed by the dropwise addition of tert-butoxycarbonyl tert-butyl carbonate (2.667 g, 12.218 mmol, 1.1 eq). The reaction mixture was then left while stirring at room temperature until gas evolution stopped. The reaction mixture was then washed with distilled water (2×75 ml). The organic layer was separated, dried over anhydrous sodium sulfate and filtered. The filtrate collected was concentrated under reduced pressure to afford 8 g of crude brown oil, which was subjected for flash chromatography purification (Interchim; 220 g SiO₂; petroleum ether/MtBE with MtBE from 0 to 65%, flow rate=100 ml/min) to give the title product (1.76 g, 40.42%) as orange oil.

### Step 3. Synthesis of 1-(tert-butoxycarbonyl)-5-((tert-butoxycarbonyl)(methyl)amino)piperidine-3-carboxylic acid

1-(tert-Butyl) 3-methyl 5-((tert-butoxycarbonyl)(methyl)amino)piperidine-1,3-dicarboxylate, obtained in the previous experiment (1.76 g, 4.489 mmol, 1 eq) was dissolved in absolute methanol (5 ml), followed by a solution of sodium hydroxide (0.539 g, 13.467 mmol, 3 eq) in distilled water (5 ml). The mixture was then heated up to 50° C. and left while stirring overnight. After full conversion was verified by LCMS analysis the reaction mixture was concentrated under reduced pressure and the residue obtained was diluted with distilled water (10 ml). The resulting aqueous solution was washed with DCM (5 ml). The aqueous solution was separated and NaHSO₄ (1.617 g, 3 eq) was added into it while stirring. The oily precipitate was formed, which was extracted with chloroform (3×7 ml). The organic layers were combined, dried over anhydrous sodium sulfate and filtered. The filtrate collected was concentrated under reduced pressure to afford the title product (1.314 g, 77.58%) as white foam. The crude product was used without additional purification.

### Preparation of rac-(3R,4R)-1-(tert-butoxycarbonyl)-4-(dimethylamino)pyrrolidine-3-carboxylic acid

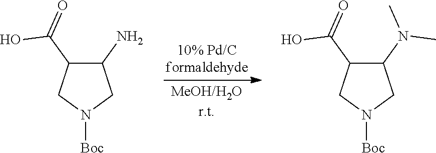 [SMILES: CC(C)(C)OC(=O)N1CC(N)C(C(=O)O)C1 ; CN(C)C1CN(C(=O)OC(C)(C)C)CC1C(=O)O]

rac-(3R,4R)-4-Amino-1-(tert-butoxycarbonyl)pyrrolidine-3-carboxylic acid (1 g, 4.34 mmol, 1 eq) was dissolved in a mixture of distilled water (10 ml) and methanol (10 ml). Formaldehyde, 35% solution (1.49 g, 17.37 mmol, 4 eq) was added, followed by the addition of palladium on carbon, 10% (0.231 g, 0.22 mmol, 0.05 eq). The reaction mixture was hydrogenated for 12 hours at room temperature in a hydrogen atmosphere of 10 atm. After that period the reaction mixture was filtered and the catalyst was washed with methanol (10 ml). The filtrates were collected, combined and concentrated under reduced pressure to afford the title product (1 g, 89.14%) as white solid, which was used without additional purification. LCMS [M+1]⁺ 259.2.

### Preparation of tert-butyl 4-(3-chloropyridin-4-yl)-4-cyanopiperidine-1-carboxylate

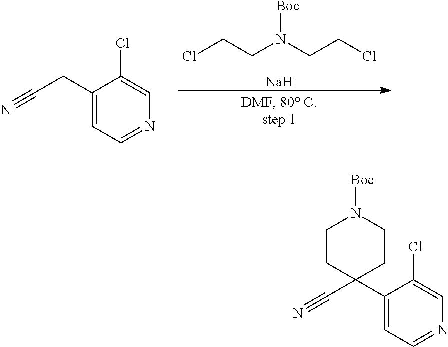

2-(3-Chloro-4-pyridyl)acetonitrile (3.4 g, 22.28 mmol, 1 eq) was dissolved in dry DMF (70 ml). Sodium hydride, 60% in mineral oil (2.67 g, 66.85 mmol, 3 eq) was slowly added in portions to the resulting solution at 0° C. under inert atmosphere (argon inlet). tert-Butyl N,N-bis(2-chloroethyl)carbamate (5.4 g, 22.28 mmol, 1 eq) was added to the reaction mixture, which was left while stirring at 80° C. overnight. After 14 hours the reaction mixture was quenched with saturated aqueous solution of NH₄Cl (50 ml), and the resulting mixture was extracted with DCM (3×30 ml). The organic layers were combined, washed with brine (2×50 ml), dried over anhydrous sodium sulfate and filtered. The filtrate collected was concentrated under reduced pressure to afford crude product (3.1 g), which was subjected for prep HPLC purification to provide 716 mg (10%) of the title product as pale brown solid. LCMS [M+1]⁺ 322.2.

### Preparation of 2-hydroxy-2-methyl-4-azaspiro[3.5]nonan-4-ium chloride

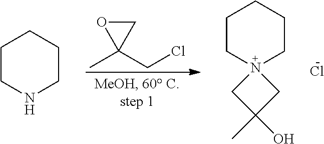

2-(Chloromethyl)-2-methyl-oxirane (10 g, 93.853 mmol, 1 eq) was added dropwise to a solution of piperidine (7.993 g, 9.271 mL, 93.8 mmol, 1 eq) in methanol (100 ml) maintaining the temperature of the reaction mixture below 5° C. After the addition was completed the reaction mixture was stirred at 0° C. for 1 hour and then allowed to warm up to room temperature. The reaction mixture was then refluxed for 24 hours. The mixture was concentrated under reduced pressure to afford the title product (19 g, 90%) as yellow solid. LCMS [M]⁺ 156.2. The crude product obtained was of sufficient purity and used without any additional purification.

### Preparation of O-(4-methylpent-1-en-3-yl)hydroxylamine hydrochloride

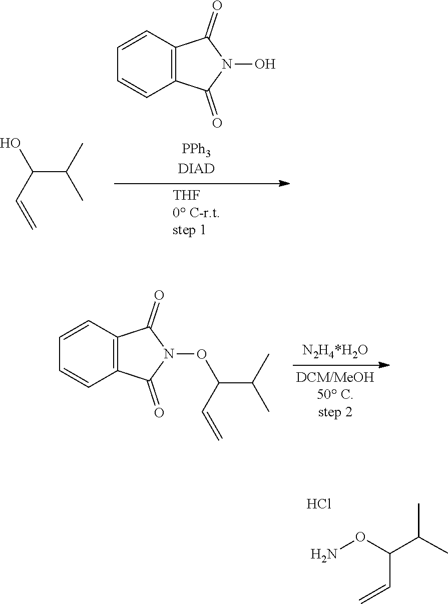

### Step 1. Synthesis of 2-((4-methylpent-1-en-3-yl)oxy)isoindoline-1,3-dione

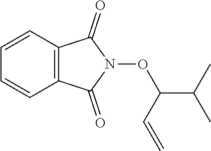 [SMILES: C=CC(ON1C(=O)c2ccccc2C1=O)C(C)C]

4-Methylpent-1-en-3-ol (6.5 g, 64.90 mmol, 1 eq) was dissolved in dry THF (400 ml). Then 2-hydroxyisoindoline-1,3-dione (12.17 g, 74.63 mmol, 1.15 eq) was added to the solution, followed by triphenylphosphane (2.43 g, 77.88 mmol, 1.2 eq). The resulting reaction mixture was cooled down using an ice bath and diisopropylazodicarboxylat (DIAD) (15.75 g, 77.88 mmol, 1.2 eq) was added dropwise to the reaction mixture at 0° C. After the addition was completed the cooling bath was removed and the mixture was allowed to warm up to room temperature and left while stirring overnight. After 14 hours the solvent was removed by evaporation and the resulting crude oily residue obtained was subjected to flash chromatography purification to yield 8.1 g (48%) of the desired product as white solid.

### Step 2. Synthesis of O-(4-methylpent-1-en-3-yl)hydroxylamine hydrochloride

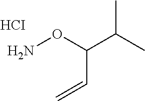

2-((4-Methylpent-1-en-3-yl)oxy)isoindoline-1,3-dione, obtained in the previous experiment (7.5 g, 29.05 mmol, 1 eq) was dissolved in the mixture of DCM (75 ml) and absolute methanol (75 ml), after that hydrazine hydrate (1.89 g, 1.80 ml, 37.77 mmol, 1.3 eq) was added to the resulting solution. The reaction mixture was then left while stirring at 50° C. for 5 hours. After that period the reaction mixture was filtered, the precipitate was additionally washed with DCM (2×50 ml). The filtrates were collected, combined and concentrated under reduced pressure to afford crude white solid residue, which was treated with 2N aqueous hydrochloric acid (10 ml). The resulting mixture was filtered and the filtrate collected was concentrated under reduced pressure (at 50° C.) to afford the title product (2 g, 43%) as white solid of satisfactorily purity.

### 1. General Synthesis Using Halo-Cyclization

### Synthesis of rac-3-(2,2-dimethyl-4-piperidyl)-5-(1-piperidylmethyl)-5,6-dihydro-1,4,2-dioxazine, ID 705

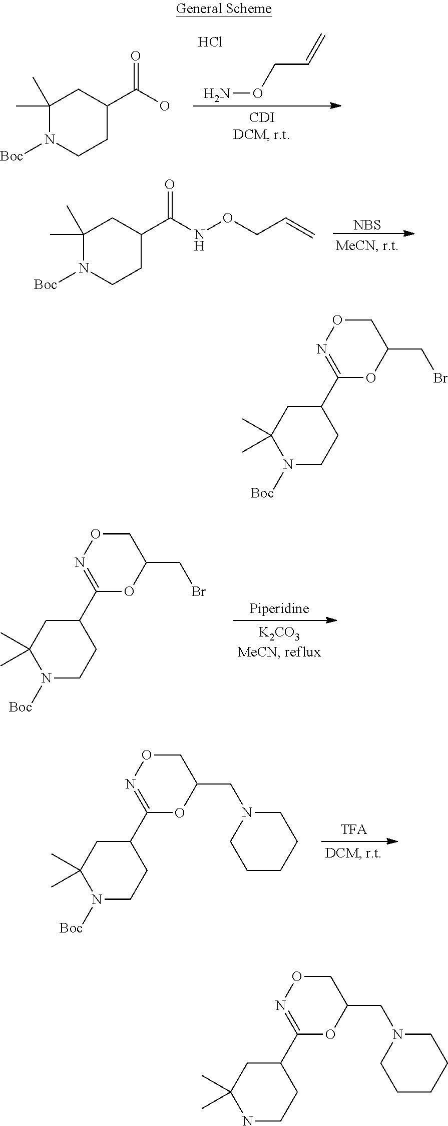

### Procedure 1.1

### Synthesis of rac-tert-butyl 4-(allyloxycarbamoyl)-2,2-dimethyl-piperidine-1-carboxylate

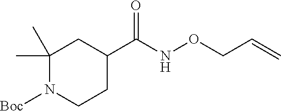 [SMILES: C=CCONC(=O)C1CCN(C(=O)OC(C)(C)C)C(C)(C)C1]

Rac-1-tert-Butoxycarbonyl-2,2-dimethyl-piperidine-4-carboxylic acid (1.0 g, 3.88 mmol, 1 eq) was dissolved in dry DCM (50 mL), followed by di(imidazol-1-yl)methanone (0.725 g, 4.47 mmol, 1.15 eq). The resulting mixture was left while stirring at room temperature for 5 hours. Then, O-allylhydroxylamine hydrochloride (0.553 g, 5.05 mmol, 1.3 eq) was added to the reaction mixture, which was left while stirring at ambient temperature overnight. After 12 hours the reaction mixture was washed with water (2×25 mL) and brine (25 mL). The organic layer was isolated, dried over anhydrous sodium sulfate and filtered. The filtrate was evaporated to afford the title compound (1.096 g, 76.8%) as yellow oil, which was used without further purification. LCMS [M−Boc+1]⁺ 213.4

### Procedure 1.2

### Synthesis of rac-tert-butyl 4-[5-(bromomethyl)-5,6-dihydro-1,4,2-dioxazin-3-yl]-2,2-dimethyl-piperidine-1-carboxylate

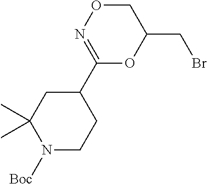 [SMILES: CC(C)(C)OC(=O)N1CCC(C2=NOCC(CBr)O2)CC1(C)C]

tert-Butyl-rac-4-(allyloxycarbamoyl)-2,2-dimethyl-piperidine-1-carboxylate (1096 mg, 3.33 mmol, 1 eq) was dissolved in dry acetonitrile (20 mL). 1-Bromo-2,5-pyrrolidinedione (889 mg, 4.99 mmol, 1.5 eq) was added to the solution. The reaction mixture was left while stirring at room temperature overnight. After 12 hours the reaction mixture was evaporated under reduced pressure to yield a residue, which was diluted with DCM (40 mL), washed with a saturated aqueous solution of sodium thiosulfate (2×25 mL), water (2×25 mL) and brine (25 mL). The organic layer was isolated, dried over anhydrous sodium sulfate and filtered. The filtrate was concentrated under reduced pressure to afford the title product as red oil (1.095 g), which was used without additional purification. LCMS [M−t-Bu+1]⁺ 337.2

### Procedure 1.3

### Synthesis of rac-tert-butyl 2,2-dimethyl-4-[5-(1-piperidylmethyl)-5,6-dihydro-1,4,2-dioxazin-3-yl]piperidine-1-carboxylate

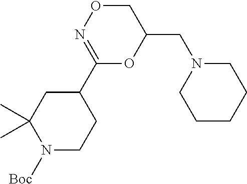 [SMILES: CC(C)(C)OC(=O)N1CCC(C2=NOCC(CN3CCCCC3)O2)CC1(C)C]

To a solution of tert-butyl 4-[5-(bromomethyl)-5,6-dihydro-1,4,2-dioxazin-3-yl]-2,2-dimethyl-piperidine-1-carboxylate (1096 mg, 2.43 mmol, 1 eq) in dry acetonitrile (50 mL) was added dipotassium carbonate (1010 mg, 7.31 mmol, 3 eq), followed by the addition of piperidine (415 mg, 4.87 mmol, 2 eq). The reaction mixture was refluxed overnight and after 15 hours cooled down and then concentrated under reduced pressure. The residue was diluted with DCM (70 mL), washed with water (3×50 mL) and brine (50 mL). The organic layer was isolated, dried over anhydrous sodium sulfate and filtered. The filtrate was evaporated under reduced pressure to afford 1.0 g of a red brown oily residue. The residue was subjected to preparative HPLC (65-80% 0-6 min water-methanol, flow: 30 ml/min; loading pump 4 ml/min methanol; target mass 396; column: SunFireC18; 100×19 mm; 5 um) to afford the title product (411 mg, 42.64%) as an yellow colored oil. LCMS [M+1]⁺ 396.4

### Procedure 1.4

### Synthesis of rac-3-(2,2-dimethyl-4-piperidyl)-5-(1-piperidylmethyl)-5,6-dihydro-1,4,2-dioxazine

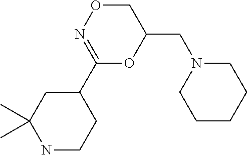 [SMILES: CC1(C)CC(C2=NOCC(CN3CCCCC3)O2)CCN1]

tert-Butyl-rac-2,2-dimethyl-4-[5-(1-piperidylmethyl)-5,6-dihydro-1,4,2-dioxazin-3-yl]piperidine-1-carboxylate (411 mg, 1.039 mmol, 1 eq) was dissolved in dry DCM (6 mL), followed by dropwise addition of 2,2,2-trifluoroacetic acid (1184 mg, 10.39 mmol, 10 eq). The reaction mixture was left while stirring at ambient temperature overnight. After 12 hours the reaction mixture was concentrated under reduced pressure to afford an orange colored oily residue. This was diluted with DCM (20 mL) and washed with 30% aqueous solution of potassium carbonate (2×15 mL). The organic layer was isolated, dried over anhydrous sodium sulfate and filtered. The filtrate was concentrated under reduced pressure to afford a crude product residue (132 mg, orange oil). The residue was subjected to preparative HPLC (95-95-40% 0-1-5 min acetonitrile-methanol, flow: 40 ml/min; loading pump 4 ml/min acetonitrile; target mass 296; column Uptisphere Strategy HILIC-HIA 100×21.2 mm; 5 um) to afford the title product (67.3 mg, 20.8%) as a yellow oil. LCMS [M+1]⁺ 296.4. ¹H NMR (400 MHz, CD₃OD) δ 4.52-4.41 (m, 1H), 4.07 (dd, J=11.5, 2.9 Hz, 1H), 3.69 (ddd, J=11.6, 6.5, 1.8 Hz, 1H), 2.89-2.79 (m, 2H), 2.60-2.42 (m, 7H), 1.81-1.71 (m, 1H), 1.70-1.54 (m, 5H), 1.51-1.41 (m, 3H), 1.41-1.32 (m, 1H), 1.13 (s, 6H).

### Synthesis of (rel-R)-3-((rel-trans-3aS,6aR)-hexahydrocyclopenta[b]pyrrol-3a(1H)-yl)-5-(piperidin-1-ylmethyl)-5,6-dihydro-1,4,2-dioxazine, ID 589

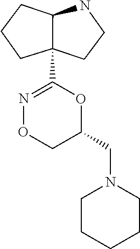 [SMILES: C1CCN(C[C@@H]2CON=C([C@]34CCC[C@H]3NCC4)O2)CC1]

The general synthesis using halo-cyclization as described herein was used to provide rel-(R)-3-((3aS,6aR)-hexahydrocyclopenta[b]pyrrol-3a(1H)-yl)-5-(piperidin-1-ylmethyl)-5,6-dihydro-1,4,2-dioxazine (single enantiomer of unknown absolute configuration, 113.9 mg, 53.04%) as a yellow oil from commercially available rel-trans-(3aS,6aR)-1-(tert-butoxycarbonyl)hexahydrocyclopenta[b]pyrrole-3a(1H)-carboxylic acid in line with the synthesis described in 1.1 to 1.4. Chiral separation was applied after the equivalent of reaction step 1.3 (the BOC protected entity). LCMS [M+1]⁺ 294.2. ¹H NMR (400 MHz, CD₃OD) δ 4.57-4.44 (m, 1H), 4.09 (dd, J=11.5, 2.9 Hz, 1H), 3.85-3.78 (m, 1H), 3.71 (dd, J=11.6, 6.5 Hz, 1H), 2.99-2.86 (m, 1H), 2.86-2.74 (m, 1H), 2.66-2.41 (m, 6H), 2.30-2.17 (m, 1H), 2.14-2.02 (m, 1H), 1.91-1.79 (m, 1H), 1.79-1.70 (m, 1H), 1.68-1.52 (m, 8H), 1.52-1.40 (m, 2H).

### Synthesis of rel-(S)-3-rel-trans-((3aS,6aR)-hexahydrocyclopenta[b]pyrrol-3a(1H)-yl)-5-(piperidin-1-ylmethyl)-5,6-dihydro-1,4,2-dioxazine, ID 590

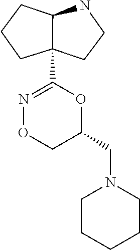 [SMILES: C1CCN(C[C@@H]2CON=C([C@]34CCC[C@H]3NCC4)O2)CC1]

The general synthesis using halo-cyclization as described herein was used to provide re/—(S)-3-((3aS,6aR)-hexahydrocyclopenta[b]pyrrol-3a(1H)-yl)-5-(piperidin-1-ylmethyl)-5,6-dihydro-1,4,2-dioxazine (118.4 mg, 48.67%) as a yellow oil from commercially available rel-trans-(3aS,6aR)-1-(tert-butoxycarbonyl)hexahydrocyclopenta[b]pyrrole-3a(1H)-carboxylic acid in line with the synthesis described in 1.1 to 1.4. The material is a single enantiomer of unknown absolute configuration. Chiral separation was applied after the equivalent of reaction step 1.3 (the BOC protected entity). LCMS [M+1]⁺ 294.2. ¹H NMR (400 MHz, CD₃OD) δ 4.55-4.45 (m, 1H), 4.09 (dd, J=11.5, 2.9 Hz, 1H), 3.83 (dd, J=7.3, 3.3 Hz, 1H), 3.71 (dd, J=11.6, 6.5 Hz, 1H), 2.97-2.87 (m, 1H), 2.87-2.77 (m, 1H), 2.67-2.42 (m, 6H), 2.33-2.21 (m, 1H), 2.13-2.00 (m, 1H), 1.91-1.80 (m, 1H), 1.78-1.70 (m, 1H), 1.70-1.51 (m, 8H), 1.51-1.37 (m, 2H).

### Synthesis of rac-3-rel-trans-((3aS,6aR)-hexahydrocyclopenta[b]pyrrol-3a(1H)-yl)-5-(piperidin-1-ylmethyl)-5,6-dihydro-1,4,2-dioxazine, ID 469

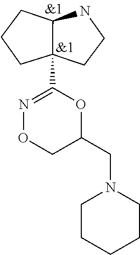 [SMILES: C1CCN(CC2CON=C([C@]34CCC[C@H]3NCC4)O2)CC1]

The general synthesis using halo-cyclization as described herein was used to provide rac-3-rel-trans-((3aR,6aS)-hexahydrocyclopenta[b]pyrrol-3a(1H)-yl)-5-(piperidin-1-ylmethyl)-5,6-dihydro-1,4,2-dioxazine (139 mg, 39.36%) as a yellow oil from commercially available rel-trans(3aS,6aR)-1-(tert-butoxycarbonyl)hexahydrocyclopenta[b]pyrrole-3a(1H)-carboxylic acid in line with the synthesis described in 1.1 to 1.4. This material is a mixture of four compounds. The hexahydrocyclopenta[b]pyrrol-3a(1H) core has a trans relationship AND dioxazine-C5 centre can be either R or S-configuration. LCMS [M+1]⁺ 294.2. ¹H NMR (400 MHz, cdcl₃) δ 4.45-4.30 (m, 1H), 4.17-4.03 (m, 1H), 4.00-3.86 (m, 1H), 3.70 (dd, J=11.4, 6.6 Hz, 1H), 3.44-2.76 (m, 4H), 2.54-2.43 (m, 4H), 2.43-2.36 (m, 2H), 2.36-2.19 (m, 2H), 2.11-1.99 (m, 1H), 1.91-1.82 (m, 1H), 1.80-1.54 (m, 7H), 1.50-1.31 (m, 2H).

### Synthesis of rac-rel-trans-3-((3aS,6aR)-hexahydrocyclopenta[b]pyrrol-3a(1H)-yl)-5-(piperidin-1-ylmethyl)-5,6-dihydro-1,4,2-dioxazine, ID 584

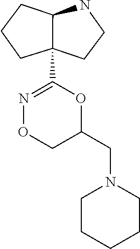 [SMILES: C1CCN(CC2CON=C([C@]34CCC[C@H]3NCC4)O2)CC1]

The general synthesis using halo-cyclization as described herein was used to provide rac-rel-trans-3-rel-trans-((3aS,6aR)-hexahydrocyclopenta[b]pyrrol-3a(1H)-yl)-5-(piperidin-1-ylmethyl)-5,6-dihydro-1,4,2-dioxazine (265.4 mg, 65.28%) as a yellow oil from commercially available rel-trans-(3aR,6aS)-1-(tert-butoxycarbonyl)hexahydrocyclopenta[b]pyrrole-3a(1H)-carboxylic acid in line with the synthesis described in 1.1 to 1.4. This material is a mixture of two compounds with a single fixed trans hexahydrocyclopenta[b]pyrrol-3a(1H) core AND a mixture of R and S configuration in the dioxazine-C5 centre (piperidylmethyl substitution) OR, a single stereoisomer in the dioxazine-C5 centre AND a mixture of TWO trans-hexahydrocyclopenta[b]pyrrol-3a(1H) cores Chiral separation was applied after the equivalent of reaction step 1.3 (the BOC protected entity). LCMS [M+1]⁺ 294.2. ¹H NMR (400 MHz, CD₃OD) δ 4.56-4.43 (m, 1H), 4.09 (dd, J=11.7, 2.9 Hz, 1H), 3.82 (dd, J=7.3, 3.3 Hz, 1H), 3.71 (dd, J=11.6, 6.5 Hz, 1H), 2.96-2.87 (m, 1H), 2.87-2.76 (m, 1H), 2.68-2.40 (m, 6H), 2.32-2.20 (m, 1H), 2.15-2.01 (m, 1H), 1.90-1.80 (m, 1H), 1.80-1.69 (m, 1H), 1.69-1.52 (m, 8H), 1.52-1.34 (m, 2H).

### Synthesis of rac-3-((2R,6S)-2,6-dimethylpiperidin-4-yl)-5-(piperidin-1-ylmethyl)-5,6-dihydro-1,4,2-dioxazine, ID 633

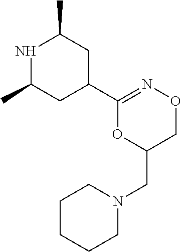 [SMILES: C[C@@H]1CC(C2=NOCC(CN3CCCCC3)O2)C[C@H](C)N1]

The general synthesis using halo-cyclization as described herein was used to provide rac-3-((2R,6S)-2,6-dimethylpiperidin-4-yl)-5-(piperidin-1-ylmethyl)-5,6-dihydro-1,4,2-dioxazine (155 mg, 36.86%) as a yellow oil from commercially available (2R,6S)-1-(tert-butoxycarbonyl)-2,6-dimethylpiperidine-4-carboxylic acid in line with the synthesis described in 1.1 to 1.4. LCMS [M+1]⁺ 296.4. ¹H NMR (400 MHz, CD₃OD) δ 4.52-4.40 (m, 1H), 4.07 (dd, J=11.7, 2.9 Hz, 1H), 3.69 (dd, J=11.6, 6.4 Hz, 1H), 2.82-2.72 (m, 2H), 2.62-2.45 (m, 6H), 2.43-2.33 (m, 1H), 1.87-1.77 (m, 2H), 1.60 (p, J=5.5, 5.5, 5.5, 5.5 Hz, 4H), 1.52-1.40 (m, 2H), 1.23 (ddd, J=24.7, 13.5, 6.1 Hz, 2H), 1.13 (d, J=6.3 Hz, 6H).

### Synthesis of rac-6-isopropyl-5-(piperidin-1-ylmethyl)-3-(piperidin-4-yl)-5,6-dihydro-1,4,2-dioxazine, ID 706

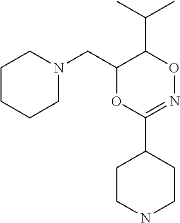 [SMILES: CC(C)C1ON=C(C2CCNCC2)OC1CN1CCCCC1]

The general synthesis using halo-cyclization as described herein was used to provide rac-6-isopropyl-5-(piperidin-1-ylmethyl)-3-(piperidin-4-yl)-5,6-dihydro-1,4,2-dioxazine (84.2 mg, 53.17%) as a yellow oil from commercially available 1-(tert-butoxycarbonyl)piperidine-4-carboxylic acid in line with the synthesis described in 1.1 to 1.4 but using O-(1-isopropylallyl)hydroxylamine hydrochloride instead of 0-allylhydroxylamine hydrochloride in experimental procedure 1.1. The synthesis of O-(1-isopropylallyl)hydroxylamine hydrochloride is described above. LCMS [M+1]⁺ 310.2. ¹H NMR (400 MHz, CD₃OD) δ 4.46-4.35 (m, 1H), 3.44 (t, J=5.4, 5.4 Hz, 1H), 3.10-2.98 (m, 2H), 2.74-2.60 (m, 2H), 2.60-2.43 (m, 6H), 2.40-2.21 (m, 1H), 2.02 (q, J=6.6, 6.6, 6.6 Hz, 1H), 1.86-1.72 (m, 2H), 1.66-1.52 (m, 6H), 1.52-1.39 (m, 2H), 1.06 (d, J=6.9 Hz, 3H), 0.98 (d, J=6.7 Hz, 3H).

### Synthesis of rac-3-(4-methylpiperidin-4-yl)-5-(piperidin-1-ylmethyl)-5,6-dihydro-1,4,2-dioxazine, ID 568

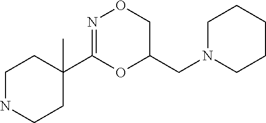 [SMILES: CC1(C2=NOCC(CN3CCCCC3)O2)CCNCC1]

The general synthesis using halo-cyclization as described herein was used to provide rac-3-(4-methylpiperidin-4-yl)-5-(piperidin-1-ylmethyl)-5,6-dihydro-1,4,2-dioxazine (135.8 mg, 18.03%) as a yellow oil from commercially available 1-(tert-butoxycarbonyl)-4-methylpiperidine-4-carboxylic acid in line with the synthesis described in 1.1 to 1.4. LCMS [M+1]⁺ 282.2. ¹H NMR (400 MHz, CDCl₃) δ 4.40-4.28 (m, 1H), 4.05 (d, J=10.7 Hz, 1H), 3.70 (dd, J=11.4, 6.4 Hz, 1H), 2.93-2.72 (m, 4H), 2.55-2.42 (m, 4H), 2.41-2.31 (m, 2H), 2.07-1.83 (m, 4H), 1.54-1.46 (m, 3H), 1.45-1.26 (m, 4H), 1.15 (s, 3H).

### Synthesis of rac-3-(3-methylpiperidin-3-yl)-5-(piperidin-1-ylmethyl)-5,6-dihydro-1,4,2-dioxazine, ID 476

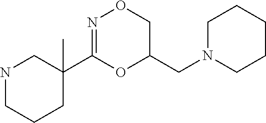 [SMILES: CC1(C2=NOCC(CN3CCCCC3)O2)CCCNC1]

The general synthesis using halo-cyclization as described herein was used to provide rac-3-(3-methylpiperidin-3-yl)-5-(piperidin-1-ylmethyl)-5,6-dihydro-1,4,2-dioxazine (194.2 mg, 65.82%) as a yellow oil from commercially available rac-1-(tert-butoxycarbonyl)-3-methylpiperidine-3-carboxylic acid in line with the synthesis described in 1.1 to 1.4. LCMS [M+1]⁺ 282.2. ¹H NMR (400 MHz, CDCl₃) δ 4.43-4.32 (m, 1H), 4.08 (td, J=11.1, 11.0, 2.9 Hz, 1H), 3.74 (dt, J=11.2, 5.6, 5.6 Hz, 1H), 3.28-3.17 (m, 1H), 3.01-2.86 (m, 1H), 2.69-2.57 (m, 1H), 2.57-2.44 (m, 4H), 2.44-2.35 (m, 3H), 2.19-1.94 (m, 3H), 1.57-1.51 (m, 3H), 1.50-1.43 (m, 2H), 1.43-1.36 (m, 2H), 1.36-1.26 (m, 1H), 1.05 (d, J=2.8 Hz, 3H).

### Synthesis of rac-6-methyl-5-(piperidin-1-ylmethyl)-3-(pyrrolidin-3-yl)-5,6-dihydro-1,4,2-dioxazine, ID 616

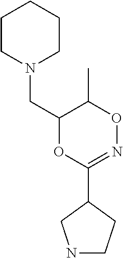 [SMILES: CC1ON=C(C2CCNC2)OC1CN1CCCCC1]

The general synthesis using halo-cyclization as described herein was used to provide rac-6-methyl-5-(piperidin-1-ylmethyl)-3-(pyrrolidin-3-yl)-5,6-dihydro-1,4,2-dioxazine (177.7 mg, 16%) as a yellow oil from commercially available rac-1-(tert-butoxycarbonyl)pyrrolidine-3-carboxylic acid in line with the synthesis described in 1.1 to 1.4 but using O-(1-methylallyl)hydroxylamine hydrochloride instead of 0-allylhydroxylamine hydrochloride in experimental procedure 1.1. LCMS [M+1]⁺ 268.4. ¹H NMR (400 MHz, CD₃OD) δ 4.19-4.06 (m, 1H), 3.73-3.62 (m, 1H), 3.06 (dd, J=11.5, 8.1 Hz, 1H), 3.02-2.92 (m, 2H), 2.92-2.81 (m, 2H), 2.61 (dt, J=14.1, 2.8, 2.8 Hz, 1H), 2.58-2.39 (m, 5H), 2.08-1.87 (m, 2H), 1.68-1.53 (m, 4H), 1.53-1.39 (m, 2H), 1.27 (d, J=6.2 Hz, 3H).

### Synthesis of rel-(S)-3-(4-methylpiperidin-4-yl)-5-(piperidin-1-ylmethyl)-5,6-dihydro-1,4,2-dioxazine, ID 567

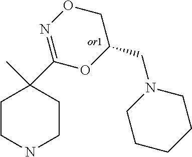 [SMILES: CC1(C2=NOC[C@H](CN3CCCCC3)O2)CCNCC1]

The general synthesis using halo-cyclization as described herein was used to provide re/—(S)-3-(4-methylpiperidin-4-yl)-5-(piperidin-1-ylmethyl)-5,6-dihydro-1,4,2-dioxazine (178.4 mg, 53.9%) as a yellow oil from commercially available 1-(tert-butoxycarbonyl)-4-methylpiperidine-4-carboxylic acid in line with the synthesis described in 1.1 to 1.4. Chiral separation was applied after the equivalent of reaction step 1.3. LCMS [M+1]⁺ 282.4. ¹H NMR (400 MHz, CDCl₃) δ 5.02-4.80 (m, 2H), 4.44-4.27 (m, 1H), 4.07 (d, J=10.9 Hz, 1H), 3.72 (dd, J=11.3, 6.2 Hz, 1H), 3.17-3.04 (m, 2H), 3.01-2.88 (m, 2H), 2.64-2.40 (m, 5H), 2.40-2.30 (m, 2H), 2.10 (d, J=13.8 Hz, 2H), 1.73-1.53 (m, 4H), 1.45-1.33 (m, 2H), 1.19 (s, 3H).

### Synthesis of rac-6-methyl-3-(4-methylpiperidin-4-yl)-5-(piperidin-1-ylmethyl)-5,6-dihydro-1,4,2-dioxazine, ID 531

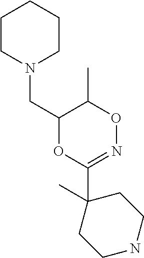 [SMILES: CC1ON=C(C2(C)CCNCC2)OC1CN1CCCCC1]

The general synthesis using halo-cyclization as described herein was used to provide rac-6-methyl-3-(4-methylpiperidin-4-yl)-5-(piperidin-1-ylmethyl)-5,6-dihydro-1,4,2-dioxazine (261.6 mg, 52.24%) as a yellow oil from commercially available 1-(tert-butoxycarbonyl)-4-methylpiperidine-4-carboxylic acid in line with the synthesis described in 1.1 to 1.4 but using O-(1-methylallyl)hydroxylamine hydrochloride instead of 0-allylhydroxylamine hydrochloride in experimental procedure 1.1. LCMS [M+1]⁺ 296.4. ¹H NMR (400 MHz, CDCl₃) δ 4.00 (q, J=6.0, 6.0, 5.9 Hz, 1H), 3.69 (p, J=6.2, 6.2, 6.2, 6.2 Hz, 1H), 2.96-2.80 (m, 4H), 2.69-2.53 (m, 4H), 2.51-2.34 (m, 6H), 2.08-1.98 (m, 2H), 1.50-1.34 (m, 5H), 1.29 (d, J=6.3 Hz, 3H), 1.18 (s, 3H).

### Synthesis of rac-3-(5-methylpyrrolidin-3-yl)-5-(piperidin-1-ylmethyl)-5,6-dihydro-1,4,2-dioxazine, ID 560

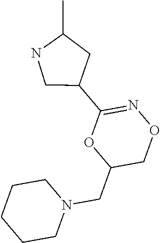 [SMILES: CC1CC(C2=NOCC(CN3CCCCC3)O2)CN1]

The general synthesis using halo-cyclization as described herein was used to provide rac-3-(5-methylpyrrolidin-3-yl)-5-(piperidin-1-ylmethyl)-5,6-dihydro-1,4,2-dioxazine (449.5 mg, 58.69%) as a yellow oil from commercially available rac-1-(tert-butoxycarbonyl)-5-methylpyrrolidine-3-carboxylic acid in line with the synthesis described in 1.1 to 1.4. LCMS [M+1]⁺ 268.4. ¹H NMR (400 MHz, CD₃OD) δ 4.57-4.46 (m, 1H), 4.14-4.04 (m, 1H), 3.82-3.64 (m, 1H), 3.29-3.11 (m, 2H), 3.08-2.98 (m, 1H), 2.98-2.87 (m, 1H), 2.67-2.57 (m, 2H), 2.57-2.42 (m, 4H), 2.30-2.10 (m, 1H), 1.70-1.59 (m, 4H), 1.58-1.51 (m, 1H), 1.51-1.41 (m, 2H), 1.29-1.17 (m, 3H).

### Synthesis of rac-rel-trans(3aR,7aS)-3a-(5-(piperidin-1-ylmethyl)-5,6-dihydro-1,4,2-dioxazin-3-yl)octahydropyrano[4,3-b]pyrrole di-2,2,2-trifluoroacetate, ID 462

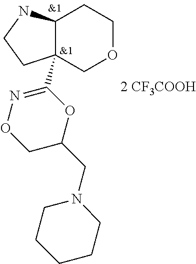 [SMILES: C1CCN(CC2CON=C([C@]34CCN[C@H]3CCOC4)O2)CC1]

The general synthesis using halo-cyclization as described herein was used to provide rac-rel-trans(3aR,7aS)-3a-(5-(piperidin-1-ylmethyl)-5,6-dihydro-1,4,2-dioxazin-3-yl)octahydropyrano[4,3-b]pyrrole di-2,2,2-trifluoroacetate (270 mg, 33.84%) as a colorless oil from commercially available rel-trans-(3aR,7aS)-1-(tert-butoxycarbonyl)hexahydropyrano[4,3-b]pyrrole-3a(4H)-carboxylic acid in line with the synthesis described in 1.1 to 1.4. LCMS [M+1]⁺ 310.4. ¹H NMR (400 MHz, CD₃OD) δ 5.01-4.93 (m, 1H), 4.28-4.16 (m, 2H), 4.13-3.91 (m, 1H), 3.91-3.72 (m, 4H), 3.68-3.50 (m, 4H), 3.50-3.41 (m, 3H), 3.23-2.98 (m, 2H), 2.38 (q, J=8.5, 8.5, 8.3 Hz, 2H), 2.17-2.04 (m, 1H), 2.01-1.73 (m, 6H), 1.70-1.46 (m, 1H).

### Synthesis of rac-5-(piperidin-1-ylmethyl)-3-(2-(piperidin-3-yl)propan-2-yl)-5,6-dihydro-1,4,2-dioxazine di-2,2,2-trifluoroacetate, ID 473

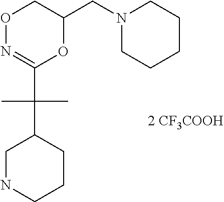 [SMILES: CC(C)(C1=NOCC(CN2CCCCC2)O1)C1CCCNC1]

The general synthesis using halo-cyclization as described herein was used to provide rac-5-(piperidin-1-ylmethyl)-3-(2-(piperidin-3-yl)propan-2-yl)-5,6-dihydro-1,4,2-dioxazine di-2,2,2-trifluoroacetate (117.2 mg, 49.61%) as a beige oil from commercially available rac-2-(1-(tert-butoxycarbonyl)piperidin-3-yl)-2-methylpropanoic acid in line with the synthesis described in 1.1 to 1.4. LCMS [M+1]⁺ 310.4. ¹H NMR (400 MHz, D₂O) δ 5.03-4.92 (m, 1H), 4.26-4.16 (m, 1H), 3.77 (dd, J=11.9, 7.1 Hz, 1H), 3.71-3.62 (m, 1H), 3.60-3.53 (m, 1H), 3.50-3.31 (m, 4H), 3.17-3.04 (m, 2H), 2.93-2.75 (m, 2H), 2.05-1.92 (m, 4H), 1.88-1.72 (m, 4H), 1.72-1.59 (m, 1H), 1.56-1.45 (m, 1H), 1.44-1.31 (m, 1H), 1.29-1.02 (m, 6H).

### Synthesis of rac-3-(3-methylpyrrolidin-3-yl)-5-(piperidin-1-ylmethyl)-5,6-dihydro-1,4,2-dioxazine, ID 465

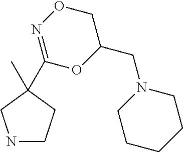 [SMILES: CC1(C2=NOCC(CN3CCCCC3)O2)CCNC1]

The general synthesis using halo-cyclization as described herein was used to provide rac-3-(3-methylpyrrolidin-3-yl)-5-(piperidin-1-ylmethyl)-5,6-dihydro-1,4,2-dioxazine (74.1 mg, 18.43%) as a yellow oil from commercially available rac-1-(tert-butoxycarbonyl)-3-methylpyrrolidine-3-carboxylic acid in line with the synthesis described in 1.1 to 1.4. LCMS [M+1]⁺ 268.4. ¹H NMR (400 MHz, cdcl₃) δ 4.46-4.31 (m, 1H), 4.06 (d, J=11.0 Hz, 1H), 3.79-3.66 (m, 1H), 3.33-3.20 (m, 1H), 3.12-2.83 (m, 2H), 2.71-2.32 (m, 10H), 2.27-2.16 (m, 1H), 1.54-1.45 (m, 3H), 1.44-1.35 (m, OH), 1.31-1.16 (m, 3H).

### Synthesis of rac-(1R,4R)-1-(5-(piperidin-1-ylmethyl)-5,6-dihydro-1,4,2-dioxazin-3-yl)-2-oxa-5-azabicyclo[2.2.1]heptane, ID 490

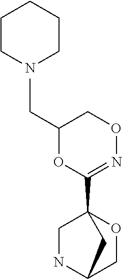

The general synthesis using halo-cyclization as described herein was used to provide rac-(1R,4R)-1-(5-(piperidin-1-ylmethyl)-5,6-dihydro-1,4,2-dioxazin-3-yl)-2-oxa-5-azabicyclo[2.2.1]heptane (146.5 mg, 36.39%) as a yellow oil from commercially available (1R,4R)-5-(tert-butoxycarbonyl)-2-oxa-5-azabicyclo[2.2.1]heptane-1-carboxylic acid in line with the synthesis described in 1.1 to 1.4. LCMS [M+1]⁺ 282.2. ¹H NMR (400 MHz, CDCl₃) δ 4.51-4.39 (m, 1H), 4.18 (dt, J=11.6, 2.9, 2.9 Hz, 1H), 4.01 (d, J=6.8 Hz, 1H), 3.85 (q, J=8.8, 8.8, 7.9 Hz, 2H), 3.73 (s, 1H), 3.29-3.15 (m, 2H), 2.69-2.54 (m, 2H), 2.54-2.43 (m, 2H), 2.43-2.33 (m, 2H), 2.05 (dd, J=9.9, 4.1 Hz, 1H), 2.00-1.93 (m, 1H), 1.92-1.67 (m, 2H), 1.65-1.54 (m, 3H), 1.41 (q, J=5.7, 5.7, 5.7 Hz, 2H).

### Synthesis of rac-5-(piperidin-1-ylmethyl)-3-(piperidin-3-ylmethyl)-5,6-dihydro-1,4,2-dioxazine, ID 554

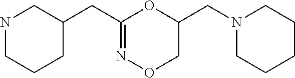 [SMILES: C1CCN(CC2CON=C(CC3CCCNC3)O2)CC1]

The general synthesis using halo-cyclization as described herein was used to provide (312.8 mg, 45.78%) as a yellow oil from commercially available rac-2-(1-(tert-butoxycarbonyl)piperidin-3-yl)acetic acid in line with the synthesis described in 1.1 to 1.4. LCMS [M+1]⁺ 282.4. ¹H NMR (400 MHz, CDCl₃) δ 4.40-4.28 (m, 1H), 4.12-4.01 (m, 1H), 3.75-3.65 (m, 1H), 3.57-3.27 (m, 3H), 3.15-3.07 (m, 1H), 3.07-3.00 (m, 2H), 2.58-2.51 (m, 1H), 2.51-2.40 (m, 4H), 2.40-2.28 (m, 3H), 2.12-1.99 (m, 2H), 1.95-1.77 (m, 2H), 1.73-1.62 (m, 1H), 1.53-1.46 (m, 3H), 1.44-1.32 (m, 2H), 1.24-1.01 (m, 1H).

### Synthesis of rac-3-(3-(2-methoxyethyl)pyrrolidin-3-yl)-5-(piperidin-1-ylmethyl)-5,6-dihydro-1,4,2-dioxazine, ID 685

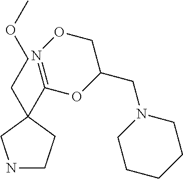 [SMILES: COCCC1(C2=NOCC(CN3CCCCC3)O2)CCNC1]

The general synthesis using halo-cyclization as described herein was used to provide rac-3-(3-(2-methoxyethyl)pyrrolidin-3-yl)-5-(piperidin-1-ylmethyl)-5,6-dihydro-1,4,2-dioxazine (92 mg, 29.43%) as a yellow oil from commercially available rac-1-(tert-butoxycarbonyl)-3-(2-methoxyethyl)pyrrolidine-3-carboxylic acid in line with the synthesis described in 1.1 to 1.4. LCMS [M+1]⁺ 312.4. ¹H NMR (400 MHz, CD₃OD) δ 4.55-4.46 (m, 1H), 4.15-4.03 (m, 1H), 3.80-3.67 (m, 1H), 3.46-3.40 (m, 2H), 3.33-3.32 (m, 4H), 2.99 (t, J=7.2, 7.2 Hz, 2H), 2.72-2.65 (m, 1H), 2.62-2.47 (m, 6H), 2.35-2.24 (m, 1H), 1.97-1.88 (m, 2H), 1.75-1.66 (m, 1H), 1.65-1.57 (m, 4H), 1.53-1.43 (m, 2H).

### Synthesis of rac-rel-trans-3-((1R,5R)-3-azabicyclo[3.1.0]hexan-1-yl)-5-(piperidin-1-ylmethyl)-5,6-dihydro-1,4,2-dioxazine, ID 482

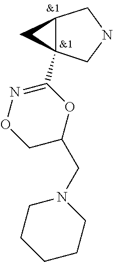 [SMILES: C1CCN(CC2CON=C([C@@]34CNC[C@@H]3C4)O2)CC1]

The general synthesis using halo-cyclization as described herein was used to provide rac-rel-trans-3-((1R,5R)-3-azabicyclo[3.1.0]hexan-1-yl)-5-(piperidin-1-ylmethyl)-5,6-dihydro-1,4,2-dioxazine (31.5 mg, 6.34%) as a yellow oil from commercially available rel-trans-(1R,5R)-3-(tert-butoxycarbonyl)-3-azabicyclo[3.1.0]hexane-1-carboxylic acid in line with the synthesis described in 1.1 to 1.4. LCMS [M+1]⁺ 266.4. ¹H NMR (400 MHz, CD₃OD) δ 4.52-4.38 (m, 1H), 4.17-4.05 (m, 1H), 3.74-3.66 (m, 1H), 3.66-3.31 (m, 1H), 3.21-2.63 (m, 4H), 2.61-2.38 (m, 6H), 1.91-1.78 (m, 1H), 1.67-1.52 (m, 4H), 1.50-1.34 (m, 2H), 1.29-1.12 (m, 1H), 0.87-0.61 (m, 1H).

### Synthesis of rac-3-(4-methylpiperidin-4-yl)-5-((5-(trifluoromethyl)-1,4-diazepan-1-yl)methyl)-5,6-dihydro-1,4,2-dioxazine, ID 500

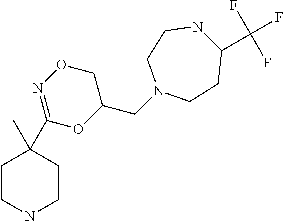 [SMILES: CC1(C2=NOCC(CN3CCNC(C(F)(F)F)CC3)O2)CCNCC1]

The general synthesis using halo-cyclization as described herein was used to provide rac-3-(4-methylpiperidin-4-yl)-5-((5-(trifluoromethyl)-1,4-diazepan-1-yl)methyl)-5,6-dihydro-1,4,2-dioxazine (23.4 mg, 11.69%) as a yellow oil from commercially available 1-(tert-butoxycarbonyl)-4-methylpiperidine-4-carboxylic acid in line with the synthesis described in 1.1 to 1.4 but using 5-(trifluoromethyl)-1,4-diazepane instead of piperidine in experimental procedure 1.3. LCMS [M+1]⁺ 365.2. ¹H NMR (400 MHz, CDCl₃) δ 4.36-4.28 (m, 1H), 4.09-4.01 (m, 1H), 3.81-3.72 (m, 1H), 3.42-3.33 (m, 1H), 3.09-3.02 (m, 1H), 2.93-2.80 (m, 6H), 2.73-2.67 (m, 2H), 2.61-2.53 (m, 1H), 2.22-1.99 (m, 7H), 1.89-1.82 (m, 1H), 1.43-1.34 (m, 2H), 1.17 (s, 3H).

### Synthesis of rac-rel-trans-3-((3R,4R)-4-methylpyrrolidin-3-yl)-5-(piperidin-1-ylmethyl)-5,6-dihydro-1,4,2-dioxazine, ID 551

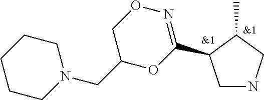 [SMILES: C[C@@H]1CNC[C@H]1C1=NOCC(CN2CCCCC2)O1]

The general synthesis using halo-cyclization as described herein was used to provide rac-rel-trans-3-((3R,4R)-4-methylpyrrolidin-3-yl)-5-(piperidin-1-ylmethyl)-5,6-dihydro-1,4,2-dioxazine (164.5 mg, 21.45%) as a yellow oil from commercially available rel-trans-(3R,4R)-1-(tert-butoxycarbonyl)-4-methylpyrrolidine-3-carboxylic acid in line with the synthesis described in 1.1 to 1.4. LCMS [M+1]⁺ 268.4. ¹H NMR (400 MHz, CDCl₃) b 4.40-4.33 (m, 1H), 4.12-4.03 (m, 1H), 3.77-3.66 (m, 1H), 3.23-3.14 (m, 1H), 3.14-2.81 (m, 2H), 2.58-2.48 (m, 2H), 2.48-2.29 (m, 7H), 2.29-2.19 (m, 1H), 1.58-1.49 (m, 4H), 1.45-1.34 (m, 2H), 1.13-1.02 (m, 3H).

### Synthesis of rac-5-(piperidin-1-ylmethyl)-3-(3-(tetrahydro-2H-pyran-4-yl)pyrrolidin-3-yl)-5,6-dihydro-1,4,2-dioxazine, ID 583

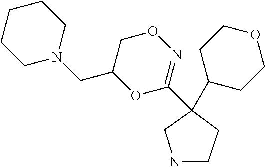 [SMILES: C1CCN(CC2CON=C(C3(C4CCOCC4)CCNC3)O2)CC1]

The general synthesis using halo-cyclization as described herein was used to provide rac-5-(piperidin-1-ylmethyl)-3-(3-(tetrahydro-2H-pyran-4-yl)pyrrolidin-3-yl)-5,6-dihydro-1,4,2-dioxazine (151.5 mg, 36.24%) as a yellow oil from commercially available rac-1-(tert-butoxycarbonyl)-3-(tetrahydro-2H-pyran-4-yl)pyrrolidine-3-carboxylic acid in line with the synthesis described in 1.1 to 1.4. LCMS [M+1]⁺ 338.2. ¹H NMR (400 MHz, CDCl₃) δ 4.40-4.31 (m, 1H), 4.06 (dd, J=11.6, 2.8 Hz, 1H), 4.01-3.93 (m, 2H), 3.77-3.68 (m, 1H), 3.41-3.25 (m, 3H), 3.03-2.93 (m, 1H), 2.93-2.83 (m, 1H), 2.61 (t, J=12.0, 12.0 Hz, 1H), 2.57-2.48 (m, 2H), 2.47-2.34 (m, 5H), 2.29-2.20 (m, 1H), 1.83-1.71 (m, 1H), 1.68-1.58 (m, 1H), 1.58-1.43 (m, 8H), 1.43-1.30 (m, 2H).

### Synthesis of rac-5-(piperidin-1-ylmethyl)-3-(2-(piperidin-4-yl)propan-2-yl)-5,6-dihydro-1,4,2-dioxazine di-2,2,2-trifluoroacetate, ID 477

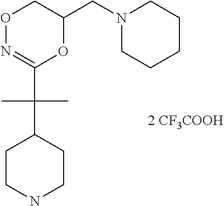 [SMILES: CC(C)(C1=NOCC(CN2CCCCC2)O1)C1CCNCC1]

The general synthesis using halo-cyclization as described herein was used to provide rac-5-(piperidin-1-ylmethyl)-3-(2-(piperidin-4-yl)propan-2-yl)-5,6-dihydro-1,4,2-dioxazine di-2,2,2-trifluoroacetate (497.3 mg, 51.17%) as a yellow oil from commercially available 2-(1-(tert-butoxycarbonyl)piperidin-4-yl)-2-methylpropanoic acid in line with the synthesis described in 1.1 to 1.4. LCMS [M+1]⁺ 310.2. ¹H NMR (400 MHz, D₂O) δ 4.89-4.80 (m, 1H), 4.07 (dd, J=11.9, 2.8 Hz, 1H), 3.65 (dd, J=11.9, 6.9 Hz, 1H), 3.53 (d, J=12.1 Hz, 1H), 3.47-3.41 (m, 1H), 3.37-3.30 (m, 4H), 3.03-2.91 (m, 2H), 2.87-2.76 (m, 2H), 1.89-1.64 (m, 8H), 1.40 (q, J=12.7, 12.6, 12.6 Hz, 3H), 1.11-0.95 (m, 6H).

### Synthesis of rac-3-(2-methylmorpholin-2-yl)-5-(piperidin-1-ylmethyl)-5,6-dihydro-1,4,2-dioxazine, ID 480

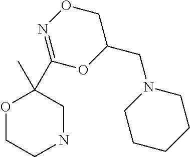 [SMILES: CC1(C2=NOCC(CN3CCCCC3)O2)CNCCO1]

The general synthesis using halo-cyclization as described herein was used to provide rac-3-(2-methylmorpholin-2-yl)-5-(piperidin-1-ylmethyl)-5,6-dihydro-1,4,2-dioxazine (145.7 mg, 25.28%) as a yellow oil from commercially available rac-4-(tert-butoxycarbonyl)-2-methylmorpholine-2-carboxylic acid in line with the synthesis described in 1.1 to 1.4. LCMS [M+1]⁺ 284.4. ¹HNMR (400 MHz, CD₃OD) δ 4.62-4.50 (m, 1H), 4.23-4.08 (m, 1H), 3.87-3.76 (m, 1H), 3.74-3.56 (m, 2H), 3.28-3.22 (m, 1H), 2.76 (d, J=5.4 Hz, 2H), 2.68-2.43 (m, 7H), 1.67-1.54 (m, 4H), 1.52-1.41 (m, 2H), 1.27 (s, 3H).

### Synthesis of rac-3-(2-methylpyrrolidin-3-yl)-5-(piperidin-1-ylmethyl)-5,6-dihydro-1,4,2-dioxazine, ID 559

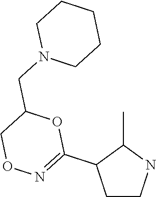 [SMILES: CC1NCCC1C1=NOCC(CN2CCCCC2)O1]

The general synthesis using halo-cyclization as described herein was used to provide rac-3-(2-methylpyrrolidin-3-yl)-5-(piperidin-1-ylmethyl)-5,6-dihydro-1,4,2-dioxazine (46.6 mg, 19.99%) as a pale brown oil from commercially available rac-1-(tert-butoxycarbonyl)-2-methylpyrrolidine-3-carboxylic acid in line with the synthesis described in 1.1 to 1.4. LCMS [M+1]⁺ 268.2. ¹H NMR (400 MHz, CDCl₃) δ 4.46-4.30 (m, 1H), 4.13-3.98 (m, 1H), 3.82-3.60 (m, 1H), 3.20-3.02 (m, 2H), 2.86-2.66 (m, 2H), 2.59-2.46 (m, 2H), 2.46-2.32 (m, 4H), 2.06-1.95 (m, 2H), 1.94-1.86 (m, 3H), 1.62-1.54 (m, 2H), 1.45-1.33 (m, 2H), 1.26-1.07 (m, 3H).

### Synthesis of rac-5-(piperidin-1-ylmethyl)-3-(pyrrolidin-3-yl)-5,6-dihydro-1,4,2-dioxazine, ID 508

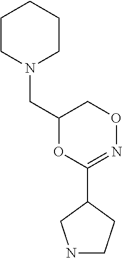 [SMILES: C1CCN(CC2CON=C(C3CCNC3)O2)CC1]

The general synthesis using halo-cyclization as described herein was used to provide rac-5-(piperidin-1-ylmethyl)-3-(pyrrolidin-3-yl)-5,6-dihydro-1,4,2-dioxazine (523.7 mg, 31.25%) as a yellow oil from commercially available rac-1-(tert-butoxycarbonyl)pyrrolidine-3-carboxylic acid in line with the synthesis described in 1.1 to 1.4. LCMS [M+1]⁺ 254.4. ¹H NMR (400 MHz, CDCl₃) δ 4.43-4.30 (m, 1H), 4.07 (d, J=11.2 Hz, 1H), 3.76-3.66 (m, 1H), 3.58-3.36 (m, 2H), 3.19-2.98 (m, 3H), 2.97-2.81 (m, 2H), 2.57-2.29 (m, 6H), 2.07-1.88 (m, 2H), 1.55-1.47 (m, 3H), 1.44-1.31 (m, 2H).

### Synthesis of rac-rel-trans-(3aR,6aR)-3a-(5-(piperidin-1-ylmethyl)-5,6-dihydro-1,4,2-dioxazin-3-yl)hexahydro-1H-furo[3,4-c]pyrrole, ID 494

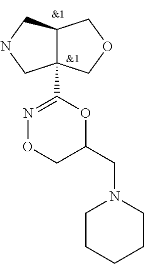 [SMILES: C1CCN(CC2CON=C([C@]34CNC[C@H]3COC4)O2)CC1]

The general synthesis using halo-cyclization as described herein was used to provide rac-rel-trans-(3aR,6aR)-3a-(5-(piperidin-1-ylmethyl)-5,6-dihydro-1,4,2-dioxazin-3-yl)hexahydro-1H-furo[3,4-c]pyrrole (19.8 mg, 7.7%) as a yellow oil from commercially available rel-trans-(3aR,6aR)-5-(tert-butoxycarbonyl)tetrahydro-1H-furo[3,4-c]pyrrole-3a(3H)-carboxylic acid in line with the synthesis described in 1.1 to 1.4. LCMS [M+1]⁺ 296.2. ¹H NMR (400 MHz, CDCl₃) δ 4.45-4.37 (m, 1H), 4.11 (dt, J=11.6, 3.3, 3.3 Hz, 1H), 3.94 (dd, J=9.2, 3.4 Hz, 1H), 3.88-3.83 (m, 1H), 3.81-3.73 (m, 2H), 3.67 (dd, J=9.0, 2.6 Hz, 1H), 3.31 (d, J=11.8 Hz, 1H), 3.27-3.18 (m, 1H), 3.07-2.97 (m, 1H), 2.87 (d, J=11.7 Hz, 1H), 2.73 (dd, J=11.6, 4.5 Hz, 1H), 2.53 (d, J=6.1 Hz, 2H), 2.52-2.45 (m, 2H), 2.45-2.36 (m, 2H), 1.61-1.50 (m, 5H), 1.47-1.39 (m, 2H).

### Synthesis of rac-rel-trans-(3aR,7aR)-3a-(5-(piperidin-1-ylmethyl)-5,6-dihydro-1,4,2-dioxazin-3-yl)octahydropyrano[3,4-c]pyrrole, ID 461

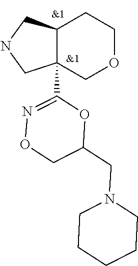 [SMILES: C1CCN(CC2CON=C([C@]34CNC[C@H]3CCOC4)O2)CC1]

The general synthesis using halo-cyclization as described herein was used to provide rac-rel-trans-(3aR,7aR)-3a-(5-(piperidin-1-ylmethyl)-5,6-dihydro-1,4,2-dioxazin-3-yl)octahydropyrano[3,4-c]pyrrole (123.8 mg, 41.18%) as a yellow oil from commercially available rel-trans-(3aR,7aR)-2-(tert-butoxycarbonyl)hexahydropyrano[3,4-c]pyrrole-3a(4H)-carboxylic acid in line with the synthesis described in 1.1 to 1.4. LCMS [M+1]⁺ 310.2. ¹H NMR (400 MHz, CDCl₃) δ 4.47-4.31 (m, 1H), 4.08 (d, J=10.6 Hz, 1H), 3.97-3.83 (m, 1H), 3.80-3.53 (m, 4H), 3.33-3.04 (m, 2H), 3.01-2.77 (m, 2H), 2.75-2.53 (m, 2H), 2.52-2.22 (m, 8H), 2.07-1.82 (m, 1H), 1.63-1.51 (m, 3H), 1.45-1.32 (m, 2H).

### Synthesis of rac-5-(piperidin-1-ylmethyl)-3-(1-(piperidin-4-yl)cyclopropyl)-5,6-dihydro-1,4,2-dioxazine, ID 521

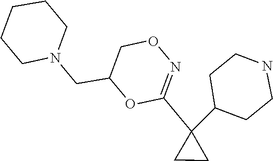

The general synthesis using halo-cyclization as described herein was used to provide rac-5-(piperidin-1-ylmethyl)-3-(1-(piperidin-4-yl)cyclopropyl)-5,6-dihydro-1,4,2-dioxazine (23.6 mg, 14.9%) as a yellow oil from commercially available 1-(1-(tert-butoxycarbonyl)piperidin-4-yl)cyclopropane-1-carboxylic acid in line with the synthesis described in 1.1 to 1.4. LCMS [M+1]⁺ 308.2. ¹H NMR (400 MHz, CDCl₃) δ 4.36-4.26 (m, 1H), 4.03 (dd, J=11.5, 2.8 Hz, 1H), 3.66 (dd, J=11.5, 6.5 Hz, 1H), 3.06 (d, J=11.6 Hz, 2H), 2.58-2.44 (m, 6H), 2.39-2.33 (m, 2H), 2.24-2.02 (m, 2H), 1.70-1.59 (m, 2H), 1.53-1.44 (m, 4H), 1.43-1.37 (m, 2H), 1.27 (qd, J=12.4, 12.4, 12.4, 3.9 Hz, 2H), 0.92-0.78 (m, 2H), 0.65-0.49 (m, 2H).

### Synthesis of rac-3-(3-methoxypyrrolidin-3-yl)-5-(piperidin-1-ylmethyl)-5,6-dihydro-1,4,2-dioxazine, ID 606

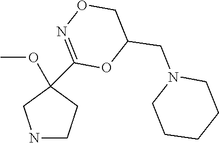 [SMILES: COC1(C2=NOCC(CN3CCCCC3)O2)CCNC1]

The general synthesis using halo-cyclization as described herein was used to provide rac-3-(3-methoxypyrrolidin-3-yl)-5-(piperidin-1-ylmethyl)-5,6-dihydro-1,4,2-dioxazine (116.5 mg, 9.99%) as a yellow oil from commercially available rac-1-(tert-butoxycarbonyl)-3-methoxypyrrolidine-3-carboxylic acid in line with the synthesis described in 1.1 to 1.4. LCMS [M+1]⁺ 284.2. ¹H NMR (400 MHz, CDCl₃) δ 4.47-4.35 (m, 1H), 4.11 (dt, J=11.6, 2.7, 2.7 Hz, 1H), 3.83 (ddd, J=11.5, 5.9, 3.7 Hz, 1H), 3.21 (s, 3H), 3.15-3.00 (m, 3H), 3.00-2.89 (m, 1H), 2.60-2.52 (m, 2H), 2.52-2.43 (m, 2H), 2.43-2.34 (m, 2H), 2.19-2.11 (m, 2H), 2.01-1.91 (m, 1H), 1.62-1.45 (m, 4H), 1.45-1.30 (m, 2H).

### Synthesis of rac-3-(5-methoxypiperidin-3-yl)-5-(piperidin-1-ylmethyl)-5,6-dihydro-1,4,2-dioxazine, ID 680

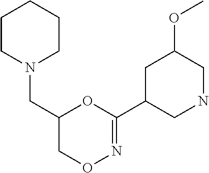 [SMILES: COC1CNCC(C2=NOCC(CN3CCCCC3)O2)C1]

The general synthesis using halo-cyclization as described herein was used to provide rac-3-(5-methoxypiperidin-3-yl)-5-(piperidin-1-ylmethyl)-5,6-dihydro-1,4,2-dioxazine (411.8 mg, 20.77%) as a yellow oil from commercially available rac-1-(tert-butoxycarbonyl)-5-methoxypiperidine-3-carboxylic acid in line with the synthesis described in 1.1 to 1.4. LCMS [M+1]⁺ 298.4. ¹H NMR (400 MHz, CD₃OD) δ 4.51 (qd, J=6.1, 6.1, 6.1, 2.9 Hz, 1H), 4.09 (dd, J=11.7, 2.9 Hz, 1H), 3.78-3.67 (m, 1H), 3.38-3.36 (m, 3H), 3.31-3.29 (m, 1H), 3.21 (dd, J=12.4, 3.8 Hz, 1H), 3.15-3.05 (m, 1H), 2.91-2.78 (m, 1H), 2.60 (d, J=5.6 Hz, 2H), 2.59-2.44 (m, 6H), 2.30-2.14 (m, 1H), 1.75-1.65 (m, 1H), 1.65-1.58 (m, 4H), 1.53-1.39 (m, 2H).

### Synthesis of rac-3-(2-azabicyclo[2.1.1]hexan-5-yl)-5-(piperidin-1-ylmethyl)-5,6-dihydro-1,4,2-dioxazine, ID 548

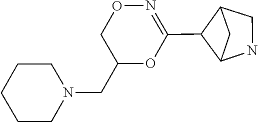

The general synthesis using halo-cyclization as described herein was used to provide rac-3-(2-azabicyclo[2.1.1]hexan-5-yl)-5-(piperidin-1-ylmethyl)-5,6-dihydro-1,4,2-dioxazine (28.3 mg, 9.26%) as a yellow oil from commercially available rac-2-(tert-butoxycarbonyl)-2-azabicyclo[2.1.1]hexane-5-carboxylic acid in line with the synthesis described in 1.1 to 1.4. LCMS [M+1]⁺ 266.2. ¹H NMR (400 MHz, CDCl₃) δ 4.34-4.28 (m, 1H), 4.09-4.02 (m, 1H), 3.79-3.73 (m, 1H), 3.73-3.67 (m, 1H), 2.91 (d, J=7.9 Hz, 2H), 2.85-2.78 (m, 1H), 2.54 (dd, J=13.3, 6.3 Hz, 1H), 2.51-2.14 (m, 11H), 1.49-1.32 (m, 3H), 1.19 (t, J=7.7, 7.7 Hz, 1H).

### Synthesis of rac-rel-trans-3-((1R,5R)-5-methyl-3-azabicyclo[3.1.0]hexan-1-yl)-5-(piperidin-1-ylmethyl)-5,6-dihydro-1,4,2-dioxazine, ID 481

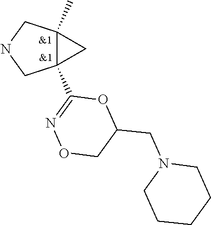 [SMILES: C[C@@]12CNC[C@]1(C1=NOCC(CN3CCCCC3)O1)C2]

The general synthesis using halo-cyclization as described herein was used to provide rac-rel-trans-3-((1R,5R)-5-methyl-3-azabicyclo[3.1.0]hexan-1-yl)-5-(piperidin-1-ylmethyl)-5,6-dihydro-1,4,2-dioxazine (22.9 mg, 4.85%) as a yellow oil from commercially available rel-trans-(1R,5R)-3-(tert-butoxycarbonyl)-5-methyl-3-azabicyclo[3.1.0]hexane-1-carboxylic acid in line with the synthesis described in 1.1 to 1.4. LCMS [M+1]⁺ 280.4. ¹H NMR (400 MHz, CD₃OD) δ 4.55-4.42 (m, 1H), 4.13-4.03 (m, 1H), 3.78-3.66 (m, 1H), 3.19 (d, J=11.5 Hz, 1H), 2.98-2.87 (m, 2H), 2.69 (d, J=11.5 Hz, 1H), 2.64-2.35 (m, 7H), 1.64-1.56 (m, 4H), 1.51-1.43 (m, 2H), 1.28-1.18 (m, 3H), 1.12-1.01 (m, 1H), 0.97-0.81 (m, 1H).

### Synthesis of rac-rel-trans-3-((1R,5R)-3-azabicyclo[3.2.0]heptan-1-yl)-5-(piperidin-1-ylmethyl)-5,6-dihydro-1,4,2-dioxazine di-2,2,2-trifluoroacetate, ID 485

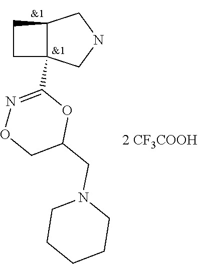 [SMILES: C1CCN(CC2CON=C([C@]34CC[C@H]3CNC4)O2)CC1]

The general synthesis using halo-cyclization as described herein was used to provide rac-rel-trans-3-((1R,5R)-3-azabicyclo[3.2.0]heptan-1-yl)-5-(piperidin-1-ylmethyl)-5,6-dihydro-1,4,2-dioxazine di-2,2,2-trifluoroacetate (32.3 mg, 14.62%) as a yellow oil from commercially available rel-trans-(1R,5R)-3-(tert-butoxycarbonyl)-3-azabicyclo[3.2.0]heptane-1-carboxylic acid in line with the synthesis described in 1.1 to 1.4. LCMS [M+1]⁺ 280.4. ¹H NMR (400 MHz, CDCl₃) δ 4.46-4.39 (m, 1H), 4.18-4.06 (m, 1H), 3.84-3.72 (m, 1H), 3.07-2.93 (m, 3H), 2.91-2.85 (m, 2H), 2.66-2.48 (m, 5H), 2.48-2.34 (m, 3H), 2.24-2.13 (m, 1H), 2.10-1.61 (m, 5H), 1.50-1.40 (m, 3H).

### Synthesis of rac-3-(3-methoxypiperidin-4-yl)-5-(piperidin-1-ylmethyl)-5,6-dihydro-1,4,2-dioxazine, ID 690

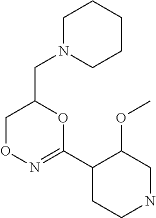

The general synthesis using halo-cyclization as described herein was used to provide rac-3-(3-methoxypiperidin-4-yl)-5-(piperidin-1-ylmethyl)-5,6-dihydro-1,4,2-dioxazine (42 mg, 2.47%) as a yellow oil from commercially available rac-1-(tert-butoxycarbonyl)-3-methoxypiperidine-4-carboxylic acid in line with the synthesis described in 1.1 to 1.4. LCMS [M+1]⁺ 298.2. ¹H NMR (400 MHz, CD₃OD) δ 4.56-4.40 (m, 1H), 4.16-4.01 (m, 1H), 3.79-3.66 (m, 1H), 3.63-3.51 (m, 1H), 3.36 (s, 3H), 3.29-3.19 (m, 1H), 3.08-2.93 (m, 1H), 2.72-2.40 (m, 9H), 1.92-1.75 (m, 1H), 1.71-1.54 (m, 5H), 1.53-1.42 (m, 2H).

### Synthesis of rac-3-(2-azabicyclo[2.1.1]hexan-4-yl)-5-(piperidin-1-ylmethyl)-5,6-dihydro-1,4,2-dioxazine, ID 527

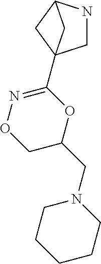 [SMILES: C1CCN(CC2CON=C(C34CNC(C3)C4)O2)CC1]

The general synthesis using halo-cyclization as described herein was used to provide rac-3-(2-azabicyclo[2.1.1]hexan-4-yl)-5-(piperidin-1-ylmethyl)-5,6-dihydro-1,4,2-dioxazine (113.7 mg, 17.5%) as a yellow oil from commercially available rac-2-(tert-butoxycarbonyl)-2-azabicyclo[2.1.1]hexane-4-carboxylic acid in line with the synthesis described in 1.1 to 1.4. LCMS [M+1]⁺ 266.2. ¹H NMR (400 MHz, cdcl₃) δ 4.41-4.34 (m, 1H), 4.12 (dd, J=11.6, 2.7 Hz, 1H), 3.80-3.72 (m, 2H), 3.12 (s, 2H), 3.09-2.82 (m, 4H), 2.52 (d, J=5.9 Hz, 2H), 2.50-2.44 (m, 2H), 2.41-2.32 (m, 2H), 2.07-1.99 (m, 2H), 1.61-1.55 (m, 3H), 1.40 (q, J=5.5, 5.5, 5.4 Hz, 2H).

### Synthesis of rac-4-(5-(piperidin-1-ylmethyl)-5,6-dihydro-1,4,2-dioxazin-3-yl)cyclohexan-1-amine, ID 611

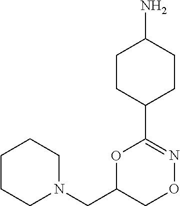 [SMILES: NC1CCC(C2=NOCC(CN3CCCCC3)O2)CC1]

The general synthesis using halo-cyclization as described herein was used to provide rac-4-(5-(piperidin-1-ylmethyl)-5,6-dihydro-1,4,2-dioxazin-3-yl)cyclohexan-1-amine (165.5 mg, 23.69%) as a yellow oil from commercially available 4-((tert-butoxycarbonyl)amino)cyclohexane-1-carboxylic acid in line with the synthesis described in 1.1 to 1.4. LCMS [M+1]⁺ 282.2. ¹H NMR (400 MHz, CD₃OD) δ 4.52-4.40 (m, 1H), 4.12-4.01 (m, 1H), 3.74-3.64 (m, 1H), 2.70-2.59 (m, 1H), 2.59-2.34 (m, 6H), 2.20-2.01 (m, 1H), 2.01-1.78 (m, 4H), 1.68-1.56 (m, 5H), 1.56-1.40 (m, 4H), 1.33-1.03 (m, 2H).

### Synthesis of rac-5-(piperidin-1-ylmethyl)-3-(2-(pyrrolidin-3-yl)propan-2-yl)-5,6-dihydro-1,4,2-dioxazine, ID 491

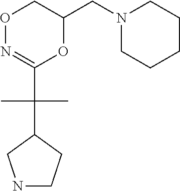 [SMILES: CC(C)(C1=NOCC(CN2CCCCC2)O1)C1CCNC1]

The general synthesis using halo-cyclization as described herein was used to provide rac-5-(piperidin-1-ylmethyl)-3-(2-(pyrrolidin-3-yl)propan-2-yl)-5,6-dihydro-1,4,2-dioxazine (71.6 mg, 23.48%) as a yellow oil from commercially available rac-2-(1-(tert-butoxycarbonyl)pyrrolidin-3-yl)-2-methylpropanoic acid in line with the synthesis described in 1.1 to 1.4. LCMS [M+1]⁺ 296.4. ¹H NMR (400 MHz, CDCl₃) δ 4.43-4.28 (m, 1H), 4.05 (d, J=11.4 Hz, 1H), 3.68 (dd, J=11.2, 6.3 Hz, 1H), 3.42-3.01 (m, 1H), 2.99-2.85 (m, 3H), 2.84-2.69 (m, 2H), 2.61-2.46 (m, 4H), 2.45-2.24 (m, 4H), 1.83-1.72 (m, 1H), 1.55-1.49 (m, 3H), 1.48-1.38 (m, 2H), 1.18-1.01 (m, 6H).

### Synthesis of rac-1-(6-isopropyl-3-(pyrrolidin-3-yl)-5,6-dihydro-1,4,2-dioxazin-5-yl)-N,N-dimethylmethanamine, ID 713

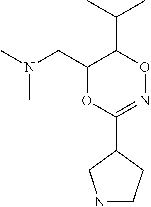 [SMILES: CC(C)C1ON=C(C2CCNC2)OC1CN(C)C]

The general synthesis using halo-cyclization as described herein was used to provide rac-1-(6-isopropyl-3-(pyrrolidin-3-yl)-5,6-dihydro-1,4,2-dioxazin-5-yl)-N,N-dimethylmethanamine (36.8 mg, 98.48%) as a yellow oil from commercially available 1-(tert-butoxycarbonyl)pyrrolidine-3-carboxylic acid in line with the synthesis described in 1.1 to 1.4 but using O-(1-isopropylallyl)hydroxylamine hydrochloride instead of 0-allylhydroxylamine hydrochloride in experimental procedure 1.1 and N-methylmethanamine hydrochloride instead of piperidine in experimental procedure 1.3. LCMS [M+1]⁺ 256.2. ¹H NMR (400 MHz, CD₃OD) δ 4.46-4.34 (m, 1H), 3.51-3.40 (m, 1H), 3.10-2.91 (m, 3H), 2.91-2.78 (m, 2H), 2.65-2.55 (m, 2H), 2.34 (s, 6H), 2.11-1.78 (m, 3H), 1.07 (d, J=6.8 Hz, 3H), 0.98 (d, J=6.8 Hz, 3H).

### Synthesis of rac-rel-cis-3-((3R,4R)-3,4-dimethylpyrrolidin-3-yl)-5-(piperidin-1-ylmethyl)-5,6-dihydro-1,4,2-dioxazine, ID 489

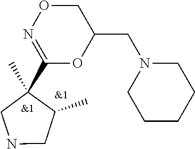 [SMILES: C[C@@H]1CNC[C@@]1(C)C1=NOCC(CN2CCCCC2)O1]

The general synthesis using halo-cyclization as described herein was used to provide rac-rel-cis-3-((3R,4R)-3,4-dimethylpyrrolidin-3-yl)-5-(piperidin-1-ylmethyl)-5,6-dihydro-1,4,2-dioxazine (99 mg, 25.31%) as a colorless oil from commercially available rel-cis-(3R,4R)-1-(tert-butoxycarbonyl)-3,4-dimethylpyrrolidine-3-carboxylic acid in line with the synthesis described in 1.1 to 1.4. LCMS [M+1]⁺ 282.2. ¹H NMR (400 MHz, CDCl₃) b 4.42-4.30 (m, 1H), 4.12-4.02 (m, 1H), 3.75-3.64 (m, 1H), 3.41-3.32 (m, 1H), 3.24-3.15 (m, 1H), 2.69-2.63 (m, 1H), 2.63-2.53 (m, 2H), 2.53-2.43 (m, 5H), 2.43-2.33 (m, 3H), 1.55-1.49 (m, 3H), 1.47-1.35 (m, 2H), 1.08 (s, 3H), 1.00-0.86 (m, 3H).

### Synthesis of rac-3-(4,4-dimethylpyrrolidin-3-yl)-5-(piperidin-1-ylmethyl)-5,6-dihydro-1,4,2-dioxazine, ID 688

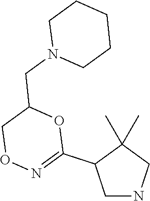 [SMILES: CC1(C)CNCC1C1=NOCC(CN2CCCCC2)O1]

The general synthesis using halo-cyclization as described herein was used to provide rac-3-(4,4-dimethylpyrrolidin-3-yl)-5-(piperidin-1-ylmethyl)-5,6-dihydro-1,4,2-dioxazine (251.5 mg, 22.65%) as an orange oil from commercially available rac-1-(tert-butoxycarbonyl)-4,4-dimethylpyrrolidine-3-carboxylic acid in line with the synthesis described in 1.1 to 1.4. LCMS [M+1]⁺ 282.4. ¹H NMR (400 MHz, CD₃OD) δ 4.56-4.43 (m, 1H), 4.17-4.05 (m, 1H), 3.81-3.70 (m, 1H), 3.24-3.07 (m, 2H), 2.81-2.66 (m, 2H), 2.66-2.43 (m, 7H), 1.67-1.54 (m, 4H), 1.54-1.40 (m, 2H), 1.18 (d, J=4.9 Hz, 3H), 1.06 (d, J=4.6 Hz, 3H).

### Synthesis of rac-5-(piperidin-1-ylmethyl)-3-(piperidin-4-ylmethyl)-5,6-dihydro-1,4,2-dioxazine, ID 479

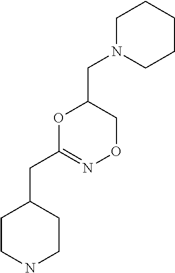 [SMILES: C1CCN(CC2CON=C(CC3CCNCC3)O2)CC1]

The general synthesis using halo-cyclization as described herein was used to provide rac-5-(piperidin-1-ylmethyl)-3-(piperidin-4-ylmethyl)-5,6-dihydro-1,4,2-dioxazine (390 mg, 35.25%) as a yellow oil from commercially available 2-(1-(tert-butoxycarbonyl)piperidin-4-yl)acetic acid in line with the synthesis described in 1.1 to 1.4. LCMS [M+1]⁺ 282.2. ¹H NMR (400 MHz, CDCl₃) δ 4.44-4.33 (m, 1H), 4.10 (dd, J=11.4, 2.3 Hz, 1H), 3.75 (dd, J=11.5, 6.4 Hz, 1H), 3.07 (d, J=12.4 Hz, 2H), 2.59 (t, J=12.2, 12.2 Hz, 2H), 2.55-2.45 (m, 4H), 2.45-2.36 (m, 2H), 2.11 (d, J=7.0 Hz, 2H), 1.88-1.76 (m, 3H), 1.76-1.63 (m, 3H), 1.57-1.51 (m, 2H), 1.49-1.38 (m, 2H), 1.26-1.11 (m, 2H).

### Synthesis of rac-3-(3-methylazetidin-3-yl)-5-(piperidin-1-ylmethyl)-5,6-dihydro-1,4,2-dioxazine, ID 549

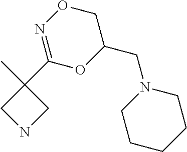 [SMILES: CC1(C2=NOCC(CN3CCCCC3)O2)CNC1]

The general synthesis using halo-cyclization as described herein was used to provide rac-3-(3-methylazetidin-3-yl)-5-(piperidin-1-ylmethyl)-5,6-dihydro-1,4,2-dioxazine (22.5 mg, 8.52%) as a yellow oil from commercially available rac-1-(tert-butoxycarbonyl)-3-methylazetidine-3-carboxylic acid in line with the synthesis described in 1.1 to 1.4. LCMS [M+1]⁺ 254.4. ¹H NMR (400 MHz, CDCl₃) δ 4.45-4.32 (m, 1H), 4.09 (dd, J=11.6, 2.8 Hz, 1H), 3.95 (d, J=7.3 Hz, 2H), 3.74 (dd, J=11.6, 6.3 Hz, 1H), 3.29 (d, J=7.9 Hz, 2H), 2.52 (d, J=6.0 Hz, 2H), 2.50-2.42 (m, 2H), 2.42-2.32 (m, 2H), 2.02-1.76 (m, 4H), 1.53-1.48 (m, 4H), 1.45-1.35 (m, 2H).

### Synthesis of rac-3-(2-methylpyrrolidin-2-yl)-5-(piperidin-1-ylmethyl)-5,6-dihydro-1,4,2-dioxazine, ID 566

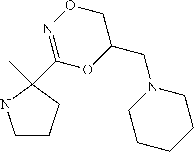 [SMILES: CC1(C2=NOCC(CN3CCCCC3)O2)CCCN1]

The general synthesis using halo-cyclization as described herein was used to provide rac-3-(2-methylpyrrolidin-2-yl)-5-(piperidin-1-ylmethyl)-5,6-dihydro-1,4,2-dioxazine (570.5 mg, 92.25%) as a yellow oil from commercially available rac-1-(tert-butoxycarbonyl)-2-methylpyrrolidine-2-carboxylic acid in line with the synthesis described in 1.1 to 1.4. LCMS [M+1]⁺ 268.2. ¹H NMR (400 MHz, dmso) δ 4.49-4.40 (m, 1H), 4.01 (dt, J=11.6, 2.3, 2.3 Hz, 1H), 3.74-3.64 (m, 1H), 2.95-2.87 (m, 2H), 2.48-2.31 (m, 7H), 2.17-2.06 (m, 1H), 1.79-1.68 (m, 2H), 1.56-1.42 (m, 5H), 1.40-1.32 (m, 2H), 1.28 (d, J=2.9 Hz, 3H).

### Synthesis of rac-3-(4-methoxy-2-methylpyrrolidin-2-yl)-5-(piperidin-1-ylmethyl)-5,6-dihydro-1,4,2-dioxazine, ID 565

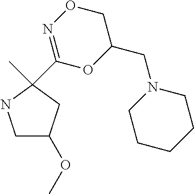 [SMILES: COC1CNC(C)(C2=NOCC(CN3CCCCC3)O2)C1]

The general synthesis using halo-cyclization as described herein was used to provide rac-3-(4-methoxy-2-methylpyrrolidin-2-yl)-5-(piperidin-1-ylmethyl)-5,6-dihydro-1,4,2-dioxazine (118.8 mg, 31.76%) as a yellow oil from commercially available rac-1-(tert-butoxycarbonyl)-4-methoxy-2-methylpyrrolidine-2-carboxylic acid in line with the synthesis described in 1.1 to 1.4. LCMS [M+1]⁺ 298.4. ¹H NMR (400 MHz, CDCl₃) δ 4.43-4.30 (m, 1H), 4.11-4.00 (m, 1H), 3.95-3.85 (m, 1H), 3.76-3.65 (m, 1H), 3.28-3.18 (m, 3H), 3.18-3.03 (m, 1H), 3.03-2.94 (m, 1H), 2.62-2.47 (m, 3H), 2.47-2.40 (m, 2H), 2.40-2.25 (m, 4H), 1.79-1.58 (m, 1H), 1.54-1.46 (m, 3H), 1.45-1.25 (m, 5H).

### Synthesis of rac-3-(3-azabicyclo[5.1.0]octan-7-yl)-5-(piperidin-1-ylmethyl)-5,6-dihydro-1,4,2-dioxazine, ID 519

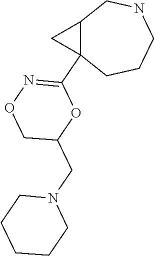 [SMILES: C1CCN(CC2CON=C(C34CCCNCC3C4)O2)CC1]

The general synthesis using halo-cyclization as described herein was used to provide rac-3-(3-azabicyclo[5.1.0]octan-7-yl)-5-(piperidin-1-ylmethyl)-5,6-dihydro-1,4,2-dioxazine (14.4 mg, 10.19%) as a brown oil from commercially available rac-3-(tert-butoxycarbonyl)-3-azabicyclo[5.1.0]octane-7-carboxylic acid in line with the synthesis described in 1.1 to 1.4. LCMS [M+1]⁺ 294.4. ¹H NMR (400 MHz, CDCl₃) δ 4.38-4.12 (m, 3H), 4.08-4.00 (m, 1H), 3.73-3.57 (m, 1H), 3.27-2.93 (m, 3H), 2.88-2.75 (m, 1H), 2.75-2.62 (m, 1H), 2.55-2.38 (m, 4H), 2.38-2.23 (m, 3H), 1.57-1.43 (m, 6H), 1.42-1.34 (m, 2H), 1.30-1.20 (m, 1H), 0.85-0.58 (m, 1H).

### Synthesis of rac-3-(3-(methoxymethyl)pyrrolidin-3-yl)-5-(piperidin-1-ylmethyl)-5,6-dihydro-1,4,2-dioxazine, ID 594

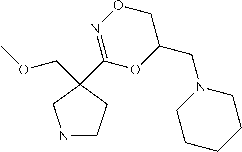 [SMILES: COCC1(C2=NOCC(CN3CCCCC3)O2)CCNC1]

The general synthesis using halo-cyclization as described herein was used to provide rac-3-(3-(methoxymethyl)pyrrolidin-3-yl)-5-(piperidin-1-ylmethyl)-5,6-dihydro-1,4,2-dioxazine (121.8 mg, 78.11%) as a yellow oil from commercially available rac-1-(tert-butoxycarbonyl)-3-(methoxymethyl)pyrrolidine-3-carboxylic acid in line with the synthesis described in 1.1 to 1.4. LCMS [M+1]⁺ 298.4. ¹H NMR (400 MHz, CD₃OD) δ 4.57-4.45 (m, 1H), 4.09 (dt, J=11.6, 3.0, 3.0 Hz, 1H), 3.74 (ddd, J=11.6, 6.4, 3.0 Hz, 1H), 3.62-3.53 (m, 1H), 3.49-3.41 (m, 2H), 3.34 (s, 3H), 3.19-3.06 (m, 2H), 3.00 (dd, J=12.0, 2.1 Hz, 1H), 2.58 (d, J=5.8 Hz, 3H), 2.53-2.43 (m, 3H), 2.29-2.18 (m, 1H), 1.96-1.84 (m, 1H), 1.65-1.54 (m, 4H), 1.54-1.41 (m, 2H).

### Synthesis of rac-3-(4-methylazepan-4-yl)-5-(piperidin-1-ylmethyl)-5,6-dihydro-1,4,2-dioxazine, ID 542

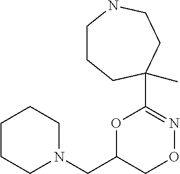 [SMILES: CC1(C2=NOCC(CN3CCCCC3)O2)CCCNCC1]

The general synthesis using halo-cyclization as described herein was used to provide 3-(4-methylazepan-4-yl)-5-(piperidin-1-ylmethyl)-5,6-dihydro-1,4,2-dioxazine (134.9 mg, 95.33%) as a yellow oil from commercially available rac-1-(tert-butoxycarbonyl)-4-methylazepane-4-carboxylic acid in line with the synthesis described in 1.1 to 1.4. LCMS [M+1]⁺ 296.4. ¹H NMR (400 MHz, CD₃OD) δ 4.57-4.48 (m, 1H), 4.15-4.07 (m, 1H), 3.79-3.66 (m, 1H), 3.27-3.23 (m, 2H), 3.20-3.16 (m, 2H), 2.71-2.40 (m, 7H), 2.37-2.29 (m, 1H), 2.27-2.17 (m, 1H), 1.93-1.82 (m, 2H), 1.76-1.68 (m, 1H), 1.64-1.56 (m, 5H), 1.51-1.42 (m, 2H), 1.21 (s, 3H).

### Synthesis of rac-3-(4-(2-methoxyethyl)piperidin-4-yl)-5-(piperidin-1-ylmethyl)-5,6-dihydro-1,4,2-dioxazine, ID 726

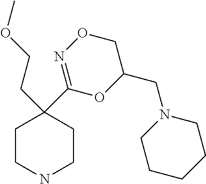 [SMILES: COCCC1(C2=NOCC(CN3CCCCC3)O2)CCNCC1]

The general synthesis using halo-cyclization as described herein was used to provide 3-(4-(2-methoxyethyl)piperidin-4-yl)-5-(piperidin-1-ylmethyl)-5,6-dihydro-1,4,2-dioxazine (166.6 mg, 54.46%) as a yellow oil from commercially available 1-(tert-butoxycarbonyl)-4-(2-methoxyethyl)piperidine-4-carboxylic acid in line with the synthesis described in 1.1 to 1.4. LCMS [M+1]⁺ 326.4. ¹H NMR (Chloroform-d, 400 MHz): δ (ppm) 4.42-4.31 (m, 1H), 4.15-4.03 (m, 1H), 3.74 (dd, J=11.4, 6.5 Hz, 1H), 3.40 (t, J=6.9, 6.9 Hz, 2H), 3.29 (s, 3H), 2.94-2.66 (m, 4H), 2.55-2.44 (m, 4H), 2.43-2.34 (m, 2H), 2.08-1.99 (m, 2H), 1.99-1.92 (m, 1H), 1.77 (t, J=6.8, 6.8 Hz, 2H), 1.62-1.47 (m, 4H), 1.48-1.23 (m, 4H).

### Synthesis of rac-N-methyl-5-(5-(piperidin-1-ylmethyl)-5,6-dihydro-1,4,2-dioxazin-3-yl)piperidin-3-amine, ID 756

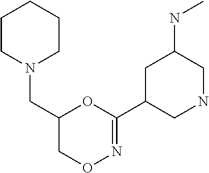 [SMILES: CNC1CNCC(C2=NOCC(CN3CCCCC3)O2)C1]

The general synthesis using halo-cyclization as described herein was used to provide N-methyl-5-(5-(piperidin-1-ylmethyl)-5,6-dihydro-1,4,2-dioxazin-3-yl)piperidin-3-amine (32.9 mg, 6.81%) as a yellow solid from the starting rac-1-(tert-butoxycarbonyl)-5-((tert-butoxycarbonyl)(methyl)amino)piperidine-3-carboxylic acid in line with the synthesis described in 1.1 to 1.4. The synthesis of the starting building block is described above. LCMS [M+1]⁺ 297.2. ¹H NMR (Methanol-d₄, 400 MHz): δ (ppm) 4.55-4.43 (m, 1H), 4.10 (d, J=13.2 Hz, 1H), 3.76-3.69 (m, 1H), 3.22-3.15 (m, 1H), 3.13-3.05 (m, 1H), 2.59-2.40 (m, 9H), 2.38 (s, 3H), 2.29-2.22 (m, 1H), 2.22-2.12 (m, 1H), 1.66-1.55 (m, 4H), 1.54-1.45 (m, 2H), 1.34-1.24 (m, 1H).

### Synthesis of rac-3-(1-methylcyclohexyl)-5-(piperidin-1-ylmethyl)-5,6-dihydro-1,4,2-dioxazine, ID 478

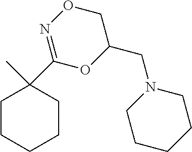 [SMILES: CC1(C2=NOCC(CN3CCCCC3)O2)CCCCC1]

The general synthesis using halo-cyclization as described herein was used to provide 3-(1-methylcyclohexyl)-5-(piperidin-1-ylmethyl)-5,6-dihydro-1,4,2-dioxazine (388.2 mg, 37.28%) as a yellow oil from commercially available 1-methylcyclohexane-1-carboxylic acid in line with the synthesis described in 1.1 to 1.3. LCMS [M+1]⁺ 281.4. ¹H NMR (400 MHz, CDCl₃) δ 4.38-4.29 (m, 1H), 4.04 (dd, J=11.4, 2.9 Hz, 1H), 3.68 (dd, J=11.4, 6.5 Hz, 1H), 2.57-2.43 (m, 4H), 2.43-2.32 (m, 2H), 1.94-1.83 (m, 2H), 1.59-1.49 (m, 4H), 1.49-1.44 (m, 4H), 1.44-1.37 (m, 3H), 1.32-1.25 (m, 1H), 1.25-1.17 (m, 2H), 1.11 (s, 3H).

### Synthesis of rac-(3aR,6aR)-3a-(5-(piperidin-1-ylmethyl)-5,6-dihydro-1,4,2-dioxazin-3-yl)hexahydro-1H-thieno[3,4-c]pyrrole 2,2-dioxide, ID 486

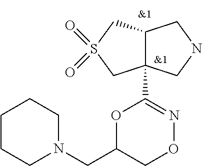 [SMILES: O=S1(=O)C[C@@H]2CNC[C@@]2(C2=NOCC(CN3CCCCC3)O2)C1]

The general synthesis using halo-cyclization as described herein was used to provide rac-(3aR,6aR)-3a-(5-(piperidin-1-ylmethyl)-5,6-dihydro-1,4,2-dioxazin-3-yl)hexahydro-1H-thieno[3,4-c]pyrrole 2,2-dioxide (25.3 mg, 43.99%) as a yellow oil from commercially available (3aR,6aR)-5-(tert-butoxycarbonyl)tetrahydro-1H-thieno[3,4-c]pyrrole-3a(3H)-carboxylic acid 2,2-dioxide in line with the synthesis described in 1.1 to 1.4. LCMS [M+1]⁺ 344.2. ¹H NMR (400 MHz, CDCl₃) δ 5.54-4.81 (m, 1H), 4.54-4.38 (m, 1H), 4.22-4.10 (m, 1H), 3.90-3.78 (m, 1H), 3.76-3.67 (m, 1H), 3.59-3.39 (m, 2H), 3.39-3.24 (m, 2H), 3.13-3.00 (m, 2H), 3.00-2.85 (m, 2H), 2.69-2.49 (m, 3H), 2.49-2.41 (m, 3H), 2.12-1.69 (m, 2H), 1.64-1.58 (m, 2H), 1.50-1.39 (m, 3H).

### Halocyclization+Additional Reductive Amination

### Synthesis of rac-3-(1-methylpiperidin-3-yl)-5-(piperidin-1-ylmethyl)-5,6-dihydro-1,4,2-dioxazine, ID 648

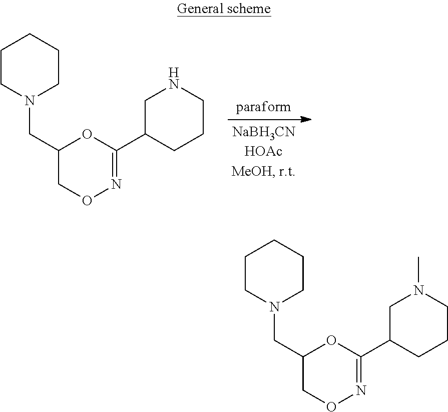 [SMILES: C1CCN(CC2CON=C(C3CCCNC3)O2)CC1 ; CN1CCCC(C2=NOCC(CN3CCCCC3)O2)C1]

### Procedure 1.5

### Synthesis of rac-3-(1-methylpiperidin-3-yl)-5-(piperidin-1-ylmethyl)-5,6-dihydro-1,4,2-dioxazine

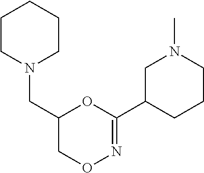 [SMILES: CN1CCCC(C2=NOCC(CN3CCCCC3)O2)C1]

rac-3-(3-Piperidyl)-5-(1-piperidylmethyl)-5,6-dihydro-1,4,2-dioxazine, obtained in a similar manner with non-critical variations in line with the synthesis described in 1.1 to 1.4 from commercially available rac-1-(tert-butoxycarbonyl)piperidine-3-carboxylic acid (200 mg, 0.7480 mmol, 1 eq) was dissolved in absolute methanol (2 mL), after that acetic acid (224.6 mg, 3.7401 mmol, 5 eq) was added, followed by the addition of paraform (235.84 mg, 2.6181 mmol, 3.5 eq) and sodium cyanoboranuide (164.52 mg, 2.6181 mmol, 3.5 eq). The reaction mixture was the left while stirring at ambient temperature overnight. After 24 hours the reaction mixture was concentrated under reduced pressure and the residue obtained was diluted with DCM (5 mL) and washed with 30% aqueous solution of potassium carbonate (2×3 mL). The organic layer was isolated and concentrated under reduced pressure to afford crude oily residue, which was purified with preparative HPLC (40-80% 0-5 min water-methanol; flow: 30 ml/min (loading pump 4 ml/min methanol); target mass 317; column SunFireC18 100×19 mm 5 um) to afford the title product (139.1 mg, 62.78%) as a yellow oil. LCMS [M+1]⁺ 282.2. ¹H NMR (400 MHz, CD₃OD) δ 4.54-4.44 (m, 1H), 4.10 (dd, J=11.5, 2.9 Hz, 1H), 3.72 (dd, J=11.6, 6.5 Hz, 1H), 3.00-2.89 (m, 1H), 2.87-2.75 (m, 1H), 2.60-2.40 (m, 7H), 2.29 (s, 3H), 2.11-2.00 (m, 1H), 2.00-1.85 (m, 2H), 1.83-1.72 (m, 1H), 1.66-1.54 (m, 5H), 1.52-1.42 (m, 2H), 1.42-1.26 (m, 1H).

### Synthesis of rac-6-isopropyl-3-(1-methylpyrrolidin-3-yl)-5-(piperidin-1-ylmethyl)-5,6-dihydro-1,4,2-dioxazine, ID 707

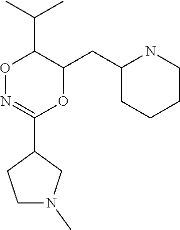 [SMILES: CC(C)C1ON=C(C2CCN(C)C2)OC1CC1CCCCN1]

The general synthesis using halo-cyclization as described herein was used to provide 6-isopropyl-3-(1-methylpyrrolidin-3-yl)-5-(piperidin-1-ylmethyl)-5,6-dihydro-1,4,2-dioxazine (20 mg, 16.8%) as a yellow oil from commercially available rac-1-(tert-butoxycarbonyl)pyrrolidine-3-carboxylic acid in line with the synthesis described in 1.1 to 1.5 but using O-(1-isopropylallyl)hydroxylamine hydrochloride instead of 0-allylhydroxylamine hydrochloride in experimental procedure 1.1. LCMS [M+1]⁺ 310.2. ¹H NMR (400 MHz, CD₃OD) δ 4.48-4.37 (m, 1H), 3.53-3.42 (m, 1H), 3.05-2.94 (m, 1H), 2.94-2.83 (m, 1H), 2.79-2.71 (m, 1H), 2.66 (dd, J=13.9, 4.2 Hz, 1H), 2.61-2.42 (m, 7H), 2.36 (s, 3H), 2.14-1.96 (m, 3H), 1.67-1.57 (m, 4H), 1.53-1.43 (m, 2H), 1.06 (d, J=6.9 Hz, 3H), 0.98 (d, J=6.8 Hz, 3H).

### Synthesis of rac-3-(1,3-dimethylpiperidin-3-yl)-5-(piperidin-1-ylmethyl)-5,6-dihydro-1,4,2-dioxazine, ID 529

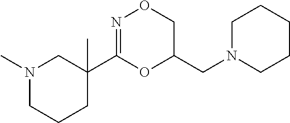 [SMILES: CN1CCCC(C)(C2=NOCC(CN3CCCCC3)O2)C1]

The general synthesis using halo-cyclization as described herein was used to provide 3-(1,3-dimethylpiperidin-3-yl)-5-(piperidin-1-ylmethyl)-5,6-dihydro-1,4,2-dioxazine (94.2 mg, 25.07%) as a yellow oil from commercially available 1-(tert-butoxycarbonyl)-3-methylpiperidine-3-carboxylic acid in line with the synthesis described in 1.1 to 1.5. LCMS [M+1]⁺ 296.4. ¹H NMR (400 MHz, cdcl₃) δ 4.48-4.32 (m, 1H), 4.08 (td, J=11.6, 11.5, 2.9 Hz, 1H), 3.77-3.61 (m, 1H), 2.74-2.58 (m, 1H), 2.58-2.47 (m, 4H), 2.47-2.26 (m, 5H), 2.26-2.21 (m, 3H), 2.21-1.98 (m, 2H), 1.91-1.66 (m, 2H), 1.66-1.57 (m, 1H), 1.57-1.54 (m, 2H), 1.45-1.35 (m, 2H), 1.34-1.24 (m, 1H), 1.19 (s, 3H).

### Synthesis of rac-3-(1-methylpyrrolidin-3-yl)-5-(piperidin-1-ylmethyl)-5,6-dihydro-1,4,2-dioxazine, ID 546

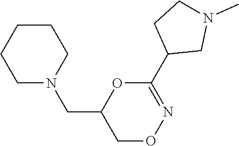 [SMILES: CN1CCC(C2=NOCC(CN3CCCCC3)O2)C1]

The general synthesis using halo-cyclization as described herein was used to provide rac-3-(1-methylpyrrolidin-3-yl)-5-(piperidin-1-ylmethyl)-5,6-dihydro-1,4,2-dioxazine (134.5 mg, 16.82%) as a pale brown oil from commercially available rac-1-(tert-butoxycarbonyl)pyrrolidine-3-carboxylic acid in line with the synthesis described in 1.1 to 1.5. LCMS [M+1]⁺ 268.2. ¹H NMR (400 MHz, CDCl₃) δ 4.40-4.35 (m, 1H), 4.06 (dd, J=11.5, 2.7 Hz, 1H), 3.77-3.68 (m, 1H), 3.04-2.91 (m, 1H), 2.78 (td, J=9.0, 9.0, 4.2 Hz, 1H), 2.64-2.42 (m, 8H), 2.41-2.36 (m, 2H), 2.33 (s, 3H), 2.06-1.96 (m, 2H), 1.96-1.82 (m, 3H), 1.44-1.38 (m, 2H).

### Synthesis of rac-3-(3-methoxy-1-methylpiperidin-4-yl)-5-(piperidin-1-ylmethyl)-5,6-dihydro-1,4,2-dioxazine, ID 720

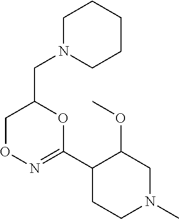 [SMILES: COC1CN(C)CCC1C1=NOCC(CN2CCCCC2)O1]

The general synthesis using halo-cyclization as described herein was used to provide rac-3-(3-methoxy-1-methylpiperidin-4-yl)-5-(piperidin-1-ylmethyl)-5,6-dihydro-1,4,2-dioxazine (71.9 mg, 16.31%) as a yellow oil from commercially available rac-1-(tert-butoxycarbonyl)-3-methoxypiperidine-4-carboxylic acid in line with the synthesis described in 1.1 to 1.5. LCMS [M+1]⁺ 312.2. ¹H NMR (Methanol-d₄, 400 MHz): δ (ppm) 4.60-4.40 (m, 1H), 4.11 (d, 1H), 3.80-3.60 (m, 2H), 3.37 (s, 3H), 3.32-3.28 (m, 1H), 3.28-3.15 (m, 1H), 2.93-2.84 (m, 1H), 2.71-2.36 (m, 7H), 2.29 (s, 3H), 2.16-1.95 (m, 3H), 1.69-1.59 (m, 4H), 1.56-1.33 (m, 2H).

### Synthesis of rac-3-(1-methylpiperidin-4-yl)-5-(piperidin-1-ylmethyl)-5,6-dihydro-1,4,2-dioxazine, ID 770

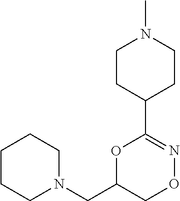 [SMILES: CN1CCC(C2=NOCC(CN3CCCCC3)O2)CC1]

The general synthesis using halo-cyclization as described herein was used to provide rac-3-(1-methylpiperidin-4-yl)-5-(piperidin-1-ylmethyl)-5,6-dihydro-1,4,2-dioxazine (73 mg, 52.72%) as a yellow oil from commercially available 1-(tert-butoxycarbonyl)-piperidine-4-carboxylic acid in line with the synthesis described in 1.1 to 1.5. LCMS [M+1]+282.2. ¹H NMR (Methanol-d₄, 400 MHz): δ (ppm) 4.50-4.40 (m, 1H), 4.07 (dd, J=12.1, 3.1 Hz, 1H), 3.75-3.63 (m, 1H), 2.94-2.82 (m, 2H), 2.62-2.41 (m, 6H), 2.25 (s, 3H), 2.20-2.12 (m, 1H), 2.03 (t, J=11.9, 11.9 Hz, 2H), 1.91-1.79 (m, 2H), 1.80-1.66 (m, 2H), 1.64-1.54 (m, 4H), 1.49-1.36 (m, 2H).

### Synthesis of rac-3-(4-methoxy-1-methylpiperidin-3-yl)-5-(piperidin-1-ylmethyl)-5,6-dihydro-1,4,2-dioxazine, ID 700

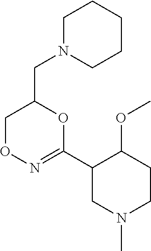 [SMILES: COC1CCN(C)CC1C1=NOCC(CN2CCCCC2)O1]

The general synthesis using halo-cyclization as described herein was used to provide rac-3-(4-methoxy-1-methylpiperidin-3-yl)-5-(piperidin-1-ylmethyl)-5,6-dihydro-1,4,2-dioxazine (31.3 mg, 7.47%) as a yellow oil from commercially available rac-1-(tert-butoxycarbonyl)-4-methoxypiperidine-3-carboxylic acid in line with the synthesis described in 1.1 to 1.5. LCMS [M+1]⁺ 312.2. ¹H NMR (Methanol-d₄, 400 MHz): δ (ppm) 4.57-4.45 (m, 1H), 4.15-4.07 (m, 1H), 3.80-3.73 (m, 1H), 3.37-3.33 (m, 5H), 2.96-2.85 (m, 1H), 2.86-2.30 (m, 9H), 2.32-2.28 (m, 3H), 2.24-2.15 (m, 1H), 2.13-2.05 (m, 1H), 1.68-1.56 (m, 4H), 1.53-1.43 (m, 2H).

### Synthesis of rac-3-(2-(1-methylpiperidin-3-yl)propan-2-yl)-5-(piperidin-1-ylmethyl)-5,6-dihydro-1,4,2-dioxazine, ID 595

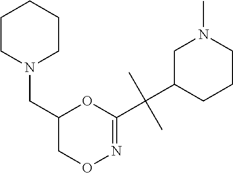 [SMILES: CN1CCCC(C(C)(C)C2=NOCC(CN3CCCCC3)O2)C1]

The general synthesis using halo-cyclization as described herein was used to provide rac-3-(2-(1-methylpiperidin-3-yl)propan-2-yl)-5-(piperidin-1-ylmethyl)-5,6-dihydro-1,4,2-dioxazine (112.3 mg, 53.72%) as a yellow oil from commercially available rac-2-(1-(tert-butoxycarbonyl)piperidin-3-yl)-2-methylpropanoic acid in line with the synthesis described in 1.1 to 1.5. LCMS [M+1]⁺ 324.2. ¹H NMR (Chloroform-d, 400 MHz): δ (ppm) 4.37-4.27 (m, 1H), 4.05 (dd, J=11.5, 2.8 Hz, 1H), 3.76-3.60 (m, 1H), 2.83-2.70 (m, 2H), 2.59-2.40 (m, 4H), 2.41-2.32 (m, 2H), 2.23 (s, 3H), 1.90-1.60 (m, 5H), 1.58-1.41 (m, 5H), 1.41-1.33 (m, 2H), 1.19-0.82 (m, 7H).

### Synthesis of rac-3-(3-(2-methoxyethyl)-1-methylpyrrolidin-3-yl)-5-(piperidin-1-ylmethyl)-5,6-dihydro-1,4,2-dioxazine, ID 701

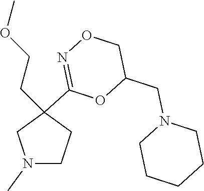 [SMILES: COCCC1(C2=NOCC(CN3CCCCC3)O2)CCN(C)C1]

The general synthesis using halo-cyclization as described herein was used to provide rac-3-(3-(2-methoxyethyl)-1-methylpyrrolidin-3-yl)-5-(piperidin-1-ylmethyl)-5,6-dihydro-1,4,2-dioxazine (22.6 mg, 27.03%) as a yellow oil from commercially available rac-1-(tert-butoxycarbonyl)-3-(2-methoxyethyl)pyrrolidine-3-carboxylic acid in line with the synthesis described in 1.1 to 1.5. LCMS [M+1]⁺ 326.2. ¹H NMR (Methanol-d₄, 400 MHz): δ (ppm) 4.55-4.42 (m, 1H), 4.14-4.04 (m, 1H), 3.80-3.70 (m, 1H), 3.41-3.35 (m, 3H), 3.29 (s, 3H), 3.04 (d, J=10.1 Hz, 1H), 2.64-2.54 (m, 5H), 2.53-2.47 (m, 2H), 2.46-2.37 (m, 2H), 2.33 (s, 3H), 2.07-1.85 (m, 2H), 1.80-1.69 (m, 1H), 1.67-1.56 (m, 4H), 1.51-1.41 (m, 2H).

### Synthesis of rac-3-(4-methoxy-1-methylpyrrolidin-3-yl)-5-(piperidin-1-ylmethyl)-5,6-dihydro-1,4,2-dioxazine, ID 821

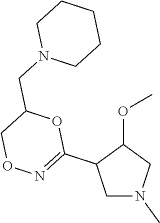 [SMILES: COC1CN(C)CC1C1=NOCC(CN2CCCCC2)O1]

The general synthesis using halo-cyclization as described herein was used to provide 3-(4-methoxy-1-methylpyrrolidin-3-yl)-5-(piperidin-1-ylmethyl)-5,6-dihydro-1,4,2-dioxazine (15.4 mg, 3.75%) as a yellow oil from commercially available 1-(tert-butoxycarbonyl)-4-methoxypyrrolidine-3-carboxylic acid in line with the synthesis described in 1.1 to 1.5. LCMS [M+1]⁺ 298.0. ¹H NMR (Methanol-d₄, 600 MHz): δ (ppm) 4.56-4.46 (m, 1H), 4.18-3.97 (m, 2H), 3.78-3.67 (m, 1H), 3.36-3.25 (m, 5H), 3.06-2.97 (m, 1H), 2.91-2.87 (m, 1H), 2.85-2.80 (m, 1H), 2.66-2.51 (m, 4H), 2.49-2.44 (m, 2H), 2.30 (s, 3H), 1.65-1.52 (m, 4H), 1.51-1.37 (m, 2H).

### Synthesis of rac-3-(1-isopropylpyrrolidin-3-yl)-6-methyl-5-(piperidin-1-ylmethyl)-5,6-dihydro-1,4,2-dioxazine, ID 621

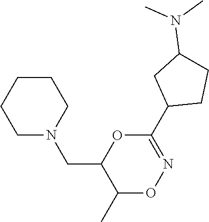 [SMILES: CC1ON=C(C2CCC(N(C)C)C2)OC1CN1CCCCC1]

The general synthesis using halo-cyclization as described herein was used to provide rac-3-(1-isopropylpyrrolidin-3-yl)-6-methyl-5-(piperidin-1-ylmethyl)-5,6-dihydro-1,4,2-dioxazine (168.1 mg, 19.71%) as a yellow oil from commercially available rac-1-(tert-butoxycarbonyl)pyrrolidine-3-carboxylic acid in line with the synthesis described in 1.1 to 1.5 but using O-(1-methylallyl)hydroxylamine hydrochloride instead of O-allylhydroxylamine hydrochloride in experimental procedure 1.1 and acetone instead of paraform in experimental procedure 1.5. LCMS [M+1]⁺ 310.2. ¹H NMR (400 MHz, MeOD) δ 4.15-4.05 (m, 1H), 3.73-3.59 (m, 1H), 3.09-2.90 (m, 2H), 2.90-2.79 (m, 1H), 2.67-2.60 (m, 1H), 2.60-2.45 (m, 7H), 2.44-2.36 (m, 1H), 2.14-1.91 (m, 2H), 1.65-1.50 (m, 4H), 1.50-1.36 (m, 2H), 1.26 (d, J=6.2 Hz, 3H), 1.10 (d, J=3.5 Hz, 6H).

### Synthesis of rac-3-(1-isopropylpiperidin-4-yl)-5-(piperidin-1-ylmethyl)-5,6-dihydro-1,4,2-dioxazine, ID 719

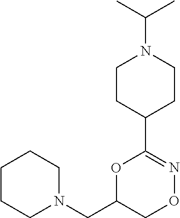 [SMILES: CC(C)N1CCC(C2=NOCC(CN3CCCCC3)O2)CC1]

The general synthesis using halo-cyclization as described herein was used to provide rac-3-(1-isopropylpiperidin-4-yl)-5-(piperidin-1-ylmethyl)-5,6-dihydro-1,4,2-dioxazine (439.9 mg, 61.57%) as a yellow oil from commercially available 1-(tert-butoxycarbonyl)piperidine-4-carboxylic acid in line with the synthesis described in 1.1 to 1.5 but using acetone instead of paraform in experimental procedure 1.5. LCMS [M+1]⁺ 310.2. ¹H NMR (400 MHz, CD3OD) δ 4.55-4.43 (m, 1H), 4.09 (dd, J=11.6, 3.1 Hz, 1H), 3.71 (dd, J=11.6, 6.4 Hz, 1H), 2.98-2.88 (m, 2H), 2.78-2.67 (m, 1H), 2.64-2.44 (m, 6H), 2.27-2.11 (m, 3H), 1.94-1.79 (m, 2H), 1.79-1.67 (m, 2H), 1.67-1.59 (m, 4H), 1.53-1.36 (m, 2H), 1.08 (d, J=6.6 Hz, 6H).

### Synthesis of rac-3-(1-isopropyl-3-methoxypiperidin-4-yl)-5-(piperidin-1-ylmethyl)-5,6-dihydro-1,4,2-dioxazine, ID 714

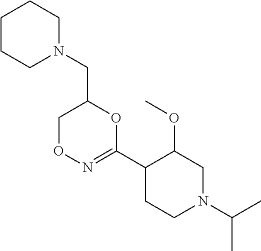 [SMILES: COC1CN(C(C)C)CCC1C1=NOCC(CN2CCCCC2)O1]

The general synthesis using halo-cyclization as described herein was used to provide rac-3-(1-isopropyl-3-methoxypiperidin-4-yl)-5-(piperidin-1-ylmethyl)-5,6-dihydro-1,4,2-dioxazine (37.2 mg, 7.74%) as a yellow oil from commercially available rac-1-(tert-butoxycarbonyl)-3-methoxypiperidine-4-carboxylic acid in line with the synthesis described in 1.1 to 1.5 but using acetone instead of paraform in experimental procedure 1.5. LCMS [M+1]⁺ 340.4. ¹H NMR (400 MHz, CD₃OD) δ 4.56-4.45 (m, 1H), 4.15-4.04 (m, 1H), 3.78-3.67 (m, 2H), 3.36 (s, 3H), 3.26-3.14 (m, 1H), 2.90-2.81 (m, 1H), 2.81-2.68 (m, 1H), 2.66-2.38 (m, 7H), 2.32-2.20 (m, 1H), 2.20-2.10 (m, 1H), 2.05-1.89 (m, 1H), 1.72-1.55 (m, 5H), 1.55-1.40 (m, 2H), 1.09 (d, J=6.7 Hz, 3H), 1.05 (d, J=6.4 Hz, 3H).

### Synthesis of rac-3-(1-isopropylpiperidin-3-yl)-5-(piperidin-1-ylmethyl)-5,6-dihydro-1,4,2-dioxazine, ID 709

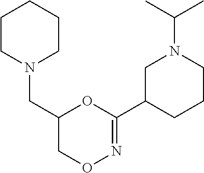 [SMILES: CC(C)N1CCCC(C2=NOCC(CN3CCCCC3)O2)C1]

The general synthesis using halo-cyclization as described herein was used to provide rac-3-(1-isopropylpiperidin-3-yl)-5-(piperidin-1-ylmethyl)-5,6-dihydro-1,4,2-dioxazine (102.4 mg, 42.03%) as a yellow oil from commercially available rac-1-(tert-butoxycarbonyl)piperidine-3-carboxylic acid in line with the synthesis described in 1.1 to 1.5 but using acetone instead of paraform in experimental procedure 1.5. LCMS [M+1]⁺ 310.2. ¹H NMR (400 MHz, CD₃OD) δ 4.54-4.43 (m, 1H), 4.09 (dd, J=11.7, 3.0 Hz, 1H), 3.72 (dd, J=11.7, 6.5 Hz, 1H), 3.05-2.93 (m, 1H), 2.90-2.81 (m, 1H), 2.81-2.70 (m, 1H), 2.64-2.37 (m, 7H), 2.28-2.07 (m, 2H), 1.97-1.85 (m, 1H), 1.82-1.71 (m, 1H), 1.67-1.52 (m, 5H), 1.52-1.42 (m, 2H), 1.42-1.27 (m, 1H), 1.15-0.97 (m, 6H).

### Synthesis of rac-3-(1-isopropyl-3-methylpiperidin-3-yl)-5-(piperidin-1-ylmethyl)-5,6-dihydro-1,4,2-dioxazine, ID 539

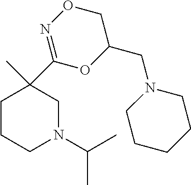 [SMILES: CC(C)N1CCCC(C)(C2=NOCC(CN3CCCCC3)O2)C1]

The general synthesis using halo-cyclization as described herein was used to provide rac-3-(1-isopropyl-3-methylpiperidin-3-yl)-5-(piperidin-1-ylmethyl)-5,6-dihydro-1,4,2-dioxazine (286 mg, 28.82%) as a yellow oil from commercially available rac-1-(tert-butoxycarbonyl)-3-methylpiperidine-3-carboxylic acid in line with the synthesis described in 1.1 to 1.5 but using acetone instead of paraform in experimental procedure 1.5. LCMS [M+1]⁺ 324.2. ¹H NMR (400 MHz, CDCl₃) δ 4.42-4.29 (m, 1H), 4.14-3.96 (m, 1H), 3.75-3.61 (m, 1H), 2.78-2.58 (m, 2H), 2.56-2.44 (m, 4H), 2.43-2.30 (m, 4H), 2.15-2.02 (m, 1H), 1.93-1.80 (m, 1H), 1.81-1.55 (m, 2H), 1.55-1.49 (m, 4H), 1.43-1.33 (m, 2H), 1.29-1.18 (m, 1H), 1.13 (s, 3H), 1.03-0.80 (m, 6H).

### Synthesis of rac-N,N-dimethyl-4-(5-(piperidin-1-ylmethyl)-5,6-dihydro-1,4,2-dioxazin-3-yl)bicyclo[2.2.2]octan-1-amine, ID 640

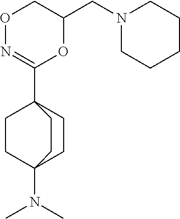 [SMILES: CN(C)C12CCC(C3=NOCC(CN4CCCCC4)O3)(CC1)CC2]

The general synthesis using halo-cyclization as described herein was used to provide N,N-dimethyl-4-(5-(piperidin-1-ylmethyl)-5,6-dihydro-1,4,2-dioxazin-3-yl)bicyclo[2.2.2]octan-1-amine (124.9 mg, 16.73%) as a yellow oil from commercially available 4-((tert-butoxycarbonyl)amino)bicyclo[2.2.2]octane-1-carboxylic acid in line with the synthesis described in 1.1 to 1.5. LCMS [M+1]⁺ 336.2. ¹H NMR (400 MHz, CD₃OD) δ 4.42 (tdd, J=6.7, 6.7, 4.7, 2.9 Hz, 1H), 4.03 (dd, J=11.6, 2.9 Hz, 1H), 3.63 (dd, J=11.6, 6.5 Hz, 1H), 2.58 (dd, J=13.7, 4.8 Hz, 2H), 2.55-2.48 (m, 2H), 2.48-2.41 (m, 2H), 2.19 (s, 6H), 1.82-1.73 (m, 6H), 1.67-1.56 (m, 10H), 1.50-1.41 (m, 2H).

### Synthesis of rac-rel-cis-(1S,4S)—N,N-dimethyl-4-(5-(piperidin-1-ylmethyl)-5,6-dihydro-1,4,2-dioxazin-3-yl)cyclohexan-1-amine, ID 569

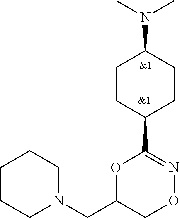 [SMILES: CN(C)[C@H]1CC[C@@H](C2=NOCC(CN3CCCCC3)O2)CC1]

The general synthesis using halo-cyclization as described herein was used to provide rac-rel-cis-(1 S,4S)—N,N-dimethyl-4-(5-(piperidin-1-ylmethyl)-5,6-dihydro-1,4,2-dioxazin-3-yl)cyclohexan-1-amine (417.1 mg, 55.43%) as a brown oil from commercially available rac-rel-cis-(1S,4S)-4-{[(tert-butoxy)carbonyl]amino}cyclohexane-1-carboxylic acid in line with the synthesis described in 1.1 to 1.5. LCMS [M+1]⁺ 310.4. ¹H NMR (400 MHz, CD₃OD) δ 4.56-4.44 (m, 1H), 4.10 (dd, J=11.8, 3.0 Hz, 1H), 3.80-3.64 (m, 1H), 2.64-2.47 (m, 7H), 2.39 (s, 6H), 2.16-2.05 (m, 2H), 1.79-1.66 (m, 4H), 1.66-1.52 (m, 7H), 1.52-1.44 (m, 2H).

### Synthesis of rac-N,N-dimethyl-2-(5-(piperidin-1-ylmethyl)-5,6-dihydro-1,4,2-dioxazin-3-yl)ethan-1-amine, ID 779

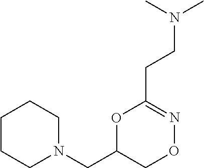 [SMILES: CN(C)CCC1=NOCC(CN2CCCCC2)O1]

The general synthesis using halo-cyclization as described herein was used to provide rac-N,N-dimethyl-2-(5-(piperidin-1-ylmethyl)-5,6-dihydro-1,4,2-dioxazin-3-yl)ethan-1-amine (76.3 mg, 7.04%) as a yellow oil from commercially available 3-((tert-butoxycarbonyl)amino)propanoic acid in line with the synthesis described in 1.1 to 1.5. LCMS [M+1]⁺ 256.2. ¹H NMR (Methanol-d₄, 400 MHz): δ (ppm) 4.60-4.46 (m, 1H), 4.12 (dd, J=11.2, 3.3 Hz, 1H), 3.81-3.67 (m, 1H), 2.76-2.46 (m, 8H), 2.40 (t, J=7.4, 7.4 Hz, 2H), 2.27 (s, 6H), 1.70-1.55 (m, 4H), 1.53-1.43 (m, 2H).

### Synthesis of rac-2-(5-(piperidin-1-ylmethyl)-5,6-dihydro-1,4,2-dioxazin-3-yl)-N-(pyridin-3-ylmethyl)ethan-1-amine, ID 797

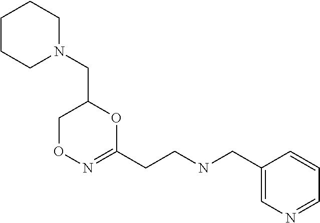 [SMILES: c1cncc(CNCCC2=NOCC(CN3CCCCC3)O2)c1]

The general synthesis using halo-cyclization as described herein was used to provide rac-2-(5-(piperidin-1-ylmethyl)-5,6-dihydro-1,4,2-dioxazin-3-yl)-N-(pyridin-3-ylmethyl)ethan-1-amine (70.9 mg, 50.61%) as a yellow oil from commercially available 3-((tert-butoxycarbonyl)amino)propanoic acid in line with the synthesis described in 1.1 to 1.5 but using nicotinaldehyde instead of paraform in experimental procedure 1.5. LCMS [M+1]⁺ 319.2. ¹H NMR (Methanol-d₄, 400 MHz): δ (ppm) 8.54 (s, 1H), 8.45 (d, J=4.9 Hz, 1H), 7.87 (d, J=7.8 Hz, 1H), 7.43 (dd, J=7.8, 4.9 Hz, 1H), 4.56-4.46 (m, 1H), 4.11 (dd, J=11.7, 2.9 Hz, 1H), 3.83 (s, 2H), 3.74 (dd, J=11.6, 6.6 Hz, 1H), 2.83 (t, J=6.9, 6.9 Hz, 2H), 2.56 (d, J=6.5 Hz, 2H), 2.54-2.43 (m, 5H), 2.43 (t, J=7.0 Hz, 2H), 1.66-1.54 (m, 4H), 1.51-1.40 (m, 2H).

### Synthesis of rac-N-((5-methoxypyridin-3-yl)methyl)-2-(5-(piperidin-1-ylmethyl)-5,6-dihydro-1,4,2-dioxazin-3-yl)ethan-1-amine, ID 806

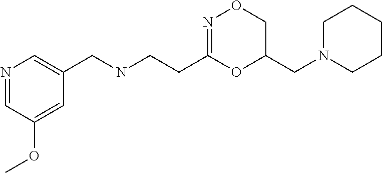 [SMILES: COc1cncc(CNCCC2=NOCC(CN3CCCCC3)O2)c1]

In a generally similar manner with non-critical variations was made rac-N-((5-methoxypyridin-3-yl)methyl)-2-(5-(piperidin-1-ylmethyl)-5,6-dihydro-1,4,2-dioxazin-3-yl)ethan-1-amine (29.8 mg, 29.12%) as a yellow oil from commercially available 3-((tert-butoxycarbonyl)amino)propanoic acid in line with the synthesis described in 1.1 to 1.5 but using 5-methoxynicotinaldehyde instead of paraform in experimental procedure 1.5. LCMS [M+1]⁺ 349.2. ¹H NMR (Methanol-d₄, 400 MHz): δ (ppm) 8.13 (d, J=8.7 Hz, 2H), 7.47 (s, 1H), 4.55-4.46 (m, 1H), 4.11 (d, J=14.7 Hz, 1H), 3.90 (s, 3H), 3.81 (s, 2H), 3.74 (dd, J=11.6, 6.5 Hz, 1H), 2.83 (t, J=6.9, 6.9 Hz, 2H), 2.61-2.33 (m, 8H), 1.68-1.51 (m, 4H), 1.54-1.37 (m, 2H).

### Synthesis of rac-N,N-dimethyl-1-(1-methyl-3-(5-(piperidin-1-ylmethyl)-5,6-dihydro-1,4,2-dioxazin-3-yl)pyrrolidin-3-yl)methanamine, ID 826

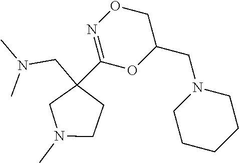 [SMILES: CN(C)CC1(C2=NOCC(CN3CCCCC3)O2)CCN(C)C1]

In a generally similar manner with non-critical variations was made rac-N,N-dimethyl-1-(1-methyl-3-(5-(piperidin-1-ylmethyl)-5,6-dihydro-1,4,2-dioxazin-3-yl)pyrrolidin-3-yl)methanamine (85.3 mg, 35.26%) as a yellow oil from the commercially available 1-(tert-butoxycarbonyl)-3-(((tert-butoxycarbonyl)amino)methyl)pyrrolidine-3-carboxylic acid in line with the synthesis described in 1.1 to 1.5. LCMS [M+1]⁺ 325.2. ¹H NMR (DMSO-d₆, 400 MHz): δ (ppm) 4.43-4.29 (m, 1H), 3.99 (dd, J=11.3, 3.3 Hz, 1H), 3.68-3.56 (m, 1H), 2.70-2.57 (m, 2H), 2.47-2.24 (m, 10H), 2.22-2.09 (m, 10H), 1.69-1.59 (m, 1H), 1.56-1.43 (m, 4H), 1.41-1.30 (m, 2H).

### Halocyclization+Additional N-Acylation

### Synthesis of rac-(2S)-2-amino-1-(3-(5-(piperidin-1-ylmethyl)-5,6-dihydro-1,4,2-dioxazin-3-yl)piperidin-1-yl)propan-1-one, ID 718

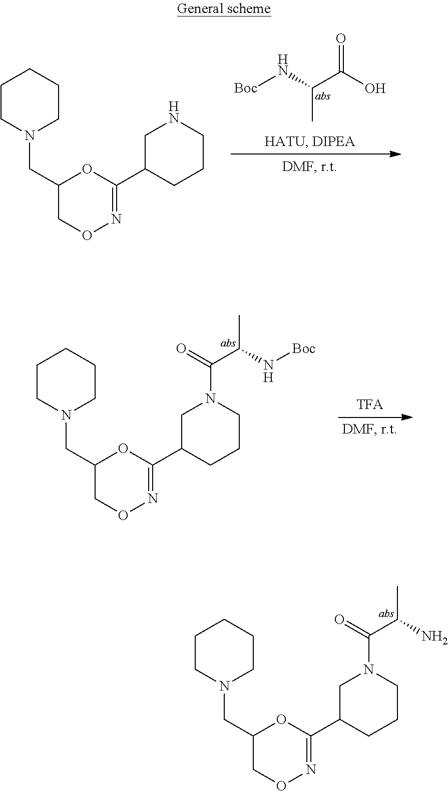 [SMILES: C1CCN(CC2CON=C(C3CCCNC3)O2)CC1 ; C[C@H](NC(=O)OC(C)(C)C)C(=O)O ; C[C@H](NC(=O)OC(C)(C)C)C(=O)N1CCCC(C2=NOCC(CN3CCCCC3)O2)C1 ; C[C@H](N)C(=O)N1CCCC(C2=NOCC(CN3CCCCC3)O2)C1]

### Procedure 1.6

### Synthesis of rac-tert-butyl ((2S)-1-oxo-1-(3-(5-(piperidin-1-ylmethyl)-5,6-dihydro-1,4,2-dioxazin-3-yl)piperidin-1-yl)propan-2-yl)carbamate

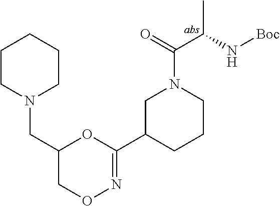 [SMILES: C[C@H](NC(=O)OC(C)(C)C)C(=O)N1CCCC(C2=NOCC(CN3CCCCC3)O2)C1]

(2S)-2-(tert-Butoxycarbonylamino)propanoic acid (155.69 mg, 0.823 mmol, 1.1 eq) and [dimethylamino-(3-oxidotriazolo[4,5-b]pyridin-3-ium-1-yl)methylene]-dimethyl-ammonium hexafluorophosphate (312.86 mg, 0.823 mmol, 1.1 eq) were mixed together in dry DMF (1 mL), followed by N,N-Diisopropylethylamine (212.69 mg, 1.646 mmol, 2.2 eq). The resulting clear solution was stirred for 20 minutes at ambient temperature, then rac-3-(3-piperidyl)-5-(1-piperidylmethyl)-5,6-dihydro-1,4,2-dioxazine, obtained in a similar manner with non-critical variations from commercially available rac-1-(tert-butoxycarbonyl)piperidine-3-carboxylic acid in line with the synthesis described in 1.1 to 1.4 (200 mg, 0.748 mmol, 1 eq) was added in a single portion. The reaction mixture was left while stirring at ambient temperature overnight. After 14 hours the reaction mixture solution was subjected for preparative HPLC without any work up (50-100% 0-5 min water-methanol, flow: 30 ml/min (loading pump 4 ml/min methanol); target mass 439; column: SunFireC18 100×19 mm 5 um) to afford the title product (197.3 mg, 57.14%) as a yellow oil. LCMS [M+1]*439.2

### Procedure 1.7

### Synthesis of rac-(2S)-2-amino-1-(3-(5-(piperidin-1-ylmethyl)-5,6-dihydro-1,4,2-dioxazin-3-yl)piperidin-1-yl)propan-1-one

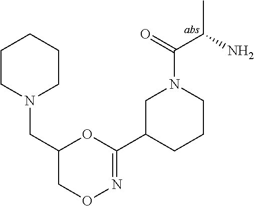 [SMILES: C[C@H](N)C(=O)N1CCCC(C2=NOCC(CN3CCCCC3)O2)C1]

rac-tert-Butyl N-[(1 S)-1-methyl-2-oxo-2-[3-[5-(1-piperidylmethyl)-5,6-dihydro-1,4,2-dioxazin-3-yl]-1-piperidyl]ethyl]carbamate (197.3 mg, 0.4499 mmol, 1 eq) was dissolved in dry DCM (1 mL), followed by dropwise addition of 2,2,2-trifluoroacetic acid (512.95 mg, 4.499 mmol, 10 eq). The reaction mixture was left while stirring at room temperature overnight. After 14 hours the reaction mixture was gently evaporated under reduced pressure at 40° C. to afford yellow coloured oily residue, which was diluted with DCM (3 mL) and washed with 30% aqueous solution of potassium carbonate (2×3 mL). The organic layer was isolated and concentrated under reduced pressure to result in 200 mg of crude oil, which was purified with preparative HPLC (40-80% 0-5 min water-methanol, flow: 30 ml/min (loading pump 4 ml/min methanol); target mass 339; column: SunFireC18 100×19 mm 5 um) to afford the title product (80.1 mg, 49.98%) as an yellow oil. LCMS [M+1]⁺ 339.2. ¹H NMR (400 MHz, CD₃OD) δ 4.57-4.36 (m, 2H), 4.20-3.94 (m, 2H), 3.93-3.76 (m, 2H), 3.77-3.63 (m, 1H), 3.26-3.06 (m, 1H), 3.06-2.59 (m, 1H), 2.59-2.21 (m, 7H), 2.12-1.97 (m, 1H), 1.97-1.67 (m, 2H), 1.67-1.53 (m, 5H), 1.53-1.38 (m, 3H), 1.25-1.19 (m, 3H).

### Synthesis of rac-2-amino-1-(3-(5-(piperidin-1-ylmethyl)-5,6-dihydro-1,4,2-dioxazin-3-yl)pyrrolidin-1-yl)propan-1-one, ID 691

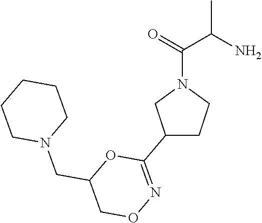 [SMILES: CC(N)C(=O)N1CCC(C2=NOCC(CN3CCCCC3)O2)C1]

rac-2-Amino-1-(3-(5-(piperidin-1-ylmethyl)-5,6-dihydro-1,4,2-dioxazin-3-yl)pyrrolidin-1-yl)propan-1-one (94.7 mg, 34.42%) was prepared as a yellow oil from commercially available rac-1-(tert-butyl) 3-methyl pyrrolidine-1,3-dicarboxylate in line with the synthesis described in 1.1 to 1.4, 1.6, 1.7 but using rac-2-(tert-butoxycarbonylamino)propanoic acid instead of (2S)-2-(tert-butoxycarbonylamino)propanoic acid in in experimental procedure 1.6. LCMS [M+1]+325.4. ¹H NMR (400 MHz, CD₃OD) δ 4.59-4.47 (m, 1H), 4.17-4.06 (m, 1H), 3.81-3.70 (m, 2H), 3.70-3.47 (m, 4H), 3.17-3.02 (m, 1H), 2.58 (d, J=5.7 Hz, 2H), 2.56-2.43 (m, 4H), 2.26-2.18 (m, 1H), 2.18-2.07 (m, 1H), 1.67-1.55 (m, 4H), 1.55-1.44 (m, 2H), 1.29-1.16 (m, 3H).

### Synthesis of rac-2-amino-1-(4-(5-(piperidin-1-ylmethyl)-5,6-dihydro-1,4,2-dioxazin-3-yl)piperidin-1-yl)propan-1-one, ID 748

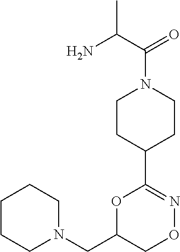 [SMILES: CC(N)C(=O)N1CCC(C2=NOCC(CN3CCCCC3)O2)CC1]

rac-2-amino-1-(4-(5-(piperidin-1-ylmethyl)-5,6-dihydro-1,4,2-dioxazin-3-yl)piperidin-1-yl)propan-1-one (69.5 mg, 45.1%) was prepared as a yellow oil from commercially available 1-(tert-butoxycarbonyl)piperidine-4-carboxylic acid in line with the synthesis described in 1.1 to 1.4, 1.6, 1.7 but using 2-(tert-butoxycarbonylamino)propanoic acid instead of (2S)-2-(tert-butoxycarbonylamino)propanoic acid in in experimental procedure 1.6. LCMS [M+1]⁺ 339.4. ¹H NMR (Methanol-d₄, 400 MHz): δ (ppm) 4.57-4.41 (m, 2H), 4.20-4.05 (m, 1H), 3.99 (d, J=13.6 Hz, 1H), 3.93-3.85 (m, 1H), 3.73 (dd, J=11.6, 6.6 Hz, 1H), 3.23-3.11 (m, 1H), 2.87-2.70 (m, 1H), 2.72-2.29 (m, 7H), 1.99-1.84 (m, 2H), 1.72-1.50 (m, 6H), 1.50-1.36 (m, 2H), 1.25-1.14 (m, 3H).

### Synthesis of rac-2-amino-1-(3-(5-(piperidin-1-ylmethyl)-5,6-dihydro-1,4,2-dioxazin-3-yl)pyrrolidin-1-yl)ethan-1-one, ID 699

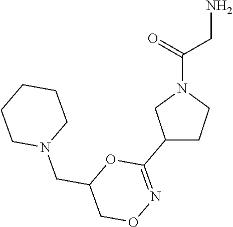 [SMILES: NCC(=O)N1CCC(C2=NOCC(CN3CCCCC3)O2)C1]

rac-2-amino-1-(3-(5-(piperidin-1-ylmethyl)-5,6-dihydro-1,4,2-dioxazin-3-yl)pyrrolidin-1-yl)ethan-1-one (64.5 mg, 25.01%) was prepared as a yellow oil from commercially available rac-1-(tert-butyl) 3-methyl pyrrolidine-1,3-dicarboxylate in line with the synthesis described in 1.1 to 1.4, 1.6, 1.7 but using 2-(tert-butoxycarbonylamino)acetic acid instead of (2S)-2-(tert-butoxycarbonylamino)propanoic acid in in experimental procedure 1.6. LCMS [M+1]⁺ 311.2. ¹H NMR (400 MHz, CD₃OD) δ 4.60-4.45 (m, 1H), 4.17-4.02 (m, 1H), 3.79-3.71 (m, 1H), 3.71-3.52 (m, 3H), 3.52-3.41 (m, 1H), 3.38 (s, 2H), 3.19-2.94 (m, 1H), 2.58 (d, J=5.8 Hz, 2H), 2.56-2.43 (m, 4H), 2.28-2.01 (m, 2H), 1.69-1.52 (m, 4H), 1.54-1.41 (m, 2H).

### Synthesis of rac-2-amino-1-(3-(5-(piperidin-1-ylmethyl)-5,6-dihydro-1,4,2-dioxazin-3-yl)piperidin-1-yl)ethan-1-one, ID 725

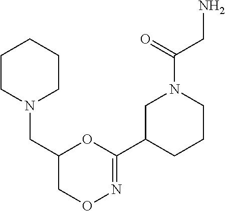 [SMILES: NCC(=O)N1CCCC(C2=NOCC(CN3CCCCC3)O2)C1]

rac-2-amino-1-(3-(5-(piperidin-1-ylmethyl)-5,6-dihydro-1,4,2-dioxazin-3-yl)piperidin-1-yl)ethan-1-one (232 mg, 86.38%) was prepared as a yellow oil from commercially available rac-1-(tert-butoxycarbonyl)piperidine-3-carboxylic acid in line with the synthesis described in 1.1 to 1.4, 1.6, 1.7 but using 2-(tert-butoxycarbonylamino)acetic acid instead of (2S)-2-(tert-butoxycarbonylamino)propanoic acid in in experimental procedure 1.6. LCMS [M+1]⁺ 325.4. ¹H NMR (Methanol-d₄, 400 MHz): δ (ppm) 4.57-4.37 (m, 2H), 4.16-3.87 (m, 2H), 3.73-3.62 (m, 3H), 3.19-2.88 (m, 2H), 2.57-2.43 (m, 7H), 2.38-2.21 (m, 1H), 2.07-1.64 (m, 3H), 1.63-1.43 (m, 8H).

### Synthesis of rac-2-amino-1-(4-(5-(piperidin-1-ylmethyl)-5,6-dihydro-1,4,2-dioxazin-3-yl)piperidin-1-yl)ethan-1-one, ID 787

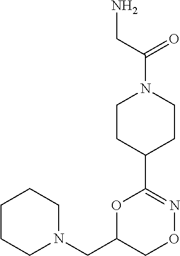 [SMILES: NCC(=O)N1CCC(C2=NOCC(CN3CCCCC3)O2)CC1]

rac-2-amino-1-(4-(5-(piperidin-1-ylmethyl)-5,6-dihydro-1,4,2-dioxazin-3-yl)piperidin-1-yl)ethan-1-one (28.8 mg, 18.93%) was prepared as a yellow oil from commercially available 1-(tert-butoxycarbonyl)piperidine-4-carboxylic acid in line with the synthesis described in 1.1 to 1.4, 1.6, 1.7 but using 2-(tert-butoxycarbonylamino)acetic acid instead of (2S)-2-(tert-butoxycarbonylamino)propanoic acid in in experimental procedure 1.6. LCMS [M+1]⁺ 325.4. ¹H NMR (Methanol-d₄, 400 MHz): δ (ppm) 4.56-4.43 (m, 2H), 4.12 (d, J=2.7 Hz, 1H), 3.89-3.80 (m, 1H), 3.72 (dd, J=12.2, 5.8 Hz, 1H), 3.55-3.40 (m, 2H), 3.09 (t, J=11.0, 11.0 Hz, 1H), 2.85-2.74 (m, 1H), 2.75-2.32 (m, 7H), 1.93-1.85 (m, 2H), 1.77-1.50 (m, 6H), 1.52-1.39 (m, 2H).

### Halocyclization+Additional N-Alkylation

### Synthesis of rac-2-(3-(5-(piperidin-1-ylmethyl)-5,6-dihydro-1,4,2-dioxazin-3-yl)piperidin-1-yl)acetamide, ID 711

### General Procedure

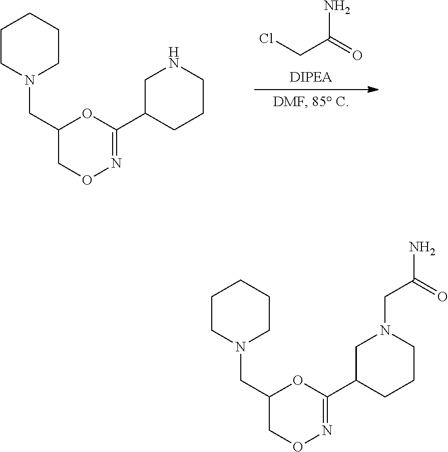 [SMILES: C1CCN(CC2CON=C(C3CCCNC3)O2)CC1 ; NC(=O)CCl ; NC(=O)CN1CCCC(C2=NOCC(CN3CCCCC3)O2)C1]

### Procedure 1.8

### Synthesis of rac-2-(3-(5-(piperidin-1-ylmethyl)-5,6-dihydro-1,4,2-dioxazin-3-yl)piperidin-1-yl)acetamide

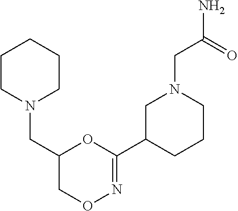 [SMILES: NC(=O)CN1CCCC(C2=NOCC(CN3CCCCC3)O2)C1]

rac-3-(3-Piperidyl)-5-(1-piperidylmethyl)-5,6-dihydro-1,4,2-dioxazine was obtained in a similar manner with non-critical variations from commercially available rac-1-(tert-butoxycarbonyl)piperidine-3-carboxylic acid in line with the synthesis described in 1.1 to 1.4 (200 mg, 0.7480 mmol, 1 eq) was dissolved in dry DMF (0.6 mL), after that 2-chloroacetamide (76.943 mg, 0.8228 mmol, 1.1 eq) was added to the resulting solution, followed by the addition of N,N-Diisopropylethylamine (116.01 mg, 0.8976 mmol, 1.2 eq). The reaction mixture was heated at 85° C. overnight. After 24 hours the reaction mixture was subjected for preparative HPLC without any work up (20-60% 0-6 min water-methanol, flow: 30 ml/min (loading pump 4 ml/min methanol); target mass 325; column SunFireC18 100×19 mm 5 um) to afford the title product (94.5 mg, 36.99%) as a yellow oil. LCMS [M+1]⁺ 325.4. ¹H NMR (400 MHz, CD₃OD) 54.53-4.42 (m, 1H), 4.15-4.03 (m, 1H), 3.72 (ddd, J=11.7, 6.5, 3.3 Hz, 1H), 3.05-2.94 (m, 2H), 2.94-2.85 (m, 1H), 2.85-2.75 (m, 1H), 2.65-2.43 (m, 7H), 2.39-2.24 (m, 1H), 2.24-2.10 (m, 1H), 1.93-1.81 (m, 1H), 1.81-1.71 (m, 1H), 1.70-1.54 (m, 5H), 1.54-1.40 (m, 3H).

### Synthesis of rac-N,N-dimethyl-2-(3-(5-(piperidin-1-ylmethyl)-5,6-dihydro-1,4,2-dioxazin-3-yl)piperidin-1-yl)acetamide, ID 710

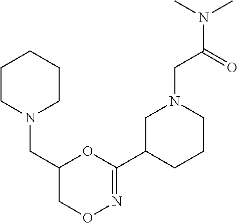 [SMILES: CN(C)C(=O)CN1CCCC(C2=NOCC(CN3CCCCC3)O2)C1]

rac-N,N-Dimethyl-2-(3-(5-(piperidin-1-ylmethyl)-5,6-dihydro-1,4,2-dioxazin-3-yl)piperidin-1-yl)acetamide (77.2 mg, 27.82%) was prepared as a yellow oil from commercially available rac-1-(tert-butoxycarbonyl)piperidine-3-carboxylic acid in line with the synthesis described in 1.1 to 1.4, 1.8 but using 2-chloro-N,N-dimethyl-acetamide instead of 2-chloroacetamide in experimental procedure 1.8. LCMS [M+1]+353.2. ¹H NMR (400 MHz, CD₃OD) δ 4.52-4.41 (m, 1H), 4.09 (dd, J=11.6, 3.0 Hz, 1H), 3.71 (ddd, J=11.8, 6.6, 2.2 Hz, 1H), 3.27-3.18 (m, 2H), 3.11 (s, 3H), 3.05-2.98 (m, 1H), 2.94 (s, 3H), 2.90-2.81 (m, 1H), 2.64-2.42 (m, 7H), 2.22-2.02 (m, 2H), 1.95-1.85 (m, 1H), 1.81-1.70 (m, 1H), 1.68-1.56 (m, 5H), 1.53-1.43 (m, 2H), 1.43-1.29 (m, 1H).

### Synthesis of rac-N-methyl-2-(3-(5-(piperidin-1-ylmethyl)-5,6-dihydro-1,4,2-dioxazin-3-yl)piperidin-1-yl)acetamide, ID 721

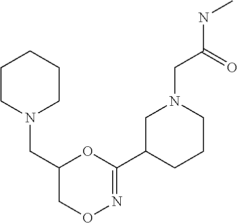 [SMILES: CNC(=O)CN1CCCC(C2=NOCC(CN3CCCCC3)O2)C1]

rac-N-Methyl-2-(3-(5-(piperidin-1-ylmethyl)-5,6-dihydro-1,4,2-dioxazin-3-yl)piperidin-1-yl)acetamide (171.8 mg, 64.47%) was prepared as a yellow oil from commercially available rac-1-(tert-butoxycarbonyl)piperidine-3-carboxylic acid in line with the synthesis described in 1.1 to 1.4, 1.8 but using 2-chloro-N-methylacetamide instead of 2-chloroacetamide in experimental procedure 1.8. LCMS [M+1]⁺ 339.2. ¹H NMR (Methanol-d₄, 400 MHz): δ (ppm) 4.54-4.45 (m, 1H), 4.10 (dd, J=11.6, 2.5 Hz, 1H), 3.79-3.65 (m, 1H), 3.05-2.93 (m, 2H), 2.79 (s, 4H), 2.76-2.68 (m, 1H), 2.63-2.43 (m, 7H), 2.40-2.30 (m, 1H), 2.22 (t, J=9.9, 9.9 Hz, 1H), 1.91-1.80 (m, 1H), 1.78-1.70 (m, 1H), 1.69-1.57 (m, 5H), 1.56-1.41 (m, 3H).

### Halocyclization+Additional TBDMS-Deprotection

### Synthesis of rac-rel-(3R,5R)-1-methyl-5-(5-(piperidin-1-ylmethyl)-5,6-dihydro-1,4,2-dioxazin-3-yl)piperidin-3-ol, ID 724

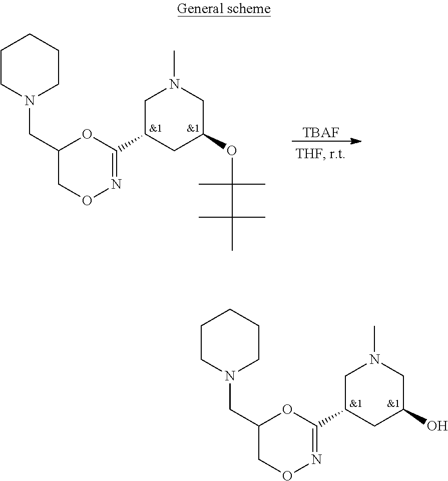

### Procedure 1.9

### Synthesis of rac-rel-(3R,5R)-1-methyl-5-(5-(piperidin-1-ylmethyl)-5,6-dihydro-1,4,2-dioxazin-3-yl)piperidin-3-ol

tert-Butyl-dimethyl-[[rac-rel-(3R,5R)-1-methyl-5-[5-(1-piperidylmethyl)-5,6-dihydro-1,4,2-dioxazin-3-yl]-3-piperidyl]oxy]silane was obtained in a similar manner with non-critical variations from the literature described rac-rel-(3R,5R)-1-(tert-butoxycarbonyl)-5-((2,3,3-trimethylbutan-2-yl)oxy)piperidine-3-carboxylic acid in line with the synthesis described in 1.1 to 1.5 (600 mg, 1.11 mmol, 1 eq) was dissolved in THF (4 ml), after that tetrabutylammonium fluoride, 1M solution in THF (5 ml, 5 mmol, 4 eq) was added to the resulting solution. The reaction mixture was stirred at room temperature for 15 hours. Then solvent was removed by evaporation to afford crude rac-rel-(3R,5R)-1-methyl-5-(5-(piperidin-1-ylmethyl)-5,6-dihydro-1,4,2-dioxazin-3-yl)piperidin-3-ol, which was submitted for prep HPLC purification (15-60% 0-5 min 0.1% NH₃-methanol, flow: 30 ml/min (loading pump 4 ml/min methanol) target mass 284 column: YMC Triart C18 100×20 mm, 5 um) to give the title product (30.5 mg, 8.72%) as yellow oil. LCMS [M+1]⁺ 298.2. ¹H NMR

### Synthesis of rac-rel-(1R,5S)—N,N-dimethyl-5-(5-(piperidin-1-ylmethyl)-5,6-dihydro-1,4,2-dioxazin-3-yl)-3-azabicyclo[3.2.0]heptane-1-carboxamide, ID 765

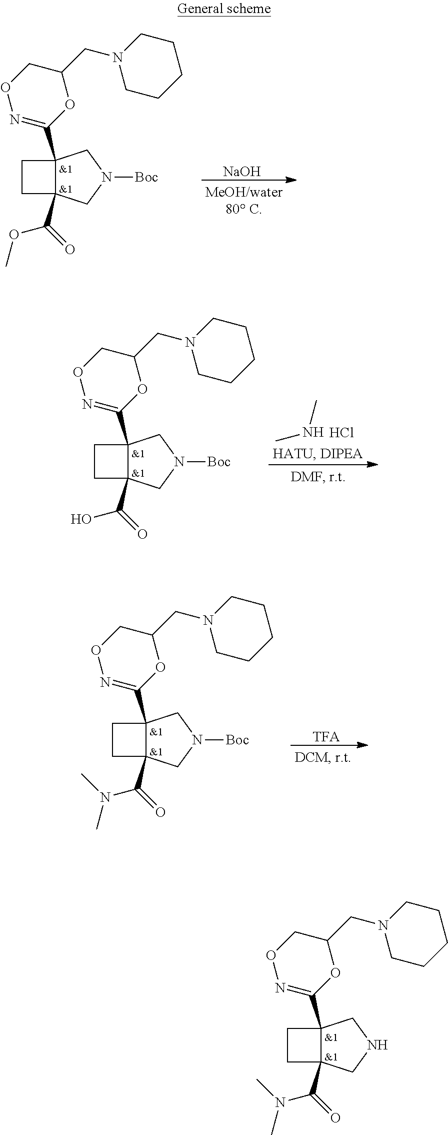

### Procedure 1.10

### Synthesis of rac-rel-cis(1R,5S)-3-(tert-butoxycarbonyl)-5-(5-(piperidin-1-ylmethyl)-5,6-dihydro-1,4,2-dioxazin-3-yl)-3-azabicyclo[3.2.0]heptane-1-carboxylic acid

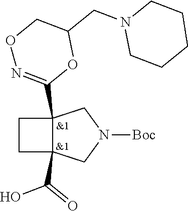 [SMILES: CC(C)(C)OC(=O)N1C[C@]2(C(=O)O)CC[C@]2(C2=NOCC(CN3CCCCC3)O2)C1]

rac-rel-cis-3-(tert-Butyl) 1-methyl (1R,5S)-5-(5-(piperidin-1-ylmethyl)-5,6-dihydro-1,4,2-dioxazin-3-yl)-3-azabicyclo[3.2.0]heptane-1,3-dicarboxylate was obtained in a similar manner with non-critical variations from commercially available rel-cis(1R,5S)-3-(tert-butoxycarbonyl)-5-(methoxycarbonyl)-3-azabicyclo[3.2.0]heptane-1-carboxylic acid in line with the synthesis described in 1.1 to 1.3 (2.9 g, 6.297 mmol, 1 eq) was dissolved in methanol (30 ml), after that a solution of sodium hydroxide (755.61 mg, 18.89 mmol, 3 eq) in water (30 ml) was added to the resulting solution. The reaction mixture was heated at 80° C. for 3 days. After full conversion of the starting material was detected by LCMS, the mixture was concentrated under reduced pressure and the residue obtained was diluted with water (30 ml). 1N aqueous solution of sodium hydrogen sulfate (2267.9 mg, 18.89 mmol, 3 eq) was added to the reaction mixture water solution to adjust p to 7. The precipitate formed was collected by filtration to afford the title product (1.38 g, 49.16%) as white solid. LCMS [M+1]⁺ 424.4.

### Procedure 1.11

### Synthesis of rac-rel-tert-butyl (1R,5S)-1-(dimethylcarbamoyl)-5-(5-(piperidin-1-ylmethyl)-5,6-dihydro-1,4,2-dioxazin-3-yl)-3-azabicyclo[3.2.0]heptane-3-carboxylate

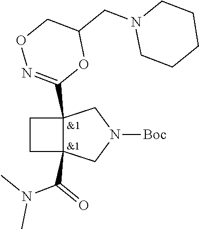 [SMILES: CN(C)C(=O)[C@@]12CC[C@]1(C1=NOCC(CN3CCCCC3)O1)CN(C(=O)OC(C)(C)C)C2]

rac-rel-(1R,5S)-3-tert-Butoxycarbonyl-1-[5-(1-piperidylmethyl)-5,6-dihydro-1,4,2-dioxazin-3-yl]-3-azabicyclo[3.2.0]heptane-5-carboxylic acid (300 mg, 0.6730 mmol, 1 eq), [dimethylamino-(3-oxidotriazolo[4,5-b]pyridin-3-ium-1-yl)methylene]-dimethyl-ammonium hexafluorophosphate (281.47 mg, 0.7403 mmol, 1.1 eq) were mixed together in dry DMF (1 ml), after that N,N-diisopropylethylamine (191.34 mg, 1.4805 mmol, 2.2 eq) was added to the resulting solution, which was stirred at room temperature for 20 minutes. After that period N-methylmethanamine hydrochloride (60.361 mg, 0.7403 mmol, 1.1 eq) was added to the reaction mixture, which was stirred at room temperature for further 14 hours. The reaction mixture solution was then subjected for prep HPLC purification without any work-up (50-90% 0-5 min water-methanol, flow: 30 ml/min (loading pump 4 ml/min methanol) target mass 451; column: SunFireC18 100×19 mm 5 um) to afford the title product (177.4 mg, 55.58%) as yellow oil. LCMS [M+1]⁺ 451.4.

### Procedure 1.12

### Synthesis of rac-rel-(1R,5S)—N,N-dimethyl-5-(5-(piperidin-1-ylmethyl)-5,6-dihydro-142-dioxazin-3-yl)-3-azabicyclo[3.2.0]heptane-1-carboxamide

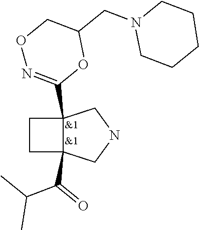 [SMILES: CC(C)C(=O)[C@@]12CC[C@]1(C1=NOCC(CN3CCCCC3)O1)CNC2]

In a generally similar manner with non-critical variations was made rac-rel-(1R,5S)—N,N-dimethyl-5-(5-(piperidin-1-ylmethyl)-5,6-dihydro-1,4,2-dioxazin-3-yl)-3-azabicyclo[3.2.0]heptane-1-carboxamide (34.6 mg, 25.08%) as a beige solid in line with the synthesis described in 1.4. LCMS [M+1]⁺ 351.4. ¹H NMR (Methanol-d₄, 400 MHz): δ (ppm) 4.60-4.55 (m, 1H), 4.18-4.08 (m, 1H), 3.76-3.64 (m, 1H), 3.36 (s, 2H), 3.18-3.14 (m, 1H), 2.90-2.89 (m, 6H), 2.84-2.83 (m, 2H), 2.64-2.54 (m, 4H), 2.51-2.46 (m, 2H), 2.27-2.15 (m, 1H), 2.09-2.03 (m, 1H), 1.89-0.79 (m, 1H), 1.62-1.61 (m, 4H), 1.50-1.48 (m, 2H).

### Synthesis of rac-(4-(5-(piperidin-1-ylmethyl)-5,6-dihydro-1,4,2-dioxazin-3-yl)piperidin-4-yl)methanol, ID 722

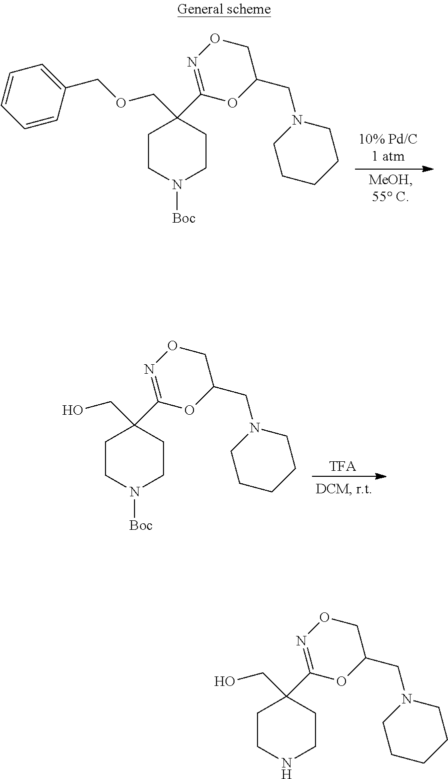 [SMILES: CC(C)(C)OC(=O)N1CCC(COCc2ccccc2)(C2=NOCC(CN3CCCCC3)O2)CC1 ; CC(C)(C)OC(=O)N1CCC(CO)(C2=NOCC(CN3CCCCC3)O2)CC1 ; OCC1(C2=NOCC(CN3CCCCC3)O2)CCNCC1]

### Procedure 1.13

### Synthesis of tert-butyl 4-(hydroxymethyl)-4-(5-(piperidin-1-ylmethyl)-5,6-dihydro-1,4,2-dioxazin-3-yl)piperidine-1-carboxylate

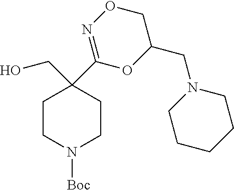 [SMILES: CC(C)(C)OC(=O)N1CCC(CO)(C2=NOCC(CN3CCCCC3)O2)CC1]

rac-tert-Butyl 4-(benzyloxymethyl)-4-[5-(1-piperidylmethyl)-5,6-dihydro-1,4,2-dioxazin-3-yl]piperidine-1-carboxylate, obtained in a similar manner with non-critical variations from commercially available 4-((benzyloxy)methyl)-1-(tert-butoxycarbonyl)piperidine-4-carboxylic acid in line with the synthesis described in 1.1 to 1.3 (1.52 g, 7.5262 mmol, 1 eq) was dissolved in absolute methanol (50 ml), after that palladium on carbon, 10% (80.094 mg, 0.0753 mmol, 0.01 eq) was added to the resulting solution. The reaction mixture was then vacuumed and hydrogen-flushed three times, a balloon with hydrogen was attached and the reaction mixture was heated at 55° C. while vigorous stirring for 14 hours. After that period of time the reaction mixture was cooled down to room temperature and filtered. The catalyst was washed with methanol (50 ml) and the filtrate collected was concentrated under reduced pressure to afford the title product (1.73 g, 35.85%) as yellow oil. The product obtained was used in further experiments without any additional purification. LCMS [M+1]⁺ 398.4.

### Procedure 1.14

### Synthesis of rac-(4-(5-(piperidin-1-ylmethyl)-5,6-dihydro-1,4,2-dioxazin-3-yl)piperidin-4-yl)methanol

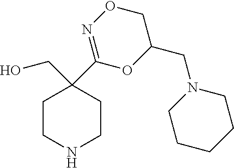 [SMILES: OCC1(C2=NOCC(CN3CCCCC3)O2)CCNCC1]

rac-(4-(5-(piperidin-1-ylmethyl)-5,6-dihydro-1,4,2-dioxazin-3-yl)piperidin-4-yl)methanol (33.8 mg, 45.19%) was prepared as a yellow oil in line with the synthesis described in 1.4. LCMS [M+1]⁺ 298.2. ¹H NMR (Methanol-d₄, 400 MHz): δ (ppm) 4.81-4.71 (m, 1H), 4.21-4.12 (m, 1H), 3.88 (dd, J=11.7, 5.0 Hz, 1H), 3.65-3.50 (m, 2H), 3.28-3.19 (m, 3H), 3.14 (d, J=12.9 Hz, 1H), 3.05-2.93 (m, 1H), 2.93-2.68 (m, 5H), 2.25 (t, J=17.4, 17.4 Hz, 2H), 1.82-1.62 (m, 6H), 1.60-1.50 (m, 2H).

### Halocyclisation+Reductive Amination

### Synthesis of rac-5-((4,4-difluoropiperidin-1-yl)methyl)-3-(4-methylpiperidin-4-yl)-5,6-dihydro-1,4,2-dioxazine, ID 673

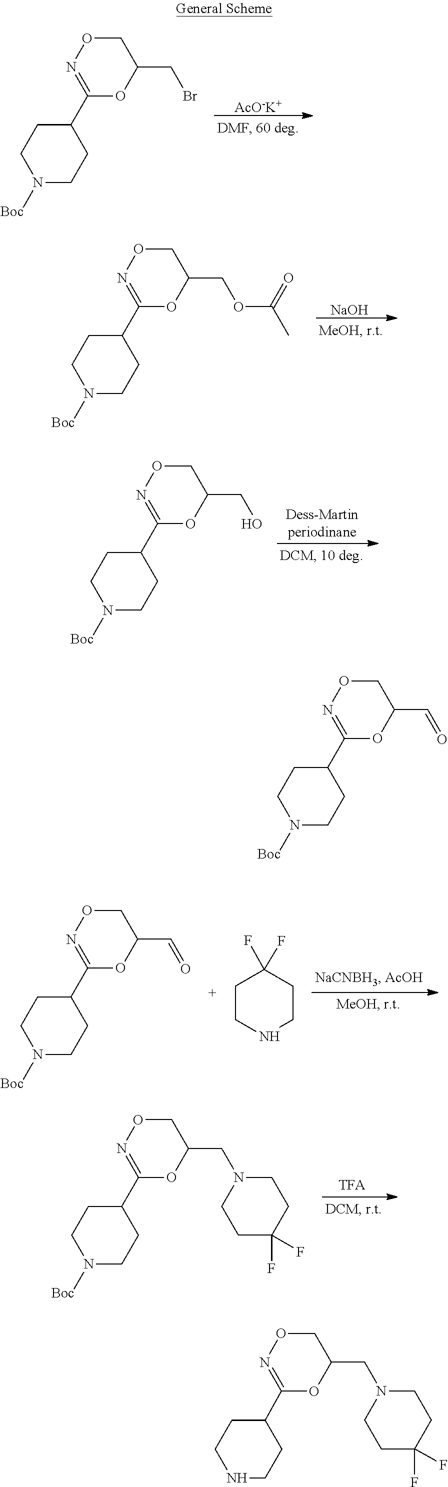

### Procedure 1.15

### Synthesis of tert-butyl 4-[5-(bromomethyl)-5,6-dihydro-1,4,2-dioxazin-3-yl]-4-methyl piperidine-1-carboxylate

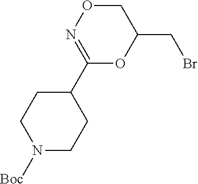 [SMILES: CC(C)(C)OC(=O)N1CCC(C2=NOCC(CBr)O2)CC1]

tert-butyl 4-[5-(bromomethyl)-5,6-dihydro-1,4,2-dioxazin-3-yl]-4-methyl piperidine-1-carboxylate (32 g 96%) was prepared as a dark yellow oil from commercially available tert-butyl 4-(allyloxycarbamoyl)-4-methyl-piperidine-1-carboxylate in line with the synthesis described in 1.1 to 1.2.

### Procedure 1.16

### Synthesis of rac-tert-butyl 4-[5-(acetoxymethyl)-5,6-dihydro-1,4,2-dioxazin-3-yl]-4-methyl-piperidine-1-carboxylate

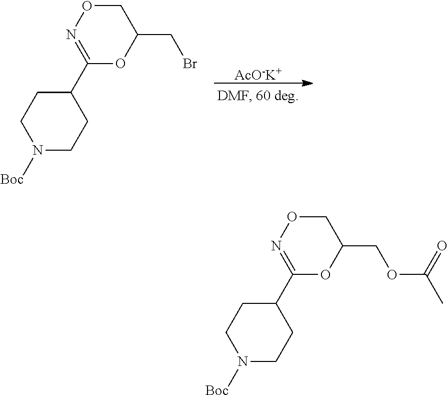 [SMILES: "AcO-K+" ; CC(C)(C)OC(=O)N1CCC(C2=NOCC(CBr)O2)CC1 ; CC(=O)OCC1CON=C(C2CCN(C(=O)OC(C)(C)C)CC2)O1]

To solution of rac-tert-butyl 4-[5-(bromomethyl)-5,6-dihydro-1,4,2-dioxazin-3-yl]-4-methyl-piperidine-1-carboxylate (19 g, 50.36 mmol, 1 eq) in DMF (80 ml) was added potassium acetate (9.88 g, 100.72 mmol, 2 eq). The resulting mixture was stirred at 60° c. for 14 h. After that time, the reaction mixture was diluted with water (250 mL) and extracted with methyl-tert-butyl ether (2×100 mL). The combined organic layers were washed with brine (150 mL), dried over sodium sulfate, filtered and concentrated under reduced pressure to obtain crude rac-tert-butyl 4-[5-(acetoxymethyl)-5,6-dihydro-1,4,2-dioxazin-3-yl]-4-methyl-piperidine-1-carboxylate (16 g, purity 90%, yield 80%), LCMS [M-butene+H]⁺ 301 which was used as such in the next step without further purification.

### Procedure 1.17

### Syntheses of rac-tert-butyl 4-[5-(hydroxymethyl)-5,6-dihydro-1,4,2-dioxazin-3-yl]-4-methyl-piperidine-1-carboxylate

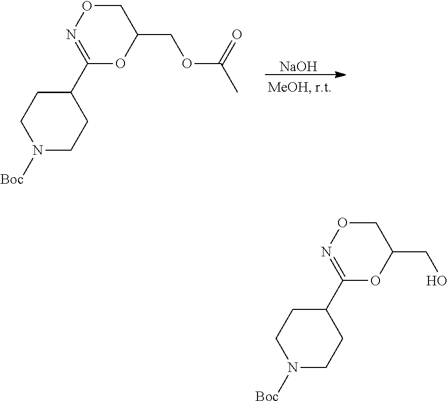

Sodium hydroxide (4.84 g, 121.2 mmol) and rac-tert-Butyl 4-[5-(acetoxymethyl)-5,6-dihydro-1,4,2-dioxazin-3-yl]-4-methyl-piperidine-1-carboxylate (16 g, 40.4 mmol) were suspended in methanol (100 ml) at 20° C. The resulting mixture was stirred at r.t. overnight. The resulting solution was concentrated under reduced pressure, added water and extracted with EtOAc (2×100 ml). The organic layer was dried over Na₂SO₄, filtered, and concentrated in vacuo to afford a light yellow oil. This crude material was purified by silica gel column chromatography (Companion combiflash, 120 g SiO2, petroleum ether/MtBE with MtBE from 0-100%, flow rate=85 mL/min, Rv=11 CV) to obtain rac-tert-butyl 4-[5-(hydroxymethyl)-5,6-dihydro-1,4,2-dioxazin-3-yl]-4-methyl-piperidine-1-carboxylate (9.5 g, 100% purity, 75% yield) as light yellow oil. LCMS [M−butene+H]⁺ 259.2.

### Procedure 1.18

### Synthesis of rac-tert-butyl 4-(5-formyl-5,6-dihydro-1,4,2-dioxazin-3-yl)-4-methyl-piperidine-1-carboxylate

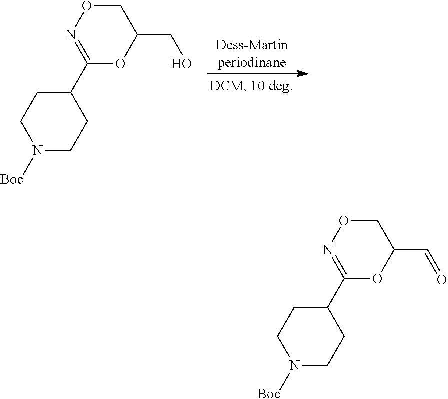 [SMILES: CC(C)(C)OC(=O)N1CCC(C2=NOCC(CO)O2)CC1 ; "Dess-Martinperiodinane" ; CC(C)(C)OC(=O)N1CCC(C2=NOCC(C=O)O2)CC1]

To a stirred solution of rac-tert-butyl 4-[5-(hydroxymethyl)-5,6-dihydro-1,4,2-dioxazin-3-yl]-4-methyl-piperidine-1-carboxylate (6.73 g, 21.40 mmol, 1 eq.) in CH₂Cl₂ at 10° C. was added (1,1,1-Triacetoxy)-1,1-dihydro-1,2-benziodoxol-3(1H)-one (9.98 g, 23.54 mmol, 1.1 eq.). The reaction mixture was left while stirring at room temperature overnight. To the reaction mixture was added saturated solution of NaHCO₃ (200 ml) and stirred for 2 h. Aqueous and organic solutions were filtered. The organic layer was dried over sodium sulfate, filtered, and evaporated under reduced pressure to obtain rac-tert-butyl 4-(5-formyl-5,6-dihydro-1,4,2-dioxazin-3-yl)-4-methyl-piperidine-1-carboxylate N83-1 (6.5 g, 77.7% yield, 80% purity), which was used in the next step without further purification.

### Procedure 1.19

### Synthesis of rac-tert-butyl 4-[5-[(4,4-difluoro-1-piperidyl)methyl]-5,6-dihydro-1,4,2-dioxazin-3-yl]-4-methyl-piperidine-1-carboxylate

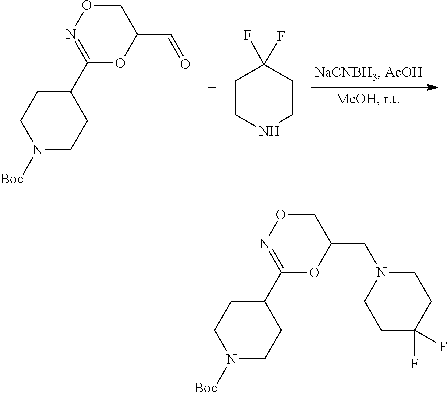 [SMILES: CC(C)(C)OC(=O)N1CCC(C2=NOCC(C=O)O2)CC1 ; FC1(F)CCNCC1 ; CC(C)(C)OC(=O)N1CCC(C2=NOCC(CN3CCC(F)(F)CC3)O2)CC1]

rac-tert-butyl 4-(5-formyl-5,6-dihydro-1,4,2-dioxazin-3-yl)-4-methyl-piperidine-1-carboxylate (500 mg, 1.60 mmol, 1 eq.) was dissolved in MeOH (20 mL) at rt followed by 4,4-difluoropiperidine (639 mg, 5.28 mmol, 3.3 eq), acetic acid (357 uL, 3.9 eq.) and NaCNBH₃ (310 mg, 4.94 mmol, 3.09 eq.) were added and stirred overnight. The reaction mixture was concentrated to dryness and the residue was added to MtBE and 6N NaOH, and extracted with MtBE. The combined organic layers were washed with brine, dried over anhydrous Na₂SO₄, filtered, and concentrated to obtain crude tert-butyl 4-[5-[(4,4-difluoro-1-piperidyl)methyl]-5,6-dihydro-1,4,2-dioxazin-3-yl]-4-methyl-piperidine-1-carboxylate (800 mg, purity), LCMS [M+H]⁺ 418.0 which was used in next step without further purification.

### Synthesis of rac-5-((4,4-difluoropiperidin-1-yl)methyl)-3-(4-methylpiperidin-4-yl)-5,6-dihydro-1,4,2-dioxazine, ID 673

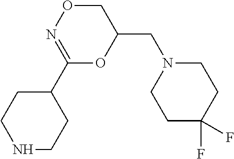 [SMILES: FC1(F)CCN(CC2CON=C(C3CCNCC3)O2)CC1]

rac-5-((4,4-difluoropiperidin-1-yl)methyl)-3-(4-methylpiperidin-4-yl)-5,6-dihydro-1,4,2-dioxazine (175.9 mg, 40%) was prepared as a yellow oil from rac-tert-butyl 4-[5-[(4,4-difluoro-1-piperidyl)methyl]-5,6-dihydro-1,4,2-dioxazin-3-yl]-4-methyl-piperidine-1-carboxylate in line with the synthesis described in 1.4. LCMS [M+1]⁺ 318.2. ¹H NMR (400 MHz, CD₃OD) δ 4.52 (qd, J=6.0, 6.0, 6.0, 2.8 Hz, 1H), 4.12 (dd, J=11.7, 2.9 Hz, 1H), 3.80 (dd, J=11.7, 6.0 Hz, 1H), 3.24 (dt, J=13.3, 4.2, 4.2 Hz, 2H), 3.19-3.08 (m, 2H), 2.72-2.61 (m, 6H), 2.24 (s, 1H), 2.21 (s, 1H), 1.97 (tt, J=12.8, 12.8, 5.6, 5.6 Hz, 4H), 1.64 (ddd, J=15.4, 11.8, 4.3 Hz, 2H), 1.27 (s, 3H).

### 2. General Synthesis Using Mitsunobu Reaction

### Synthesis of rac-3-(4-((5-fluoropyridin-3-yl)methyl)piperidin-4-yl)-5-(piperidin-1-ylmethyl)-5,6-dihydro-1,4,2-dioxazine, ID 528

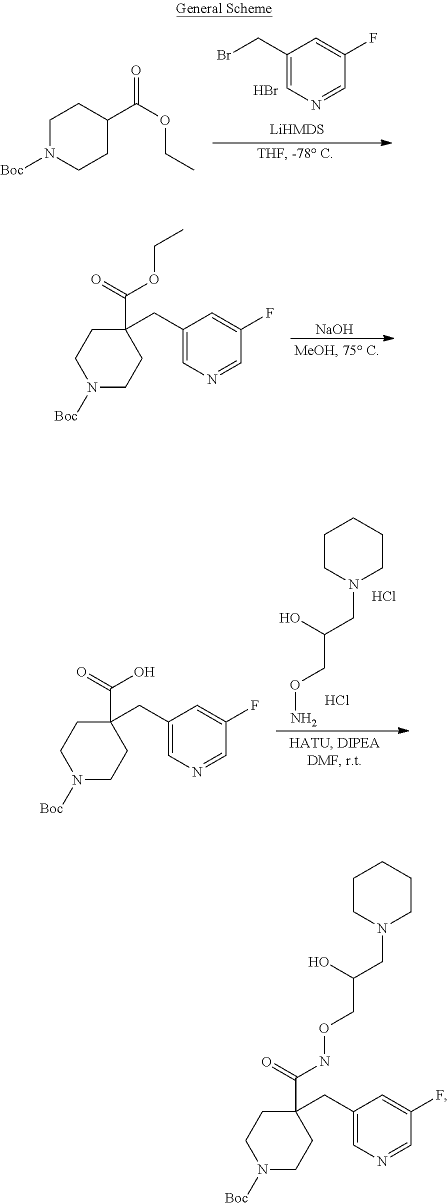

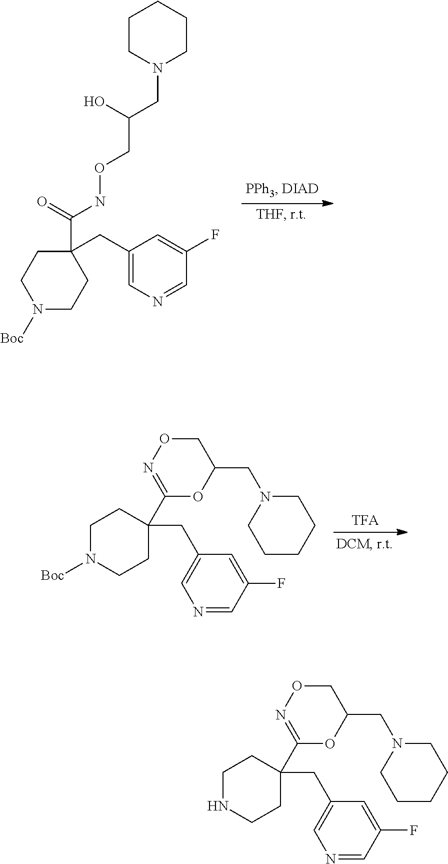 [SMILES: CC(C)(C)OC(=O)N1CCC(Cc2cncc(F)c2)(C(=O)NOCC(O)CN2CCCCC2)CC1 ; CC(C)(C)OC(=O)N1CCC(Cc2cncc(F)c2)(C2=NOCC(CN3CCCCC3)O2)CC1 ; Fc1cncc(CC2(C3=NOCC(CN4CCCCC4)O3)CCNCC2)c1]

### Procedure 2.1

### Synthesis of rac-1-(tert-butyl) 4-ethyl 4-((5-fluoropyridin-3-yl)methyl)piperidine-1,4-dicarboxylate

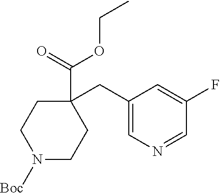 [SMILES: CCOC(=O)C1(Cc2cncc(F)c2)CCN(C(=O)OC(C)(C)C)CC1]

To a solution of lithium bis(trimethylsilyl)azanide, 1M (18 ml, 17.88 mmol, 2.3 eq) in THF (40 mL) in a round-bottomed three necked flask was added the solution of 1-(tert-butyl) 4-ethyl piperidine-1,4-dicarboxylate (2 g, 7.77 mmol, 1 eq) in THF (10 mL) at −78° C. under inert atmosphere (argon inlet). After the mixture was stirred for 30 minutes at −78° C., 3-(bromomethyl)-5-fluoro-pyridine hydrobromide (2.316 g, 8.55 mmol, 1.1 eq) was added in portions direct into the system during 30 minutes. The reaction mixture stirred at −78° C. for additional 30 minutes and then allowed to warm up to room temperature and stirred at ambient temperature overnight. After 14 hours the reaction mixture was quenched with saturated aqueous solution of NH₄Cl (100 mL) and extracted with ethyl acetate (3×30 mL). The organic layers were isolated, combined, washed with water (100 mL) and brine (100 mL), then dried over anhydrous sodium sulfate and filtered. The filtrate was concentrated under reduced pressure to afford 3.65 g of crude orange oil. The crude obtained was purified with FC (Companion combiflash; 80 g SiO₂, chloroform/acetonitrile with acetonitrile from 0-35%, flow rate=60 mL/min, Rv=13 CV) to afford the title product (530 mg, 16.75%) as pale yellow oil with 90% purity, which was used without additional purification. LCMS [M−butene+H]⁺ 311.2

### Procedure 2.2

### Synthesis of rac-1-(tert-butoxycarbonyl)-4-((5-fluoropyridin-3-yl)methyl)piperidine-4-carboxylic acid

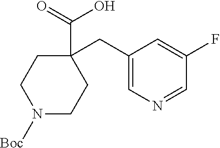 [SMILES: CC(C)(C)OC(=O)N1CCC(Cc2cncc(F)c2)(C(=O)O)CC1]

rac-1-(tert-Butyl) 4-ethyl 4-((5-fluoropyridin-3-yl)methyl)piperidine-1,4-dicarboxylate (530 mg, 1.302 mmol, 1 eq) was dissolved in the mixture of distilled water (3 mL) and methanol (5 mL), after that sodium hydroxide (104.14 mg, 2.604 mmol, 2 eq) was added to the resulting solution. The reaction mixture was then heated up to 75° C. and left while stirring. After 48 hours the reaction mixture was cooled down to ambient temperature and concentrated under reduced pressure to afford a yellow semi-solid residue, which was diluted with distilled water (7 mL) and extracted with DCM (2×5 mL). The aqueous layer was isolated and sodium hydrogen sulfate (312 mg, 2.604 mmol, 2 eq) was added to the solution while stirring. The resulting mixture was stirred at room temperature for additional 30 minutes, and then the precipitate formed was collected by filtration and dried at 65° C. to afford the title product (300 mg, 68.11%) as white solid, which was used without further purification. LCMS [M−1]-337.2.

### Procedure 2.3

### Synthesis of rac-tert-butyl 4-((5-fluoropyridin-3-yl)methyl)-4-((2-hydroxy-3-(piperidin-1-yl)propoxy)carbamoyl)piperidine-1-carboxylate

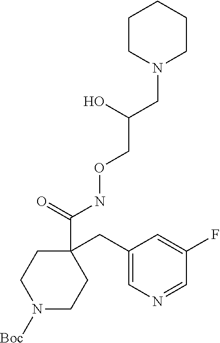 [SMILES: CC(C)(C)OC(=O)N1CCC(Cc2cncc(F)c2)(C(=O)NOCC(O)CN2CCCCC2)CC1]

rac-1-tert-Butoxycarbonyl-4-[(5-fluoro-3-pyridyl)methyl]piperidine-4-carboxylic acid (300 mg, 0.575 mmol, 1 eq) was dissolved in dry DMF (5 mL), after that [dimethylamino(triazolo[4,5-b]pyridin-3-yloxy)methylene]-dimethyl-ammonium hexafluorophosphate (371 mg, 0.975 mmol, 1.1 eq) was added to the resulting solution, followed by the addition of N,N-Diisopropylethylamine (0.772 ml, 572.92 mg, 4.43 mmol, 5 eq) and 1-aminooxy-3-(1-piperidyl)propan-2-ol dihydrochloride (285 mg, 1.15 mmol, 1.3 eq). The reaction mixture was left while stirring at ambient temperature overnight. After 14 hours the reaction mixture solution was subjected to preparative HPLC without any work up (45-80% 0-5 min water-methanol, flow: 30 ml/min (loading pump 4 ml/min methanol), target mass 495; column: SunFire C18 100×19 mm, 5 um) to afford the title product (254.2 mg, 52.17%) as a yellow oil with 90% purity, which was used in further experiment without additional purification. LCMS [M+1]⁺ 495.4

### Procedure 2.4

### Synthesis of rac-tert-butyl 4-((5-fluoropyridin-3-yl)methyl)-4-(5-(piperidin-1-ylmethyl)-5,6-dihydro-1,4,2-dioxazin-3-yl)piperidine-1-carboxylate

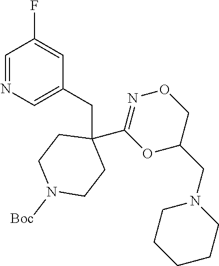 [SMILES: CC(C)(C)OC(=O)N1CCC(Cc2cncc(F)c2)(C2=NOCC(CN3CCCCC3)O2)CC1]

rac-tert-Butyl 4-[(5-fluoro-3-pyridyl)methyl]-4-[[2-hydroxy-3-(1-piperidyl)propoxy]carbamoyl]piperidine-1-carboxylate (254.2 mg, 0.463 mmol, 1 eq) was dissolved in dry THF (5 mL), after that triphenylphosphane (242.65 mg, 0.925 mmol, 2 eq) was added to the resulting solution, followed by the addition of diisopropylazodicarboxylate (DIAD) (187.07 mg, 0.925 mmol, 2 eq). The reaction mixture was left while stirring at ambient temperature overnight. After 12 hours the reaction mixture was concentrated under reduced pressure to afford a yellow coloured oily residue (705 mg). The crude obtained was purified with preparative HPLC (50-85% 0-6 min water-methanol, flow: 30 ml/min (loading pump 4 ml/min methanol) target mass 477; column: SunFireC18 100×19 mm 5 um) to afford the title product as a yellow oil (129 mg, 53.84%), which was used without additional purification. LCMS [M+1]⁺ 477.2

### Procedure 2.5

### Synthesis of rac-3-(4-((5-fluoropyridin-3-yl)methyl)piperidin-4-yl)-5-(piperidin-1-ylmethyl)-5,6-dihydro-1,4,2-dioxazine ID 528

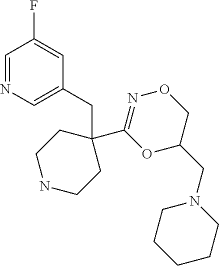 [SMILES: Fc1cncc(CC2(C3=NOCC(CN4CCCCC4)O3)CCNCC2)c1]

rac-tert-Butyl 4-[(5-fluoro-3-pyridyl)methyl]-4-[5-(1-piperidylmethyl)-5,6-dihydro-1,4,2-dioxazin-3-yl]piperidine-1-carboxylate (129 mg, 0.249 mmol, 1 eq) was dissolved in dry DCM (2 mL), followed by dropwise addition of 2,2,2-trifluoroacetic acid (308.6 mg, 2.49 mmol, 10 eq). The reaction mixture was left while stirring at ambient temperature overnight. After 12 hours the reaction mixture was concentrated under reduced pressure to afford a yellow coloured oily residue, which was diluted with DCM (10 mL) and washed with 30% aqueous solution of potassium carbonate (2×10 mL). The organic layer was isolated, dried over anhydrous sodium sulfate and filtered. The filtrate was concentrated under reduced pressure to afford a crude product residue (120 mg, pale yellow oil). The residue obtained was subjected to preparative HPLC (95-95-75% 0-1-6 min water-acetonitrile, flow: 30 ml/min (loading pump 4 ml/min acetonitrile) target mass 377; column Uptisphere Strategy HILIC-HIA 100×21.2 mm 5 um) to afford the title product (94.1 mg, 95.35%). LCMS [M+1]⁺ 377.4. ¹H NMR (400 MHz, CDCl₃) δ 8.34 (d, J=2.4 Hz, 1H), 8.19 (s, 1H), 7.23-7.17 (m, 1H), 4.37-4.28 (m, 1H), 4.04 (dd, J=11.5, 2.7 Hz, 1H), 3.69 (dd, J=11.7, 6.4 Hz, 1H), 3.14 (d, J=12.8 Hz, 2H), 2.96 (t, J=11.9, 11.9 Hz, 2H), 2.82 (s, 2H), 2.46 (d, J=5.9 Hz, 2H), 2.44-2.33 (m, 4H), 2.22-2.11 (m, 2H), 1.79-1.62 (m, 2H), 1.59-1.48 (m, 4H), 1.47-1.35 (m, 2H).

### Synthesis of rac-3-(4-((2-methylpyridin-3-yl)methyl)piperidin-4-yl)-5-(piperidin-1-ylmethyl)-5,6-dihydro-1,4,2-dioxazine, ID 561

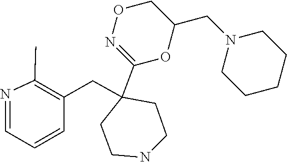

rac-3-(4-((2-methylpyridin-3-yl)methyl)piperidin-4-yl)-5-(piperidin-1-ylmethyl)-5,6-dihydro-1,4,2-dioxazine (153.1 mg, 76.34%) was prepared as a yellow oil from commercially available 1-(tert-butyl) 4-ethyl piperidine-1,4-dicarboxylate in line with the synthesis described in 2.1 to 2.5 but using 3-(chloromethyl)-2-methyl-pyridine hydrochloride instead of 3-(bromomethyl)-5-fluoro-pyridine hydrobromide in experimental procedure 2.1. LCMS [M+1]⁺ 373.2. ¹H NMR (400 MHz, CD₃OD) δ 8.30 (d, J=4.4 Hz, 1H), 7.60 (d, J=7.8 Hz, 1H), 7.27-7.13 (m, 1H), 4.43-4.32 (m, 1H), 4.12 (dd, J=11.7, 3.1 Hz, 1H), 3.61 (dd, J=11.5, 7.5 Hz, 1H), 3.00-2.80 (m, 4H), 2.80-2.61 (m, 2H), 2.59-2.47 (m, 4H), 2.46-2.26 (m, 5H), 2.23-1.99 (m, 2H), 1.65-1.48 (m, 6H), 1.48-1.38 (m, 2H).

### Synthesis of rac-5-(piperidin-1-ylmethyl)-3-(3-(pyridin-3-ylmethyl)pyrrolidin-3-yl)-5,6-dihydro-1,4,2-dioxazine, ID 643

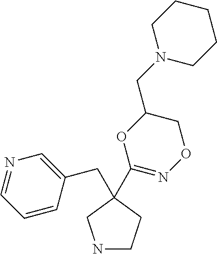 [SMILES: c1cncc(CC2(C3=NOCC(CN4CCCCC4)O3)CCNC2)c1]

rac-5-(piperidin-1-ylmethyl)-3-(3-(pyridin-3-ylmethyl)pyrrolidin-3-yl)-5,6-dihydro-1,4,2-dioxazine (17.8 mg, 26.95%) was prepared as a yellow oil from commercially available 1-(tert-butyl) 3-methyl pyrrolidine-1,3-dicarboxylate in line with the synthesis described in 2.1 to 2.5 but using 3-(chloromethyl)pyridine hydrochloride instead of 3-(bromomethyl)-5-fluoro-pyridine hydrobromide in experimental procedure 2.1. LCMS [M+1]⁺ 345.4. ¹H NMR (400 MHz, CD₃OD) δ 8.49 (d, J=3.2 Hz, 1H), 8.45 (s, 1H), 7.77 (t, J=6.3, 6.3 Hz, 1H), 7.48-7.40 (m, 1H), 4.77-4.67 (m, 1H), 4.15-4.08 (m, 1H), 3.78-3.61 (m, 2H), 3.56-3.46 (m, 1H), 3.41-3.34 (m, 1H), 3.33-3.30 (m, 2H), 3.28-3.19 (m, 2H), 3.17-3.09 (m, 1H), 3.00-2.89 (m, 2H), 2.87-2.81 (m, 3H), 2.58-2.47 (m, 1H), 2.18-2.05 (m, 1H), 1.82-1.69 (m, 4H), 1.66-1.52 (m, 2H).

### Synthesis of rac-5-(piperidin-1-ylmethyl)-3-(4-(pyridin-3-ylmethyl)piperidin-4-yl)-5,6-dihydro-1,4,2-dioxazine, ID 550

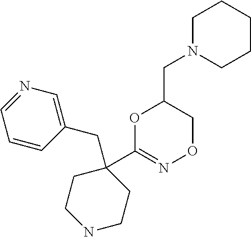 [SMILES: c1cncc(CC2(C3=NOCC(CN4CCCCC4)O3)CCNCC2)c1]

rac-5-(Piperidin-1-ylmethyl)-3-(4-(pyridin-3-ylmethyl)piperidin-4-yl)-5,6-dihydro-1,4,2-dioxazine (100 mg, 83.07%) was prepared as a yellow oil from commercially available 1-(tert-butyl) 4-ethyl piperidine-1,4-dicarboxylate in line with the synthesis described in 2.1 to 2.5 but using 3-(chloromethyl)pyridine hydrochloride instead of 3-(bromomethyl)-5-fluoro-pyridine hydrobromide in experimental procedure 2.1. LCMS [M+1]⁺ 359.4. ¹H NMR (400 MHz, CDCl₃) δ 8.48 (d, J=4.2 Hz, 1H), 8.37 (s, 1H), 7.43 (d, J=7.8 Hz, 1H), 7.22-7.18 (m, 1H), 4.37-4.28 (m, 1H), 4.05 (dd, J=11.6, 2.5 Hz, 1H), 3.68 (dd, J=11.5, 6.5 Hz, 1H), 3.23 (d, J=12.4 Hz, 2H), 3.08-2.97 (m, 2H), 2.87-2.78 (m, 2H), 2.45 (d, J=6.0 Hz, 2H), 2.44-2.30 (m, 5H), 2.21 (d, J=13.8 Hz, 2H), 1.83 (t, J=13.7, 13.7 Hz, 2H), 1.61-1.45 (m, 5H), 1.43-1.35 (m, 2H).

### Synthesis of rac-3-(4-((1-methyl-1H-pyrazol-5-yl)methyl)piperidin-4-yl)-5-(piperidin-1-ylmethyl)-5,6-dihydro-1,4,2-dioxazine, ID 547

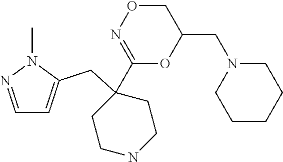 [SMILES: Cn1nccc1CC1(C2=NOCC(CN3CCCCC3)O2)CCNCC1]

rac-3-(4-((1-methyl-1H-pyrazol-5-yl)methyl)piperidin-4-yl)-5-(piperidin-1-ylmethyl)-5,6-dihydro-1,4,2-dioxazine (36.9 mg, 90.21%) was prepared as a yellow solid from commercially available 1-(tert-butyl) 4-ethyl piperidine-1,4-dicarboxylate in line with the synthesis described in 2.1 to 2.5 but using 5-(chloromethyl)-1-methyl-1H-pyrazole hydrochloride instead of 3-(bromomethyl)-5-fluoro-pyridine hydrobromide in experimental procedure 2.1. LCMS [M+1]⁺ 362.2. ¹H NMR (400 MHz, DMSO-d₆) δ 7.38-7.31 (m, 1H), 6.06-6.00 (m, 1H), 4.29-4.21 (m, 1H), 4.07-3.98 (m, 1H), 3.76 (s, 3H), 3.62 (dd, J=10.9, 7.3 Hz, 1H), 2.90-2.82 (m, 2H), 2.81-2.73 (m, 4H), 2.47-2.35 (m, 4H), 2.35-2.25 (m, 3H), 2.11-2.02 (m, 2H), 1.49-1.46 (m, 3H), 1.42-1.31 (m, 5H).

### Synthesis of rac-5-(piperidin-1-ylmethyl)-3-(quinuclidin-4-yl)-5,6-dihydro-1,4,2-dioxazine, ID 642

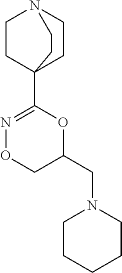 [SMILES: C1CCN(CC2CON=C(C34CCN(CC3)CC4)O2)CC1]

rac-5-(Piperidin-1-ylmethyl)-3-(quinuclidin-4-yl)-5,6-dihydro-1,4,2-dioxazine (149 mg, 34.76%) was prepared as a beige solid from commercially available quinuclidine-4-carboxylic acid in line with the synthesis described in 2.3 to 2.4. LCMS [M+1]⁺ 294.2. ¹H NMR (400 MHz, CD₃OD) δ 4.86-4.84 (m, 2H), 4.50-4.40 (m, 1H), 4.05 (dd, J=11.6, 3.0 Hz, 1H), 3.72-3.61 (m, 1H), 2.95-2.87 (m, 5H), 2.64-2.55 (m, 2H), 2.55-2.40 (m, 4H), 1.77-1.69 (m, 5H), 1.66-1.54 (m, 4H), 1.51-1.40 (m, 2H).

### Synthesis of rac-3-(1-azabicyclo[2.2.1]heptan-4-yl)-5-(piperidin-1-ylmethyl)-5,6-dihydro-1,4,2-dioxazine, ID 641

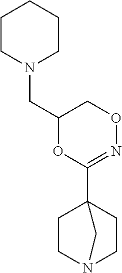 [SMILES: C1CCN(CC2CON=C(C34CCN(CC3)C4)O2)CC1]

rac-3-(1-Azabicyclo[2.2.1]heptan-4-yl)-5-(piperidin-1-ylmethyl)-5,6-dihydro-1,4,2-dioxazine (79.6 mg, 62.86%) was prepared as a yellow oil from commercially available 1-azabicyclo[2.2.1]heptane-4-carboxylic acid in line with the synthesis described in 2.3 to 2.4. LCMS [M+1]⁺ 280.2. ¹H NMR (400 MHz, CD₃OD) δ 4.68-4.60 (m, 1H), 4.18 (dd, J=11.8, 2.9 Hz, 1H), 3.81 (dd, J=11.7, 6.3 Hz, 1H), 3.58-3.43 (m, 2H), 3.31-3.25 (m, 4H), 2.76 (d, J=5.7 Hz, 2H), 2.74-2.59 (m, 4H), 2.34-2.21 (m, 2H), 2.07-1.89 (m, 2H), 1.73-1.61 (m, 4H), 1.60-1.45 (m, 2H).

### Synthesis of rac-3-(1-azabicyclo[3.2.1]octan-5-yl)-5-(piperidin-1-ylmethyl)-5,6-dihydro-1,4,2-dioxazine, ID 553

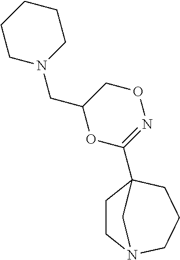 [SMILES: C1CCN(CC2CON=C(C34CCCN(CC3)C4)O2)CC1]

rac-3-(1-Azabicyclo[3.2.1]octan-5-yl)-5-(piperidin-1-ylmethyl)-5,6-dihydro-1,4,2-dioxazine (1185 mg, 28.04%) was prepared as a yellow oil from commercially available rac-1-azabicyclo[3.2.1]octane-5-carboxylic acid in line with the synthesis described in 2.3 to 2.4. LCMS [M+1]⁺ 294.2 ¹H NMR (400 MHz, CDCl₃) δ 4.39-4.23 (m, 1H), 4.05 (d, J=11.4 Hz, 1H), 3.75-3.59 (m, 1H), 3.07-2.97 (m, J=11.6, 5.7 Hz, 1H), 2.91-2.57 (m, 6H), 2.55-2.42 (m, 4H), 2.36 (s, 2H), 2.05-1.92 (m, J=11.7 Hz, 1H), 1.87-1.64 (m, 4H), 1.53-1.47 (m, 3H), 1.46-1.31 (m, 3H).

### Synthesis of rac-5-(piperidin-1-ylmethyl)-3-(quinuclidin-3-yl)-5,6-dihydro-1,4,2-dioxazine, ID 644

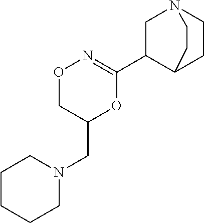 [SMILES: C1CCN(CC2CON=C(C3CN4CCC3CC4)O2)CC1]

rac-5-(Piperidin-1-ylmethyl)-3-(quinuclidin-3-yl)-5,6-dihydro-1,4,2-dioxazine (151.4 mg, 20.82%) was prepared as a yellow oil from commercially available rac-quinuclidine-3-carboxylic acid in line with the synthesis described in 2.3 to 2.4. LCMS [M+1]⁺ 294.2. ¹H NMR (400 MHz, CD₃OD) δ 4.55-4.46 (m, 1H), 4.17-4.08 (m, 1H), 3.74 (dd, J=11.5, 6.4 Hz, 1H), 3.20-3.14 (m, 1H), 3.01-2.72 (m, 5H), 2.66-2.56 (m, 3H), 2.56-2.43 (m, 5H), 2.09-2.04 (m, 1H), 1.91-1.78 (m, 1H), 1.73-1.69 (m, 1H), 1.64-1.58 (m, 4H), 1.51-1.42 (m, 3H).

### Synthesis of rac-5-(piperidin-1-ylmethyl)-3-(5,6,7,8-tetrahydroimidazo[1,5-a]pyridin-7-yl)-5,6-dihydro-1,4,2-dioxazine, ID 655

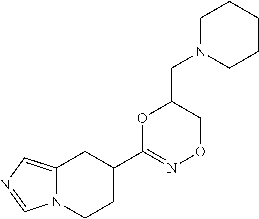 [SMILES: c1ncn2c1CC(C1=NOCC(CN3CCCCC3)O1)CC2]

rac-5-(piperidin-1-ylmethyl)-3-(5,6,7,8-tetrahydroimidazo[1,5-a]pyridin-7-yl)-5,6-dihydro-1,4,2-dioxazine (54 mg, 7.76%) was prepared as a yellow oil from commercially available rac-5,6,7,8-tetrahydroimidazo[1,5-a]pyridine-7-carboxylic acid in line with the synthesis described in 2.3 to 2.4. LCMS [M+1]⁺ 305.2. ¹H NMR (400 MHz, CD₃OD) δ 7.52 (s, 1H), 6.69 (s, 1H), 4.60-4.46 (m, 1H), 4.33-4.21 (m, 1H), 4.20-4.07 (m, 1H), 4.05-3.92 (m, 1H), 3.79-3.66 (m, 1H), 3.12-3.02 (m, 1H), 2.88-2.81 (m, 1H), 2.81-2.73 (m, 1H), 2.64-2.56 (m, 2H), 2.55-2.46 (m, 3H), 2.31-2.21 (m, 1H), 2.09-1.98 (m, 1H), 1.68-1.54 (m, 4H), 1.53-1.40 (m, 2H), 1.28-1.23 (m, 1H).

### Synthesis of rac-5-(piperidin-1-ylmethyl)-3-(5,6,7,8-tetrahydroimidazo[1,2-a]pyridin-7-yl)-5,6-dihydro-1,4,2-dioxazine, ID 660

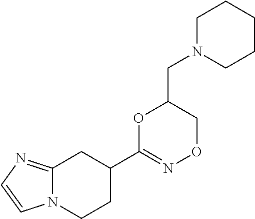 [SMILES: c1cn2c(n1)CC(C1=NOCC(CN3CCCCC3)O1)CC2]

rac-5-(piperidin-1-ylmethyl)-3-(5,6,7,8-tetrahydroimidazo[1,2-a]pyridin-7-yl)-5,6-dihydro-1,4,2-dioxazine (76 mg, 16.1%) was prepared as a yellow oil from commercially available rac-5,6,7,8-tetrahydroimidazo[1,2-a]pyridine-7-carboxylic acid in line with the synthesis described in 2.3 to 2.4. LCMS [M+1]⁺ 305.2. ¹H NMR (400 MHz, CD₃OD) δ 6.94 (s, 1H), 6.87 (s, 1H), 4.57-4.47 (m, 1H), 4.19-4.08 (m, 2H), 4.02-3.92 (m, 1H), 3.73 (dd, J=11.7, 6.5 Hz, 1H), 3.11-2.98 (m, 1H), 2.98-2.83 (m, 2H), 2.61-2.55 (m, 2H), 2.52-2.45 (m, 3H), 2.33-2.23 (m, 1H), 2.13-2.02 (m, 1H), 1.60 (p, J=5.7, 5.7, 5.6, 5.6 Hz, 4H), 1.52-1.40 (m, 2H), 1.29-1.21 (m, 1H).

### Synthesis of rac-N,N-dimethyl-3-(5-(piperidin-1-ylmethyl)-5,6-dihydro-1,4,2-dioxazin-3-yl)propan-1-amine, ID 768

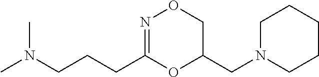 [SMILES: CN(C)CCCC1=NOCC(CN2CCCCC2)O1]

rac-N,N-dimethyl-3-(5-(piperidin-1-ylmethyl)-5,6-dihydro-1,4,2-dioxazin-3-yl)propan-1-amine (127 mg, 44.85%) was prepared as a yellow oil from commercially available 4-(dimethylamino)butanoic acid in line with the synthesis described in 2.3 to 2.4. LCMS [M+1]⁺ 270.4. ¹H NMR (Methanol-d₄, 400 MHz): δ (ppm) 4.56-4.47 (m, 1H), 4.11 (dd, J=11.6, 2.7 Hz, 1H), 3.73 (dd, J=11.6, 6.5 Hz, 1H), 2.58 (d, J=5.8 Hz, 2H), 2.57-2.41 (m, 4H), 2.41-2.34 (m, 2H), 2.26 (s, 6H), 2.25-2.15 (m, 2H), 1.85-1.72 (m, 2H), 1.67-1.56 (m, 4H), 1.54-1.41 (m, 2H).

### Synthesis of rac-3-(2-(piperidin-1-yl)ethyl)-5-(piperidin-1-ylmethyl)-5,6-dihydro-1,4,2-dioxazine, ID 816

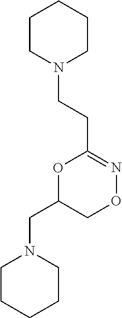 [SMILES: C1CCN(CCC2=NOCC(CN3CCCCC3)O2)CC1]

rac-3-(2-(piperidin-1-yl)ethyl)-5-(piperidin-1-ylmethyl)-5,6-dihydro-1,4,2-dioxazine (62.8 mg, 32.09%) was prepared as a yellow oil from commercially available 3-(piperidin-1-yl)propanoic acid in line with the synthesis described in 2.3 to 2.4. LCMS [M+1]⁺ 296.4. ¹H NMR (Chloroform-d, 400 MHz): δ (ppm) 4.51-4.35 (m, 1H), 4.12 (d, J=13.6 Hz, 1H), 3.78-3.70 (m, 1H), 2.78-2.68 (m, 1H), 2.64-2.14 (m, 11H), 1.82-1.34 (m, 12H), 1.32-0.92 (m, 1H).

### Synthesis of rac-3-(2-(4-methylpiperazin-1-yl)ethyl)-5-(piperidin-1-ylmethyl)-5,6-dihydro-1,4,2-dioxazine, ID 823

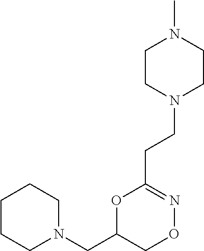 [SMILES: CN1CCN(CCC2=NOCC(CN3CCCCC3)O2)CC1]

rac-3-(2-(4-methylpiperazin-1-yl)ethyl)-5-(piperidin-1-ylmethyl)-5,6-dihydro-1,4,2-dioxazine (72.9 mg, 35.96%) was prepared as a yellow oil from commercially available 3-(4-methylpiperazin-1-yl)propanoic acid in line with the synthesis described in 2.3 to 2.4. LCMS [M+1]⁺ 311.4. ¹H NMR (Methanol-d₄, 400 MHz): δ (ppm) 4.54-4.46 (m, 1H), 4.11 (dd, J=11.6, 2.9 Hz, 1H), 3.74 (dd, J=11.6, 6.5 Hz, 1H), 2.63 (t, J=7.4, 7.4 Hz, 3H), 2.58 (d, J=5.8 Hz, 2H), 2.58-2.43 (m, 10H), 2.41 (t, J=7.4, 7.4 Hz, 3H), 2.29 (s, 3H), 1.68-1.59 (m, 4H), 1.53-1.42 (m, 2H).

### Synthesis of rac-rel-cis-(3R,4R)—N,N-dimethyl-4-(5-(piperidin-1-ylmethyl)-5,6-dihydro-1,4,2-dioxazin-3-yl)pyrrolidin-3-amine, ID 766

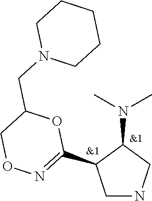 [SMILES: CN(C)[C@H]1CNC[C@H]1C1=NOCC(CN2CCCCC2)O1]

In a generally similar manner with non-critical variations was made rac-rel-cis-(3R,4R)—N,N-dimethyl-4-(5-(piperidin-1-ylmethyl)-5,6-dihydro-1,4,2-dioxazin-3-yl)pyrrolidin-3-amine (18 mg, 46.12%) as a yellow oil from the starting rac-rel-cis-1-(tert-butoxycarbonyl)-4-((R)-dimethylamino)pyrrolidine-3-(R)carboxylic acid in line with the synthesis described in 2.3 to 2.4. The synthesis of the starting building block is described above. LCMS [M+1]⁺ 297.4. ¹H NMR (Methanol-d₄, 400 MHz): δ (ppm) 4.61-4.50 (m, 1H), 4.16-4.06 (m, 1H), 3.77 (dd, J=11.7, 6.2 Hz, 1H), 3.31-2.85 (m, 4H), 2.87-2.68 (m, 2H), 2.65-2.58 (m, 2H), 2.55-2.37 (m, 4H), 2.31 (s, 6H), 1.69-1.59 (m, 4H), 1.54-1.45 (m, 2H).

### 3. General Synthesis Using Base Induced Cyclization from Chloroamidoxime

### Synthesis of rac-3-(1-methylcyclobutyl)-5-(1-piperidylmethyl)-5,6-dihydro-1,4,2-dioxazine ID 423

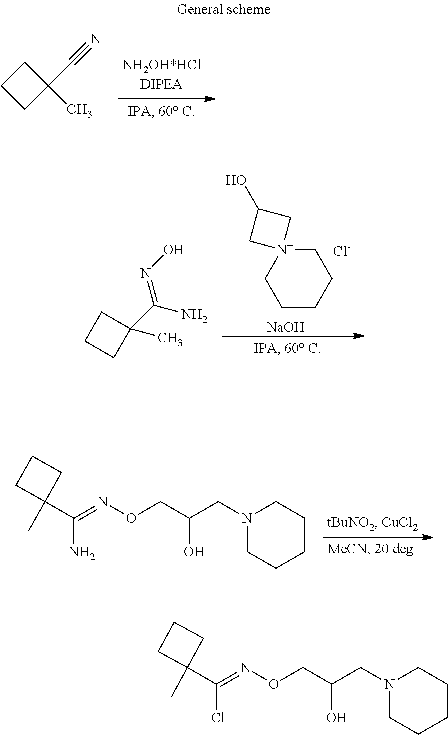

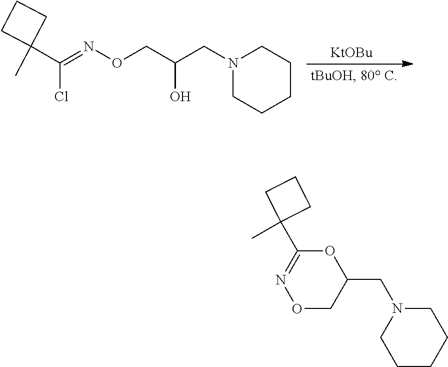 [SMILES: CC1(/C(Cl)=N/OCC(O)CN2CCCCC2)CCC1 ; CC1(C2=NOCC(CN3CCCCC3)O2)CCC1]

### Procedure 3.1

### Synthesis of N′-hydroxy-1-methyl-cyclobutanecarboxamidine

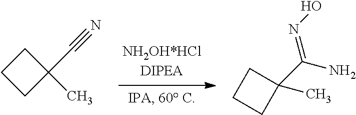

1-Methylcyclobutanecarbonitrile (5.0 g, 52.55 mmol, 1 eq), hydroxylamine hydrochloride (7.30 g, 105.11 mmol, 2 eq) and N,N-diethylethanamine (10.64 g, 105.11 mmol, 2 eq) were dissolved in 75 ml of IPA. The mixture was stirred at 60 C for 36 h after which isopropanol was removed in vacuo. The crude product (21 g), was then purified by flash column chromatography to yield 1.32 g of pure title compound LCMS [M+H]⁺ 129.2)

### Procedure 3.2

### Synthesis of rac-N′-[2-hydroxy-3-(1-piperidyl)propoxy]-1-methyl-cyclobutanecarboxamidine

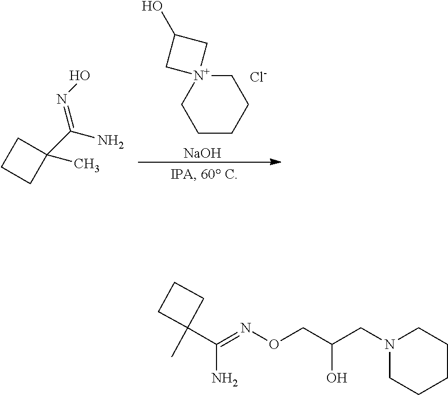

Sodium hydroxide (0.406 g (10.14 mmol, 1 eq), 4-azoniaspiro[3.5]nonan-2-ol chloride (1.80 g (10.14 mmol, 1 eq), and N′-hydroxy-1-methyl-cyclobutanecarboxamidine 1.30 g (10.14 mmol, 1 eq) was mixed in 50 ml of IPA and stirred for 36 hours at 60° C. The solution was filtered and isopropanol removed in vacuo to yield the crude title product (2.70 g) which was used without additional purification LCMS [M+1]⁺ 270.2.

### Procedure 3.3

### Synthesis of rac-(1Z)—N-[2-hydroxy-3-(1-piperidyl)propoxy]-1-methyl-cyclobutanecarboximidoyl chloride

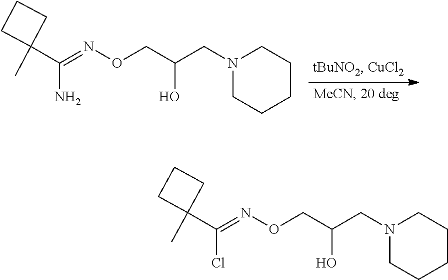 [SMILES: CC1(/C(N)=N/OCC(O)CN2CCCCC2)CCC1 ; CC1(/C(Cl)=N/OCC(O)CN2CCCCC2)CCC1]

A mixture of N′-[2-hydroxy-3-(1-piperidyl)propoxy]-1-methyl-cyclobutanecarboxamidine (1.70 g, 5.68 mmol, 1 eq), tBuONO (1.76 g, 17.04 mmol, 3 eq), and CuCl₂ (2.29 g, 17.04 mmol, 3 eq) in MeCN (50 ml) was stirred at rt for 2 days with exclusion of light. The reaction mixture was concentrated under vacuum, and the remaining residue was suspended in a 2.0 M aqueous sodium carbonate solution (50 ml), and extracted with AcOEt (2×30 ml). The combined organic fractions were dried over sodium sulphate, filtered, and evaporated, resulting in 1.3 g of the title compound as a yellow viscous oil which was used in next step without additional purification. LCMS: [M+1]⁺ 289.2)

### Procedure 3.4

### Synthesis of rac-3-(1-methylcyclobutyl)-5-(1-piperidylmethyl)-5,6-dihydro-1,4,2-dioxazine ID 423

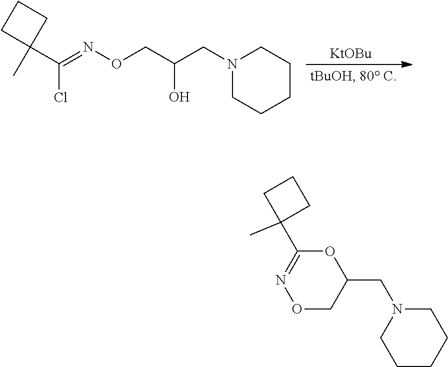 [SMILES: CC1(/C(Cl)=N/OCC(O)CN2CCCCC2)CCC1 ; CC1(C2=NOCC(CN3CCCCC3)O2)CCC1]

(1Z)—N-[2-hydroxy-3-(1-piperidyl)propoxy]-1-methyl-cyclobutanecarboximidoyl chloride, from above (800 mg, 2.77 mmol, 1 eq) was dissolved in absolute tert-BuOH (30 ml), followed by potassium 2-methylpropan-2-olate (932 mg, 8.31 mmol, 3 eq). The reaction mixture was then heated up to 80 C and stirred over night. After 24 hours the reaction mass was concentrated to dryness, diluted with water (30 ml) and extracted with ethyl acetate (3×20 ml). The organic layers were washed with brine, dried over sodium sulfate, filtered, and concentrated to afford crude product as a light yellow oil (0.5 g). The crude product was purified by HPLC to yield 67 mg pure title compound. LCMS [M+1]⁺ 253.4. ¹H NMR (500 MHz, cdcl₃) δ 4.47-4.34 (m, 1H), 4.20-4.06 (m, 1H), 3.81-3.70 (m, 1H), 2.61-2.49 (m, 4H), 2.48-2.37 (m, 4H), 2.05-1.93 (m, 1H), 1.91-1.74 (m, 3H), 1.63-1.55 (m, 4H), 1.50-1.42 (m, 2H), 1.38 (s, 3H).

### Example 2: Determination of Potencies and Efficacies of Dioxazines Using GCase Assay

### Materials

Human fibroblast cell line GM10915 harboring the L444P GBA mutation was obtained from Coriell Biorepositories.

All chemicals (Glacial acetic acid, Glycine, 4-Methylumbelliferyl b-D-glucopyranoside (4-MUG), Sodium acetate trihydrate, Sodium hydroxide, Crystal violet, SDS, Ammonium hydroxide) were obtained from Sigma-Aldrich (Denmark) Compounds tested for GCase activity were dissolved in H₂O or DMSO.

### Methods

The GM10915 cell line was cultured under standard cell culture conditions (37° C. and 5% CO₂) in complete DMEM medium supplemented with nonessential amino acids (NEAA), 1% Pen-Strep and 12% FCS. Cells were seeded at a density of 10⁴ cells/well in 100 μL complete medium in one black 96-well plate for glucosylceramidase (GCase) activity measurement and in one clear 96-well plate for crystal violet staining to correct for cell density. Crystal violet staining is performed to obtain quantitative information about the relative density of cells adhering to multi-wells plates.

### Assay of GCase Activity

The assay was adapted from Sawkar et al (2002) and briefly described in the following. The day after seeding of cells, the medium was replaced with fresh medium containing the compounds to be tested. Compounds were tested in duplicate and in an 8-point diluted dose range to obtain a dose response. Cells were exposed with compounds for five days. Fresh compound was added every 2-3 days. PBS was included to define the basal level of GCase activity.

Cells were washed three times with 200 μL PBS per well and 50 μL of 2.5 mM 4-MUG buffer (4-MUG dissolved in 0.2 M acetate buffer pH 4.0) was added and the cells were incubated at 37° C., 5% CO₂ for 23 hours. The reaction was stopped by adding 150 μL 0.2 M glycine buffer (pH 10.8). Fluorescence was measured with a Varioskan® Flash reader (Thermo Scientific) at an excitation/emission setting of 365/445 nm.

### Crystal Violet Staining

Cells were treated with compounds in a parallel setup identical to the setup to test for GCase activity. At the end of compound treatment, cells were washed once with 200 μL PBS per well and 50 μL 0.1% w/v crystal violet (in H₂O) was added. Following 10 min. of incubation, the crystal violet solution was removed, and the cells were washed three times with 200 μL PBS and 100 μL 1% SDS was added to solubilize the stain. The plate was agitated on an orbital shaker for 10-30 min. Absorbance (A) is measured at 570 nM using a Varioskan® Flash reader (Thermo Scientific).

### Calculations

The fluorescence signal (F) derived from the GCase measurement is normalized to the absorbance signal (A) derived from the crystal violet staining. The percent GCase activity resulting from compound treatment is calculated relative to the basal activity obtained from untreated cells.

$$Percent GCase activity = 100 * \frac{{( F / A )}_{c o mpound}}{{( F / A )}_{u n t reated}}$$

The potency, EC_{1.5}, is determined based on the dose response effects of the compounds as the concentration where “Percent GCase activity”=150% corresponding to at 1.5-fold induction of GCase activity. Maximal effect of compounds (Emax) is determined from the dose response effects as the maximum “Percent GCase activity” achieved in the dose range tested.

### Results

The GBA potencies and Emax were determined as described above in the present example and the results are shown in Table 1 below.

| ID | Structure | GBA EC_{1.5} (μM) | GBA Emax (%) |
|---|---|---|---|
| Ambroxol reference | 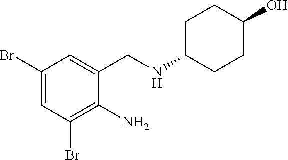 [SMILES: Nc1c(Br)cc(Br)cc1CN[C@H]1CC[C@H](O)CC1] | >20* *The 1.5-fold induction criteria was not reached within the tested concentration range | 127 |
| LTI-291 reference | 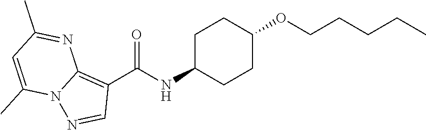 [SMILES: CCCCCO[C@H]1CC[C@H](NC(=O)c2cnn3c(C)cc(C)nc23)CC1] | 112 | 232 |
| 545 | 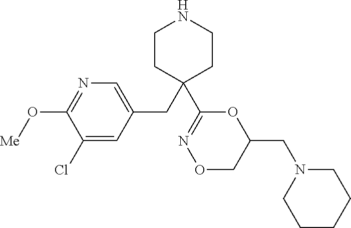 [SMILES: COc1ncc(CC2(C3=NOCC(CN4CCCCC4)O3)CCNCC2)cc1Cl] | 1 | 140 |
| 538 | 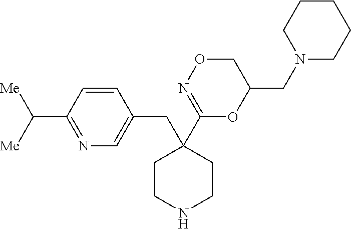 [SMILES: CC(C)c1ccc(CC2(C3=NOCC(CN4CCCCC4)O3)CCNCC2)cn1] | 1 | 147 |
| 598 | 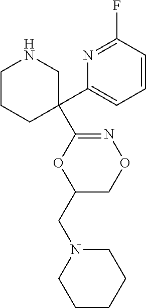 [SMILES: Fc1cccc(C2(C3=NOCC(CN4CCCCC4)O3)CCCNC2)n1] | 1 | 179 |
| 642 | 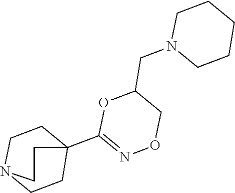 [SMILES: C1CCN(CC2CON=C(C34CCN(CC3)CC4)O2)CC1] | 3 | 282 |
| 543 | 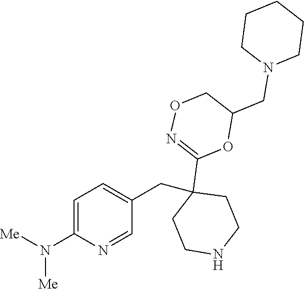 [SMILES: CN(C)c1ccc(CC2(C3=NOCC(CN4CCCCC4)O3)CCNCC2)cn1] | 3 | 138 |
| 641 | 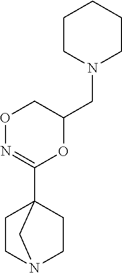 [SMILES: C1CCN(CC2CON=C(C34CCN(CC3)C4)O2)CC1] | 3 | 275 |
| 553 | 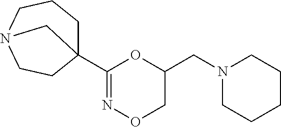 [SMILES: C1CCN(CC2CON=C(C34CCCN(CC3)C4)O2)CC1] | 4 | 341 |
| 558 | 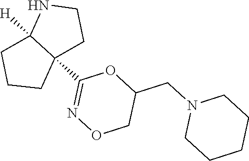 [SMILES: C1CCN(CC2CON=C([C@]34CCC[C@H]3NCC4)O2)CC1] | 4 | 225 |
| 707 | 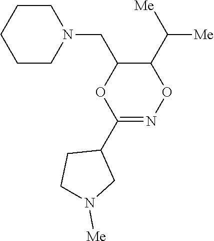 [SMILES: CC(C)C1ON=C(C2CCN(C)C2)OC1CN1CCCCC1] | 4 | 249 |
| 589 | 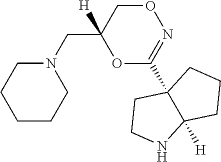 [SMILES: C1CCN(C[C@@H]2CON=C([C@]34CCC[C@H]3NCC4)O2)CC1] | 4 | 216 |
| 590 | 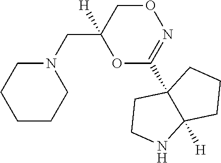 [SMILES: C1CCN(C[C@H]2CON=C([C@]34CCC[C@H]3NCC4)O2)CC1] | 4 | 211 |
| 816 | 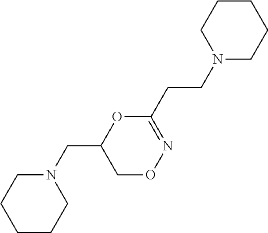 [SMILES: C1CCN(CCC2=NOCC(CN3CCCCC3)O2)CC1] | 4 | 181 |
| 648 | 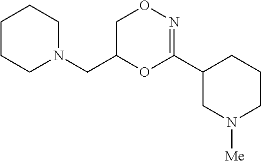 [SMILES: CN1CCCC(C2=NOCC(CN3CCCCC3)O2)C1] | 4.3 | 201 |
| 561 | 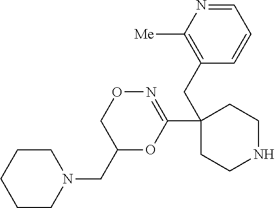 [SMILES: Cc1ncccc1CC1(C2=NOCC(CN3CCCCC3)O2)CCNCC1] | 5 | 184 |
| 644 | 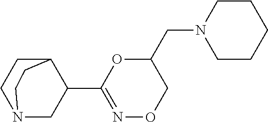 [SMILES: C1CCN(CC2CON=C(C3CN4CCC3CC4)O2)CC1] | 5 | 264 |
| 705 | 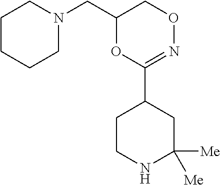 [SMILES: CC1(C)CC(C2=NOCC(CN3CCCCC3)O2)CCN1] | 5 | 180 |
| 469 | 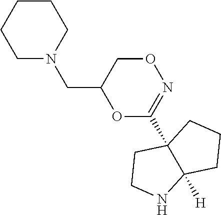 [SMILES: C1CCN(CC2CON=C([C@]34CCC[C@H]3NCC4)O2)CC1] | 5 | 204 |
| 584 | 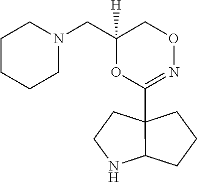 [SMILES: C1CCN(C[C@H]2CON=C(C34CCCC3NCC4)O2)CC1] | 5 | 204 |
| 633 | 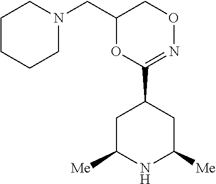 [SMILES: C[C@@H]1C[C@H](C2=NOCC(CN3CCCCC3)O2)C[C@H](C)N1] | 5 | 226 |
| 706 | 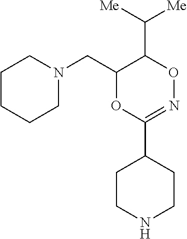 [SMILES: CC(C)C1ON=C(C2CCNCC2)OC1CN1CCCCC1] | 6 | 180 |
| 568 | 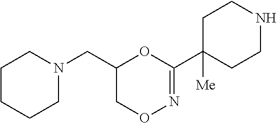 [SMILES: CC1(C2=NOCC(CN3CCCCC3)O2)CCNCC1] | 6 | 281 |
| 476 | 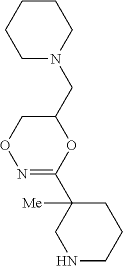 [SMILES: CC1(C2=NOCC(CN3CCCCC3)O2)CCCNC1] | 6 | 253 |
| 528 | 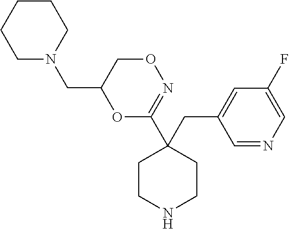 [SMILES: Fc1cncc(CC2(C3=NOCC(CN4CCCCC4)O3)CCNCC2)c1] | 6 | 181 |
| 616 | 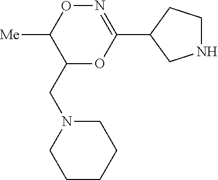 [SMILES: CC1ON=C(C2CCNC2)OC1CN1CCCCC1] | 6 | 390 |
| 595 | 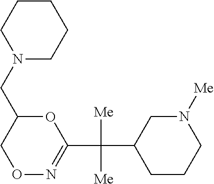 [SMILES: CN1CCCC(C(C)(C)C2=NOCC(CN3CCCCC3)O2)C1] | 6 | 172 |
| 640 | 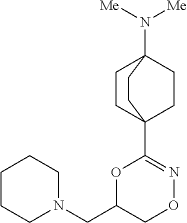 [SMILES: CN(C)C12CCC(C3=NOCC(CN4CCCCC4)O3)(CC1)CC2] | 6 | 183 |
| 768 | 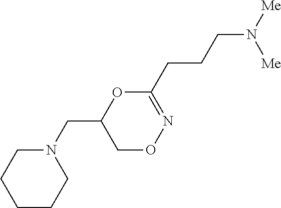 [SMILES: CN(C)CCCC1=NOCC(CN2CCCCC2)O1] | 6 | 238 |
| 826 | 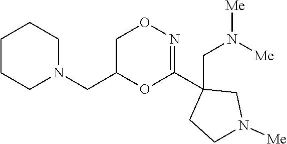 [SMILES: CN(C)CC1(C2=NOCC(CN3CCCCC3)O2)CCN(C)C1] | 7 | 190 |
| 567 | 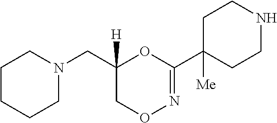 [SMILES: CC1(C2=NOC[C@H](CN3CCCCC3)O2)CCNCC1] | 7 | 296 |
| 531 | 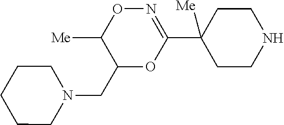 [SMILES: CC1ON=C(C2(C)CCNCC2)OC1CN1CCCCC1] | 7 | 282 |
| 569 | 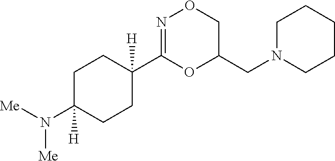 [SMILES: CN(C)[C@H]1CC[C@@H](C2=NOCC(CN3CCCCC3)O2)CC1] | 7 | 187 |
| 560 | 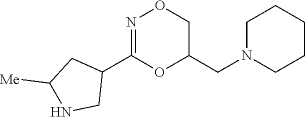 [SMILES: CC1CC(C2=NOCC(CN3CCCCC3)O2)CN1] | 8 | 244 |
| 655 | 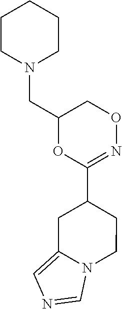 [SMILES: c1ncn2c1CC(C1=NOCC(CN3CCCCC3)O1)CC2] | 8 | 232 |
| 726 | 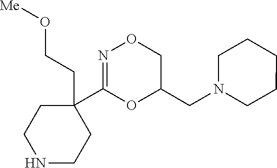 [SMILES: COCCC1(C2=NOCC(CN3CCCCC3)O2)CCNCC1] | 8 | 269 |
| 770 | 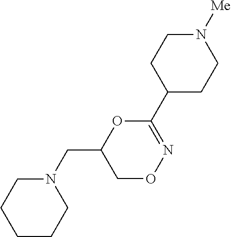 [SMILES: CN1CCC(C2=NOCC(CN3CCCCC3)O2)CC1] | 9 | 265 |
| 462 | 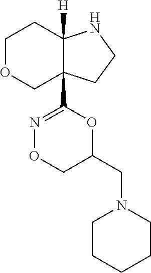 [SMILES: C1CCN(CC2CON=C([C@]34CCN[C@H]3CCOC4)O2)CC1] | 9 | 241 |
| 473 | 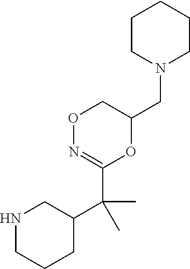 [SMILES: CC(C)(C1=NOCC(CN2CCCCC2)O1)C1CCCNC1] | 9 | 181 |
| 660 | 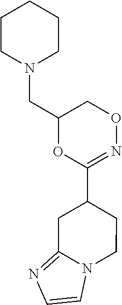 [SMILES: c1cn2c(n1)CC(C1=NOCC(CN3CCCCC3)O1)CC2] | 9 | 212 |
| 465 | 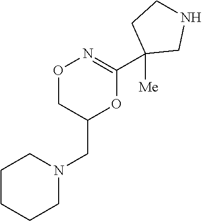 [SMILES: CC1(C2=NOCC(CN3CCCCC3)O2)CCNC1] | 10 | 283 |
| 621 | 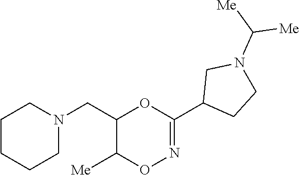 [SMILES: CC1ON=C(C2CCN(C(C)C)C2)OC1CN1CCCCC1] | 10 | 276 |
| 719 | 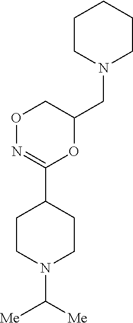 [SMILES: CC(C)N1CCC(C2=NOCC(CN3CCCCC3)O2)CC1] | 10 | 242 |
| 779 | 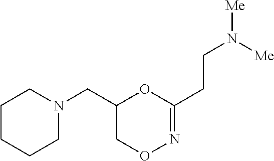 [SMILES: CN(C)CCC1=NOCC(CN2CCCCC2)O1] | 11 | 259 |
| 490 | 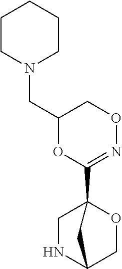 [SMILES: C1CCN(CC2CON=C([C@@]34CN[C@@H](CO3)C4)O2)CC1] | 11 | 215 |
| 529 | 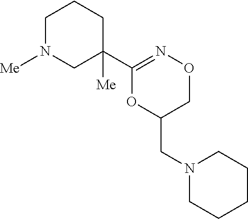 [SMILES: CN1CCCC(C)(C2=NOCC(CN3CCCCC3)O2)C1] | 11 | 222 |
| 554 | 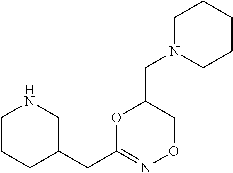 [SMILES: C1CCN(CC2CON=C(CC3CCCNC3)O2)CC1] | 11 | 271 |
| 629 | 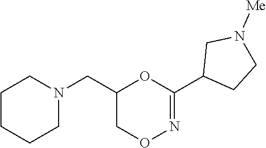 [SMILES: CN1CCC(C2=NOCC(CN3CCCCC3)O2)C1] | 11 | 247 |
| 720 | 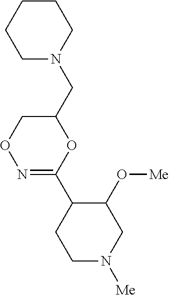 [SMILES: COC1CN(C)CCC1C1=NOCC(CN2CCCCC2)O1] | 11 | 346 |
| 685 | 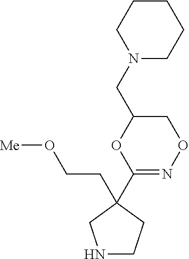 [SMILES: COCCC1(C2=NOCC(CN3CCCCC3)O2)CCNC1] | 12 | 270 |
| 520 | 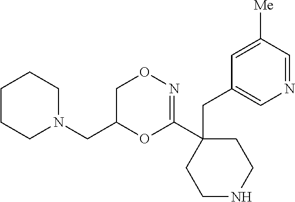 [SMILES: Cc1cncc(CC2(C3=NOCC(CN4CCCCC4)O3)CCNCC2)c1] | 13 | 136 |
| 482 | 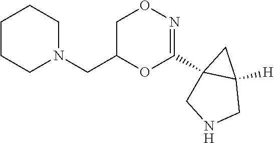 [SMILES: C1CCN(CC2CON=C([C@@]34CNC[C@@H]3C4)O2)CC1] | 14 | 268 |
| 500 | 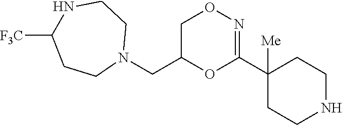 [SMILES: CC1(C2=NOCC(CN3CCNC(C(F)(F)F)CC3)O2)CCNCC1] | 14 | 166 |
| 551 | 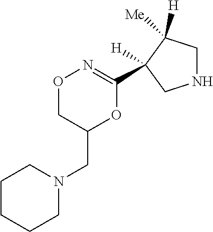 [SMILES: C[C@@H]1CNC[C@H]1C1=NOCC(CN2CCCCC2)O1] | 14 | 185 |
| 583 | 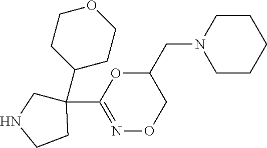 [SMILES: C1CCN(CC2CON=C(C3(C4CCOCC4)CCNC3)O2)CC1] | 14 | 188 |
| 700 | 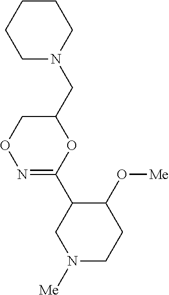 [SMILES: COC1CCN(C)CC1C1=NOCC(CN2CCCCC2)O1] | 15 | 270 |
| 477 | 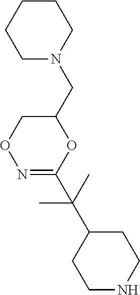 [SMILES: CC(C)(C1=NOCC(CN2CCCCC2)O1)C1CCNCC1] | 15 | 181 |
| 724 | 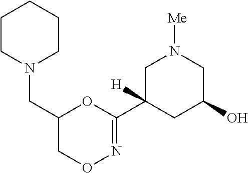 [SMILES: CN1C[C@@H](O)C[C@H](C2=NOCC(CN3CCCCC3)O2)C1] | 16 | 274 |
| 748 | 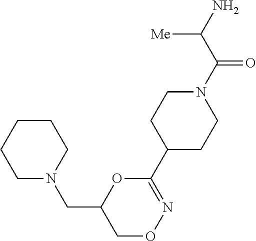 [SMILES: CC(N)C(=O)N1CCC(C2=NOCC(CN3CCCCC3)O2)CC1] | 16 | 283 |
| 480 | 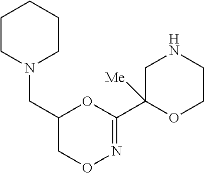 [SMILES: CC1(C2=NOCC(CN3CCCCC3)O2)CNCCO1] | 16 | 273 |
| 559 | 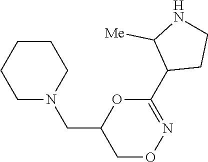 [SMILES: CC1NCCC1C1=NOCC(CN2CCCCC2)O1] | 16 | 251 |
| 714 | 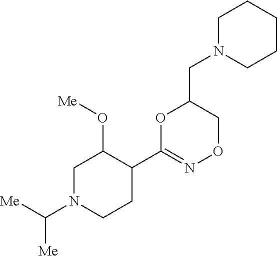 [SMILES: COC1CN(C(C)C)CCC1C1=NOCC(CN2CCCCC2)O1] | 16 | 213 |
| 508 | 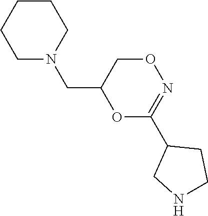 [SMILES: C1CCN(CC2CON=C(C3CCNC3)O2)CC1] | 17 | 226 |
| 718 | 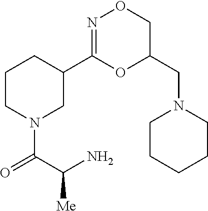 [SMILES: C[C@H](N)C(=O)N1CCCC(C2=NOCC(CN3CCCCC3)O2)C1] | 17 | 341 |
| 823 | 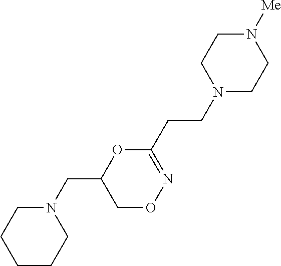 [SMILES: CN1CCN(CCC2=NOCC(CN3CCCCC3)O2)CC1] | 18 | 312 |
| 494 | 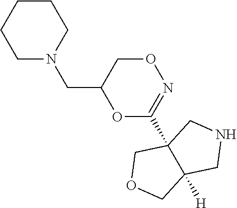 [SMILES: C1CCN(CC2CON=C([C@]34CNC[C@H]3COC4)O2)CC1] | 18 | 307 |
| 546 | 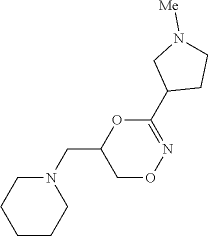 [SMILES: CN1CCC(C2=NOCC(CN3CCCCC3)O2)C1] | 18 | 253 |
| 643 | 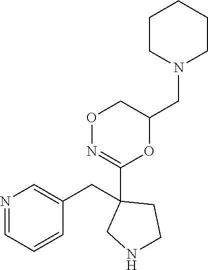 [SMILES: c1cncc(CC2(C3=NOCC(CN4CCCCC4)O3)CCNC2)c1] | 18 | 178 |
| 461 | 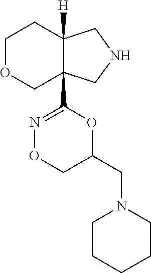 [SMILES: C1CCN(CC2CON=C([C@]34CNC[C@H]3CCOC4)O2)CC1] | 19 | 203 |
| 521 | 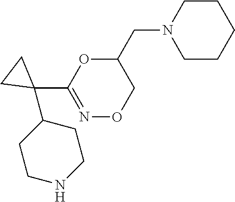 [SMILES: C1CCN(CC2CON=C(C3(C4CCNCC4)CC3)O2)CC1] | 19 | 336 |
| 606 | 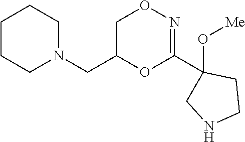 [SMILES: COC1(C2=NOCC(CN3CCCCC3)O2)CCNC1] | 20 | 292 |
| 680 | 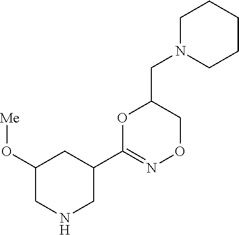 [SMILES: COC1CNCC(C2=NOCC(CN3CCCCC3)O2)C1] | 20 | 209 |
| 548 | 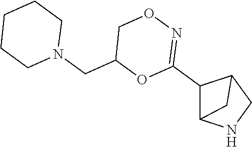 [SMILES: C1CCN(CC2CON=C(C3C4CNC3C4)O2)CC1] | 21 | 281 |
| 701 | 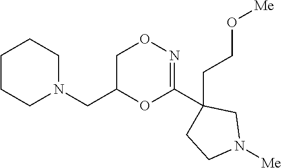 [SMILES: COCCC1(C2=NOCC(CN3CCCCC3)O2)CCN(C)C1] | 21 | 225 |
| 481 | 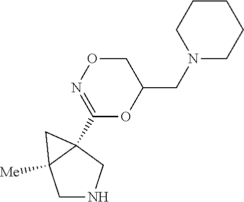 [SMILES: C[C@@]12CNC[C@]1(C1=NOCC(CN3CCCCC3)O1)C2] | 22 | 174 |
| 709 | 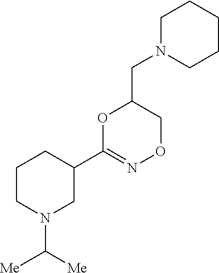 [SMILES: CC(C)N1CCCC(C2=NOCC(CN3CCCCC3)O2)C1] | 22 | 178 |
| 485 | 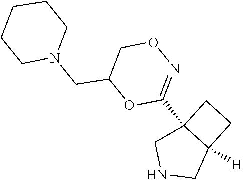 [SMILES: C1CCN(CC2CON=C([C@]34CC[C@H]3CNC4)O2)CC1] | 25 | 174 |
| 690 | 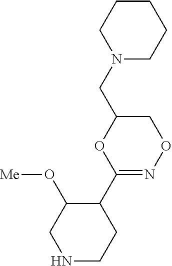 [SMILES: COC1CNCCC1C1=NOCC(CN2CCCCC2)O1] | 25 | 205 |
| 691 | 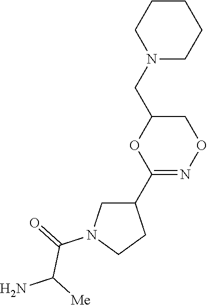 [SMILES: CC(N)C(=O)N1CCC(C2=NOCC(CN3CCCCC3)O2)C1] | 25 | 252 |
| 722 | 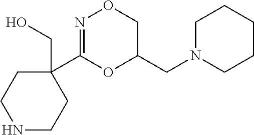 [SMILES: OCC1(C2=NOCC(CN3CCCCC3)O2)CCNCC1] | 26 | 153 |
| 527 | 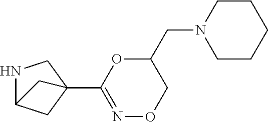 [SMILES: C1CCN(CC2CON=C(C34CNC(C3)C4)O2)CC1] | 26 | 187 |
| 611 | 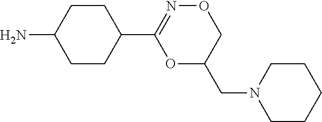 [SMILES: NC1CCC(C2=NOCC(CN3CCCCC3)O2)CC1] | 26 | 299 |
| 550 | 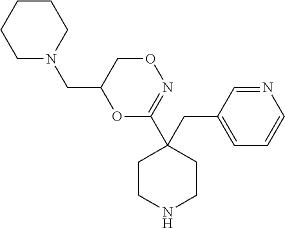 [SMILES: c1cncc(CC2(C3=NOCC(CN4CCCCC4)O3)CCNCC2)c1] | 27 | 159 |
| 491 | 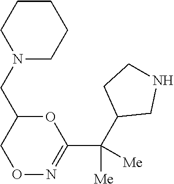 [SMILES: CC(C)(C1=NOCC(CN2CCCCC2)O1)C1CCNC1] | 28 | 230 |
| 710 | 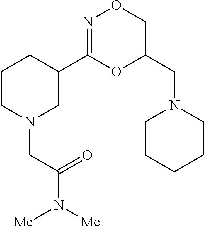 [SMILES: CN(C)C(=O)CN1CCCC(C2=NOCC(CN3CCCCC3)O2)C1] | 28 | 234 |
| 713 | 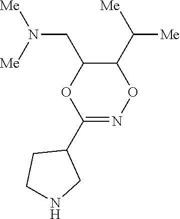 [SMILES: CC(C)C1ON=C(C2CCNC2)OC1CN(C)C] | 28 | 212 |
| 765 | 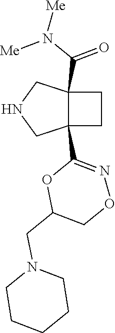 [SMILES: CN(C)C(=O)[C@@]12CC[C@]1(C1=NOCC(CN3CCCCC3)O1)CNC2] | 29 | 151 |
| 489 | 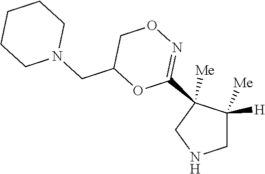 [SMILES: C[C@@H]1CNC[C@@]1(C)C1=NOCC(CN2CCCCC2)O1] | 29 | 242 |
| 821 | 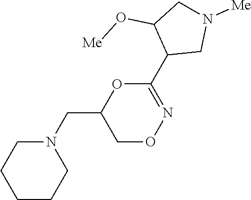 [SMILES: COC1CN(C)CC1C1=NOCC(CN2CCCCC2)O1] | 29 | 213 |
| 721 | 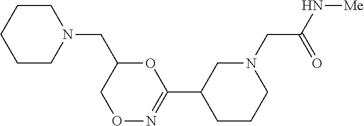 [SMILES: CNC(=O)CN1CCCC(C2=NOCC(CN3CCCCC3)O2)C1] | 33 | 175 |
| 688 | 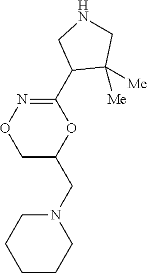 [SMILES: CC1(C)CNCC1C1=NOCC(CN2CCCCC2)O1] | 34 | 152 |
| 479 | 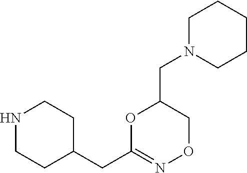 [SMILES: C1CCN(CC2CON=C(CC3CCNCC3)O2)CC1] | 35 | 184 |
| 507 | 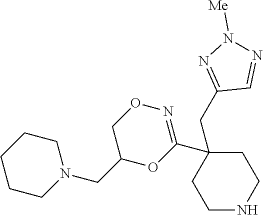 [SMILES: Cn1ncc(CC2(C3=NOCC(CN4CCCCC4)O3)CCNCC2)n1] | 35 | 149 |
| 549 | 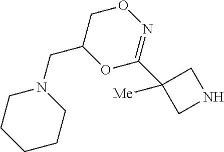 [SMILES: CC1(C2=NOCC(CN3CCCCC3)O2)CNC1] | 36 | 232 |
| 566 | 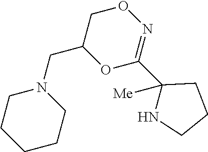 [SMILES: CC1(C2=NOCC(CN3CCCCC3)O2)CCCN1] | 36 | 164 |
| 725 | 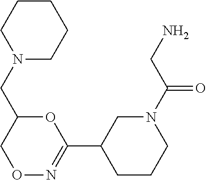 [SMILES: NCC(=O)N1CCCC(C2=NOCC(CN3CCCCC3)O2)C1] | 36 | 168 |
| 565 | 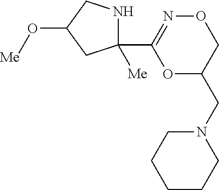 [SMILES: COC1CNC(C)(C2=NOCC(CN3CCCCC3)O2)C1] | 37 | 168 |
| 766 | 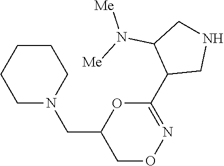 [SMILES: CN(C)C1CNCC1C1=NOCC(CN2CCCCC2)O1] | 38 | 136 |
| 699 | 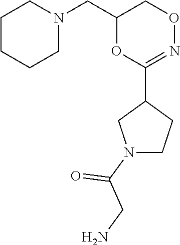 [SMILES: NCC(=O)N1CCC(C2=NOCC(CN3CCCCC3)O2)C1] | 38 | 203 |
| 711 | 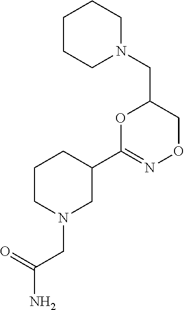 [SMILES: NC(=O)CN1CCCC(C2=NOCC(CN3CCCCC3)O2)C1] | 38 | 198 |
| 756 | 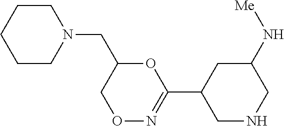 [SMILES: CNC1CNCC(C2=NOCC(CN3CCCCC3)O2)C1] | 39 | 121 |
| 806 | 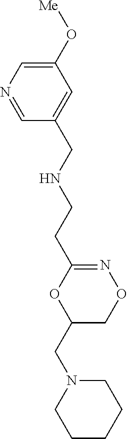 [SMILES: COc1cncc(CNCCC2=NOCC(CN3CCCCC3)O2)c1] | 40 | 183 |
| 519 | 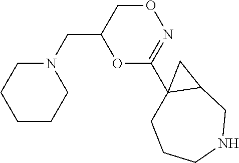 [SMILES: C1CCN(CC2CON=C(C34CCCNCC3C4)O2)CC1] | 40 | 159 |
| 787 | 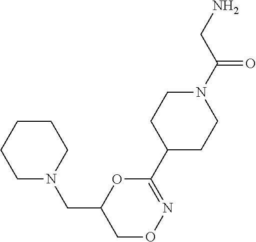 [SMILES: NCC(=O)N1CCC(C2=NOCC(CN3CCCCC3)O2)CC1] | 42 | 193 |
| 628 | 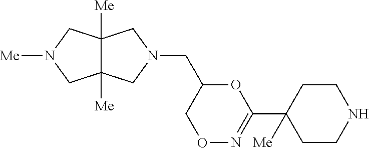 [SMILES: CN1CC2(C)CN(CC3CON=C(C4(C)CCNCC4)O3)CC2(C)C1] | 43 | 136 |
| 605 | 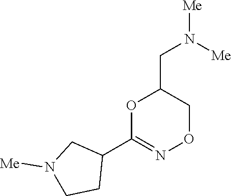 [SMILES: CN(C)CC1CON=C(C2CCN(C)C2)O1] | 45 | 148 |
| 797 | 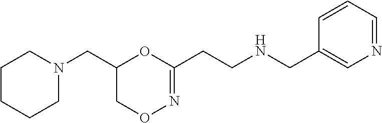 [SMILES: c1cncc(CNCCC2=NOCC(CN3CCCCC3)O2)c1] | 46 | 149 |
| 627 | 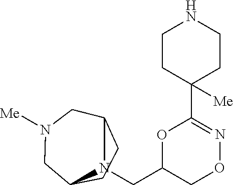 [SMILES: CN1CC2CC[C@H](C1)N2CC1CON=C(C2(C)CCNCC2)O1] | 48 | 133 |
| 594 | 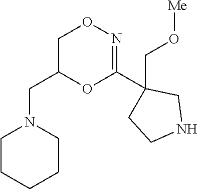 [SMILES: COCC1(C2=NOCC(CN3CCCCC3)O2)CCNC1] | 49 | 152 |
| 673 | 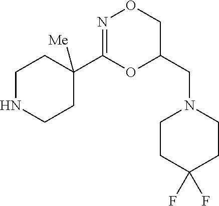 [SMILES: CC1(C2=NOCC(CN3CCC(F)(F)CC3)O2)CCNCC1] | 49 | 151 |
| 552 | 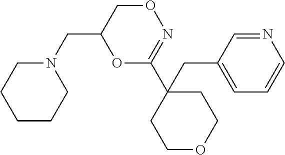 [SMILES: c1cncc(CC2(C3=NOCC(CN4CCCCC4)O3)CCOCC2)c1] | 50 | 115 |
| 555 | 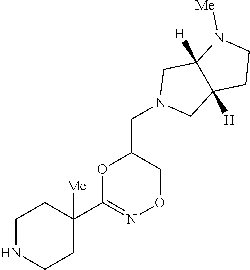 [SMILES: CN1CC[C@@H]2CN(CC3CON=C(C4(C)CCNCC4)O3)C[C@@H]21] | 50 | 114 |
| 556 | 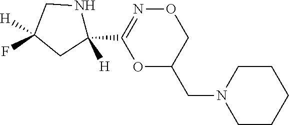 [SMILES: F[C@H]1CN[C@H](C2=NOCC(CN3CCCCC3)O2)C1] | 50 | 114 |
| 557 | 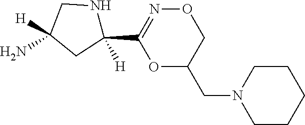 [SMILES: N[C@@H]1CN[C@@H](C2=NOCC(CN3CCCCC3)O2)C1] | 50 | 115 |
| 564 | 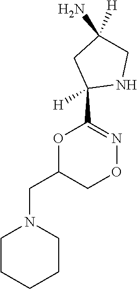 [SMILES: N[C@H]1CN[C@@H](C2=NOCC(CN3CCCCC3)O2)C1] | 50 | 104 |
| 570 | 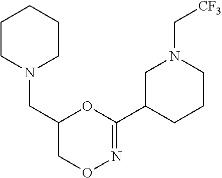 [SMILES: FC(F)(F)CN1CCCC(C2=NOCC(CN3CCCCC3)O2)C1] | 50 | 107 |
| 580 | 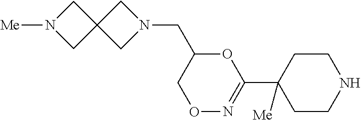 [SMILES: CN1CC2(C1)CN(CC1CON=C(C3(C)CCNCC3)O1)C2] | 50 | 105 |
| 582 | 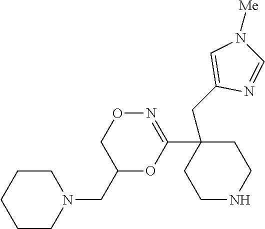 [SMILES: Cn1cnc(CC2(C3=NOCC(CN4CCCCC4)O3)CCNCC2)c1] | 50 | 114 |
| 587 | 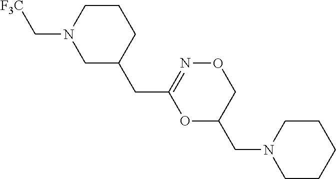 [SMILES: FC(F)(F)CN1CCCC(CC2=NOCC(CN3CCCCC3)O2)C1] | 50 | 100 |
| 592 | 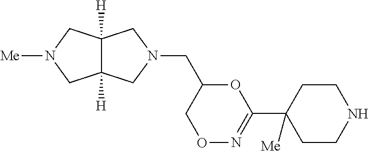 [SMILES: CN1C[C@@H]2CN(CC3CON=C(C4(C)CCNCC4)O3)C[C@@H]2C1] | 50 | 110 |
| 596 | 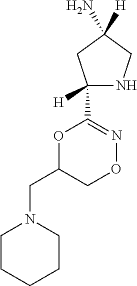 [SMILES: N[C@@H]1CN[C@H](C2=NOCC(CN3CCCCC3)O2)C1] | 50 | 105 |
| 597 | 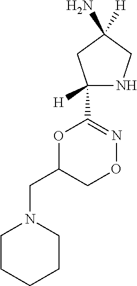 [SMILES: N[C@H]1CN[C@H](C2=NOCC(CN3CCCCC3)O2)C1] | 50 | 106 |
| 601 | 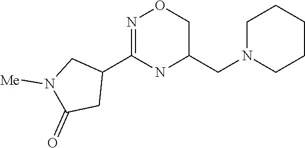 [SMILES: CN1CC(C2=NOCC(CN3CCCCC3)N2)CC1=O] | 50 | 111 |
| 602 | 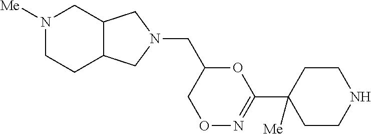 [SMILES: CN1CCC2CN(CC3CON=C(C4(C)CCNCC4)O3)CC2C1] | 50 | 121 |
| 603 | 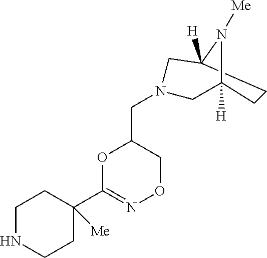 [SMILES: CN1[C@@H]2CC[C@H]1CN(CC1CON=C(C3(C)CCNCC3)O1)C2] | 50 | 102 |
| 604 | 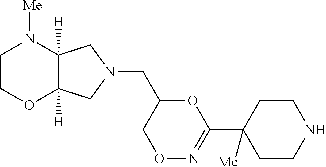 [SMILES: CN1CCO[C@@H]2CN(CC3CON=C(C4(C)CCNCC4)O3)C[C@@H]21] | 50 | 119 |
| 607 | 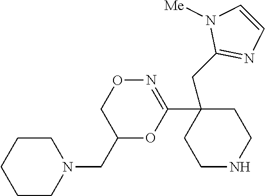 [SMILES: Cn1ccnc1CC1(C2=NOCC(CN3CCCCC3)O2)CCNCC1] | 50 | 123 |
| 609 | 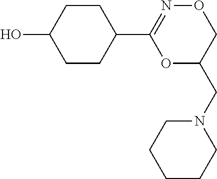 [SMILES: OC1CCC(C2=NOCC(CN3CCCCC3)O2)CC1] | 50 | 119 |
| 615 | 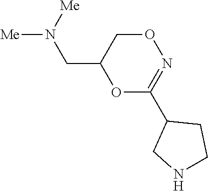 [SMILES: CN(C)CC1CON=C(C2CCNC2)O1] | 50 | 108 |
| 617 | 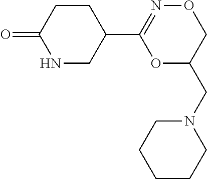 [SMILES: O=C1CCC(C2=NOCC(CN3CCCCC3)O2)CN1] | 50 | 113 |
| 635 | 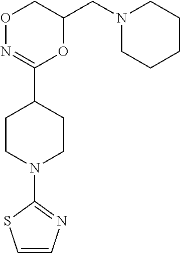 [SMILES: c1csc(N2CCC(C3=NOCC(CN4CCCCC4)O3)CC2)n1] | 50 | 135 |
| 636 | 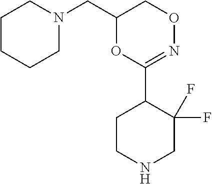 [SMILES: FC1(F)CNCCC1C1=NOCC(CN2CCCCC2)O1] | 50 | 126 |
| 637 | 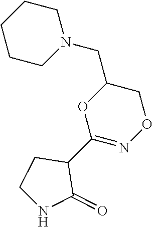 [SMILES: O=C1NCCC1C1=NOCC(CN2CCCCC2)O1] | 50 | 111 |
| 639 | 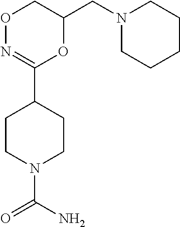 [SMILES: NC(=O)N1CCC(C2=NOCC(CN3CCCCC3)O2)CC1] | 50 | 112 |
| 646 | 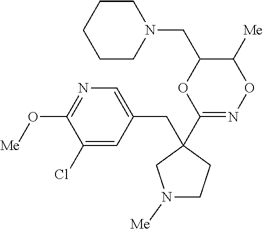 [SMILES: COc1ncc(CC2(C3=NOC(C)C(CN4CCCCC4)O3)CCN(C)C2)cc1Cl] | 50 | 112 |
| 647 | 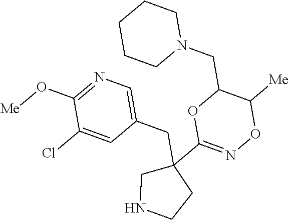 [SMILES: COc1ncc(CC2(C3=NOC(C)C(CN4CCCCC4)O3)CCNC2)cc1Cl] | 50 | 103 |
| 649 | 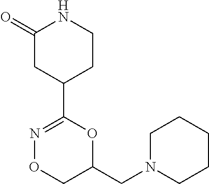 [SMILES: O=C1CC(C2=NOCC(CN3CCCCC3)O2)CCN1] | 50 | 103 |
| 650 | 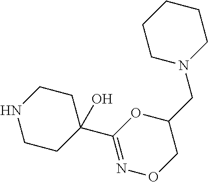 [SMILES: OC1(C2=NOCC(CN3CCCCC3)O2)CCNCC1] | 50 | 141 |
| 656 | 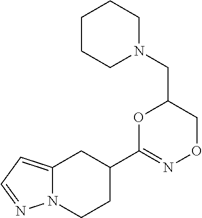 [SMILES: c1cc2n(n1)CCC(C1=NOCC(CN3CCCCC3)O1)C2] | 50 | 123 |
| 658 | 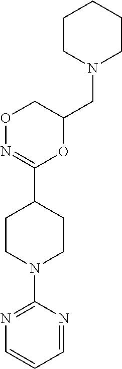 [SMILES: c1cnc(N2CCC(C3=NOCC(CN4CCCCC4)O3)CC2)nc1] | 50 | 116 |
| 661 | 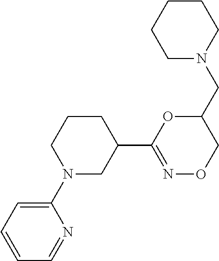 [SMILES: c1ccc(N2CCCC(C3=NOCC(CN4CCCCC4)O3)C2)nc1] | 50 | 140 |
| 662 | 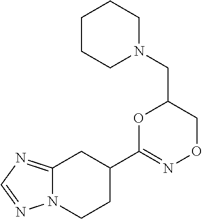 [SMILES: c1nc2n(n1)CCC(C1=NOCC(CN3CCCCC3)O1)C2] | 50 | 138 |
| 664 | 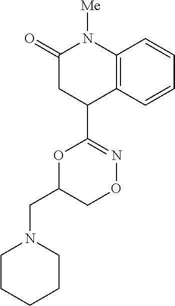 [SMILES: CN1C(=O)CC(C2=NOCC(CN3CCCCC3)O2)c2ccccc21] | 50 | 137 |
| 668 | 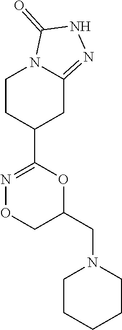 [SMILES: O=c1[nH]nc2n1CCC(C1=NOCC(CN3CCCCC3)O1)C2] | 50 | 116 |
| 670 | 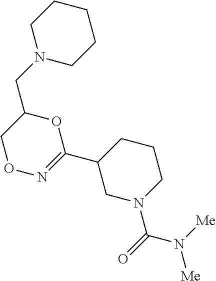 [SMILES: CN(C)C(=O)N1CCCC(C2=NOCC(CN3CCCCC3)O2)C1] | 50 | 126 |
| 671 | 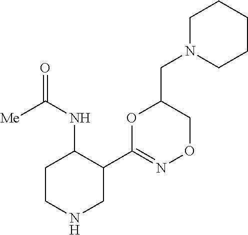 [SMILES: CC(=O)NC1CCNCC1C1=NOCC(CN2CCCCC2)O1] | 50 | 146 |
| 672 | 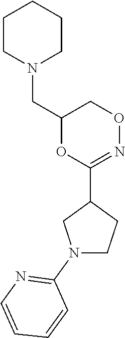 [SMILES: c1ccc(N2CCC(C3=NOCC(CN4CCCCC4)O3)C2)nc1] | 50 | 127 |
| 674 | 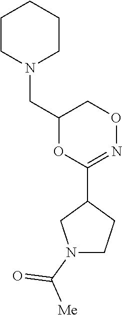 [SMILES: CC(=O)N1CCC(C2=NOCC(CN3CCCCC3)O2)C1] | 50 | 123 |
| 675 | 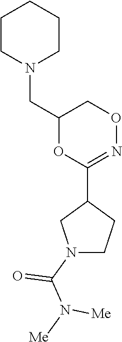 [SMILES: CN(C)C(=O)N1CCC(C2=NOCC(CN3CCCCC3)O2)C1] | 50 | 102 |
| 676 | 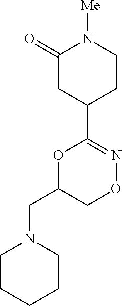 [SMILES: CN1CCC(C2=NOCC(CN3CCCCC3)O2)CC1=O] | 50 | 120 |
| 678 | 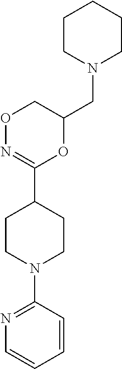 [SMILES: c1ccc(N2CCC(C3=NOCC(CN4CCCCC4)O3)CC2)nc1] | 50 | 106 |
| 679 | 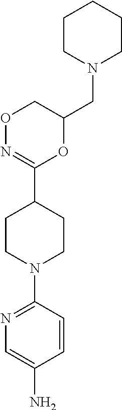 [SMILES: Nc1ccc(N2CCC(C3=NOCC(CN4CCCCC4)O3)CC2)nc1] | 50 | 107 |
| 683 | 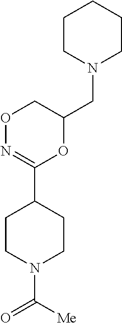 [SMILES: CC(=O)N1CCC(C2=NOCC(CN3CCCCC3)O2)CC1] | 50 | 113 |
| 684 | 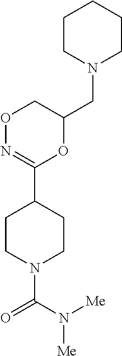 [SMILES: CN(C)C(=O)N1CCC(C2=NOCC(CN3CCCCC3)O2)CC1] | 50 | 109 |
| 686 | 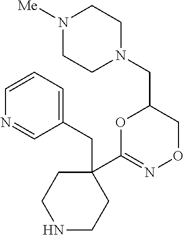 [SMILES: CN1CCN(CC2CON=C(C3(Cc4cccnc4)CCNCC3)O2)CC1] | 50 | 97 |
| 687 | 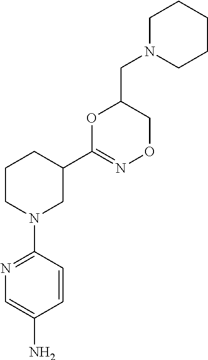 [SMILES: Nc1ccc(N2CCCC(C3=NOCC(CN4CCCCC4)O3)C2)nc1] | 50 | 110 |
| 689 | 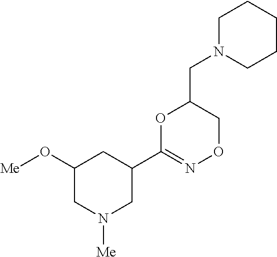 [SMILES: COC1CC(C2=NOCC(CN3CCCCC3)O2)CN(C)C1] | 50 | 137 |
| 692 | 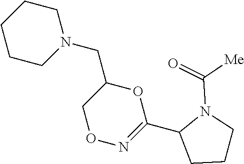 [SMILES: CC(=O)N1CCCC1C1=NOCC(CN2CCCCC2)O1] | 50 | 107 |
| 693 | 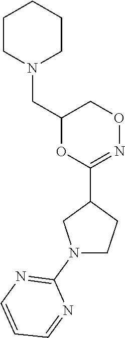 [SMILES: c1cnc(N2CCC(C3=NOCC(CN4CCCCC4)O3)C2)nc1] | 50 | 112 |
| 694 | 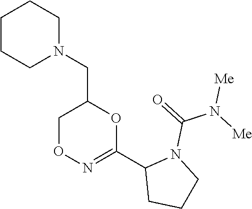 [SMILES: CN(C)C(=O)N1CCCC1C1=NOCC(CN2CCCCC2)O1] | 50 | 115 |
| 695 | 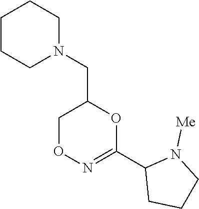 [SMILES: CN1CCCC1C1=NOCC(CN2CCCCC2)O1] | 50 | 111 |
| 697 | 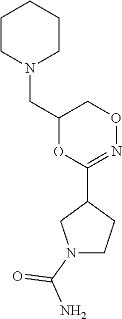 [SMILES: NC(=O)N1CCC(C2=NOCC(CN3CCCCC3)O2)C1] | 50 | 107 |
| 698 | 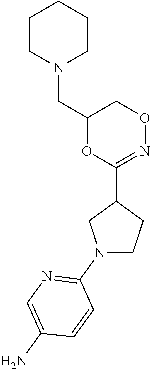 [SMILES: Nc1ccc(N2CCC(C3=NOCC(CN4CCCCC4)O3)C2)nc1] | 50 | 137 |
| 702 | 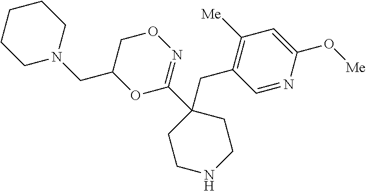 [SMILES: COc1cc(C)c(CC2(C3=NOCC(CN4CCCCC4)O3)CCNCC2)cn1] | 50 | 110 |
| 703 | 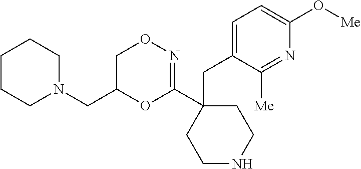 [SMILES: COc1ccc(CC2(C3=NOCC(CN4CCCCC4)O3)CCNCC2)c(C)n1] | 50 | 113 |
| 704 | 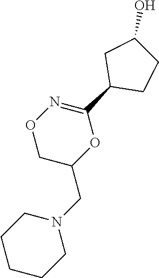 [SMILES: O[C@@H]1CC[C@@H](C2=NOCC(CN3CCCCC3)O2)C1] | 50 | 117 |
| 717 | 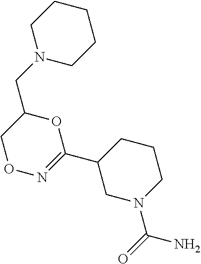 [SMILES: NC(=O)N1CCCC(C2=NOCC(CN3CCCCC3)O2)C1] | 50 | 111 |
| 829 | 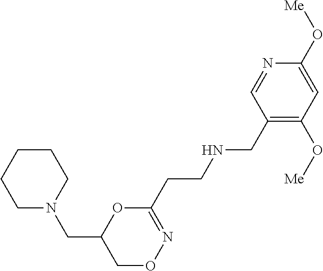 [SMILES: COc1cc(OC)c(CNCCC2=NOCC(CN3CCCCC3)O2)cn1] | 5 | 175 |
| 584 | 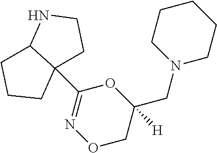 [SMILES: C1CCN(C[C@H]2CON=C(C34CCCC3NCC4)O2)CC1] | 10 | 253 |
| 589 | 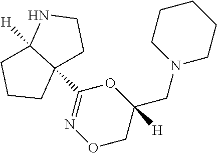 [SMILES: C1CCN(C[C@@H]2CON=C([C@]34CCC[C@H]3NCC4)O2)CC1] | 5 | 272 |
| 590 | 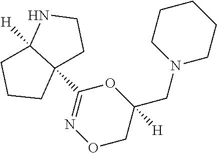 [SMILES: C1CCN(C[C@H]2CON=C([C@]34CCC[C@H]3NCC4)O2)CC1] | 3 | 273 |
| 852 | 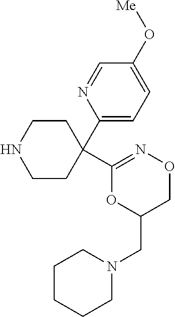 [SMILES: COc1ccc(C2(C3=NOCC(CN4CCCCC4)O3)CCNCC2)nc1] | 4 | 203 |
| 707 | 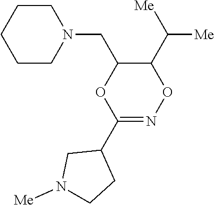 [SMILES: CC(C)C1ON=C(C2CCN(C)C2)OC1CN1CCCCC1] | 2 | 230 |
| 848 | 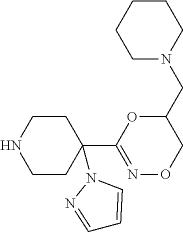 [SMILES: c1cnn(C2(C3=NOCC(CN4CCCCC4)O3)CCNCC2)c1] | 2 | 311 |
| 856 | 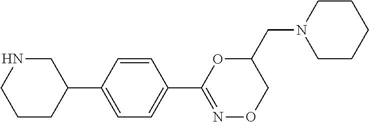 [SMILES: c1cc(C2CCCNC2)ccc1C1=NOCC(CN2CCCCC2)O1] | 2 | 134 |
| 862 | 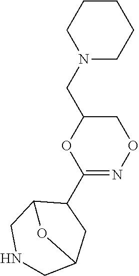 [SMILES: C1CCN(CC2CON=C(C3CC4CNCC3O4)O2)CC1] | 15 | 212 |
| 863 | 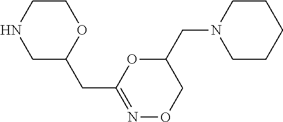 [SMILES: C1CCN(CC2CON=C(CC3CNCCO3)O2)CC1] | 6 | 253 |
| 870 | 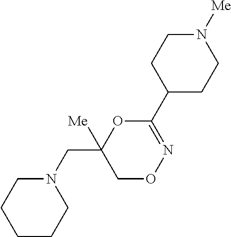 [SMILES: CN1CCC(C2=NOCC(C)(CN3CCCCC3)O2)CC1] | 6 | 217 |
| 874 | 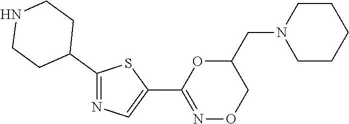 [SMILES: c1nc(C2CCNCC2)sc1C1=NOCC(CN2CCCCC2)O1] | 5 | 194 |
| 875 | 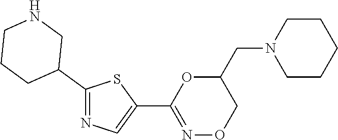 [SMILES: c1nc(C2CCCNC2)sc1C1=NOCC(CN2CCCCC2)O1] | 5 | 165 |
| 876 | 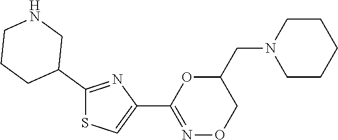 [SMILES: c1sc(C2CCCNC2)nc1C1=NOCC(CN2CCCCC2)O1] | 4 | 180 |
| 878 | 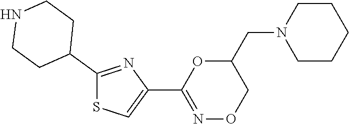 [SMILES: c1sc(C2CCNCC2)nc1C1=NOCC(CN2CCCCC2)O1] | 3 | 231 |
| 886 | 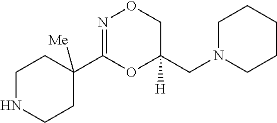 [SMILES: CC1(C2=NOC[C@@H](CN3CCCCC3)O2)CCNCC1] | 7 | 203 |
| 881 | 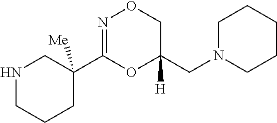 [SMILES: C[C@]1(C2=NOC[C@H](CN3CCCCC3)O2)CCCNC1] | 5 | 219 |
| 882 | 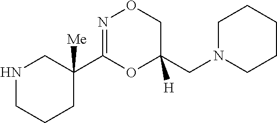 [SMILES: C[C@@]1(C2=NOC[C@H](CN3CCCCC3)O2)CCCNC1] | 5 | 214 |
| 883 | 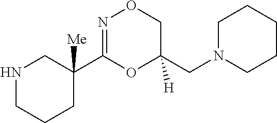 [SMILES: C[C@@]1(C2=NOC[C@@H](CN3CCCCC3)O2)CCCNC1] | 7 | 201 |
| 884 | 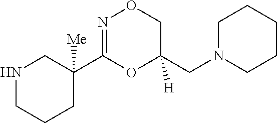 [SMILES: C[C@]1(C2=NOC[C@@H](CN3CCCCC3)O2)CCCNC1] | 6 | 207 |
| 887 | 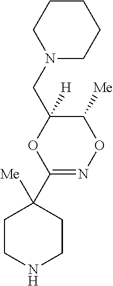 [SMILES: C[C@@H]1ON=C(C2(C)CCNCC2)O[C@H]1CN1CCCCC1] | 5 | 233 |
| 890 | 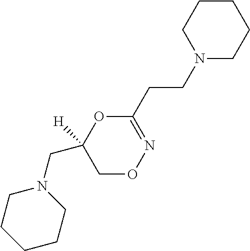 [SMILES: C1CCN(CCC2=NOC[C@@H](CN3CCCCC3)O2)CC1] | 8 | 168 |
| 891 | 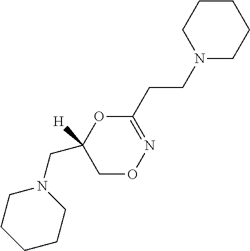 [SMILES: C1CCN(CCC2=NOC[C@H](CN3CCCCC3)O2)CC1] | 15 | 209 |
| 892 | 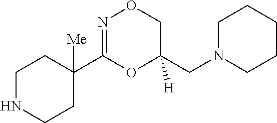 [SMILES: CC1(C2=NOC[C@@H](CN3CCCCC3)O2)CCNCC1] | 13 | 274 |

### CONCLUSION

This example demonstrates that the dioxazines of the present disclosure are highly potent and efficacious in comparison with state-of-the-art GBA inducers like Ambroxol and LTI-291. These effects render the dioxazines of the present disclosure promising candidates for treatment of GBA-mediated disorders.

1. A compound of formula (Ia), 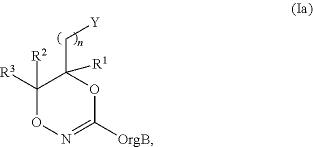
or a pharmaceutically acceptable salt thereof; wherein
n is 1 or 2;
R¹, R², and R³ are independently selected from the group consisting of: hydrogen, alkyl, and halogen;
Y is a nitrogen-containing ring or a nitrogen-containing chain;
OrgB is an organic basic moiety attached via a sp³ hybridised carbon to the rest of the compound; and
OrgB and Y are optionally substituted.

2. The compound according to claim 1, wherein the compound is of formula (Ib), 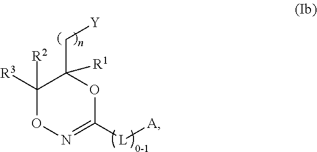
wherein
A is a monocyclic ring, a bicyclic ring, or a tricyclic ring, and A is attached via a sp³ hybridised carbon to the rest of the compound;
L is a C₁₋₆ alkyl linker or L is absent; if L is absent, A is directly attached to the cyclic oxime; and
A and Y are optionally substituted.

3. The compound according to claim 2, wherein A is selected from the group consisting of: a monocyclic ring and a bicyclic ring; and
b) comprises 1, 2 or 3 nitrogen atoms; and/or
c) comprises 0, 1, 2 or 3 oxygen atoms.

4. The compound according to any one of claims 2-3, wherein A is of formula (II): 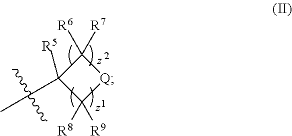
wherein
z¹ and z² are independently selected from the group consisting of: 0, 1, 2, and 3;
Q is selected from the group consisting of: 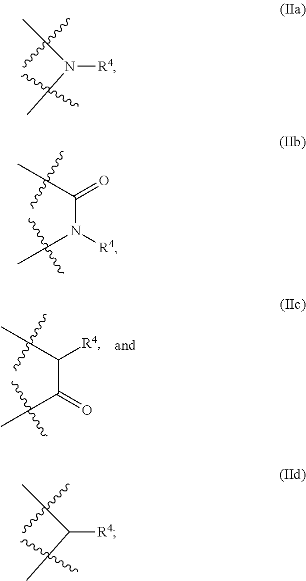
R⁴ is selected from the group consisting of: hydrogen, alkyl, amino, alkoxy, acyl, amido, aralkyl, aryl, heteroaryl, cycloalkyl, heterocycloalkyl;
R⁵ is selected from the group consisting of: hydrogen, alkyl, aralkyl, hydroxy, alkoxy, and amino;
each R⁶ is independently selected from the group consisting of: hydrogen, alkyl, alkoxy, hydroxy, amino, amido, and halogen;
each R⁷ is independently selected from the group consisting of: hydrogen, alkyl, alkoxy, hydroxy, amino, amido, and halogen;
each R⁸ is independently selected from the group consisting of: hydrogen, alkyl, alkoxy, hydroxy, amino, amido, and halogen; and
each R⁹ is independently selected from the group consisting of: hydrogen, alkyl, alkoxy, hydroxy, amino, amido, and halogen.

5. The compound according to any one of claims 2-4, wherein A is of formula (II) and Q is of formula (IIa).

6. The compound according to any one of claims 2-5, wherein L is of formula (III) 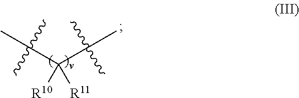
wherein
v is 0 or 1; if v is 0, L is absent;
each R¹⁰ is independently selected from the group consisting of: hydrogen and alkyl;
each R¹¹ is independently selected from the group consisting of: hydrogen and alkyl;
if both R¹⁰ and R¹¹ are alkyl, R¹⁰ and R¹¹ are optionally connected to form a C₃₋₆ ring.

7. The compound according to any one of claims 3-6, wherein R⁵ is selected from the group consisting of: 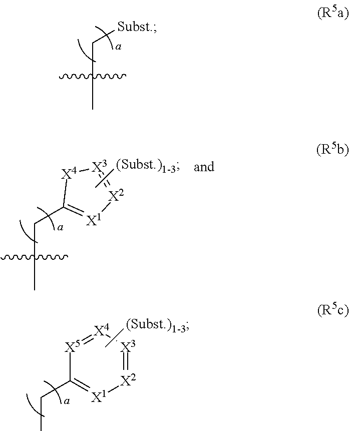
or any tautomer thereof,
wherein a is 0, 1, 2, or 3;
X¹, X², X³, X⁴, and X⁵ independently are selected from the group consisting of: C, CH, and N; and
each one, two, or three Subst. is independently selected from the group consisting of: hydrogen, alkyl, halogen, hydroxy, alkoxy, amino, amido, acyl, cycloalkyl, and heterocycloalkyl.

8. The compound according to any one of claims 2-7, wherein A is of formula (II) and L is of formula (III), wherein L-A is selected from the group consisting of:

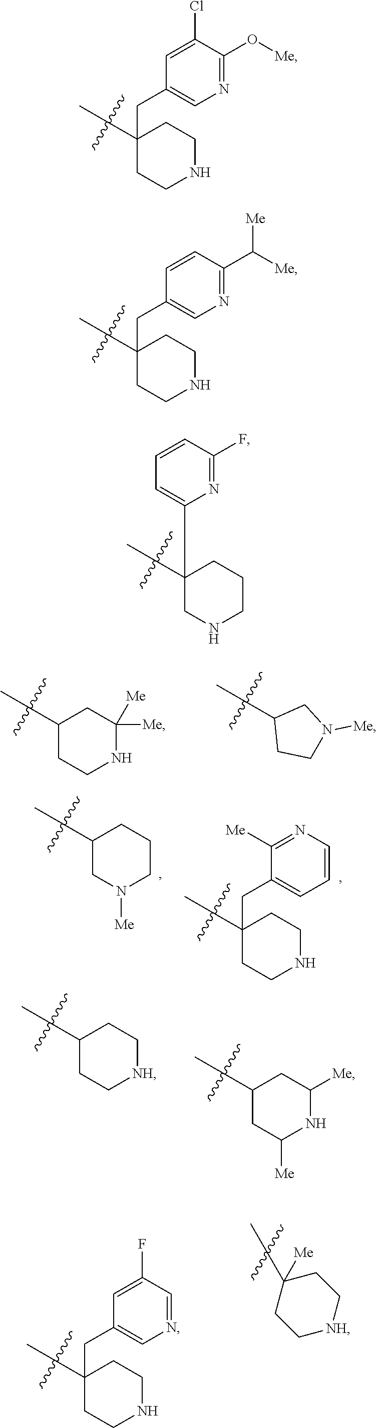

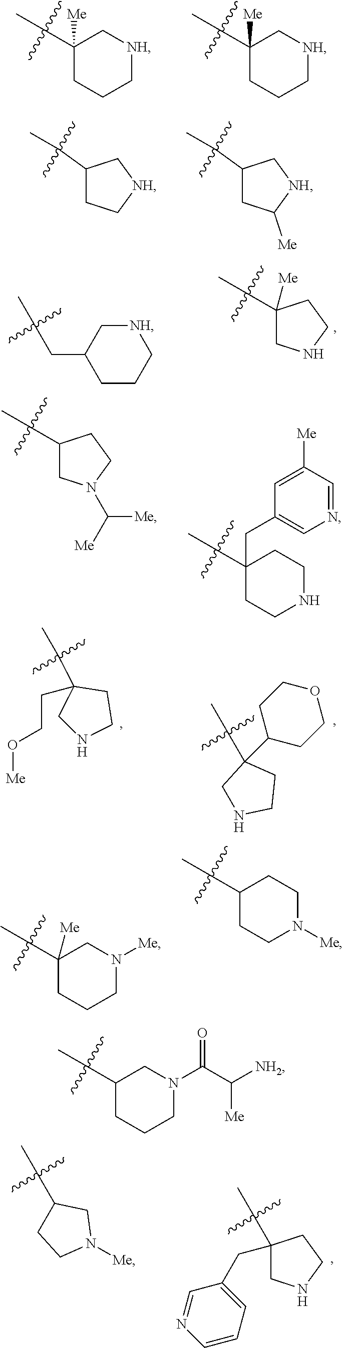

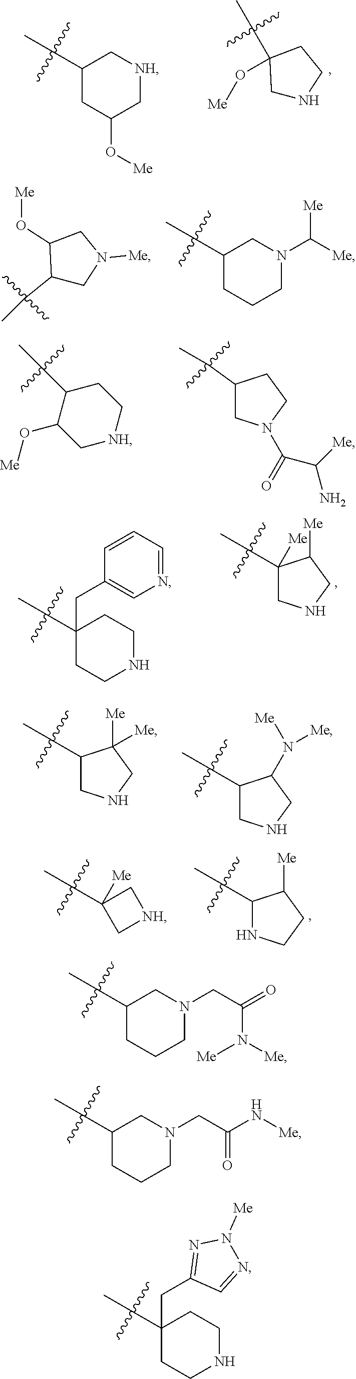

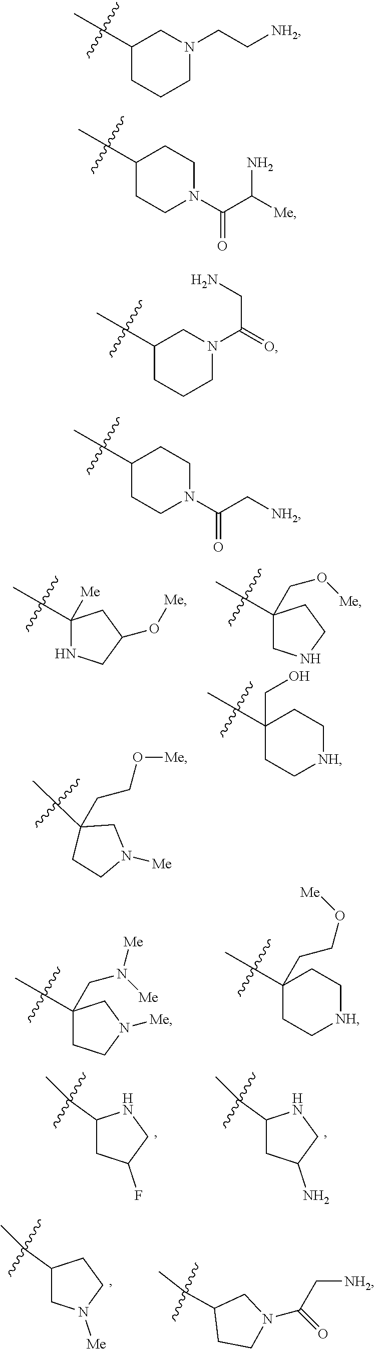

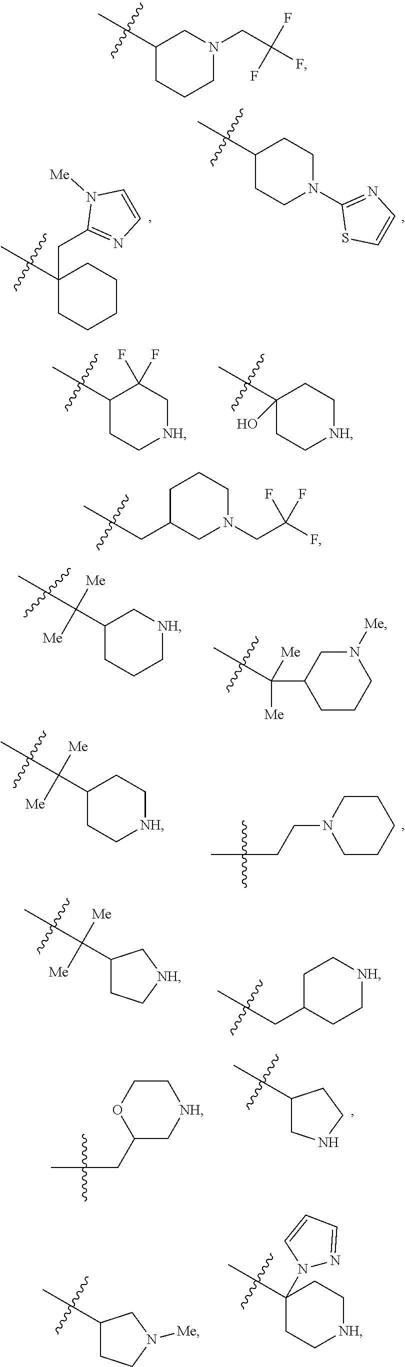

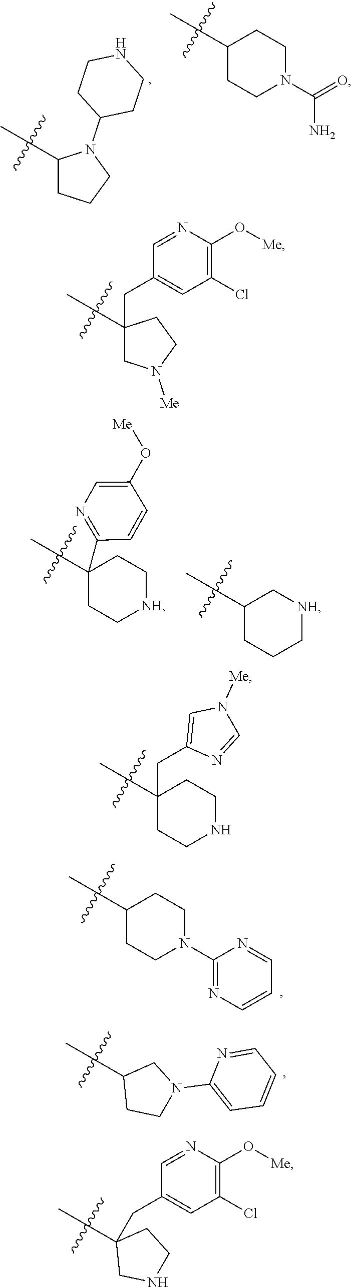

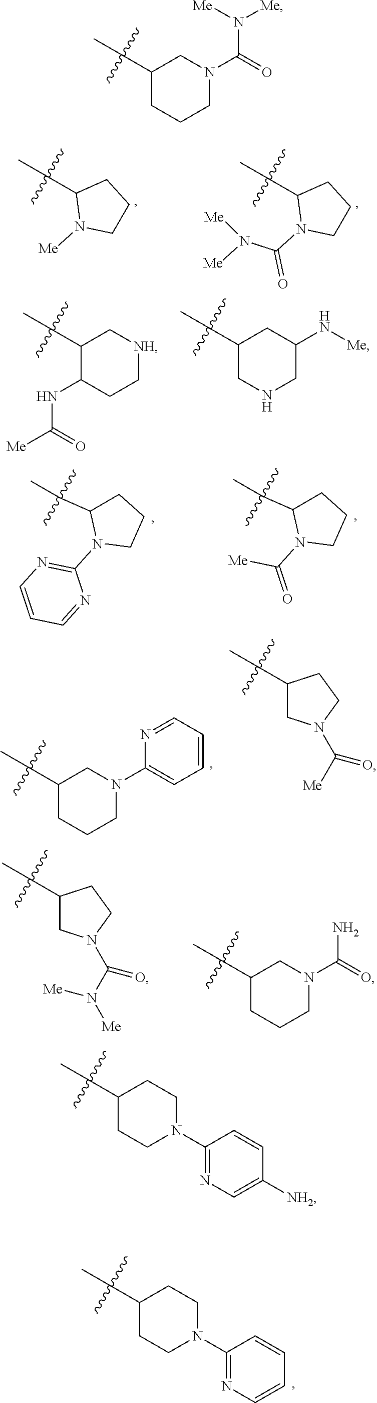

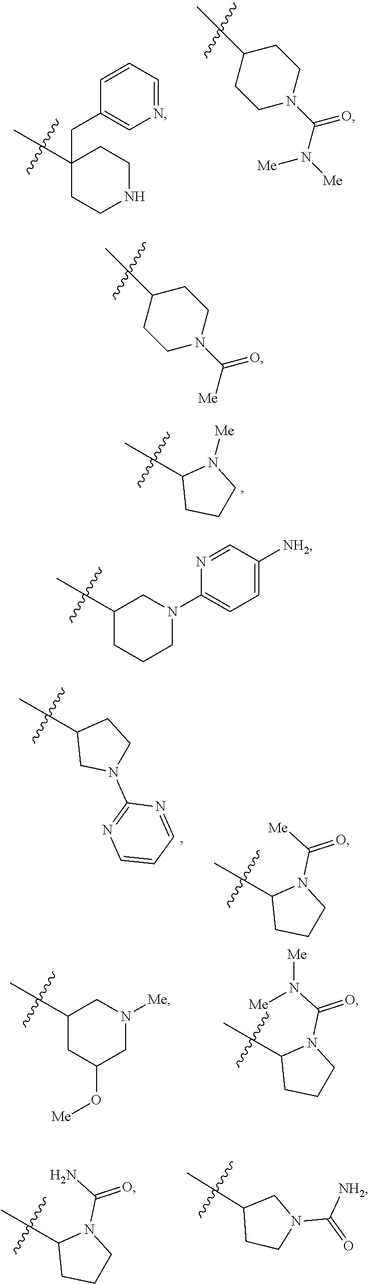

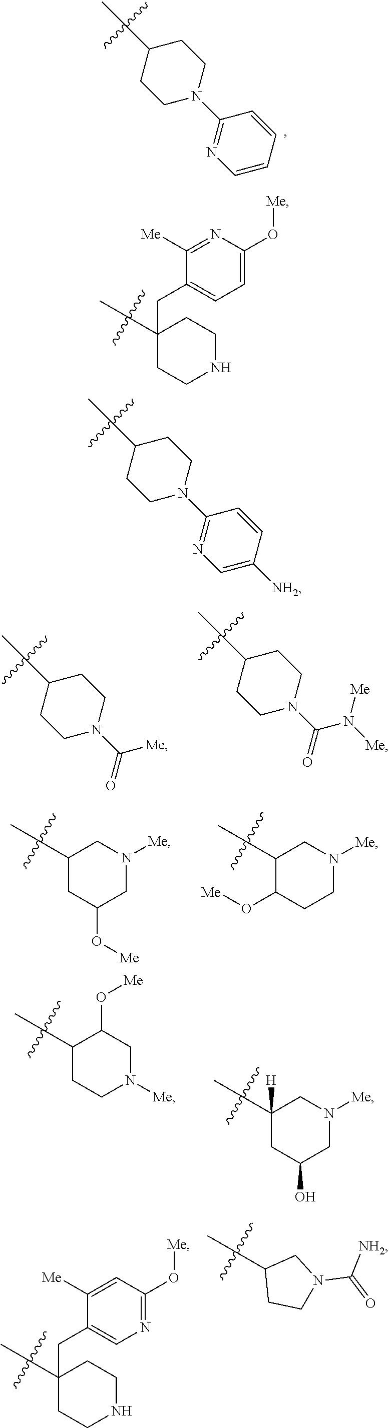

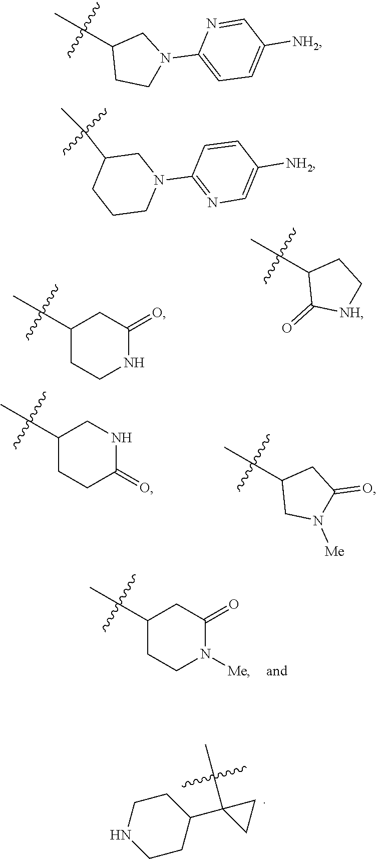

9. The compound according to any one of claims 2-8, wherein A is selected from the group consisting of:

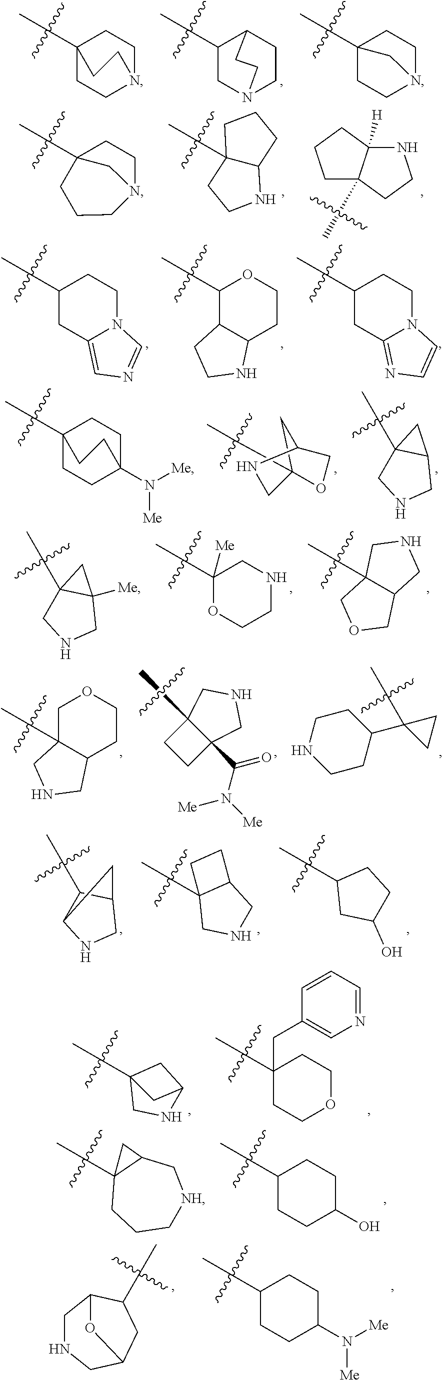

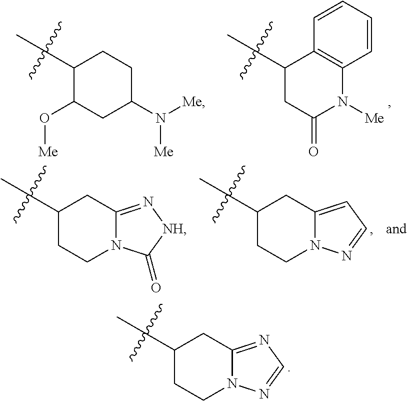

10. The compound according to claim 1, wherein OrgB is selected from the group consisting of:

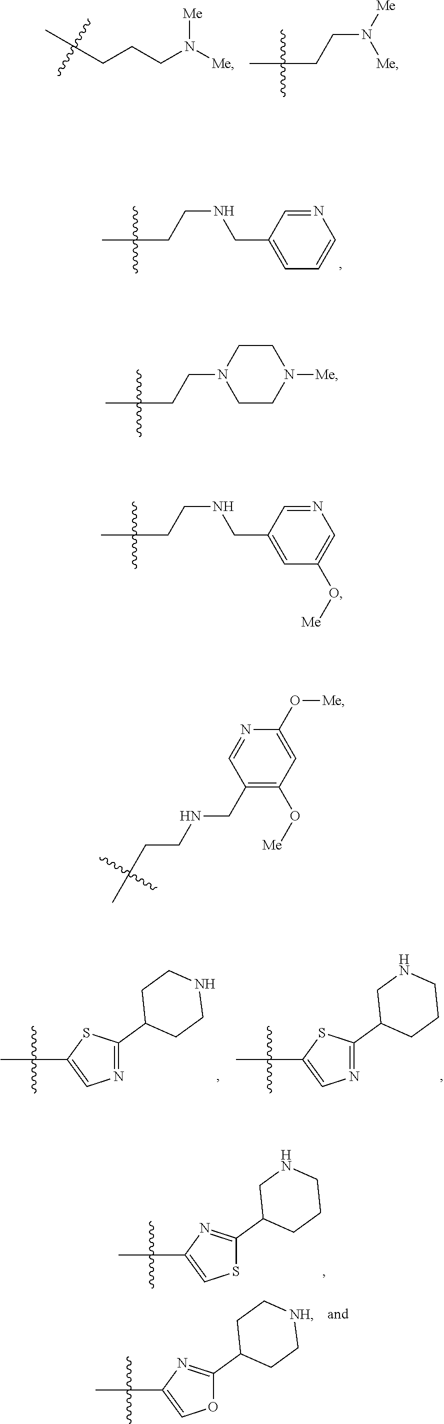

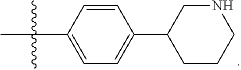 [SMILES: *c1ccc(C2CCCNC2)cc1]

11. The compound according to any one of the preceding claims, wherein Y is a nitrogen-containing ring, wherein the nitrogen-containing ring is monocyclic or bicyclic.

12. The compound according to any one of the preceding claims, wherein Y is selected from the group consisting of:

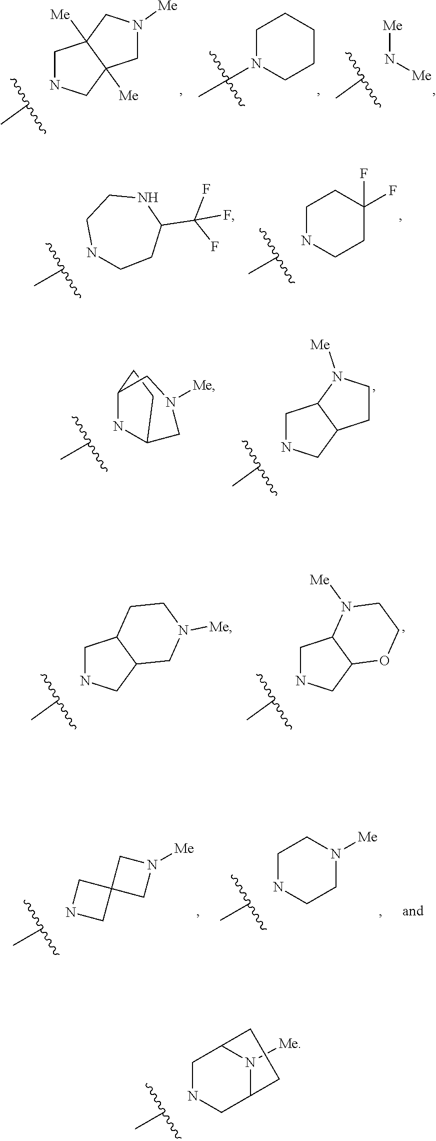

13. The compound according to any one of the preceding claims, wherein the compound is selected from the group consisting of:

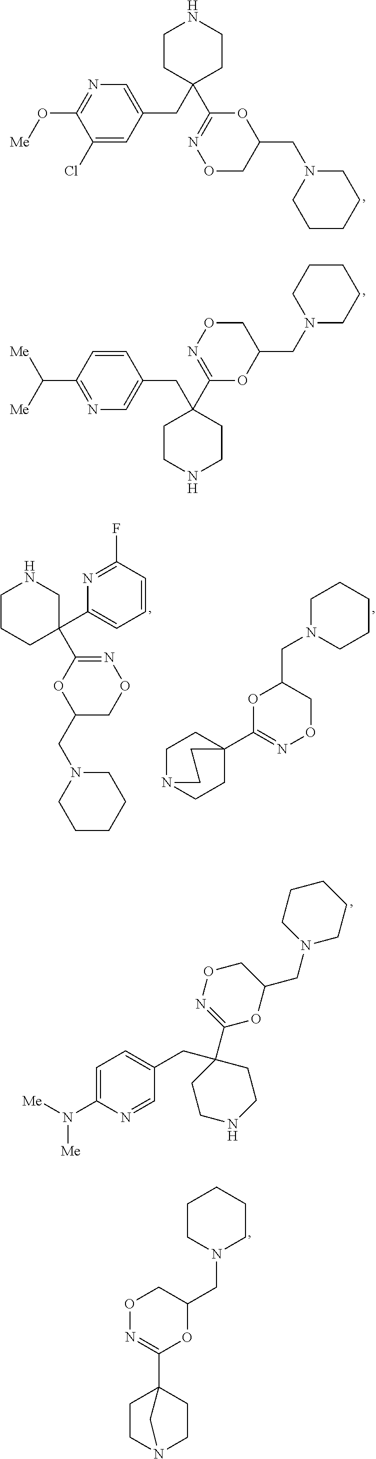

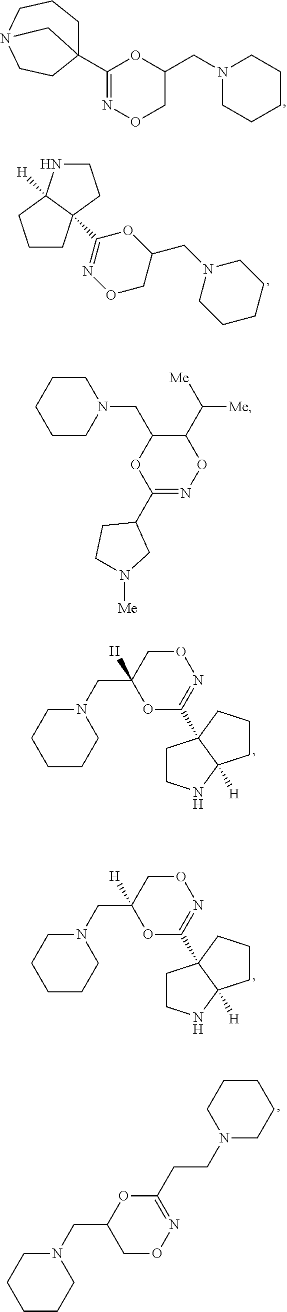

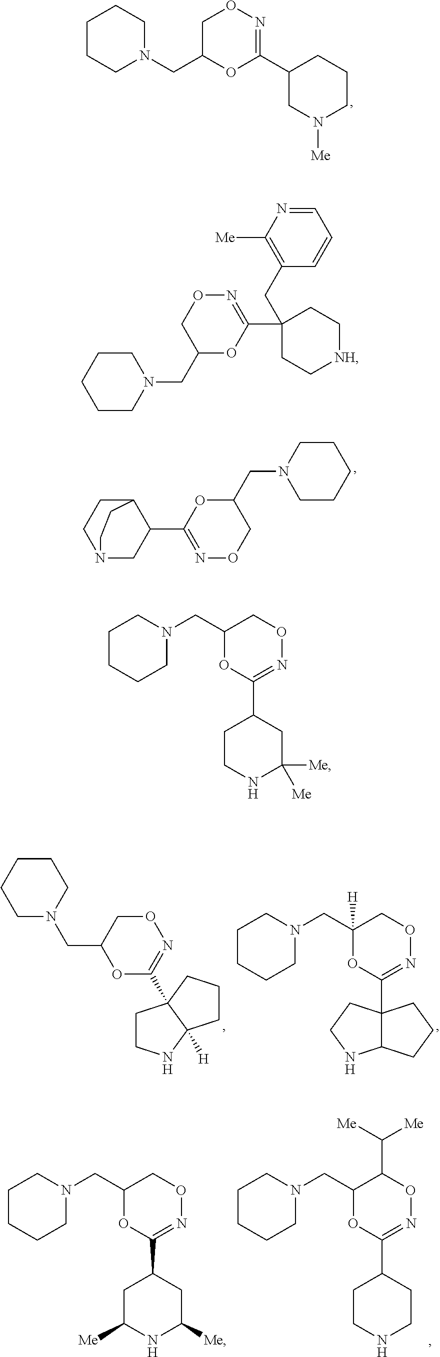

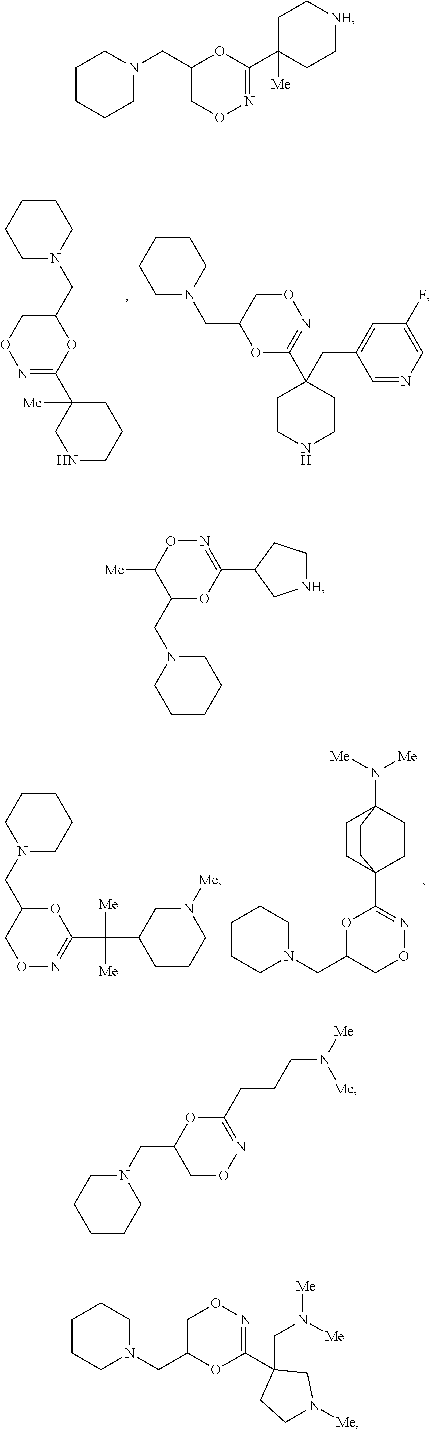

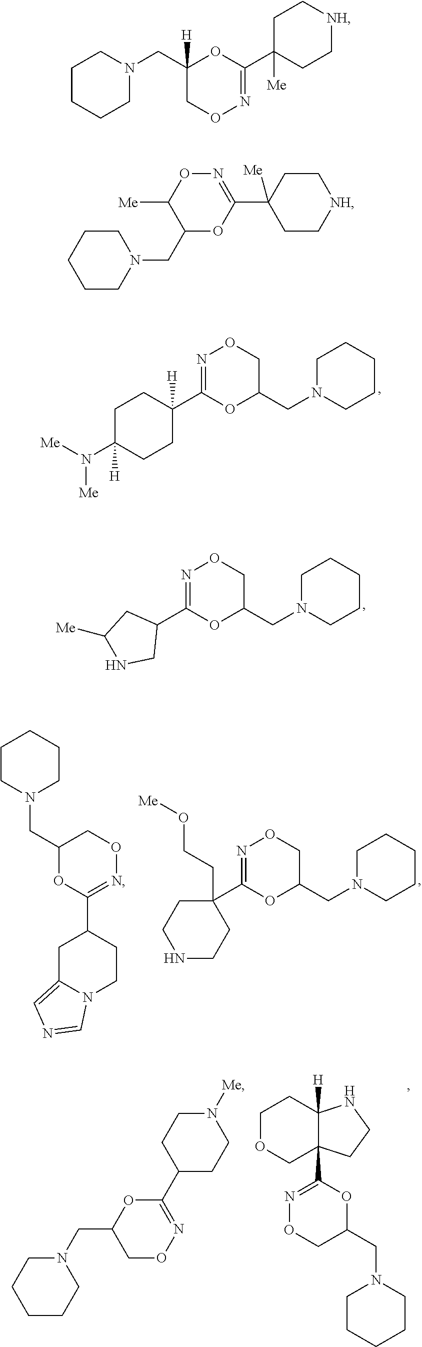

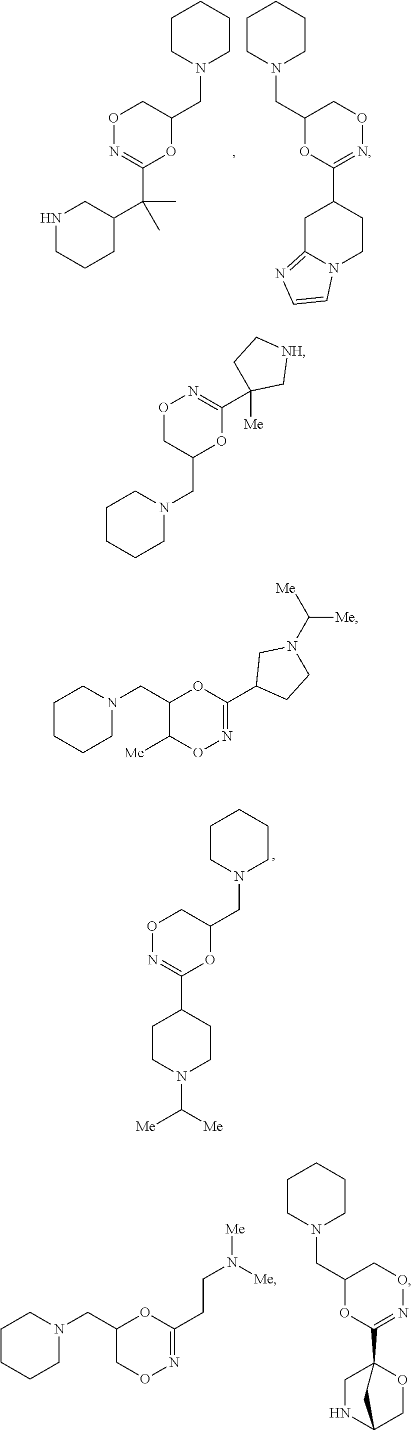

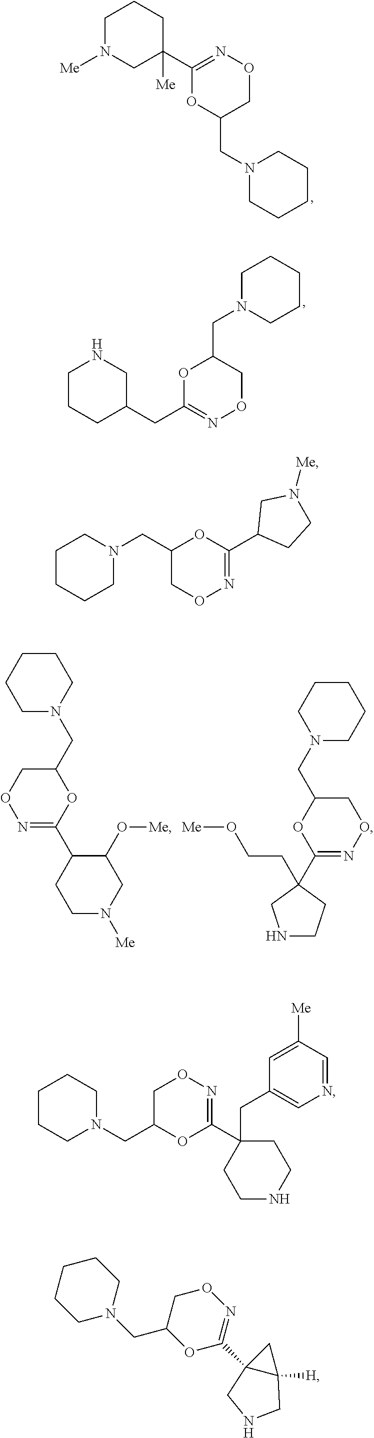

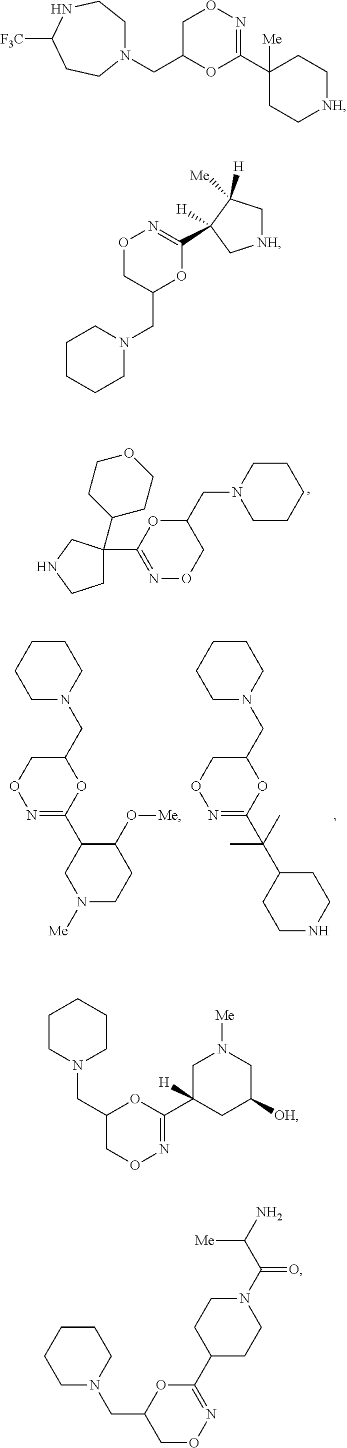

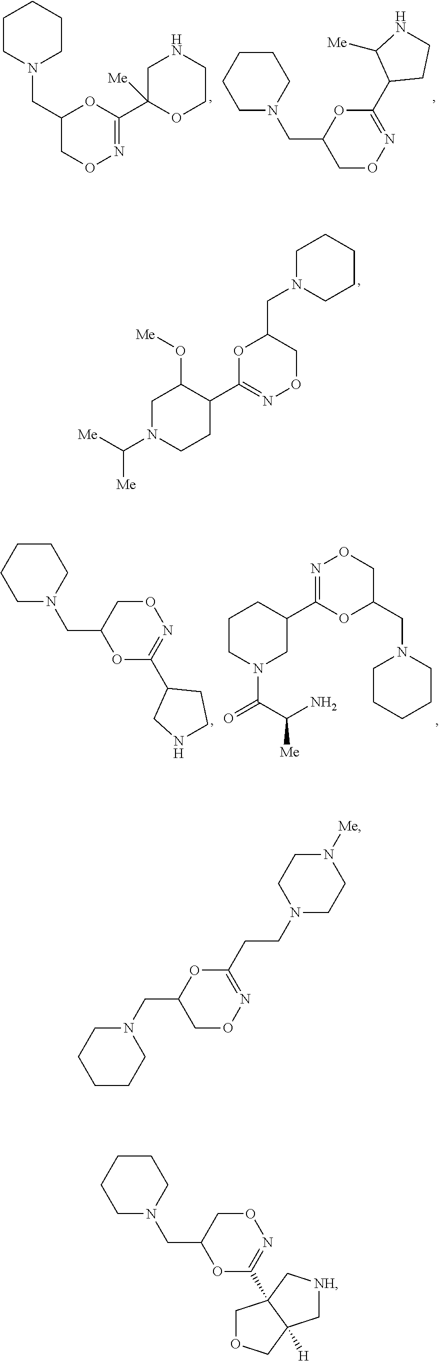

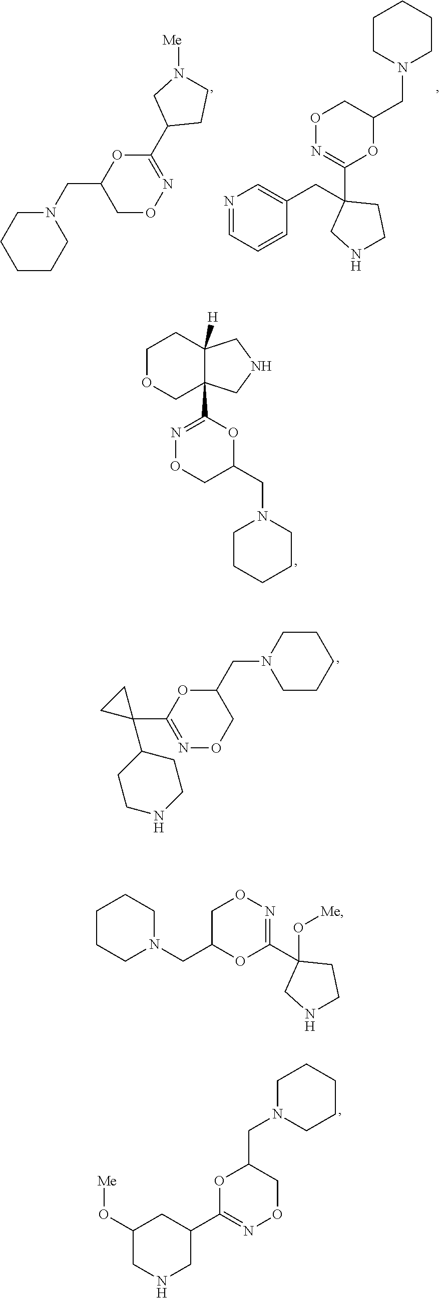 [SMILES: CN1CCC(C2=NOCC(CN3CCCCC3)O2)C1 ; c1cncc(CC2(C3=NOCC(CN4CCCCC4)O3)CCNC2)c1 ; C1CCN(CC2CON=C([C@]34CNC[C@H]3CCOC4)O2)CC1 ; C1CCN(CC2CON=C(C3(C4CCNCC4)CC3)O2)CC1 ; COC1(C2=NOCC(CN3CCCCC3)O2)CCNC1 ; COC1CNCC(C2=NOCC(CN3CCCCC3)O2)C1]

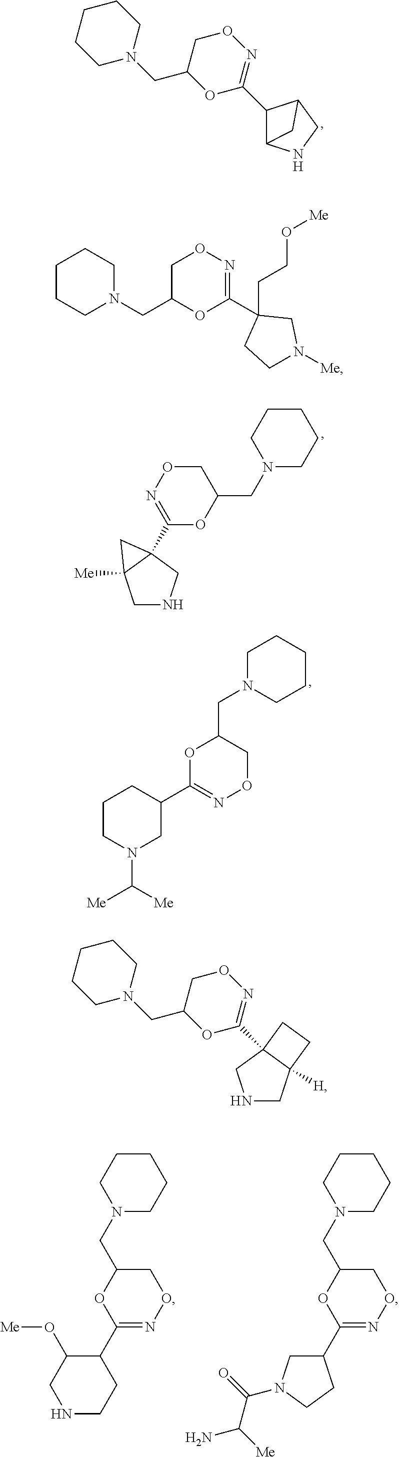

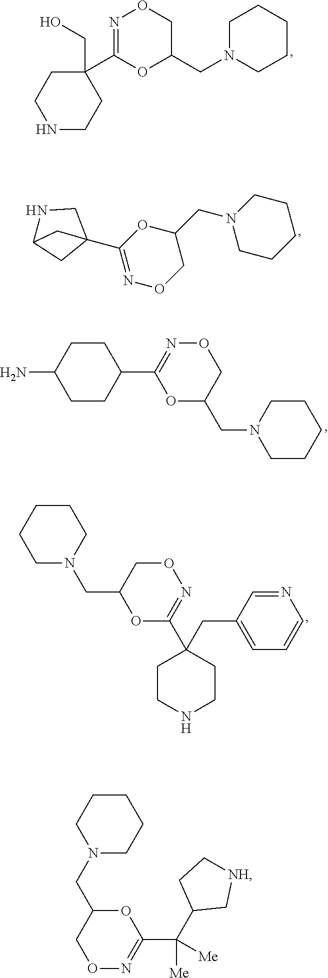 [SMILES: OCC1(C2=NOCC(CN3CCCCC3)O2)CCNCC1 ; C1CCN(CC2CON=C(C34CNC(C3)C4)O2)CC1 ; NC1CCC(C2=NOCC(CN3CCCCC3)O2)CC1 ; c1cncc(CC2(C3=NOCC(CN4CCCCC4)O3)CCNCC2)c1 ; CC(C)(C1=NOCC(CN2CCCCC2)O1)C1CCNC1]

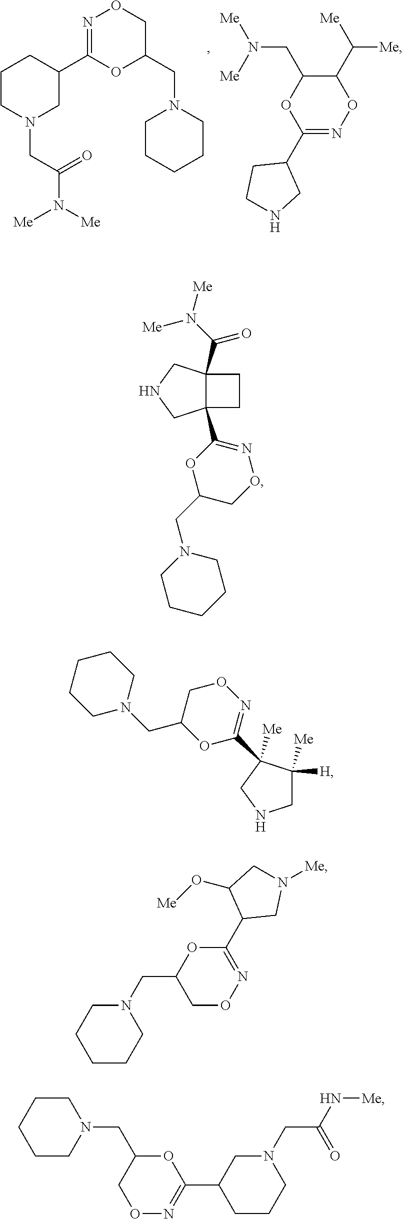

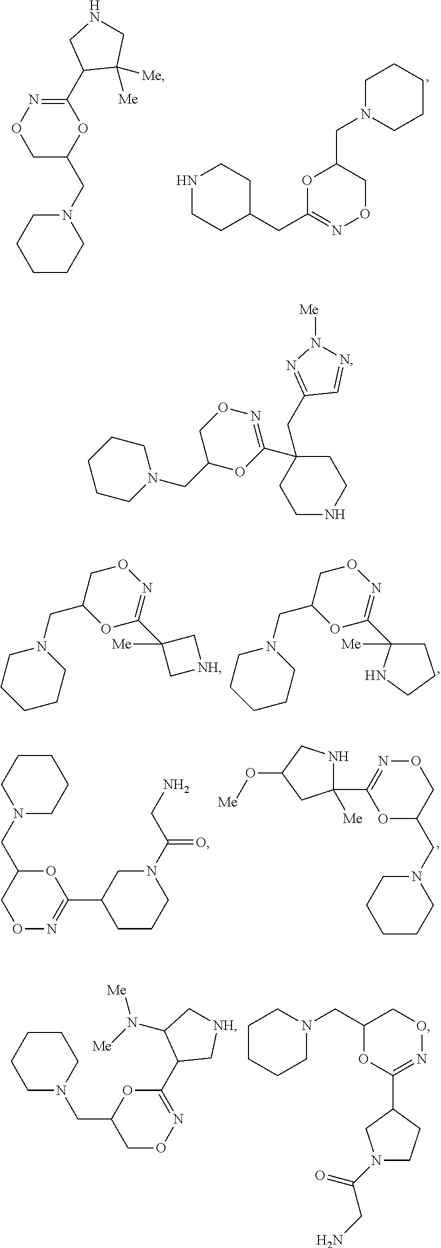

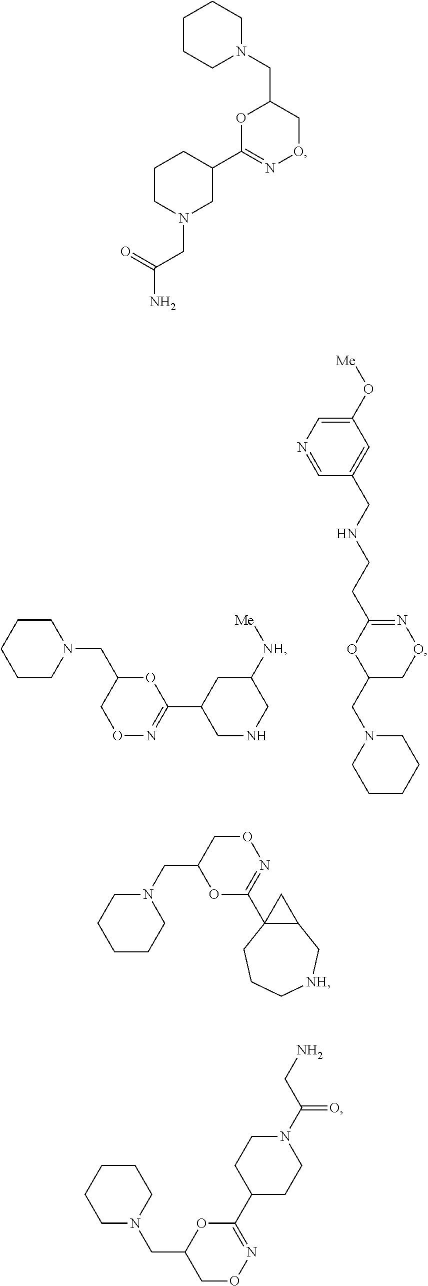 [SMILES: NC(=O)CN1CCCC(C2=NOCC(CN3CCCCC3)O2)C1 ; CNC1CNCC(C2=NOCC(CN3CCCCC3)O2)C1 ; COc1cncc(CNCCC2=NOCC(CN3CCCCC3)O2)c1 ; C1CCN(CC2CON=C(C34CCCNCC3C4)O2)CC1 ; NCC(=O)N1CCC(C2=NOCC(CN3CCCCC3)O2)CC1]

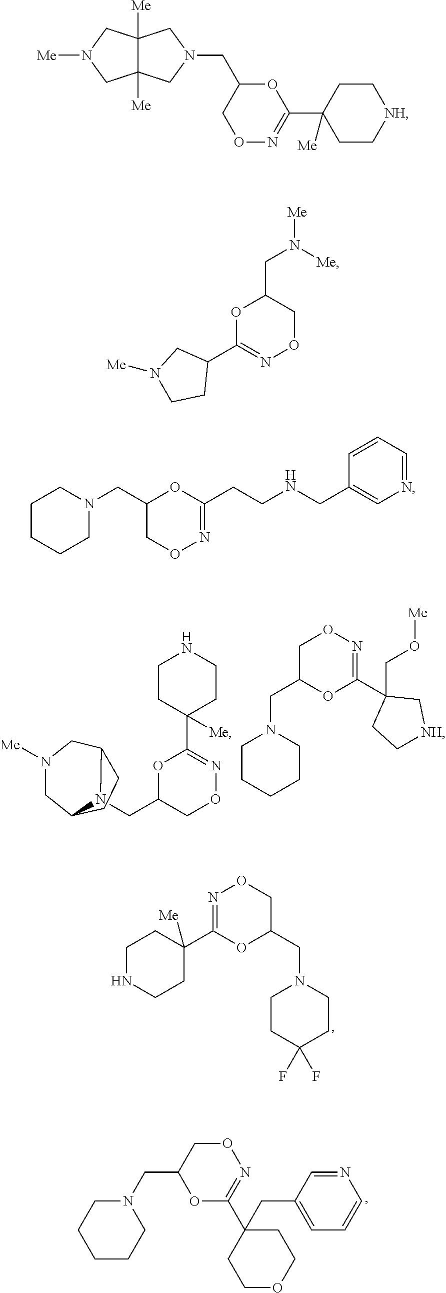

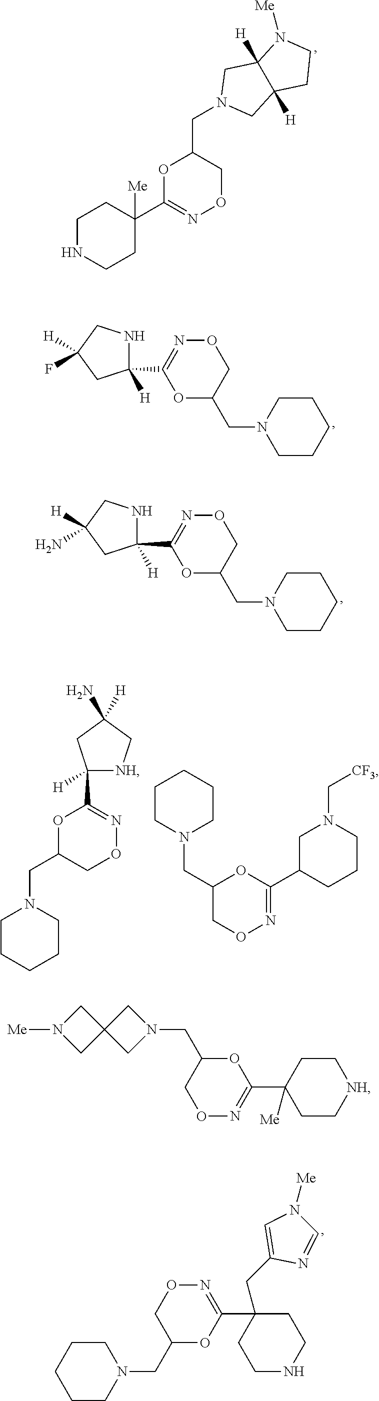

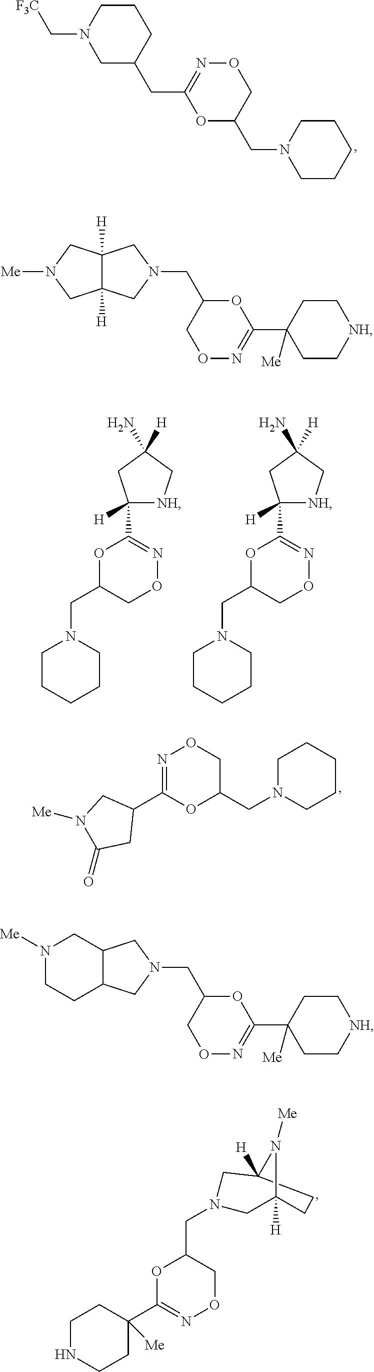

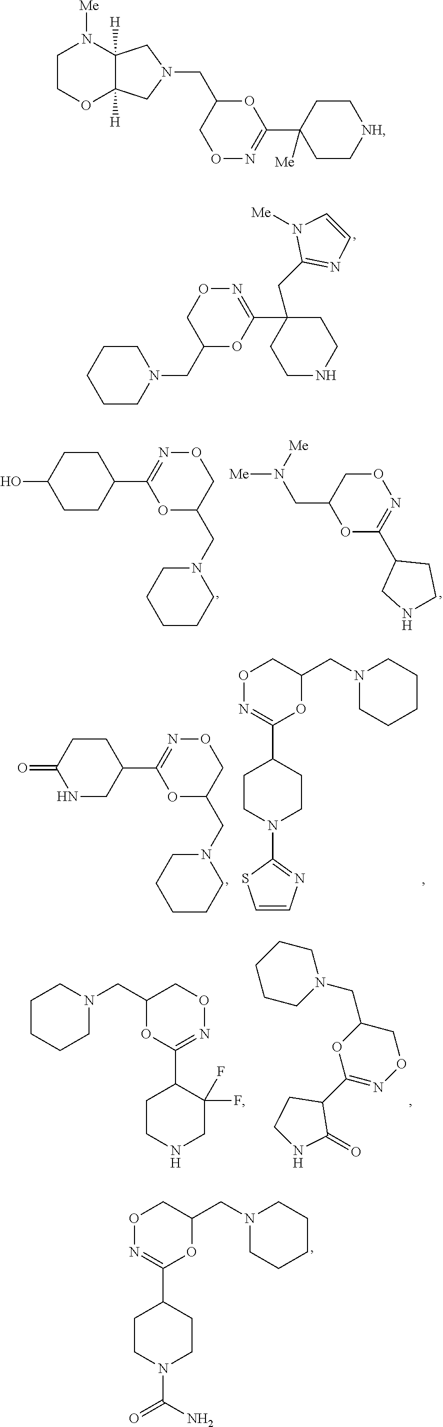

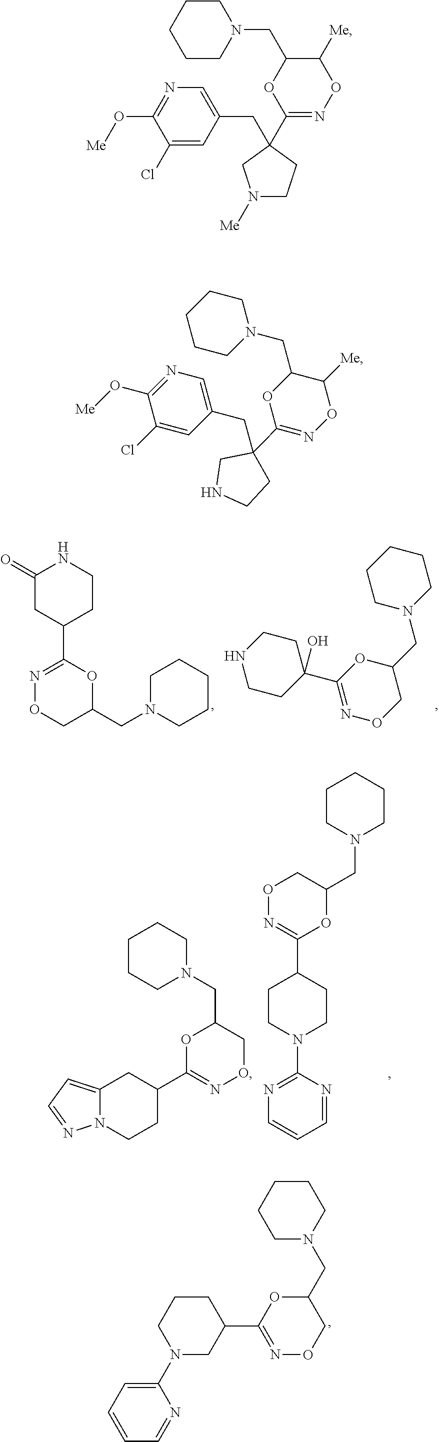

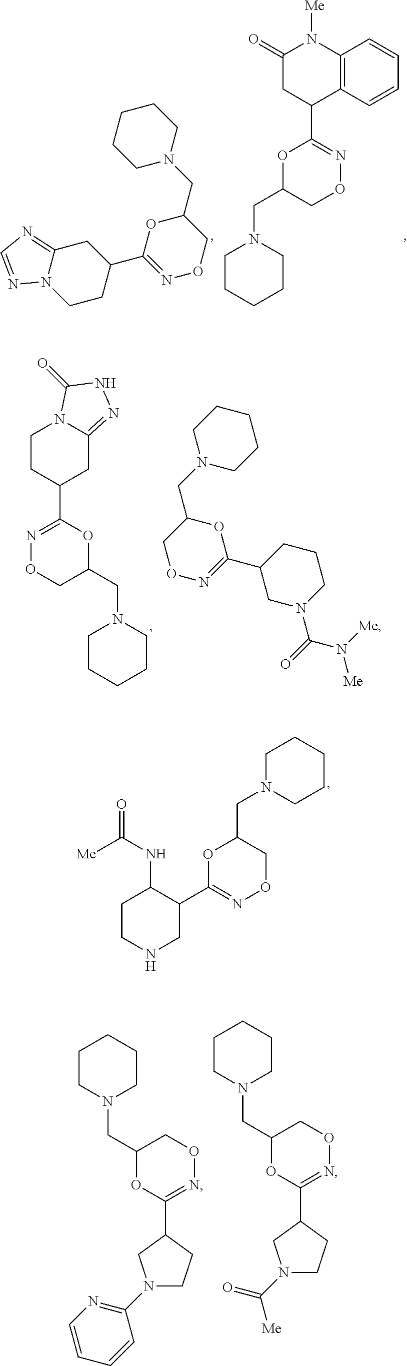

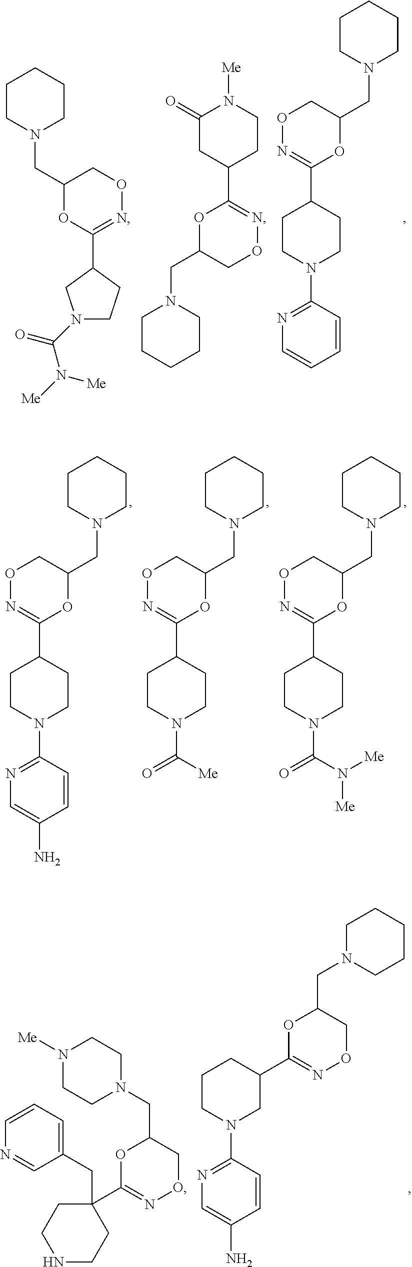

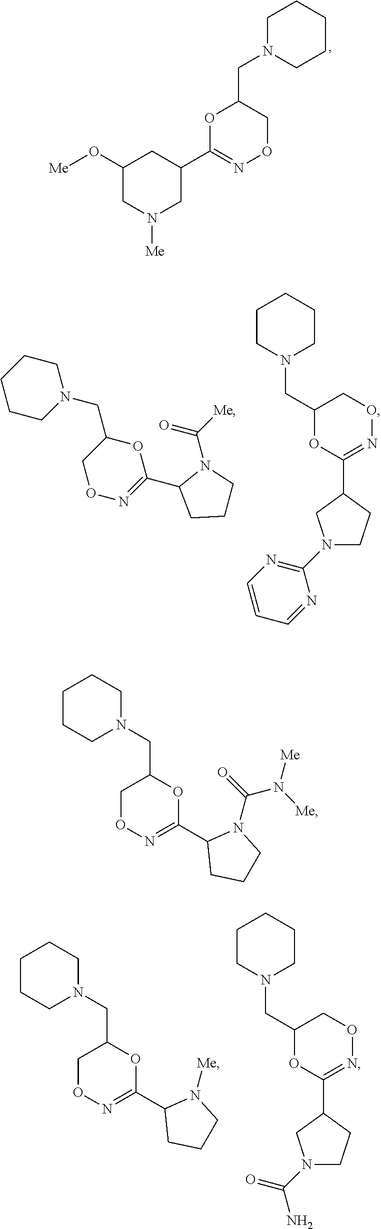

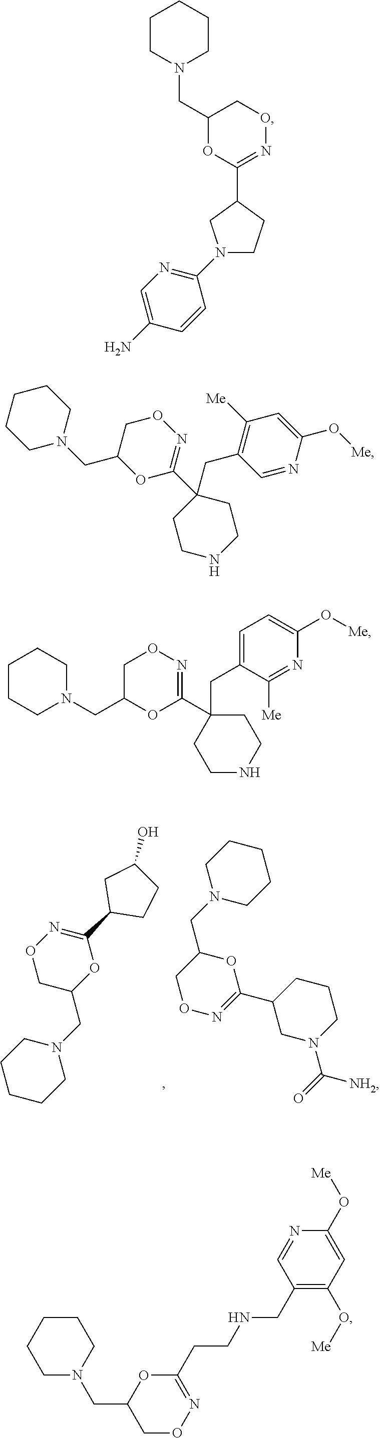

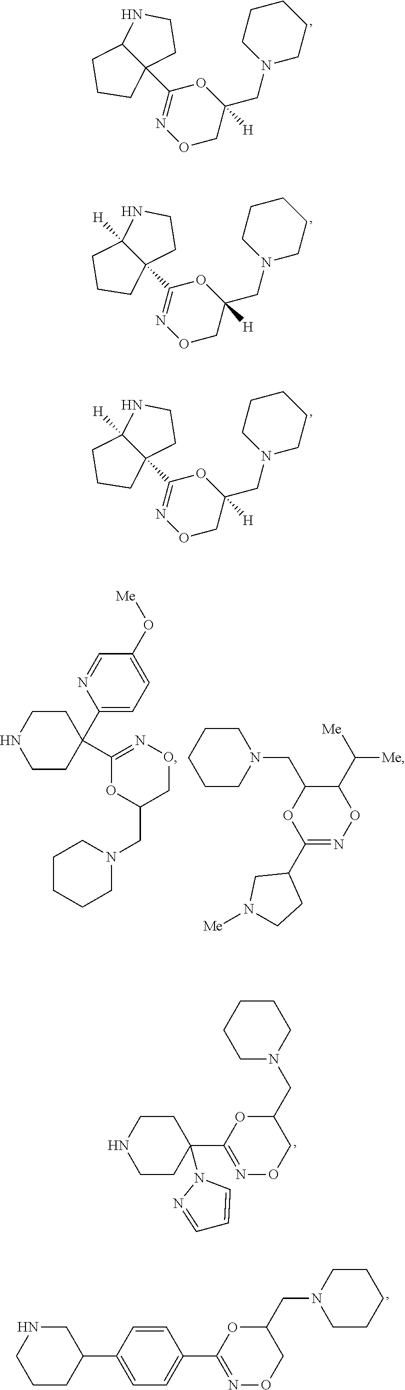

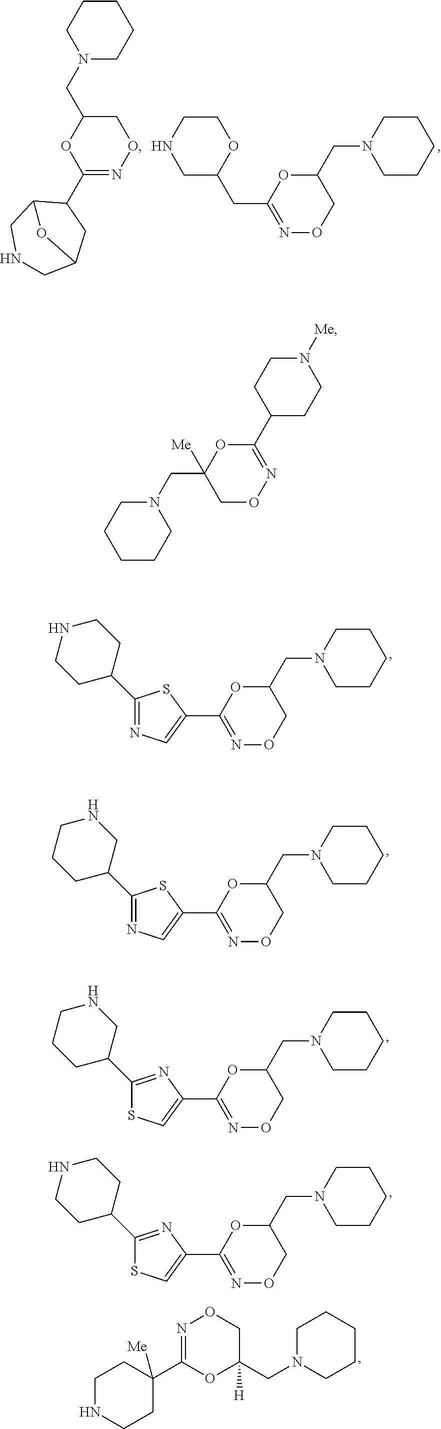

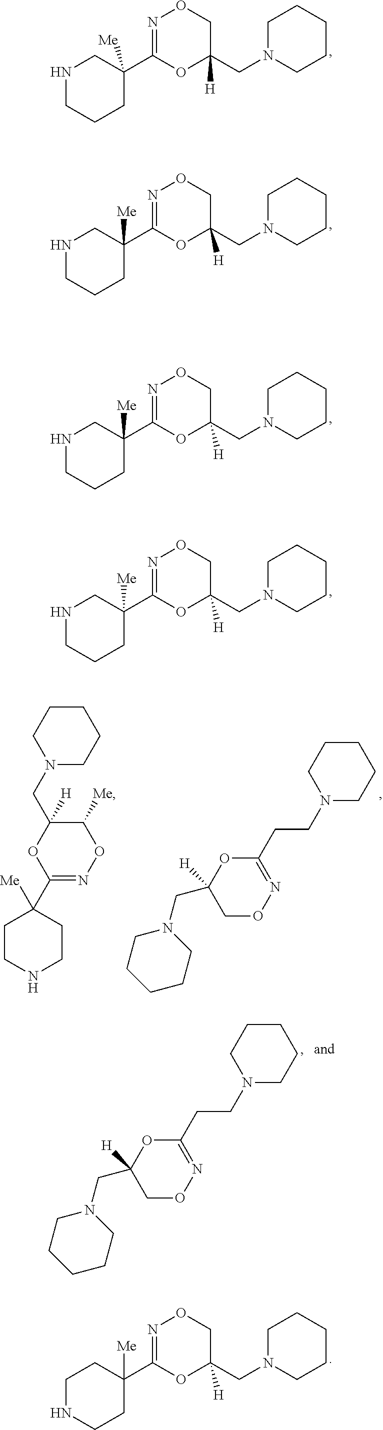

14. The compound according to any one of the preceding claims, wherein the compound is a GBA inducer and increases glucocerebrosidase (GBA) enzyme levels and/or GBA enzyme activity.

15. A compound as defined in any one of the preceding claims for use in the treatment of Parkinson's disease (PD) in a subject.
